SEQUENCE LISTING (193 sequences)

SEQ ID NO: 1 - DNA, 3607 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagatcgtcgacctagctctgtcttagtccctatcagtgatagagataacatgcctctcactaacatggtccctatcagtgatagagactactggggccacgattcgtgtgtccctatcagtgatagagatctgcgtaatactactcgcgtgttccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 2 - DNA, 4152 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatggacatgccaaggataaaacctggacagcgtgtgatgatggctctaaggaaaatgatcgcctccggcgaaatcaaatctggcgaaagaatagcagaaatacccacagctgctgcattgggtgtgtcaaggatgcctgtgcgtatcgcactaaggagtttggagcaagaaggtctagttgtgaggttgggagcaaggggttacgccgccaggggagtttcttccgatcagattagagacgctatcgaagtgagaggtgtattagaaggcttcgcagcccgtcgtttagccgaaaggggtatgactgctgagactcacgcaaggttcgtggtcttgattgctgagggcgaggccttattcgctgcaggtaggctaaatggtgaagacctagaccgttacgctgcttacaatcaagccttccatgataccctggtctcagcagctggcaatggagcagtagaatctgccctagccaggaacggatttgagccattcgcagcagcaggcgcattagccttggacttaatggacttatccgctgagtatgagcatttactggccgctcacaggcaacatcaagccgtactagatgctgtatcatgcggcgatgcagaaggtgcagaaaggattatgcgtgatcacgctctggcagccataagaaacgcaaaggttttcgaagccgcagcatccgcaggagccccccttggtgccgcatggtctatacgtgccgacgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgattggatccaatcttgccccattcgctaagcccacattggatccaatagctagaccttacggattggtgcattggatccaatggtcgaacatctgctataagcgcattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 3 - DNA, 2975 bp, Artificial Sequence
  Synthetic polynucleotide
tctgtttcagaaagaacattttgactggaatgttttcaagaaggctgaacttattgagatggaaggcagtgaagcttcatctccctcagaagtcgatgttgtatatgaaagtggatctgtctcatctccaactgaagactcagaaaagagatccgtggacaagtctgagaatgttcaacttcaagaaactgagcttggagtgtcaagtggtgacgagtacgacgaacaagagcaaaaattgatcaagcgttacatcaagatagcatacgcttggtgtatacttgtggttctcgttacttcggtgttgttcccaatgtcactgtaccgtaactggatcatatctttgagtttctttagaggatatactggattgtccatgttctggttatacggagtcttcttagttatcgcagtctatcctctttatgatgggcgacattcgctaggccgaattggtcagggtttgtggaaagacttcaaaaggatcttcaaatctaaacgatgatttaaggctacaagagtgtaacagtcaaatatgtatttagtatgccagtaatatgacattagcttttgtaccgagagcaacaatgctctgaaatttgttcttgaatagattaaactgatagaatagcactgttaccactaacctctatatatgaacgttcttgtatctgtgctcccgattcattagatatgaacgctttgaaacacgctttttggagtagctttaggataaacctaattgtgactcccaaagcaattcgcatagataaccccagttcgagaaaataaattgcggagaaacttttcttctttctgcagtttcaatgtgagatttagtgatgacctaggcgattaactttaatttgcttttgcttgcgctcttgatatagtacgaaagcttggctctggcggggtcaaaaggtgaacatgactgacccatttgcaatgataaaagagatacctttcactgtagcttcttggggagaataactacgatatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggttccagtacagcaccccctaccgacgttagtctgggtgacgaactacatttggacggcgaagatgtcgctatggctcatgcagacgcattagacgactttgacttggacatgctgggagatggagattctcctggtccaggattcacgccccatgacagtgccccttacggagccctggatatggcagatttcgagttcgagcaaatgtttactgatgctctgggcattgatgagtacggtggttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 4 - DNA, 3541 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgtccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 5 - DNA, 4060 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtaaagtgaaagtcgagctcggtaccctcttttcatctataaatacaagacgagtgcgtccttttctagactcacccataaacaaataatcaataaat

SEQ ID NO: 6 - DNA, 3404 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 7 - DNA, 4125 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagacgccttagacgacttcgatttagacatgctttccatgcaaccctcattgagatcagagtatgaataccccgtattcagtcacgtccaagctggtatgttctctccagaactaaggacatttacgaaaggagatgccgagcgttgggtcagtgacgctttagatgactttgacctagatatgctttctatgcaaccatcattaagatccgaatacgaatatcctgtattttcacacgttcaggccggcatgttttcccccgaattacgtacgttcacgaaaggcgacgcagaaagatgggtatccgatgcactagatgattttgacttagatatgttgtcaatgcagccctctttaaggtccgaatacgagtaccccgtcttctctcacgttcaagccggcatgttttctcccgagctaagaacctttacgaaaggtgacgctgaaagatgggtgtcagatgcccttgatgattttgacttggatatgttataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 8 - DNA, 4203 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 9 - DNA, 3854 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaatgccaccaagaccactggacgtactaaatcgttcactgaaatcccctgtgatagtgaggctaaagggaggccgtgagtttcgtggaaccttagatggatacgatattcacatgaacttggtactgttagacgccgaggagattcaaaacggtgaagttgtgagaaaggtgggatcagttgtgattagaggagataccgtcgtctttgttagtccagcccctggtggtgaaggtggcacgtctggaggcacatccggttcaacatccggtacgggatcttcaggctccggcggctcaattaacaaggacatagaggaatgtaacgctattatcgagcaatttatcgattatcttagaactggtcaggaaatgcctatggagatggcagatcaggcaattaacgtcgtgcctggaatgactccaaagactattttgcacgcaggtcctcctatacaaccagattggcttaaatctaacggttttcatgaaattgaggcagacgttaatgacacatctctactactaagtggcgattaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 10 - DNA, 3933 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 11 - DNA, 3570 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgtcgtcgacctagctctgtcttagcggaatgaactttcattccgtaacatgcctctcactaacatggcggaatgaactttcattccgctactggggccacgattcgtgtgcggaatgaactttcattccgtctgcgtaatactactcgcgtgtcggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 12 - DNA, 4487 bp, Artificial Sequence
  Synthetic polynucleotide
ttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 13 - DNA, 3677 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtaccctgtggagaagggtgaacaatataaaaggctggagagatgtcaatgaagcagctggatagatttcaaattttctagatttcagagtaatcgcacaaaacgaaggaatcccaccaagacaaaaaaaaaaattctaag

SEQ ID NO: 14 - DNA, 3621 bp, Artificial Sequence
  Synthetic polynucleotide
ccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatcatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 15 - DNA, 4755 bp, Artificial Sequence
  Synthetic polynucleotide
atcttttcagcttcatcgtcagtgatatttctcagcccacagaccaagtcaactttggaatctaacaaccttgttcttacaatgttagaactcttaagtcgcatgccatgatcttcaagctgaattttgtgaaggaggtcaaaccccacaatggcatctagttgtttagaatacatgccttcgacaagtgtttgagtgtccaaaatcaagagctcaaaattattgaatttgtctgccaataacgccgtaaattgattagtgtccagcccaccaacaataggagcacctatagttaatttttcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatcatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagcgcttacagagctccctaggtgcggaatgaactttcattccgcttgccccattcgctaagcccaccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 16 - DNA, 350 bp, Unknown
  May be komagataella phaffi or picha pastoris
ccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatc

SEQ ID NO: 17 - DNA, 1000 bp, Unknown
  May be komagataella phaffi or picha pastoris
atcttttcagcttcatcgtcagtgatatttctcagcccacagaccaagtcaactttggaatctaacaaccttgttcttacaatgttagaactcttaagtcgcatgccatgatcttcaagctgaattttgtgaaggaggtcaaaccccacaatggcatctagttgtttagaatacatgccttcgacaagtgtttgagtgtccaaaatcaagagctcaaaattattgaatttgtctgccaataacgccgtaaattgattagtgtccagcccaccaacaataggagcacctatagttaatttttcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatc

SEQ ID NO: 18 - DNA, 1000 bp, Unknown
  May be komagataella phaffi or picha pastoris
tgctgttttgggctcgtacggatgtttcttaggtccgatgattggtgttatgacttgtgactactacttcgtacgtcatcagaaactcaagctgacagacctctacaaagccgacaagagttctatttactggttctacaaaggattcaactggagaggtttcgttgcctggatttgcggttttactccaggtattacagggtttcctagcgtcaaccccaacttgactggggttcctacagcctgtatcaagatgttctacatttcgtttatcattggttacccgatcggattcttagttcatctggcactcaataagctattccctccaccaggtcttggtgaagtcgatgagtatgactactaccactctttcaccgaaaaggaagcactgaaattaggaatggcccctagttccgagttggacagagtcagcaccgatgacccgatcaatattccttacgacgagaagtctttaggctaatgtagttaaatagttaatcgaaacaatcgtgtatcctctttatcgtaccagcgggattcgctgcttggatgggtgactcctgtccagttgactcaaagtagtcaaaataggcctggagacccttaacaggtcgatgagtagcctactatgagaaaacccctcaccacaactggactataaaagggcacgtcaatccccaaagcaactcttttctttcatccctactttattactttatcctttgatcttcattgaagaaaatctgaaacaattgtaagcagcaatccacacctccccagcaatgactcaatttactaaccccattgacagaaaatgtgagcatcttttttagatgtcatgatgataggtggagtattcttaattattgctttcagcaaaccggtgcccataaagtgtttcccattaaatcaatgagaggcattaaggctgagattaaacggttgaacttgaactagataattctagcggaaagaattgctctttttattacgtcgt

SEQ ID NO: 19 - DNA, 1000 bp, Unknown
  May be komagataella phaffi or picha pastoris
atctatccttttacaaacaacaacaacaacaacaacaacaacagcagcatcaatatcaaaatcagcagtttcaggggcttcacaacaaccagaactgataatcaatctttcttatttttatgggtttttttagagcaaataggtgtttaactggaactgagatacagtaatcaatgtgtgttgccatacattctcgaagagttccttgtggggaaaattgactgcggggaaagatttgtggtatgtgttgctttatccttaagataatcctccgtatcgaatatgaggcattcggaaaatctaaataatggggtgcaaaaccgttgcctagatcgtgccaagcacaaaagcttatacacgatctgaaacacaaatttcgtatagcaagcatttttaggtagtatcacgacctacgacttgtattcatgttggcatatcgaagcattctccacgctgtaggtaggtaccaatggaccaatccttgaacagggttcaaccgctcctttcctttatctttttctcccgactccgagtctattgcagcaacacgcatgttattgctcgattgcaagcaacctgtttaccgtcgtgcagatcagtcgaaaagtggagaaattttcccaacaaaaaatgggaaaggcatcactttactagaccacgattcggaatgaaactagaaagaactttggtcttcgttgaatcgtttaagcaatcaagaaaaactagcagttcttactgttgttttcaatatcgaatgacctagcacgcacattgcataaatgccaagccatttttcaggtcgggcgcacccccatttttatcgctccgaaccccgtaattccatctttcatacaatgccccatagtaaaatctccgcagctccagaatgttgacacgcatgactgtaaagagttcggtgtccaccgttctcttgttcgtgacttgttctggctataaatgtagtagtttcttccatatcctctagacagagtctaacgcc

SEQ ID NO: 20 - DNA, 1000 bp, Unknown
  May be komagataella phaffi or picha pastoris
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaac

SEQ ID NO: 21 - DNA, 1000 bp, Unknown
  May be komagataella phaffi or picha pastoris
gaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacgtgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 22 - DNA, 1000 bp, Unknown
  May be komagataella phaffi or picha pastoris
tctgtttcagaaagaacattttgactggaatgttttcaagaaggctgaacttattgagatggaaggcagtgaagcttcatctccctcagaagtcgatgttgtatatgaaagtggatctgtctcatctccaactgaagactcagaaaagagatccgtggacaagtctgagaatgttcaacttcaagaaactgagcttggagtgtcaagtggtgacgagtacgacgaacaagagcaaaaattgatcaagcgttacatcaagatagcatacgcttggtgtatacttgtggttctcgttacttcggtgttgttcccaatgtcactgtaccgtaactggatcatatctttgagtttctttagaggatatactggattgtccatgttctggttatacggagtcttcttagttatcgcagtctatcctctttatgatgggcgacattcgctaggccgaattggtcagggtttgtggaaagacttcaaaaggatcttcaaatctaaacgatgatttaaggctacaagagtgtaacagtcaaatatgtatttagtatgccagtaatatgacattagcttttgtaccgagagcaacaatgctctgaaatttgttcttgaatagattaaactgatagaatagcactgttaccactaacctctatatatgaacgttcttgtatctgtgctcccgattcattagatatgaacgctttgaaacacgctttttggagtagctttaggataaacctaattgtgactcccaaagcaattcgcatagataaccccagttcgagaaaataaattgcggagaaacttttcttctttctgcagtttcaatgtgagatttagtgatgacctaggcgattaactttaatttgcttttgcttgcgctcttgatatagtacgaaagcttggctctggcggggtcaaaaggtgaacatgactgacccatttgcaatgataaaagagatacctttcactgtagcttcttggggagaataactacgat

SEQ ID NO: 23 - DNA, 1000 bp, Unknown
  May be komagataella phaffi or picha pastoris
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 24 - DNA, 1529 bp, Unknown
  May be komagataella phaffi or picha pastoris
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaa

SEQ ID NO: 25 - DNA, 1000 bp, Unknown
  May be komagataella phaffi or picha pastoris
ttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 26 - DNA, 1599 bp, Artificial Sequence
  Synthetic polynucleotide
atgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 27 - DNA, 2340 bp, Artificial Sequence
  Synthetic polynucleotide
atggacatgccaaggataaaacctggacagcgtgtgatgatggctctaaggaaaatgatcgcctccggcgaaatcaaatctggcgaaagaatagcagaaatacccacagctgctgcattgggtgtgtcaaggatgcctgtgcgtatcgcactaaggagtttggagcaagaaggtctagttgtgaggttgggagcaaggggttacgccgccaggggagtttcttccgatcagattagagacgctatcgaagtgagaggtgtattagaaggcttcgcagcccgtcgtttagccgaaaggggtatgactgctgagactcacgcaaggttcgtggtcttgattgctgagggcgaggccttattcgctgcaggtaggctaaatggtgaagacctagaccgttacgctgcttacaatcaagccttccatgataccctggtctcagcagctggcaatggagcagtagaatctgccctagccaggaacggatttgagccattcgcagcagcaggcgcattagccttggacttaatggacttatccgctgagtatgagcatttactggccgctcacaggcaacatcaagccgtactagatgctgtatcatgcggcgatgcagaaggtgcagaaaggattatgcgtgatcacgctctggcagccataagaaacgcaaaggttttcgaagccgcagcatccgcaggagccccccttggtgccgcatggtctatacgtgccgacgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaa

SEQ ID NO: 28 - DNA, 879 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggttccagtacagcaccccctaccgacgttagtctgggtgacgaactacatttggacggcgaagatgtcgctatggctcatgcagacgcattagacgactttgacttggacatgctgggagatggagattctcctggtccaggattcacgccccatgacagtgccccttacggagccctggatatggcagatttcgagttcgagcaaatgtttactgatgctctgggcattgatgagtacggtggttaa

SEQ ID NO: 29 - DNA, 1827 bp, Artificial Sequence
  Synthetic polynucleotide
atgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 30 - DNA, 2391 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 31 - DNA, 1626 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaa

SEQ ID NO: 32 - DNA, 1818 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagacgccttagacgacttcgatttagacatgctttccatgcaaccctcattgagatcagagtatgaataccccgtattcagtcacgtccaagctggtatgttctctccagaactaaggacatttacgaaaggagatgccgagcgttgggtcagtgacgctttagatgactttgacctagatatgctttctatgcaaccatcattaagatccgaatacgaatatcctgtattttcacacgttcaggccggcatgttttcccccgaattacgtacgttcacgaaaggcgacgcagaaagatgggtatccgatgcactagatgattttgacttagatatgttgtcaatgcagccctctttaaggtccgaatacgagtaccccgtcttctctcacgttcaagccggcatgttttctcccgagctaagaacctttacgaaaggtgacgctgaaagatgggtgtcagatgcccttgatgattttgacttggatatgttataa

SEQ ID NO: 33 - DNA, 1830 bp, Artificial Sequence
  Synthetic polynucleotide
atggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 34 - DNA, 1758 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaatgccaccaagaccactggacgtactaaatcgttcactgaaatcccctgtgatagtgaggctaaagggaggccgtgagtttcgtggaaccttagatggatacgatattcacatgaacttggtactgttagacgccgaggagattcaaaacggtgaagttgtgagaaaggtgggatcagttgtgattagaggagataccgtcgtctttgttagtccagcccctggtggtgaaggtggcacgtctggaggcacatccggttcaacatccggtacgggatcttcaggctccggcggctcaattaacaaggacatagaggaatgtaacgctattatcgagcaatttatcgattatcttagaactggtcaggaaatgcctatggagatggcagatcaggcaattaacgtcgtgcctggaatgactccaaagactattttgcacgcaggtcctcctatacaaccagattggcttaaatctaacggttttcatgaaattgaggcagacgttaatgacacatctctactactaagtggcgattaa

SEQ ID NO: 35 - DNA, 1626 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 36 - DNA, 1554 bp, Artificial Sequence
  Synthetic polynucleotide
atggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaa

SEQ ID NO: 37 - DNA, 2391 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaa

SEQ ID NO: 38 - DNA, 1626 bp, Artificial Sequence; identical to SEQ ID NO: 31
  Synthetic polynucleotide

SEQ ID NO: 39 - DNA, 2493 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 40 - DNA, 2997 bp, Artificial Sequence
  Synthetic polynucleotide
atggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaa

SEQ ID NO: 41 - protein, 532 aa, Artificial Sequence
  Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSEFPPKKKRKVGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 42 - protein, 779 aa, Artificial Sequence
  Synthetic polypeptide
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADEFPPKKKRKVGSDALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 43 - protein, 292 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSSTAPPTDVSLGDELHLDGEDVAMAHADALDDFDLDMLGDGDSPGPGFTPHDSAPYGALDMADFEFEQMFTDALGIDEYGG

SEQ ID NO: 44 - protein, 608 aa, Artificial Sequence
  Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSEFPPKKKRKVGSDALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 45 - protein, 796 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSDALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 46 - protein, 541 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 47 - protein, 605 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGVPTIVMVDAYKRYKGTGSGATAGSAATGGATGGSVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSDALDDFDLDMLSMQPSLRSEYEYPVFSHVQAGMFSPELRTFTKGDAERWVSDALDDFDLDMLSMQPSLRSEYEYPVFSHVQAGMFSPELRTFTKGDAERWVSDALDDFDLDMLSMQPSLRSEYEYPVFSHVQAGMFSPELRTFTKGDAERWVSDALDDFDLDML

SEQ ID NO: 48 - protein, 609 aa, Artificial Sequence
  Synthetic polynucleotide
MESTPTKQKAIFSASLLLFAERGFDATTMPMIAENAKVGAGTIYRYFKNKESLVNELFQQHVNEFLQCIESGLANERDGYRDGFHHIFEGMVTFTKNHPRALGFIKTHSQGTFLTEESRLAYQKLVEFVCTFFREGQKQGVIRNLPENALIAILFGSFMEVYEMIENDYLSLTDELLTGVEESLWAALSRQSEFPPKKKRKVGSTSGSGKPGSGEGSTKGDALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 49 - protein, 585 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSMPPRPLDVLNRSLKSPVIVRLKGGREFRGTLDGYDIHMNLVLLDAEEIQNGEVVRKVGSVVIRGDTVVFVSPAPGGEGGTSGGTSGSTSGTGSSGSGGSINKDIEECNAIIEQFIDYLRTGQEMPMEMADQAINVVPGMTPKTILHAGPPIQPDWLKSNGFHEIEADVNDTSLLLSGD

SEQ ID NO: 50 - protein, 541 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 51 - protein, 517 aa, Artificial Sequence
  Synthetic polypeptide
MESTPTKQKAIFSASLLLFAERGFDATTMPMIAENAKVGAGTIYRYFKNKESLVNELFQQHVNEFLQCIESGLANERDGYRDGFHHIFEGMVTFTKNHPRALGFIKTHSQGTFLTEESRLAYQKLVEFVCTFFREGQKQGVIRNLPENALIAILFGSFMEVYEMIENDYLSLTDELLTGVEESLWAALSRQSEFPPKKKRKVGSDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 52 - protein, 796 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSNLVTAFSNMDDMLQKAHLVIEGTFIYLRDSTEFFIRVRDGWKKLQLGELIPIPAGGTSGGTSGSTSGTGSSGSGGSDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 53 - protein, 541 aa, Artificial Sequence; identical to SEQ ID NO: 46
  Synthetic polypeptide

SEQ ID NO: 54 - protein, 830 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCEFPPKKKRKVGSTSGSGKPGSGEGSTKGVPTIVMVDAYKRYKGTGSGATAGSAATGGATGGSVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSDALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 55 - protein, 998 aa, Artificial Sequence
  Synthetic polypeptide
MESTPTKQKAIFSASLLLFAERGFDATTMPMIAENAKVGAGTIYRYFKNKESLVNELFQQHVNEFLQCIESGLANERDGYRDGFHHIFEGMVTFTKNHPRALGFIKTHSQGTFLTEESRLAYQKLVEFVCTFFREGQKQGVIRNLPENALIAILFGSFMEVYEMIENDYLSLTDELLTGVEESLWAALSRQSEFPPKKKRKVGSTSGSGKPGSGEGSTKGVPTIVMVDAYKRYKGATNFSLLKLAGDVELNPGPVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTEFPPKKKRKVGSSTGSSTGSSTGPGSTSGGGSNLVTAFSNMDDMLQKAHLVIEGTFIYLRDSTEFFIRVRDGWKKLQLGELIPIPAGGTSGGTSGSTSGTGSSGSGGSDALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 532 bp, Artificial Sequence
  Synthetic polynucleotide
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagatcgtcgacctagctctgtcttagtccctatcagtgatagagataacatgcctctcactaacatggtccctatcagtgatagagactactggggccacgattcgtgtgtccctatcagtgatagagatctgcgtaatactactcgcgtgttccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 57 - DNA, 336 bp, Artificial Sequence
  Synthetic polynucleotide
gagctccctaggtgattggatccaatcttgccccattcgctaagcccacattggatccaatagctagaccttacggattggtgcattggatccaatggtcgaacatctgctataagcgcattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 58 - DNA, 620 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 59 - DNA, 238 bp, Artificial Sequence
  Synthetic polynucleotide
gagctccctaggtgtccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 60 - DNA, 193 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtaaagtgaaagtcgagctcggtaccctcttttcatctataaatacaagacgagtgcgtccttttctagactcacccataaacaaataatcaataaat

SEQ ID NO: 61 - DNA, 302 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 62 - DNA, 302 bp, Artificial Sequence; identical to SEQ ID NO: 61
  Synthetic polynucleotide

SEQ ID NO: 63 - DNA, 368 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 64 - DNA, 620 bp, Artificial Sequence; identical to SEQ ID NO: 58
  Synthetic polynucleotide

SEQ ID NO: 65 - DNA, 302 bp, Artificial Sequence; identical to SEQ ID NO: 61
  Synthetic polynucleotide

SEQ ID NO: 66 - DNA, 540 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgtcgtcgacctagctctgtcttagcggaatgaactttcattccgtaacatgcctctcactaacatggcggaatgaactttcattccgctactggggccacgattcgtgtgcggaatgaactttcattccgtctgcgtaatactactcgcgtgtcggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 67 - DNA, 620 bp, Artificial Sequence; identical to SEQ ID NO: 58
  Synthetic polynucleotide

SEQ ID NO: 68 - DNA, 575 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtaccctgtggagaagggtgaacaatataaaaggctggagagatgtcaatgaagcagctggatagatttcaaattttctagatttcagagtaatcgcacaaaacgaaggaatcccaccaagacaaaaaaaaaaattctaag

SEQ ID NO: 69 - DNA, 302 bp, Artificial Sequence; identical to SEQ ID NO: 61
  Synthetic polynucleotide

SEQ ID NO: 70 - DNA, 282 bp, Artificial Sequence
  Synthetic polynucleotide
gagctccctaggtgcggaatgaactttcattccgcttgccccattcgctaagcccaccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 71 - DNA, 3068 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 72 - DNA, 3809 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatggacatgccaaggataaaacctggacagcgtgtgatgatggctctaaggaaaatgatcgcctccggcgaaatcaaatctggcgaaagaatagcagaaatacccacagctgctgcattgggtgtgtcaaggatgcctgtgcgtatcgcactaaggagtttggagcaagaaggtctagttgtgaggttgggagcaaggggttacgccgccaggggagtttcttccgatcagattagagacgctatcgaagtgagaggtgtattagaaggcttcgcagcccgtcgtttagccgaaaggggtatgactgctgagactcacgcaaggttcgtggtcttgattgctgagggcgaggccttattcgctgcaggtaggctaaatggtgaagacctagaccgttacgctgcttacaatcaagccttccatgataccctggtctcagcagctggcaatggagcagtagaatctgccctagccaggaacggatttgagccattcgcagcagcaggcgcattagccttggacttaatggacttatccgctgagtatgagcatttactggccgctcacaggcaacatcaagccgtactagatgctgtatcatgcggcgatgcagaaggtgcagaaaggattatgcgtgatcacgctctggcagccataagaaacgcaaaggttttcgaagccgcagcatccgcaggagccccccttggtgccgcatggtctatacgtgccgacgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 73 - DNA, 2348 bp, Artificial Sequence
  Synthetic polynucleotide
tctgtttcagaaagaacattttgactggaatgttttcaagaaggctgaacttattgagatggaaggcagtgaagcttcatctccctcagaagtcgatgttgtatatgaaagtggatctgtctcatctccaactgaagactcagaaaagagatccgtggacaagtctgagaatgttcaacttcaagaaactgagcttggagtgtcaagtggtgacgagtacgacgaacaagagcaaaaattgatcaagcgttacatcaagatagcatacgcttggtgtatacttgtggttctcgttacttcggtgttgttcccaatgtcactgtaccgtaactggatcatatctttgagtttctttagaggatatactggattgtccatgttctggttatacggagtcttcttagttatcgcagtctatcctctttatgatgggcgacattcgctaggccgaattggtcagggtttgtggaaagacttcaaaaggatcttcaaatctaaacgatgatttaaggctacaagagtgtaacagtcaaatatgtatttagtatgccagtaatatgacattagcttttgtaccgagagcaacaatgctctgaaatttgttcttgaatagattaaactgatagaatagcactgttaccactaacctctatatatgaacgttcttgtatctgtgctcccgattcattagatatgaacgctttgaaacacgctttttggagtagctttaggataaacctaattgtgactcccaaagcaattcgcatagataaccccagttcgagaaaataaattgcggagaaacttttcttctttctgcagtttcaatgtgagatttagtgatgacctaggcgattaactttaatttgcttttgcttgcgctcttgatatagtacgaaagcttggctctggcggggtcaaaaggtgaacatgactgacccatttgcaatgataaaagagatacctttcactgtagcttcttggggagaataactacgatatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggttccagtacagcaccccctaccgacgttagtctgggtgacgaactacatttggacggcgaagatgtcgctatggctcatgcagacgcattagacgactttgacttggacatgctgggagatggagattctcctggtccaggattcacgccccatgacagtgccccttacggagccctggatatggcagatttcgagttcgagcaaatgtttactgatgctctgggcattgatgagtacggtggttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 74 - DNA, 3296 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 75 - DNA, 3860 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 76 - DNA, 3095 bp, Artificial Sequence
  Synthetic polynucleotide
ttattaggaacttcgtctattccgacgccaacgtccgacaataatgaagattcaccagtgaaggtgtactctgtgaaacaatcatctaccaattattcgtcaaatactaataattccaacatattcaacgttgacaagttgaaaaggctccgggatgtcatcaccttgaatagcgaagtggtaattggtacatatagaagagtcgaacgtgcctcaagagcccgatctcaagcttcgtcaatgcaaaatagtcctggagacattcaagaaggagttgctttcgaatcaacaacagcaataagaccaaacaggcaatacgaggttaccgattctagacaccattatactcagcctccacctcttgaagagtatccatactcatacccagaaccaatagaggacagtccaatttattcatcattggcagacgaacagtatccatccgttccaggtcaatcaggattaacagaaaagcaaatacttcaaatacaggagaatgctctcttaccgtcagagccctttattgagccagaactcaatgattcagaatcgttaagggtacccgagctccctgcaagtccagaactctagcttgggatttttcatccaaggaagggcccctctgctacttggttgctatttacgttttgtttttgcgttttttttttgaaacatttaataatagggggcgatttactaaatattaagcaatgcagttgtatcactggactaagtaggagaatataattctgaaactggaaagattcccagattccactctaacacataacatagttgtacaacattgtcctcgtggttgtgtatatttctcgatttcacgttaccccagattttcgtaactagggagtgagtaaaaagcaactgaaagcaactctataaatggaaatagcaagacagtgtattttgagttttcaggatcgagctatataaaggataagtttgctttccttccatctttagcaaatcgtccaactgaaacatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 77 - DNA, 3816 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagacgccttagacgacttcgatttagacatgctttccatgcaaccctcattgagatcagagtatgaataccccgtattcagtcacgtccaagctggtatgttctctccagaactaaggacatttacgaaaggagatgccgagcgttgggtcagtgacgctttagatgactttgacctagatatgctttctatgcaaccatcattaagatccgaatacgaatatcctgtattttcacacgttcaggccggcatgttttcccccgaattacgtacgttcacgaaaggcgacgcagaaagatgggtatccgatgcactagatgattttgacttagatatgttgtcaatgcagccctctttaaggtccgaatacgagtaccccgtcttctctcacgttcaagccggcatgttttctcccgagctaagaacctttacgaaaggtgacgctgaaagatgggtgtcagatgcccttgatgattttgacttggatatgttataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 78 - DNA, 3828 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 79 - DNA, 3227 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaatgccaccaagaccactggacgtactaaatcgttcactgaaatcccctgtgatagtgaggctaaagggaggccgtgagtttcgtggaaccttagatggatacgatattcacatgaacttggtactgttagacgccgaggagattcaaaacggtgaagttgtgagaaaggtgggatcagttgtgattagaggagataccgtcgtctttgttagtccagcccctggtggtgaaggtggcacgtctggaggcacatccggttcaacatccggtacgggatcttcaggctccggcggctcaattaacaaggacatagaggaatgtaacgctattatcgagcaatttatcgattatcttagaactggtcaggaaatgcctatggagatggcagatcaggcaattaacgtcgtgcctggaatgactccaaagactattttgcacgcaggtcctcctatacaaccagattggcttaaatctaacggttttcatgaaattgaggcagacgttaatgacacatctctactactaagtggcgattaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 80 - DNA, 3624 bp, Artificial Sequence
  Synthetic polynucleotide
gaattctttttttcagaccatatgaccggtccatcttctacggggggattatctatgctttgacctctatcttgattcttttatgattcaaatcacttttacgttatttattacttactggttatttacttagcgccttttctgaaaaacatttactaaaaatcatacatcggcactctcaaacacgacagattgtgatcaagaagcagagacaatcaccactaaggttgcacatttgagccagtaggctcctaatagaggttcgatacttattttgataatacgacatattgtcttacctctgaatgtgtcaatactctctcgttcttcgtctcgtcagctaaaaatataacacttcgagtaagatacgcccaattgaaggctacgagataccagactatcactagtagaactttgacatctgctaaagcagatcaaatatccatttatccagaatcaattaccttcctttagcttgtcgaaggcatgaaaaagctacatgaaaatccccatccttgaagttttgtcagcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 81 - DNA, 3023 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggttccgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 82 - DNA, 3860 bp, Artificial Sequence
  Synthetic polynucleotide
ttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 83 - DNA, 3095 bp, Artificial Sequence
  Synthetic polynucleotide
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacaatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 84 - DNA, 3312 bp, Artificial Sequence
  Synthetic polynucleotide
ccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatcatggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgtgagttcccaccaaaaaaaaagaggaaagtcggaagtacttctggatcaggaaagccaggttctggtgagggttctacgaagggtgtccctacaattgttatggtagacgcttacaagagatacaagggcacgggaagtggagccacagcaggttccgccgccacgggtggagccactggcggttctgtacccacgatagtaatggtcgatgcctataagagatataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 85 - DNA, 4466 bp, Artificial Sequence
  Synthetic polynucleotide
atcttttcagcttcatcgtcagtgatatttctcagcccacagaccaagtcaactttggaatctaacaaccttgttcttacaatgttagaactcttaagtcgcatgccatgatcttcaagctgaattttgtgaaggaggtcaaaccccacaatggcatctagttgtttagaatacatgccttcgacaagtgtttgagtgtccaaaatcaagagctcaaaattattgaatttgtctgccaataacgccgtaaattgattagtgtccagcccaccaacaataggagcacctatagttaatttttcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatcatggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtccgagttcccaccaaaaaaaaagaggaaagtcggaagtacttctggatcaggaaagccaggttctggtgagggttctacgaagggtgtccccactatagtgatggtggatgcctacaaaaggtataaaggtgcaacgaatttttcccttttaaaattagctggagacgttgagcttaaccctggcccagtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacacagagttcccccccaagaaaaagaggaaagttggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctcagatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaatagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 86 - DNA, 576 bp, Artificial Sequence
  Synthetic polynucleotide
atggaatccacgcccacgaagcaaaaagctattttttcagcttccctgctgctttttgccgaaaggggattcgatgctacgacaatgcctatgatagccgagaatgccaaagttggtgccggaaccatctataggtacttcaaaaacaaagaatccctggttaatgaactgttccagcagcacgtaaacgaatttttgcagtgcattgaaagtggattagcaaacgaacgtgacggctacagagatggatttcatcacatttttgaaggcatggtcacttttacgaaaaaccatccccgtgctctaggatttataaagactcattctcaaggcacgttcctaaccgaggaatcacgtttagcataccaaaagttagtcgaattcgtatgtactttcttccgtgagggtcaaaagcagggtgtgattcgtaacctacccgagaacgctttgattgccatacttttcggttcatttatggaagtttacgaaatgattgagaacgattacttgtccttaacggacgagctgctaaccggagtcgaggaatcactgtgggccgctttatcacgtcagtcc

SEQ ID NO: 87 - DNA, 600 bp, Artificial Sequence
  Synthetic polynucleotide
atggcaagaactccctcacgttcttctataggtagtctacgtagtccccacacacataaggcaattctaacgtcaaccattgaaatattaaaagagtgtggatattccggcttgtcaatagagagtgtggcccgtcgtgcaggcgctggaaaacctacgatatacagatggtggacaaataaggctgccctaattgctgaggtctatgagaatgagattgaacaagtaaggaagtttccagacttaggttctttcaaggctgatcttgacttccttcttcataacctttggaaagtctggagggaaactatatgcggcgaggctttcagatgtgtcatagccgaggctcaattagatccagttaccctgacacagttaaaggaccaattcatggaaaggagacgtgaaattccaaaaaaacttgtagaggatgccatttcaaatggtgaattaccaaaggatattaacagggagctattgctggacatgatattcggtttttgctggtataggctactgacagagcaacttaccgtagagcaggatatcgaggagtttacgttcttgctaattaatggagtctgccccggaactcagtgt

SEQ ID NO: 88 - DNA, 621 bp, Artificial Sequence
  Synthetic polynucleotide
atgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatca

SEQ ID NO: 89 - DNA, 732 bp, Artificial Sequence
  Synthetic polynucleotide
atggacatgccaaggataaaacctggacagcgtgtgatgatggctctaaggaaaatgatcgcctccggcgaaatcaaatctggcgaaagaatagcagaaatacccacagctgctgcattgggtgtgtcaaggatgcctgtgcgtatcgcactaaggagtttggagcaagaaggtctagttgtgaggttgggagcaaggggttacgccgccaggggagtttcttccgatcagattagagacgctatcgaagtgagaggtgtattagaaggcttcgcagcccgtcgtttagccgaaaggggtatgactgctgagactcacgcaaggttcgtggtcttgattgctgagggcgaggccttattcgctgcaggtaggctaaatggtgaagacctagaccgttacgctgcttacaatcaagccttccatgataccctggtctcagcagctggcaatggagcagtagaatctgccctagccaggaacggatttgagccattcgcagcagcaggcgcattagccttggacttaatggacttatccgctgagtatgagcatttactggccgctcacaggcaacatcaagccgtactagatgctgtatcatgcggcgatgcagaaggtgcagaaaggattatgcgtgatcacgctctggcagccataagaaacgcaaaggttttcgaagccgcagcatccgcaggagccccccttggtgccgcatggtctatacgtgccgac

SEQ ID NO: 90 - protein, 192 aa, Artificial Sequence
  Synthetic polypeptide
MESTPTKQKAIFSASLLLFAERGFDATTMPMIAENAKVGAGTIYRYFKNKESLVNELFQQHVNEFLQCIESGLANERDGYRDGFHHIFEGMVTFTKNHPRALGFIKTHSQGTFLTEESRLAYQKLVEFVCTFFREGQKQGVIRNLPENALIAILFGSFMEVYEMIENDYLSLTDELLTGVEESLWAALSRQS

SEQ ID NO: 91 - protein, 200 aa, Artificial Sequence
  Synthetic polypeptide
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQC

SEQ ID NO: 92 - protein, 207 aa, Artificial Sequence
  Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 93 - protein, 244 aa, Artificial Sequence
  Synthetic polypeptide
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRAD

SEQ ID NO: 94 - DNA, 363 bp, Artificial Sequence
  Synthetic polynucleotide
gagtttcctggcataaccctaaggattcaggagacagacatgctatataaaggagacactttgtacctggattggctagaggatggcatcgctgagttagtatttgacgccccaggatctgtcaataaactagacactgccgtggcttcactgggtgaagcaataggagtgttagagcaacaatcagaccttatctgggaaacactaactgttaaagacgccaaagtgaacttcgattctggattagaaaagttcgaggaagccattccctctgctgatgatttcgaccccgttgccgagcgtaggtcatctggagaatttcgtgcagaaaggcacagtggcggaactgatctttgcttctaa

SEQ ID NO: 95 - DNA, 240 bp, Artificial Sequence
  Synthetic polynucleotide
attaacaaggacatagaggaatgtaacgctattatcgagcaatttatcgattatcttagaactggtcaggaaatgcctatggagatggcagatcaggcaattaacgtcgtgcctggaatgactccaaagactattttgcacgcaggtcctcctatacaaccagattggcttaaatctaacggttttcatgaaattgaggcagacgttaatgacacatctctactactaagtggcgattaa

SEQ ID NO: 96 - DNA, 321 bp, Artificial Sequence
  Synthetic polynucleotide
ggcggagccaattttaatcaatctggcaatatagcagattctagtctatcctttacgtttactaattcatccaacggcccaaatctgataacaacccaaacgaatagtcaagctctatcccagcccatcgctagttccaacgtgcatgataattttatgaacaatgagataactgcatccaaaatcgatgatggtaacaattcaaagcccctttctccaggatggaccgatcaaacagcctacaacgccttcggcatcacgactggcatgtttaacaccacaacgatggatgatgtttacaattatttatttgatgactaa

SEQ ID NO: 97 - DNA, 942 bp, Artificial Sequence
  Synthetic polynucleotide
gatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggaagtggcggcggatccggtggctcaggatccgtattaccacaagccccagctccagctcctgcaccagcaatggtgagtgccctggcccaagctcctgctccagtgcctgtccttgctccaggtcctccccaggctgtagcacctcctgcaccaaagcccacacaagccggtgagggcacacttagtgaagctctgcttcaattgcagtttgatgacgaagaccttggagccctattaggcaattccaccgacccagcagtgtttacagatttagcaagtgtggacaactctgagtttcagcagctacttaaccagggaatacccgttgcaccccatactacggagccaatgttaatggagtatcccgaggccataaccaggcttgttactggagcacagaggccaccagacccagctcccgcacccttgggcgctccaggactacccaatggactactatctggcgacgaagatttttcctccatcgccgacatggatttttcagccctgttatcaggtggtggtagtggaggctccggcagtgacctttcccaccctccccccaggggacacctggacgagttaaccaccactttagagagtatgaccgaagatctaaacctggacagtccactgacaccagagcttaatgaaattctagatacattcttaaatgacgagtgcctgctacatgccatgcatattagtacaggtttgtcaatttttgacacgtctttgttttaa

SEQ ID NO: 98 - DNA, 108 bp, Artificial Sequence
  Synthetic polynucleotide
aatgctgagtttgaaaatatagaattttctacacctcagatgatgcccgttgaagacgctgagacttggatgaataatatgggtccaattcccaacttttctctgtaa

SEQ ID NO: 99 - DNA, 942 bp, Artificial Sequence
  Synthetic polynucleotide
ccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 100 - DNA, 243 bp, Artificial Sequence
  Synthetic polynucleotide
agtacagcaccccctaccgacgttagtctgggtgacgaactacatttggacggcgaagatgtcgctatggctcatgcagacgcattagacgactttgacttggacatgctgggagatggagattctcctggtccaggattcacgccccatgacagtgccccttacggagccctggatatggcagatttcgagttcgagcaaatgtttactgatgctctgggcattgatgagtacggtggttaa

SEQ ID NO: 101 - DNA, 153 bp, Artificial Sequence
  Synthetic polynucleotide
gatgccctggacgactttgatttggacatgctgggctccgacgcactggatgactttgatttggatatgctgggtagtgatgccctagacgattttgacctggacatgttgggaagtgacgcccttgacgacttcgatcttgatatgttataa

SEQ ID NO: 102 - DNA, 495 bp, Artificial Sequence
  Synthetic polynucleotide
gacgccttagacgacttcgatttagacatgctttccatgcaaccctcattgagatcagagtatgaataccccgtattcagtcacgtccaagctggtatgttctctccagaactaaggacatttacgaaaggagatgccgagcgttgggtcagtgacgctttagatgactttgacctagatatgctttctatgcaaccatcattaagatccgaatacgaatatcctgtattttcacacgttcaggccggcatgttttcccccgaattacgtacgttcacgaaaggcgacgcagaaagatgggtatccgatgcactagatgattttgacttagatatgttgtcaatgcagccctctttaaggtccgaatacgagtaccccgtcttctctcacgttcaagccggcatgttttctcccgagctaagaacctttacgaaaggtgacgctgaaagatgggtgtcagatgcccttgatgattttgacttggatatgttataa

SEQ ID NO: 103 - DNA, 1170 bp, Artificial Sequence
  Synthetic polynucleotide
gatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 104 - DNA, 1572 bp, Artificial Sequence
  Synthetic polynucleotide
gatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaactcaaggagttctggcagtcccaagaaaaaacgtaaagtaggatctcagtatctgcctgacacggacgatcgtcatagaattgaagaaaagcgtaagaggacgtatgaaacgttcaagtctataatgaaaaaatctcccttctctggtcccaccgatcccagacctcccccaagaaggatagcagtgccctcaagaagttctgctagtgtacccaagccagccccccaaccctatcctttcacctcttctctttcaacaataaattacgacgagttccccacaatggtttttccttcaggtcagatctcccaagcatctgcattagctcctgcacctccccaagtcctgcctcaagcccctgctcctgcacccgctccagccatggtatcagcacttgctcaagcacccgcacccgtgcctgtattagctcccggcccacctcaagctgtagccccccctgctccaaaacccacccaggccggagaaggaacactttcagaagcattacttcagcttcagtttgacgacgaagacttgggcgcattattaggcaactctacggatcccgctgtttttactgacttggcaagtgtggataacagtgagttccagcagctattgaaccaaggtatccccgtcgctccccatacgacagaacctatgcttatggaatatcctgaggcaatcactaggctggtcacaggtgcacaacgtcccccagaccccgcacccgccccattgggcgctcccggcttaccaaatggcttactatcaggtgatgaagatttctcttccatagccgacatggacttctctgccttactgggatcaggtagtggatcccgtgactcaagagagggaatgttcctaccaaaaccagaagcaggatccgccatcagtgacgtctttgaaggcagggaggtatgtcaacctaaaaggataagacccttccatccacctggtagtccatgggcaaacaggccacttcccgcctctctggcacccactcctacaggccctgtacacgaacctgttggaagtcttacccccgctccagtgccccagcccttagaccctgcccccgcagtcacccccgaggctagtcatctattggaagatcctgacgaggagacaagtcaagccgtcaaagccctaagagaaatggctgacacggtgattccacagaaggaagaagccgccatctgcggtcaaatggatctatctcatccaccacccaggggccatttagatgagttaacgactactctggaatctatgacggaagaccttaaccttgattccccattaactccagagctaaacgaaatcttggacactttcttaaatgatgaatgtctgctgcatgctatgcatatttccactggcttgtcaatattcgacacaagtctattttaa

SEQ ID NO: 105 - protein, 120 aa, Artificial Sequence
  Synthetic polypeptide
EFPGITLRIQETDMLYKGDTLYLDWLEDGIAELVFDAPGSVNKLDTAVASLGEAIGVLEQQSDLIWETLTVKDAKVNFDSGLEKFEEAIPSADDFDPVAERRSSGEFRAERHSGGTDLCF

SEQ ID NO: 106 - protein, 79 aa, Artificial Sequence
  Synthetic polypeptide
INKDIEECNAIIEQFIDYLRTGQEMPMEMADQAINVVPGMTPKTILHAGPPIQPDWLKSNGFHEIEADVNDTSLLLSGD

SEQ ID NO: 107 - protein, 106 aa, Artificial Sequence
  Synthetic polypeptide
GGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDD

SEQ ID NO: 108 - protein, 313 aa, Artificial Sequence
  Synthetic polypeptide
DALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 109 - protein, 35 aa, Artificial Sequence
  Synthetic polypeptide
NAEFENIEFSTPQMMPVEDAETWMNNMGPIPNFSL

SEQ ID NO: 110 - protein, 313 aa, Artificial Sequence
  Synthetic polypeptide
PSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 111 - protein, 80 aa, Artificial Sequence
  Synthetic polypeptide
STAPPTDVSLGDELHLDGEDVAMAHADALDDFDLDMLGDGDSPGPGFTPHDSAPYGALDMADFEFEQMFTDALGIDEYGG

SEQ ID NO: 112 - protein, 50 aa, Artificial Sequence
  Synthetic polypeptide
DALDDFDLDMLGSDALDDFDLDMLGSDALDDFDLDMLGSDALDDFDLDML

SEQ ID NO: 113 - protein, 164 aa, Artificial Sequence
  Synthetic polypeptide
DALDDFDLDMLSMQPSLRSEYEYPVFSHVQAGMFSPELRTFTKGDAERWVSDALDDFDLDMLSMQPSLRSEYEYPVFSHVQAGMFSPELRTFTKGDAERWVSDALDDFDLDMLSMQPSLRSEYEYPVFSHVQAGMFSPELRTFTKGDAERWVSDALDDFDLDML

SEQ ID NO: 114 - protein, 389 aa, Artificial Sequence
  Synthetic polypeptide
DALDDFDLDMLGSDALDDFDLDMLGSDALDDFDLDMLGSDALDDFDLDMLINSRSSGSPKKKRKVGSGGGSGGSGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 115 - protein, 523 aa, Artificial Sequence
  Synthetic polypeptide
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 116 - DNA, 30 bp, Artificial Sequence
  Synthetic polynucleotide
gagttcccaccaaaaaaaaagaggaaagtc

SEQ ID NO: 117 - DNA, 30 bp, Artificial Sequence
  Synthetic polynucleotide
gagttcccccccaagaaaaagaggaaagtt

SEQ ID NO: 118 - protein, 10 aa, Artificial Sequence
  Synthetic polypeptide
EFPPKKKRKV

SEQ ID NO: 119 - protein, 7 aa, Artificial Sequence
  Synthetic polypeptide
PKKKRKV

SEQ ID NO: 120 - DNA, 54 bp, Artificial Sequence
  Synthetic polynucleotide
ggaagtacttctggatcaggaaagccaggttctggtgagggttctacgaagggt

SEQ ID NO: 121 - DNA, 6 bp, Artificial Sequence
  Synthetic polynucleotide
ggttcc

SEQ ID NO: 122 - protein, 18 aa, Artificial Sequence
  Synthetic polypeptide
GSTSGSGKPGSGEGSTKG

SEQ ID NO: 123 - protein, 2 aa, Artificial Sequence
  Synthetic polypeptide
GS

SEQ ID NO: 124 - DNA, 60 bp, Artificial Sequence
  Synthetic polynucleotide
ggtgcaacgaatttttcccttttaaaattagctggagacgttgagcttaaccctggccca

SEQ ID NO: 125 - protein, 20 aa, Artificial Sequence
  Synthetic polypeptide
GATNFSLLKLAGDVELNPGP

SEQ ID NO: 126 - DNA, 59 bp, Artificial Sequence
  Synthetic polynucleotide
gagctccctaggtgcggaatgaactttcattccgaaagtgaaagtcgagctcggtaccc

SEQ ID NO: 127 - DNA, 69 bp, Artificial Sequence
  Synthetic polynucleotide
gagctccctaggtgatgatacgaaacgtaccgtatcgttaaggtaaagtgaaagtcgagctcggtaccc

SEQ ID NO: 128 - DNA, 58 bp, Artificial Sequence
  Synthetic polynucleotide
gagctccctaggtgtccctatcagtgatagagaaaagtgaaagtcgagctcggtaccc

SEQ ID NO: 129 - DNA, 51 bp, Artificial Sequence
  Synthetic polynucleotide
gagctccctaggtgattggatccaataaagtgaaagtcgagctcggtaccc

SEQ ID NO: 130 - DNA, 20 bp, Unknown
  May be bacillus megaterium; Bm3R1 operator
cggaatgaactttcattccg

SEQ ID NO: 131 - DNA, 19 bp, Unknown
  May be streptomyces, escherichia coli; TetR operator
tccctatcagtgatagaga

SEQ ID NO: 132 - DNA, 30 bp, Unknown
  May be pseudomonas; Ph1F operator
atgatacgaaacgtaccgtatcgttaaggt

SEQ ID NO: 133 - DNA, 12 bp, Unknown
  organism unknown; VanR operator
attggatccaat

SEQ ID NO: 134 - DNA, 102 bp, Artificial Sequence
  Synthetic polynucleotide
gagctccctaggtgcggaatgaactttcattccgcttgccccattcgctaagcccaccggaatgaactttcattccgaaagtgaaagtcgagctcggtaccc

SEQ ID NO: 135 - DNA, 188 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgaaagtgaaagtcgagctcggtaccc

SEQ ID NO: 136 - DNA, 360 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgtcgtcgacctagctctgtcttagcggaatgaactttcattccgtaacatgcctctcactaacatggcggaatgaactttcattccgctactggggccacgattcgtgtgcggaatgaactttcattccgtctgcgtaatactactcgcgtgtcggaatgaactttcattccgaaagtgaaagtcgagctcggtaccc

SEQ ID NO: 137 - DNA, 122 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtaaagtgaaagtcgagctcggtaccc

SEQ ID NO: 138 - DNA, 228 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggtaaagtgaaagtcgagctcggtaccc

SEQ ID NO: 139 - DNA, 440 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtaccc

SEQ ID NO: 140 - DNA, 100 bp, Artificial Sequence
  Synthetic polynucleotide
gagctccctaggtgtccctatcagtgatagagacttgccccattcgctaagcccactccctatcagtgatagagaaaagtgaaagtcgagctcggtaccc

SEQ ID NO: 141 - DNA, 184 bp, Artificial Sequence
  Synthetic polynucleotide
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagaaaagtgaaagtcgagctcggtaccc

SEQ ID NO: 142 - DNA, 352 bp, Artificial Sequence
  Synthetic polynucleotide
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagatcgtcgacctagctctgtcttagtccctatcagtgatagagataacatgcctctcactaacatggtccctatcagtgatagagactactggggccacgattcgtgtgtccctatcagtgatagagatctgcgtaatactactcgcgtgttccctatcagtgatagagaaaagtgaaagtcgagctcggtaccc

SEQ ID NO: 143 - DNA, 86 bp, Artificial Sequence
  Synthetic polynucleotide
gagctccctaggtgattggatccaatcttgccccattcgctaagcccacattggatccaataaagtgaaagtcgagctcggtaccc

SEQ ID NO: 144 - DNA, 156 bp, Artificial Sequence
  Synthetic polynucleotide
gagctccctaggtgattggatccaatcttgccccattcgctaagcccacattggatccaatagctagaccttacggattggtgcattggatccaatggtcgaacatctgctataagcgcattggatccaataaagtgaaagtcgagctcggtaccc

SEQ ID NO: 145 - DNA, 296 bp, Artificial Sequence
  Synthetic polynucleotide
gagctccctaggtgattggatccaatcttgccccattcgctaagcccacattggatccaatagctagaccttacggattggtgcattggatccaatggtcgaacatctgctataagcgcattggatccaattcgtcgacctagctctgtcttagattggatccaattaacatgcctctcactaacatggattggatccaatctactggggccacgattcgtgtgattggatccaattctgcgtaatactactcgcgtgtattggatccaataaagtgaaagtcgagctcggtaccc

SEQ ID NO: 146 - DNA, 469 bp, Unknown
  May be komagataella phaffi or picha pastoris
tagtcaaatattaatctatttcacctgttcaaactttacttaatgtacaaatgtggtagttattagttttgcaacggaacttgttccataatctggtcctctgggacagcaaactgtctttcactagtagcgccagtttcgggagtccacacagcattagtcaccggtgcaccagcactaatctcacgaccttctgggtgtttaaatgggcagttagggttgcggcatccagctgcaaacttacaatcctcatcaattggatgagtgaaaaaacagtttggtctggtacaactgttgccttcacgacacagtacaggagtagttgcgtgacgtcttgggcacttgtaattacggcatgatttaccaaatcgacattgttccaaagccctctgttgtttttgttgtttctcttcttcggtgatcttgtgttcaggtgatcgatgagcctttggacagtccggattagagc

SEQ ID NO: 147 - DNA, 261 bp, Unknown
  May be komagataella phaffi or picha pastoris
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 148 - DNA, 42 bp, Artificial Sequence
  Synthetic polynucleotide
gtccccactatagtgatggtggatgcctacaaaaggtataaa

SEQ ID NO: 149 - DNA, 144 bp, Artificial Sequence
  Synthetic polynucleotide
gtccctacaattgttatggtagacgcttacaagagatacaagggcacgggaagtggagccacagcaggttccgccgccacgggtggagccactggcggttctgtacccacgatagtaatggtcgatgcctataagagatataaa

SEQ ID NO: 150 - DNA, 552 bp, Artificial Sequence
  Synthetic polynucleotide
gtgcccaccattgtaatggttgatgcctacaagaggtacaaaggaaccggatcaggtgccacggccggttctgcagccaccggaggagcaacaggaggaagtgtgcctactatcgtaatggtcgacgcttataaacgttacaaatctggcgcaggaggctctggtggctcatccggttctgacggcgcaagtggatcaagagacgttccaaccattgtgatggtcgatgcttataagcgttataagggtggcggttcaacagcttcaggcggtacatcctcaggaaccggctccgcaacttctggagtaccaacaatcgtcatggtagatgcatataaaaggtataaggccggtacagctagtggtggttcaggctcagcaagtgccagtggtggcacgggtggtggtgtgccaactatcgttatggtcgatgcatacaaacgttacaagggcccccagccccaaccaaagccacaaccaaaacctgaaccagagccccaacctcagggagtgcctactattgttatggtggacgcctataaacgttataag

SEQ ID NO: 151 - protein, 14 aa, Artificial Sequence
  Synthetic polypeptide
VPTIVMVDAYKRYK

SEQ ID NO: 152 - protein, 48 aa, Artificial Sequence
  Synthetic polypeptide
VPTIVMVDAYKRYKGTGSGATAGSAATGGATGGSVPTIVMVDAYKRYK

SEQ ID NO: 153 - protein, 184 aa, Artificial Sequence
  Synthetic polypeptide
VPTIVMVDAYKRYKGTGSGATAGSAATGGATGGSVPTIVMVDAYKRYKSGAGGSGGSSGSDGASGSRDVPTIVMVDAYKRYKGGGSTASGGTSSGTGSATSGVPTIVMVDAYKRYKAGTASGGSGSASASGGTGGGVPTIVMVDAYKRYKGPQPQPKPQPKPEPEPQPQGVPTIVMVDAYKRYK

SEQ ID NO: 154 - DNA, 339 bp, Artificial Sequence
  Synthetic polynucleotide
gtaaccactttatctggcctatcaggcgagcagggaccaagtggagatatgacgacagaggaggattccgcaacgcatatcaaattctccaaaagggatgaagacggacgtgaattggccggtgcaacgatggagttacgtgactccagtggtaagacaatttccacctggatctcagacggtcatgtaaaggatttttacctgtatcccggcaaatatactttcgtagagaccgcagcccccgacggttatgaagtcgctactgctatcaccttcacggttaacgagcagggacaggtaactgtaaatggagaagccaccaaaggtgacgcacacaca

SEQ ID NO: 155 - protein, 113 aa, Artificial Sequence
  Synthetic polypeptide
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 156 - DNA, 363 bp, Artificial Sequence
  Synthetic polynucleotide
ggttcaagtaccggctcctccactggatcatcaacgggtccaggttctacgtccggtggcggttcaatgccaccaagaccactggacgtactaaatcgttcactgaaatcccctgtgatagtgaggctaaagggaggccgtgagtttcgtggaaccttagatggatacgatattcacatgaacttggtactgttagacgccgaggagattcaaaacggtgaagttgtgagaaaggtgggatcagttgtgattagaggagataccgtcgtctttgttagtccagcccctggtggtgaaggtggcacgtctggaggcacatccggttcaacatccggtacgggatcttcaggctccggcggctca

SEQ ID NO: 157 - DNA, 66 bp, Artificial Sequence
  Synthetic polynucleotide
ggttcaagtaccggctcctccactggatcatcaacgggtccaggttctacgtccggtggcggttca

SEQ ID NO: 158 - DNA, 294 bp, Artificial Sequence
  Synthetic polynucleotide
ggttcaagtaccggctcctccactggatcatcaacgggtccaggttctacgtccggtggcggttcaaacttagtgacagccttttccaacatggatgatatgcttcaaaaagcacacttagtaattgaaggcacctttatttatttaagagactctacagagttcttcattagggtaagggacggttggaaaaaacttcagttaggagaattgattcccatacccgctggtggcacgtctggaggcacatccggttcaacatccggtacgggatcttcaggctccggcggctca

SEQ ID NO: 159 - protein, 121 aa, Artificial Sequence
  Synthetic polypeptide
GSSTGSSTGSSTGPGSTSGGGSMPPRPLDVLNRSLKSPVIVRLKGGREFRGTLDGYDIHMNLVLLDAEEIQNGEVVRKVGSVVIRGDTVVFVSPAPGGEGGTSGGTSGSTSGTGSSGSGGS

SEQ ID NO: 160 - protein, 22 aa, Artificial Sequence
  Synthetic polypeptide
GSSTGSSTGSSTGPGSTSGGGS

SEQ ID NO: 161 - protein, 98 aa, Artificial Sequence
  Synthetic polypeptide
GSSTGSSTGSSTGPGSTSGGGSNLVTAFSNMDDMLQKAHLVIEGTFIYLRDSTEFFIRVRDGWKKLQLGELIPIPAGGTSGGTSGSTSGTGSSGSGGS

SEQ ID NO: 162 - DNA, 180 bp, Unknown
  May be komagataella phaffi or picha pastoris
aacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 163 - DNA, 195 bp, Unknown
  May be komagataella phaffi or picha pastoris
taagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 164 - DNA, 71 bp, Unknown
  May be komagataella phaffi or picha pastoris
tcttttcatctataaatacaagacgagtgcgtccttttctagactcacccataaacaaataatcaataaat

SEQ ID NO: 165 - DNA, 135 bp, Unknown
  May be komagataella phaffi or picha pastoris
tgtggagaagggtgaacaatataaaaggctggagagatgtcaatgaagcagctggatagatttcaaattttctagatttcagagtaatcgcacaaaacgaaggaatcccaccaagacaaaaaaaaaaattctaag

SEQ ID NO: 166 - DNA, 7 bp, Artificial Sequence
  Synthetic polynucleotide
gcttaca

SEQ ID NO: 167 - DNA, 1504 bp, Artificial Sequence
  Synthetic polynucleotide
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagatcgtcgacctagctctgtcttagtccctatcagtgatagagataacatgcctctcactaacatggtccctatcagtgatagagactactggggccacgattcgtgtgtccctatcagtgatagagatctgcgtaatactactcgcgtgttccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 168 - DNA, 1308 bp, Artificial Sequence
  Synthetic polynucleotide
gagctccctaggtgattggatccaatcttgccccattcgctaagcccacattggatccaatagctagaccttacggattggtgcattggatccaatggtcgaacatctgctataagcgcattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 169 - DNA, 1592 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 170 - DNA, 1210 bp, Artificial Sequence
  Synthetic polynucleotide
gagctccctaggtgtccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 171 - DNA, 1165 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtaaagtgaaagtcgagctcggtaccctcttttcatctataaatacaagacgagtgcgtccttttctagactcacccataaacaaataatcaataaatatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 172 - DNA, 1274 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 173 - DNA, 1274 bp, Artificial Sequence; identical to SEQ ID NO: 172
  Synthetic polynucleotide

SEQ ID NO: 174 - DNA, 1340 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 175 - DNA, 1592 bp, Artificial Sequence; identical to SEQ ID NO: 169
  Synthetic polynucleotide

SEQ ID NO: 176 - DNA, 1274 bp, Artificial Sequence; identical to SEQ ID NO: 172
  Synthetic polynucleotide

SEQ ID NO: 177 - DNA, 1512 bp, Artificial Sequence
  Synthetic polynucleotide
gagctccctaggtgcggaatgaactttcattccgcttgccccattcgctaagcccaccggaatgaactttcattccgagctagaccttacggattggtgccggaatgaactttcattccgggtcgaacatctgctataagcgccggaatgaactttcattccgtcgtcgacctagctctgtcttagcggaatgaactttcattccgtaacatgcctctcactaacatggcggaatgaactttcattccgctactggggccacgattcgtgtgcggaatgaactttcattccgtctgcgtaatactactcgcgtgtcggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 178 - DNA, 1592 bp, Artificial Sequence; identical to SEQ ID NO: 169
  Synthetic polynucleotide

SEQ ID NO: 179 - DNA, 1547 bp, Artificial Sequence
  Synthetic polynucleotide
gagctccctaggtgatgatacgaaacgtaccgtatcgttaaggtcttgccccattcgctaagcccacatgatacgaaacgtaccgtatcgttaaggtagctagaccttacggattggtgcatgatacgaaacgtaccgtatcgttaaggtggtcgaacatctgctataagcgcatgatacgaaacgtaccgtatcgttaaggttcgtcgacctagctctgtcttagatgatacgaaacgtaccgtatcgttaaggttaacatgcctctcactaacatggatgatacgaaacgtaccgtatcgttaaggtctactggggccacgattcgtgtgatgatacgaaacgtaccgtatcgttaaggttctgcgtaatactactcgcgtgtatgatacgaaacgtaccgtatcgttaaggtaaagtgaaagtcgagctcggtaccctgtggagaagggtgaacaatataaaaggctggagagatgtcaatgaagcagctggatagatttcaaattttctagatttcagagtaatcgcacaaaacgaaggaatcccaccaagacaaaaaaaaaaattctaagatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 180 - DNA, 1274 bp, Artificial Sequence; identical to SEQ ID NO: 172
  Synthetic polynucleotide

SEQ ID NO: 181 - DNA, 1254 bp, Artificial Sequence
  Synthetic polynucleotide
gagctccctaggtgcggaatgaactttcattccgcttgccccattcgctaagcccaccggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggtgtcaaagggagaggaagataatatggctattattaaggagtttatgcgttttaaggtacatatggaaggttctgtcaacggtcacgaattcgaaattgaaggtgagggggaggggaggccatacgagggaactcagactgctaagttaaaggtcactaaaggtggtcctttacctttcgcctgggatatcctgtctccacagtttatgtacggttcaaaggcttatgtgaaacatcctgccgatatcccagattatcttaaactttctttccctgagggttttaagtgggagagggtaatgaactttgaagacggtggtgtggtcactgttactcaggactcaagtctgcaggacggtgagttcatctacaaggtgaagctgagaggtaccaattttccatcagatggtcccgtgatgcaaaaaaagacaatgggttgggaagcttctagtgaacgtatgtatcccgaagatggagctttgaaaggtgaaattaagcaaagactaaaacttaaggatggtggacattacgatgctgaagttaagacgacctacaaggccaaaaagccagtccagttgcctggagcatacaatgttaacatcaaattggatataacttcccataatgaagactataccatcgtcgagcaatacgagcgggccgaagggagacacagtactggtggtatggatgaactttataaataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 182 - DNA, 1179 bp, Artificial Sequence
  Synthetic
atggacatgccaaggataaaacctggacagcgtgtgatgatggctctaaggaaaatgatcgcctccggcgaaatcaaatctggcgaaagaatagcagaaatacccacagctgctgcattgggtgtgtcaaggatgcctgtgcgtatcgcactaaggagtttggagcaagaaggtctagttgtgaggttgggagcaaggggttacgccgccaggggagtttcttccgatcagattagagatgctatcgaagtgagaggtgtattagaaggcttcgcagcccgtcgtttagccgaaaggggtatgactgctgagactcacgcaaggttcgtggtcttgattgctgagggcgaggccttattcgctgcaggtaggctaaatggtgaagacctagaccgttacgctgcttacaatcaagccttccatgataccctggtcagcgcagctggcaatggagcagtagaatctgccctagccaggaacggatttgagccattcgcagcagcaggcgcattagccttggacttaatggacttatccgctgagtatgagcatttactggccgctcacaggcaacatcaagccgtactagatgctgtatcatgcggcgatgcagaaggtgcagaaaggattatgcgtgatcacgctctggcagccataagaaacgcaaaggttttcgaagccgcagcatccgcaggagccccccttggtgccgcatggtctatacgtgccgacgagttcccaccaaaaaaaaagaggaaagtcggaagtacttctggatcaggaaagccaggttctggtgagggttctacgaagggtgagtttcctggcataaccctaaggattcaggagacagacatgctatataaaggagacactttgtacctggattggctagaggatggcatcgctgagttagtatttgacgccccaggatctgtcaataaactagacactgccgtggcttcactgggtgaagcaataggagtgttagagcaacaatcagaccttatctgggaaacactaactgttaaagacgccaaagtgaacttcgattctggattagaaaagttcgaggaagccattccctctgctgatgatttcgaccccgttgccgagcgtaggtcatctggagaatttcgtgcagaaaggcacagtggcggaactgatctttgcttctaa

SEQ ID NO: 183 - DNA, 1830 bp, Artificial Sequence; identical to SEQ ID NO: 33
  Synthetic

SEQ ID NO: 184 - DNA, 1875 bp, Artificial Sequence
  Synthetic
atgagtaggctagataaaagtaaggtaattaactccgccctggagcttttaaatgaagtaggtatagaaggtcttacgactcgtaaattagctcaaaaactaggagtggagcaacccactttatattggcatgttaagaacaagagggccttgctggacgcactggccatcgagatgttagaccgtcaccacacgcacttctgcccattagagggtgaatcctggcaagacttcttgagaaataatgccaagtctttccgttgcgctctgttgagtcaccgtgatggcgctaaagtgcatcttggaactaggcccacggagaaacagtatgagacactggaaaatcaactagcattcttgtgtcaacagggatttagtcttgagaatgccttgtacgctctatccgctgtgggccattttactctaggttgcgtacttgaagatcaggaacaccaagtagccaaagaagaacgtgagacgcctacgacagactccatgcctcctctacttcgtcaagccatcgagctttttgaccaccagggagctgagcctgccttcttattcggattagaactaattatttgcggtttagaaaagcaactaaaatgcgaaagtggatcagagttcccaccaaaaaaaaagaggaaagtcggaagtacttctggatcaggaaagccaggttctggtgagggttctacgaagggtgatgccctggacgactttgatttggacatgctgggctccgacgcactggatgactttgatttggatatgctgggtagtgatgccctagacgattttgacctggacatgttgggaagtgacgcccttgacgacttcgatcttgatatgttaataaattcaagaagttccggctcacctaaaaaaaaaagaaaggtaggttcaggaggcggaagtggcggttctggtagtccttcaggtcaaatctcaaatcaagctcttgcactggctccttcttcagcccctgttttggcccaaaccatggtgcccagttcagccatggtccctttggcacagcctcctgctccagcacccgttttgaccccaggtcctccacaatccttatcagcaccagtgcctaagtctacacaggcaggagagggtactctttcagaagccctgctacatcttcaatttgatgctgacgaggatttaggcgctttgcttggcaattctaccgatccaggagtgtttactgaccttgcatccgtagacaactccgagtttcaacaactgctaaaccagggagtgtctatgtctcattcaacagctgaacctatgttaatggagtatccagaagccataactcgtctggtaaccggttctcagcgtcctcccgatccagcacccacacctctgggtactagtggtttgcccaacggtttgtccggcgatgaagacttttcctccattgcagatatggactttagtgctctgttatctcagatctcaagttccggacaaggaggtggcggtagtggcttttctgtagacacttccgctttgctggatctgttctctccttccgttactgttcctgacatgtcccttcccgacctagactcatcattagcctcaattcaggaacttttaagtccacaagagccaccaagacccccagaagcagagaacagttcacccgatagtggcaaacaattggttcactataccgcccagccactgttcttactagacccaggtagtgtggacactggaagtaatgacctgcccgttcttttcgagctgggcgaaggctcttatttctcagaaggcgacggattcgccgaggaccccacaatatcactactaacgggctctgaacctcctaaagcaaaggaccccactgtttcataa

SEQ ID NO: 185 - DNA, 1179 bp, Artificial Sequence; identical to SEQ ID NO: 182
  Synthetic

SEQ ID NO: 186 - DNA, 266 bp, Artificial Sequence
  Synthetic
gagctccctaggtgattggatccaatcttgccccattcgctaagcccacattggatccaataaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 187 - DNA, 336 bp, Artificial Sequence; identical to SEQ ID NO: 57
  Synthetic

SEQ ID NO: 188 - DNA, 239 bp, Artificial Sequence
  Synthetic
gagctccctaggtgcggaatgaactttcattccgaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 189 - DNA, 282 bp, Artificial Sequence; identical to SEQ ID NO: 70
  Synthetic

SEQ ID NO: 190 - DNA, 368 bp, Artificial Sequence; identical to SEQ ID NO: 63
  Synthetic

SEQ ID NO: 191 - DNA, 238 bp, Artificial Sequence; identical to SEQ ID NO: 59
  Synthetic

SEQ ID NO: 192 - DNA, 364 bp, Artificial Sequence
  Synthetic
gagctccctaggtgtccctatcagtgatagagacttgccccattcgctaagcccactccctatcagtgatagagaagctagaccttacggattggtgctccctatcagtgatagagaggtcgaacatctgctataagcgctccctatcagtgatagagaaaagtgaaagtcgagctcggtacccaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 193 - DNA, 532 bp, Artificial Sequence; identical to SEQ ID NO: 56
  Synthetic

(19) United States
(12) Patent Application Publication
(10) Pub. No.: US 2023/0257793 A1
(43) Pub. Date: Aug. 17, 2023

(54) SYNTHETIC EXPRESSION SYSTEMS

(71) Applicant: Ginkgo Bioworks, Inc., Boston, MA (US)

(72) Inventors: Nikos Reppas, Cambridge, MA (US); Swaminath Srinivas, Boston, MA (US); Adam Meyer, Boston, MA (US); Alex Tucker, Boston, MA (US); Punita Manga, Boston, MA (US); Sneha Srikrishnan, Boston, MA (US)

(73) Assignee: Ginkgo Bioworks, Inc., Boston, MA (US)

(21) Appl. No.: 18/024,706

(22) PCT Filed: Sep. 5, 2021
(86) PCT No.: PCT/US2021/049180

§ 371 Date: Mar. 3, 2023

Related U.S. Application Data
(60) Provisional application No. 63/075,134, filed on Sep. 5, 2020.

(51) Int. Cl. C12P 21/02; C12N 15/63; C12N 15/81; C12N 9/26; C12N 9/10; C12N 15/52; C07K 14/795; C07K 14/77

(52) U.S. Cl. CPC C12P 21/02; C12N 15/635; C12N 15/815; C12N 9/2414; C12N 9/1029; C12Y 203/01037; C12N 15/52; C07K 14/795; C07K 14/77; C12N 2830/001; C12N 2800/102

(57) ABSTRACT

This application describes transcriptional units, synthetic expression systems, and host cells comprising transcriptional units and synthetic expression systems, wherein the synthetic expression system is capable of expressing a gene of interest. Also described are methods for the production of bioproducts (including, but not limited to, proteins or RNA expressed from the gene of interest). In some embodiments, bioproducts are produced from host cells under culture conditions without addition of methanol.

### CROSS REFERENCE TO RELATED APPLICATIONS

This application claims priority to U.S. provisional Application No. 63/075,134, filed Sep. 5, 2020, the content of which is herein incorporated by reference in its entirety.

### SEQUENCE LISTING

In accordance with 37 CFR 1.52(e)(5), the present specification makes reference to a Sequence Listing (submitted electronically as a .txt file named “G091970067W000-SEQ”). The .txt file was generated on Aug. 26, 2021, and is 401,042 bytes in size. The Sequence Listing is herein incorporated by reference in its entirety.

### FIELD OF THE INVENTION

This disclosure relates to synthetic expression systems comprising transcriptional units, host cells comprising synthetic expression systems, and methods for the methanol-independent bioproduction of proteins and other desired molecules.

### BACKGROUND

Certain methylotrophic yeast cells have been used in the production of bioproducts (e.g., proteins, nucleic acids, small molecules, etc.) due, in part, to the strong and regulatable characteristics of their native promoter systems. For example, many recombinant proteins have been successfully produced in Pichia pastoris, a methylotrophic yeast in which recombinant protein production is typically driven by its endogenous methanol-regulated AOX1 promoter, P(AOX1). Though P(AOX1)-based production systems are well characterized, optimized, robust, and have an extensive history of industrial use, the methanol dependence of P(AOX1) limits the use of P. pastoris expression systems to restrictive process conditions. This is a particularly acute issue in large-scale production environments because methanol is a highly toxic and flammable compound that is dangerous and undesirable at large scale.

### SUMMARY

A solution is needed that matches or exceeds the production capabilities of existing methanol-dependent expression systems at industrial scale. This disclosure describes transcriptional units and synthetic expression systems, host cells comprising transcriptional units and synthetic expression systems, and methods that facilitate high-yield synthesis of proteins and molecules, including under methanol-independent conditions.

Aspects of the disclosure relate to a methylotrophic host cell comprising a synthetic expression system that comprises the following elements: (1) a first transcriptional unit comprising an input promoter comprising an upstream activating sequence (UAS) and a core promoter element, and a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell; and (2) a second transcriptional unit comprising a synthetic output promoter operably linked to a gene of interest, wherein the synthetic transcription factor is an activator of the synthetic output promoter, wherein the gene of interest is expressed in the absence of exogenously provided methanol. In some embodiments, the input promoter drives expression of the at least one component of the synthetic transcription factor.

In some embodiments, the polynucleotide of the first transcriptional unit encodes all components of the transcription factor.

In some embodiments, the input promoter is naturally occurring. In some embodiments, the input promoter has at least 90% sequence identity to a naturally occurring promoter. In some embodiments, the input promoter is synthetic. In some embodiments, the input promoter is a constitutive promoter.

In some embodiments, the input promoter is a regulatable input promoter. In some embodiments, the regulatable input promoter is inducible. In some embodiments, the regulatable input promoter is repressible. In some embodiments, the regulatable input promoter is responsive to nutrient addition, limitation, or depletion with respect to a cognate cultivation process. In some embodiments, the regulatable input promoter is responsive to thiamine depletion. In some embodiments, the regulatable input promoter is responsive to glycerol limitation. In some embodiments, the regulatable input promoter is responsive to monosaccharide limitation. In some embodiments, the regulatable input promoter is responsive to the limitation of a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate. In some embodiments, the regulatable input promoter is responsive in the absence of exogenously provided methanol. In some embodiments, the regulatable input promoter is responsive to the limitation or depletion of a combination of any two or more nutrients. In some embodiments, activity of the regulatable input promoter is increased by the presence of exogenously provided formic acid. In some embodiments, the regulatable input promoter is regulatable in the absence of exogenously provided methanol. In some embodiments, the input promoter is not methanol inducible.

In some embodiments, the upstream activating sequence (UAS) and/or the core promoter element of the input promoter is not native to the methylotrophic host cell.

In some embodiments, the input promoter is P(JEN1), P(GQ6704499), P(GQ6700926), P(HGT1), P(FDH1), P(AOX2), P(RGI2), P(THI13)_short, P(THI13)_long, or P(THI4). In some embodiments, the input promoter is P(JEN1). In some embodiments, the input promoter is P(GQ6704499). In some embodiments, the input promoter is P(GQ6700926). In some embodiments, the input promoter is P(HGT1). In some embodiments, the input promoter is P(FDH1). In some embodiments, the input promoter is P(AOX2). In some embodiments, the input promoter is P(RGI2). In some embodiments, the input promoter is P(THI13)_short. In some embodiments, the input promoter is P(THI13)_long. In some embodiments, the input promoter is P(THI4).

In some embodiments, the input promoter is a polynucleotide having at least 90%, at least 95%, or at least 99% identity to a nucleic acid sequence of any one of SEQ ID NOs: 16-25. In some embodiments, the input promoter is a polynucleotide having a nucleic acid sequence of any one of SEQ ID NOs: 16-25.

In some embodiments, the DNA-binding domain (DBD) of the synthetic transcription factor is Bm3R1, TetR, PhlF_AM, or VanR_AM. In some embodiments, the DNA-binding domain (DBD) of the synthetic transcription factor is Bm3R1. In some embodiments, the DNA-binding domain (DBD) of the synthetic transcription factor is TetR. In some embodiments, the DNA-binding domain (DBD) of the synthetic transcription factor is PhlF_AM. In some embodiments, the DNA-binding domain (DBD) of the synthetic transcription factor is VanR_AM.

In some embodiments, the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD. In some embodiments, the transcriptional activation domain is (TAD) of the synthetic transcription factor B112_TAD. In some embodiments, the transcriptional activation domain (TAD) of the synthetic transcription factor is B42_TAD. In some embodiments, the transcriptional activation domain (TAD) of the synthetic transcription factor is GAL4_TAD. In some embodiments, the transcriptional activation domain (TAD) of the synthetic transcription factor is miniVPR_TAD. In some embodiments, the transcriptional activation domain (TAD) of the synthetic transcription factor is Mxr1_TAD. In some embodiments, the transcriptional activation domain (TAD) of the synthetic transcription factor is PH_TAD. In some embodiments, the transcriptional activation domain (TAD) of the synthetic transcription factor is VP16_TAD. In some embodiments, the transcriptional activation domain (TAD) of the synthetic transcription factor is VP64_TAD. In some embodiments, the transcriptional activation domain (TAD) of the synthetic transcription factor is VP64v2_TAD. In some embodiments, the transcriptional activation domain (TAD) of the synthetic transcription factor is VPH_TAD. In some embodiments, the transcriptional activation domain (TAD) of the synthetic transcription factor is VPR_TAD.

In some embodiments, the DNA-binding domain (DBD) of the synthetic transcription factor is Bm3R1, TetR, PhlF_AM, or VanR_AM, and the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD.

In some embodiments, the synthetic transcription factor is not an activator of the input promoter.

In some embodiments, the synthetic transcription factor is a one-component synthetic transcription factor. In some embodiments, the synthetic transcription factor is a two-component synthetic transcription factor. In some embodiments, the synthetic transcription factor is a multi-component synthetic transcription factor.

In some embodiments, the synthetic transcription factor comprises a nuclear localization signal. In some embodiments, the nuclear localization signal is an SV40 nuclear localization signal.

In some embodiments, the synthetic transcription factor comprises a linker.

In some embodiments, the two-component or multi-component synthetic transcription factor comprises a bioconjugate protein product part 1 (BPP1) and a bioconjugate protein part 2 (BPP2). In some embodiments, the BPP1 is SpyTag002 and the BPP2 is SpyCatcher002.

In some embodiments, the synthetic transcription factor comprises a self-cleaving polypeptide. In some embodiments, the self-cleaving polypeptide is a 2A peptide. In some embodiments, the self-cleaving polypeptide is ERBV_1_P2A.

In some embodiments, the synthetic transcription factor comprises an oligomerization domain. In some embodiments, the oligomerization domain is Linker_only_for_oligomerization; Trimerization_domain; or Heptamerization_domain.

In some embodiments, the first transcriptional unit comprises or consists of a polynucleotide having the nucleic acid sequence of any one of SEQ ID NOs: 26-40 or 182-185. In some embodiments, the synthetic transcription factor comprises or consists of a polypeptide having the amino acid sequence of any one of SEQ ID NOs: 41-55 or is encoded by a polynucleotide having the nucleic acid sequence of any one of SEQ ID NOs: 182-185.

In some embodiments, the synthetic output promoter is not methanol inducible.

In some embodiments, the synthetic output promoter comprises an upstream activating sequence and a core promoter element. In some embodiments, the upstream activating sequence (UAS) of the synthetic output promoter is not native to the methylotrophic host cell.

In some embodiments, the core promoter element of the synthetic output promoter has a nucleic acid sequence that is no more than 300 base pairs in length. In some embodiments, the core promoter element of the synthetic output promoter has a nucleic acid sequence that is from about 6 base pairs to about 300 base pairs, from about 25 base pairs to about 250 base pairs, from about 75 to about 225 base pairs, or from about 100 base pairs to about 175 base pairs in length. In some embodiments, the distance between the 3′ end of the upstream activating sequence (UAS) and the 5′ end of the core promoter element of the synthetic output promoter is from 0 to 200 base pairs in length. In some embodiments, the distance between the 3′ end of the upstream activating sequence (UAS) and the 5′ end of the core promoter element of the synthetic output promoter is a nucleic acid sequence having from about 6 base pairs to about 200 base pairs, from about 6 base pairs to about 53 base pairs, from about 20 base pairs to about 150 base pairs, from about 50 base pairs to about 125 base pairs, or from about 50 base pairs to about 100 base pairs in length.

In some embodiments, the core promoter element of the synthetic output promoter comprises a core promoter sequence that is at least 90%, at least 95%, or 100% identical to a naturally occurring core promoter sequence. In some embodiments, the core promoter element of the synthetic output promoter comprises a core promoter sequence that is at least 90%, at least 95%, or 100% identical to a core promoter sequence from P(AOX1) (SEQ ID NO: 162), P(DAS2) (SEQ ID NO: 163), P(HHF2) (SEQ ID NO: 164), or P(PMP20) (SEQ ID NO: 165). In some embodiments, the core promoter element of the synthetic output promoter comprises a core promoter sequence that is at least 90%, at least 95%, or 100% identical to a core promoter sequence from P(AOX1). In some embodiments, the core promoter element of the synthetic output promoter comprises a core promoter sequence that is at least 90%, at least 95%, or 100% identical to a core promoter sequence from P(DAS2). In some embodiments, the core promoter element of the synthetic output promoter comprises a core promoter sequence that is at least 90%, at least 95%, or 100% identical to a core promoter sequence from P(HHF2). In some embodiments, the core promoter element of the synthetic output promoter comprises a core promoter sequence that is at least 90%, at least 95%, or 100% identical to a core promoter sequence from P(PMP20).

In some embodiments, the upstream activating sequence (UAS) of the synthetic output promoter comprises bmO, tetO, phlO, or vanO. In some embodiments, the upstream activating sequence (UAS) of the synthetic output promoter comprises bmO. In some embodiments, the upstream activating sequence (UAS) of the synthetic output promoter comprises tetO. In some embodiments, the upstream activating sequence (UAS) of the synthetic output promoter comprises phlO. In some embodiments, the upstream activating sequence (UAS) of the synthetic output promoter comprises vanO.

In some embodiments, the synthetic output promoter further comprises one or more operators. In some embodiments, the one or more operators of the synthetic output promoter are not native to the methylotrophic host cell.

In some embodiments, the synthetic transcription factor comprises the DNA-binding domain (DBD) Bm3R1, and the upstream activating sequence (UAS) of the synthetic output promoter comprises one or more copies of bmO. In some embodiments, the synthetic transcription factor comprises the DNA-binding domain (DBD) PhlF_AM and the upstream activating sequence (UAS) of the synthetic output promoter comprises one or more copies of phlO. In some embodiments, the synthetic transcription factor comprises the DNA-binding domain (DBD) TetR and the upstream activating sequence (UAS) of the synthetic output promoter comprises one or more copies of tetO. In some embodiments, the synthetic transcription factor comprises the DNA-binding domain (DBD) VanR_AM and the upstream activating sequence (UAS) of the synthetic output promoter comprises one or more copies of vanO.

In some embodiments, the synthetic output promoter comprises or consists of a polynucleotide having the nucleic acid sequence of any one of SEQ ID NOs: 56-70 or 186-193.

In some embodiments, the gene of interest is expressed as an RNA. In some embodiments, the gene of interest encodes a protein. In some embodiments, the gene of interest encodes an enzyme, a structural protein, a signaling protein, a regulatory protein, a transport protein, a sensory protein, a motor protein, a defense protein, or a storage protein. In some embodiments, the protein synthesizes, modifies, or converts a molecule. In some embodiments, the molecule is heme or an intermediate in a heme biosynthesis pathway. In some embodiments, the protein is a heme-binding protein. In some embodiments, the heme-binding protein is hemoglobin, neuroglobin, cytoglobin, leghemoglobin, or myoglobin. In some embodiments, the protein is vaccinia capping enzyme, T7 polymerase, or O-methyltransferase. In some embodiments, the protein is an enzyme of a heme biosynthesis pathway. In some embodiments, the enzyme of a heme biosynthesis pathway is cytochrome P450, 9-adenylate cyclase, soluble guanylate cyclase, peroxidase, catalase, and/or cytochrome oxidase.

In some embodiments, the methylotrophic host cell further comprises in the second transcriptional unit a polynucleotide encoding a secretion tag. In some embodiments, the secretion tag is an α-amylase secretion tag, an Sc Mf α1 secretion tag, or a pre-inulinase secretion tag. In some embodiments wherein the second transcriptional unit further comprises a secretion tag and wherein the gene of interest encodes a protein, the protein is secreted from the methylotrophic host cell. In some embodiments, the secreted protein is an α-amylase, a β-lactoglobulin, or an ovalbumin.

In some embodiments, the first and/or second transcriptional units further comprise a transcriptional terminator. In some embodiments, the transcriptional terminator of the first and/or second transcriptional unit is naturally occurring. In some embodiments, the transcriptional terminator of the first and/or second transcriptional unit is synthetic. In some embodiments, the transcriptional terminator of the first and/or second transcriptional unit is from a gene encoding a ribosomal protein. In some embodiments, the gene encodes the ribosomal protein S2 (RPS2).

In some embodiments, the transcriptional terminator comprises or consists of a polynucleotide having the nucleic acid sequence of either SEQ ID NO: 146 or 147.

In some embodiments, the first transcriptional unit and the second transcriptional unit are separated by a spacer.

In some embodiments, the first and/or second transcriptional unit is present in the methylotrophic host cell in multiple copies. In some embodiments, the copy number ratio of the first transcriptional unit to the second transcriptional unit is 1:1. In some embodiments, the copy number ratio of the first transcriptional unit to the second transcriptional unit is at least 2:1, at least 4:1, or at least 10:1. In some embodiments, the copy number ratio of the second transcriptional unit to the first transcriptional unit is at least 2:1, at least 4:1, or at least 10:1.

In some embodiments, the first transcriptional unit is present in a single copy and the second transcriptional unit is present in multiple copies. In some embodiments, at least two of the multiple second transcriptional units comprise different genes of interest. In some embodiments, the synthetic transcription factor of the first transcriptional unit is an activator of each synthetic output promoter of the multiple second transcriptional units.

In some embodiments, the synthetic expression system comprises one or more sequences that are endogenous to the methylotrophic host cell.

In some embodiments, the first and second transcriptional units are located on a single plasmid. In some embodiments, the first and second transcriptional units are located on different plasmids. In some embodiments, the first and/or second transcriptional units are integrated into the genome of the methylotrophic host cell. In some embodiments, the first and second transcriptional units are located on the same chromosome in the methylotrophic host cell genome. In some embodiments, the first and second transcriptional units are oriented in the same direction. In some embodiments, the first and second transcriptional units are oriented in different directions. In some embodiments, the first and second transcriptional units are located on different chromosomes in the methylotrophic host cell genome.

In some embodiments, the methylotrophic host cell is a methylotrophic yeast cell. In some embodiments, the methylotrophic host cell is from a genus selected from: Pichia, Komagataella, Hansenula, or Candida. In some embodiments, the methylotrophic host cell is Pichia pastoris, Pichia pseudopastoris, Komagataella phaffii, Pichia stipitis, Pichia membranifaciens, Komagataella pseudopastoris, Komagataella pastoris, Komagataella kurtzmanii, Komagataella mondaviorum, Hansenula polymorpha, Candida boidinii, or Pichia methanolica. In some embodiments, the methylotrophic host cell is Pichia pastoris.

In some embodiments, the synthetic expression system provides for production of a bioproduct encoded by the gene of interest at a level that is higher than the level of the bioproduct produced in a control host cell (i.e., a host cell that does not contain the same synthetic expression system). In some embodiments, the control host cell is a cell of the same species as the methylotrophic host cell. In some embodiments, the control host cell is P. Pastoris. In some embodiments, the control host cell has a native input promoter. In some embodiments, the control host cell has a methanol-inducible promoter operably linked to a gene of interest. In some embodiments, the methanol-inducible promoter of the control host cell is P(AOX1) of P. pastoris. In some embodiments, the control host cell is cultured in the presence of exogenously-added methanol. In some embodiments, the gene of interest encoded by the control host cell is the same gene of interest encoded by the methylotrophic host cell comprising the synthetic expression system.

In some embodiments, the methylotrophic host cell is cultured under conditions comprising a growth phase and a production phase. In some embodiments, the quantity of transcripts of the gene of interest produced in the methylotrophic host cell in the production phase is at least 100% higher than the quantity of transcripts of the gene of interest produced in the methylotrophic host cell in the growth phase. In some embodiments, the quantity of transcripts of the gene of interest produced in the methylotrophic host cell in the production phase is at least 200%, at least 300%, at least 400%, or at least 500% higher than the quantity of transcripts of the gene of interest produced in the methylotrophic host cell in the growth phase.

In some embodiments, the synthetic expression system provides for production of a bioproduct encoded by the gene of interest at a level that is at least 200% higher than the level of the bioproduct produced in a control host cell. In some embodiments, the synthetic expression system provides for production of a bioproduct encoded by the gene of interest at a level that is at least 600%, at least 900%, at least 1200%, at least 1500%, at least 1800%, at least 2100%, at least 2400%, at least 2700%, at least 3000%, at least 5000%, or at least 10,000% higher than the level of the bioproduct produced in a control host cell. In some embodiments, the synthetic expression system provides for production of a bioproduct encoded by the gene of interest at a level that is more than 10,000% higher than the level of the bioproduct produced in a control host cell. In some embodiments, the synthetic expression system provides for production of a bioproduct encoded by the gene of interest at a level that is from about 300% to about 600%, from about 500% to about 1000%, from about 800% to about 1500%, from about 1000% to about 2000%, from about 1200% to about 2000%, from about 1800% to about 2500%, from about 2000% to about 2500%, from about 2200% to about 3000%, from about 3000% to about 5000%, or from about 5000% to about 10,000% higher than the level of the bioproduct produced in a control host cell.

Some aspects of the present invention describe a method of engineering a host cell for protein expression comprising transforming the host cell with the synthetic expression system according to any embodiment of this disclosure.

Other aspects contemplate a method of expressing a gene of interest comprising culturing a methylotrophic host cell comprising a synthetic expression system, transcriptional unit, or component thereof as described in this document. In some embodiments, the gene of interest encodes a heme-binding protein or one or more enzymes of a heme biosynthesis pathway. In some embodiments, the heme-binding protein is hemoglobin, myoglobin, neuroglobin, cytoglobin, or leghemoglobin. In some embodiments, the heme-binding protein is myoglobin. In some embodiments, the one or more enzymes of a heme biosynthesis pathway is cytochrome P450, 9-adenylate cyclase, soluble guanylate cyclase, peroxidase, catalase, and/or cytochrome oxidase. In some embodiments, the gene of interest encodes a vaccinia capping enzyme, T7 polymerase enzyme, or O-methyltransferase enzyme.

Certain aspects of the present invention describe a method of manufacturing a molecule of interest comprising culturing a methylotrophic host cell comprising a synthetic expression system, transcriptional unit, or component thereof as described in this document and obtaining the molecule of interest from biomass or culture. In some embodiments, the molecule of interest is extracted from biomass. In some embodiments, the molecule is collected from culture, culture medium, cell-free spent culture medium, and/or cell-containing culture medium. In some embodiments, wherein the gene of interest encodes an enzyme, the method comprises: (1) purifying the enzyme encoded by the gene of interest; and (2) using the purified enzyme for bioconversion of a substrate to the molecule of interest. In some embodiments, the molecule of interest is heme.

Other aspects contemplate a method of expressing a gene of interest or producing a molecule of interest comprising steps of: (a) culturing a host cell according to the methods of the disclosure in a suitable medium for a period of time to allow cell growth, and (b) changing one or more culture conditions to facilitate expression of the gene of interest or production of the molecule of interest.

In some embodiments, changing one or more culture conditions comprises changing the composition of the culture medium. In some embodiments, step (b) comprises limiting, adding, and/or depleting a nutrient. In some embodiments, step (b) comprises thiamine depletion. In some embodiments, step (b) comprises glycerol limitation. In some embodiments, step (b) comprises monosaccharide limitation. In some embodiments, step (b) comprises formic acid addition. In some embodiments, step (b) comprises limitation of any a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate. In some embodiments, step (b) comprises the limitation of a combination of any two nutrients. In some embodiments, step (b) comprises limitation of glucose and depletion of thiamine.

In some aspects, a synthetic expression system comprises or consists of a polynucleotide having at least 90%, at least 95%, or at least 99% identity to the nucleic acid sequence of any one of SEQ ID NOs: 1-15. In some embodiments, the synthetic expression system comprises or consists of an input promoter comprising a polynucleotide having at least 90%, at least 95%, or at least 99% identity to the nucleic acid sequence of any one of SEQ ID NOs: 16-25. In some embodiments, the synthetic expression system comprises or consists of a polynucleotide encoding at least one component of a synthetic transcription factor. In some embodiments, the polynucleotide comprises or consists of a polynucleotide having at least 90%, at least 95%, or at least 99% identity to the nucleic acid sequence of any one of SEQ ID NOs: 26-40 or 182-185. In some embodiments, the encoded synthetic transcription factor comprises or consists of a polypeptide having at least 90%, at least 95%, or at least 99% identity to the amino acid sequence of any one of SEQ ID NOs: 41-55. In some embodiments, the synthetic expression system comprises or consists of a synthetic output promoter comprising a polynucleotide having at least 90%, at least 95%, or at least 99% identity to the nucleic acid sequence of any one of SEQ ID NOs: 56-70 or 186-193.

In some aspects, a synthetic expression system comprises or consists of a polynucleotide having at least 90%, at least 95%, or at least 99% identity to the nucleic acid sequence of any one of SEQ ID NOs: 16-25.

In some aspects, a synthetic expression system comprises or consists of a polynucleotide having at least 90%, at least 95%, or at least 99% identity to the nucleic acid sequence of any one of SEQ ID NOs: 56-70 or 186-193.

In some aspects, a synthetic expression system comprises or consists of a synthetic transcription factor encoded by a polynucleotide having at least 90%, at least 95%, or at least 99% identity to the nucleic acid sequence of any one of SEQ ID NOs: 26-40 or 182-185.

In some aspects, a synthetic expression system encodes or comprises a synthetic transcription factor comprising or consisting of a polypeptide having at least 90%, at least 95%, or at least 99% identity to the amino acid sequence of any one of SEQ ID NOs: 41-55.

Some aspects of the present invention describe a synthetic expression system comprising: (1) a first transcriptional unit comprising a polynucleotide encoding one or more components of a transcription factor, and (2) a second transcriptional unit comprising a synthetic output promoter. In some embodiments, the transcription factor is an activator of the synthetic output promoter.

In some embodiments, the synthetic expression system is for use in a methylotrophic host cell, such as a methylotrophic yeast. In some embodiments, the synthetic expression system is expressed in a methylotrophic host cell. In some embodiments, the synthetic expression system is expressed in a methylotrophic yeast. In some embodiments, the synthetic expression system is for use or expressed in a methylotrophic host cell. In some embodiments, the methylotrophic host cell is a yeast cell of the genera Pichia or Komagataella. In some embodiments, the synthetic expression system is a methanol-independent expression system for use or expressed in a methylotrophic host cell. In some embodiments, the synthetic expression system is a methanol-independent expression system for use in or expressed in methylotrophic yeast. In some embodiments the yeast is of the genera Pichia or Komagataella.

Some aspects of the present invention describe a synthetic expression system comprising: (1) a first transcriptional unit comprising a polynucleotide encoding at least one component of a transcription factor, and (2) a second transcriptional unit comprising a synthetic output promoter. In some embodiments, the transcription factor is an activator of the synthetic output promoter.

Some aspects of the present invention describe a synthetic, methanol-independent expression system comprising: (1) a first transcriptional unit comprising a polynucleotide encoding one or more components of a transcription factor, and (2) a second transcriptional unit comprising a synthetic output promoter. In some embodiments, the transcription factor is an activator of the synthetic output promoter.

Some aspects of the present invention describe a synthetic, methanol-independent expression system comprising: (1) a first transcriptional unit comprising a polynucleotide encoding one or more components of a transcription factor, and (2) a second transcriptional unit comprising a synthetic output promoter. In some embodiments, the transcription factor is an activator of the synthetic output promoter. In some embodiments, the synthetic, methanol-independent expression system is expressed in a host cell of the genera Pichia or Komagataella.

Each feature of the invention can be encompassed by various aspects of the invention. It is contemplated that each feature of the invention involving any one element or combinations of elements can be included in each embodiment of the invention. This invention is not limited in its application to the details of construction and the arrangement of components set forth in the following description or illustrated in the drawings. The invention is capable of other embodiments and of being practiced or carried out in various ways.

### BRIEF DESCRIPTION OF DRAWINGS

The accompanying drawings are not intended to be drawn to scale. The drawings are illustrative and non-limiting examples only and are not required for enablement of the disclosure. For purposes of clarity, not every component may be labeled in every drawing. In the drawings:

FIGS. 1A-1B each show a schematic for a non-limiting example of a synthetic expression system of this disclosure, for example, one that is inducible under a methanol-independent fermentation process. FIG. 1A shows that the first transcriptional unit is made up of an input promoter (P(in)) operably linked to a polynucleotide encoding a synthetic transcription factor (sTF) and, optionally, a first transcriptional terminator (TT). FIG. 1B shows that the first transcriptional unit is made up of a P(in) comprising an upstream activating sequence (UAS) and core promoter element operably linked to a polynucleotide encoding an sTF and, optionally, a first TT. In some embodiments, the P(in) comprises only a core promoter element (e.g., no UAS). In some embodiments as shown in FIG. 1A or FIG. 1B, P(in) becomes activated in response to a manipulation of the fermentation process (culturing) conditions. The manipulation activates transcription of the sTF-encoding gene. Both FIGS. 1A and 1B show that the second synthetic transcriptional unit comprises a synthetic output promoter (P(out)) operably linked to a gene of interest (GOI) and, optionally, a second TT, where the first and second TT are the same or different. P(out) comprises an upstream activating sequence (UAS) operably linked to a core promoter (CP) sequence. Once expressed, the sTF protein binds to one or more cognate sites (e.g., operators) in the upstream activating sequence of P(out), thereby activating transcription from the core promoter. P(out) is operably linked to the gene of interest and drives its expression.

FIG. 2 depicts components of two non-limiting examples of a synthetic transcription factor (sTF). An sTF may be a one-component sTF, a two-component sTF, or a multi-component (e.g., three or more component) sTF. The one-component sTF may comprise a DNA binding domain (DBD), a nuclear localization signal (NLS, optional), a linker (L, optional), and a transcriptional activation domain (TAD). The two-component sTF may comprise a DBD, NLS (optional), L (optional), a Bioconjugate Protein Part 1 (BPP1, optional), a 2A peptide (2A, optional), a Bioconjugate Protein Part 2 (BPP2, optional), an NLS (optional), an Oligomerization Domain (OD, optional), and a TAD. Together, in the two-component sTF, the DBD, NLS (optional), linker (optional), and BPP1 form Component 1. Together, in the two-component sTF, the BPP2, NLS (optional), OD (optional), and TAD form Component 2. Component 1 and Component 2 come together to form a “Bioconjugated (synthetic) Transcription Factor” (B(s)TF) comprising the Component 1-Component 2 adduct.

FIG. 3 shows a non-limiting example of a fermentation (culturing) flow diagram. Three stages of fermentation are shown. The batch phase of fermentation begins (Stage I) with a starting culture medium containing a fixed mass of carbon added in the beginning. Stage II, the fed-batch phase, is a biomass generation phase, in which a carbon feed is maintained at a rate sufficient to sustain growth while maintaining a desired residual carbon level. Stage III is the production phase. In Stage III the carbon feed rate can be adjusted to sustain production while maintaining a desired residual carbon level. Addition (e.g., either as a one-time bolus or a feed) or limitation or depletion (e.g., of a nutrient) may be made throughout the fermentation process as necessary.

FIG. 4 depicts the test and control constructs that were integrated into the host cell genome in Example 1. The upper construct depicts the synthetic expression systems (SESs) that were integrated and tested, while the lower construct depicts a control.

FIG. 5 shows an assay of expression in deepwell plates (Assay 3). Each strain contains one synthetic expression system (SES) expressing one of three proteins, each with a luminescent tag. Each strain is assayed in a deepwell process corresponding to its P(in). One to three colonies were picked and assayed per unique SES. The performance of 241 picks is summarized. Average background luminescence is approximately 337 Luminescence Units.

FIG. 6 shows an assay of expression in deepwell plate format in Process 3. Each dot represents a unique strain producing nine proteins. The values in the upper panel represents the amount of intracellular myoglobin while those in the lower panel represents the amount of heme in the same cell.

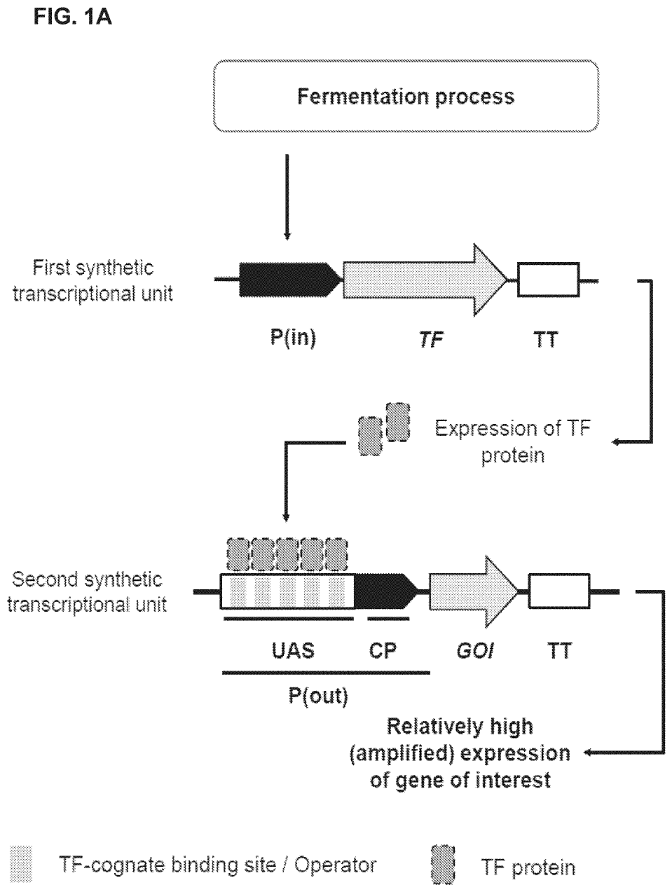

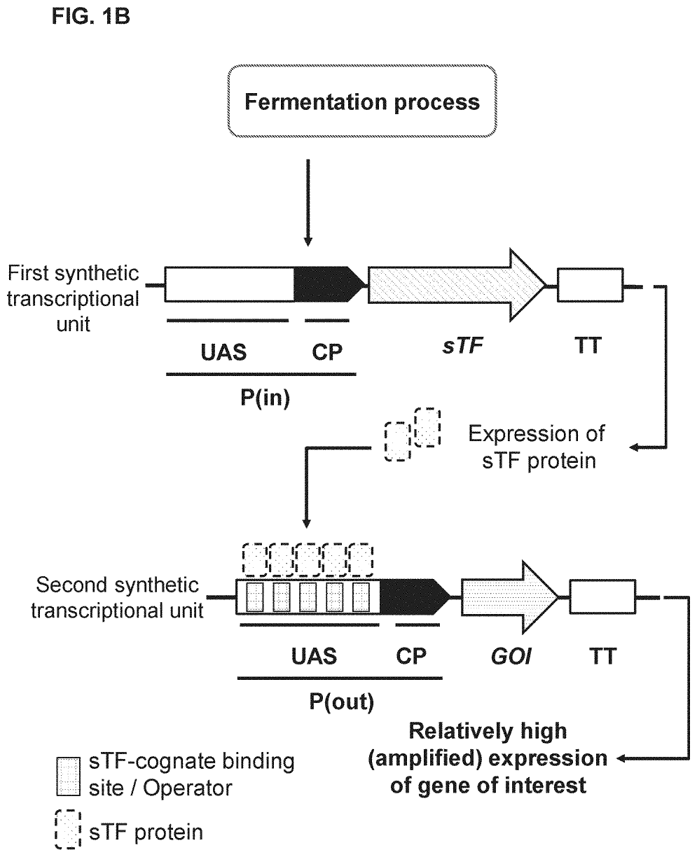

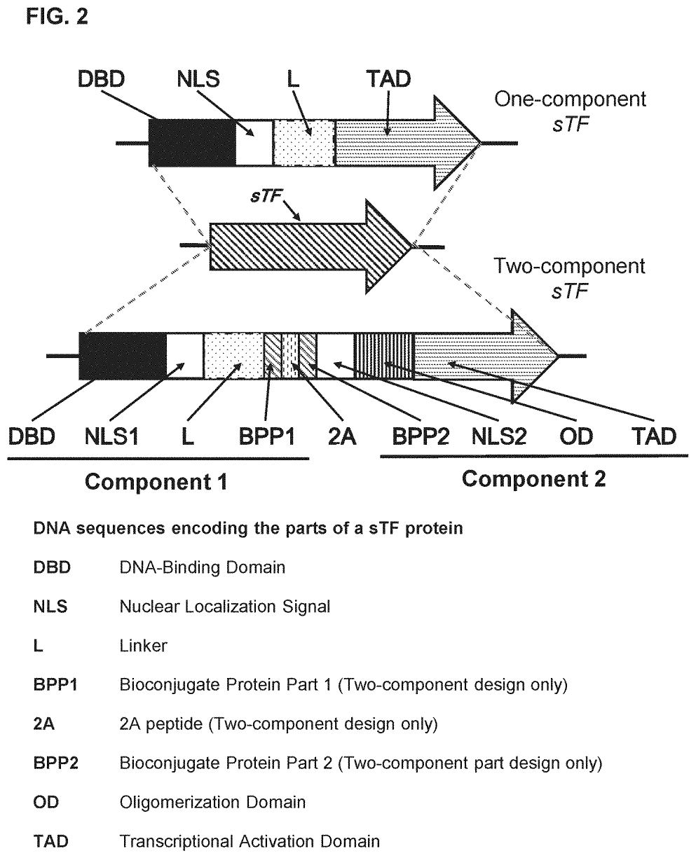

### DETAILED DESCRIPTION

This disclosure provides synthetic expression systems, transcriptional units, host cells comprising synthetic expression systems and transcriptional units, and methods that facilitate high-yield production of desired bioproducts (e.g., without limitation, enzymes or other proteins, RNA, small molecules, etc.), for example, in methanol-independent conditions. “Synthetic” refers to a sequence (e.g., a nucleic acid sequence or an amino acid sequence) that is not naturally occurring, or to a component which includes one or more sequences that are not naturally occurring. “Naturally occurring” refers to something (e.g., a nucleic acid or polypeptide) that can be found in nature. For example, a naturally occurring nucleic acid or polypeptide sequence is one that can be isolated from a source in nature and has not otherwise been modified by a human in a laboratory. In some embodiments, a sequence that is not naturally occurring includes two or more naturally occurring sequences that are combined to form a new sequence.

The transcriptional units and synthetic expression systems of the present disclosure may comprise several components, which may include input promoters, synthetic output promoters, polynucleotides encoding transcription factors (e.g., transcriptional activators), genes of interest to be expressed, and optional transcriptional terminators. These components may be used in conjunction with other components to create a transcriptional unit or system.

In some embodiments, a synthetic expression system comprises a first transcriptional unit. In some embodiments, the first transcriptional unit comprises a polynucleotide encoding at least one component of a transcription factor. In some embodiments, the first transcriptional unit further comprises an input promoter operably linked to and capable of expressing the polynucleotide encoding the at least one component of a transcription factor. In some embodiments, the first transcriptional unit comprises a polynucleotide encoding a transcription factor. In some embodiments, the first transcriptional unit comprises a polynucleotide encoding a transcription factor or at least one component of a transcription factor, and an insertion site. In some embodiments, in a first transcriptional unit, an insertion site is located such that a promoter (e.g., an input promoter) inserted into the insertion site is operably linked to and capable of expressing the polynucleotide encoding the transcription factor or at least one component of a transcription factor. In some embodiments, the first transcriptional unit comprises an input promoter that has been inserted into the insertion site. In some embodiments, the input promoter is operably linked to and regulates transcription of the polynucleotide encoding a transcription factor or at least one component of a transcription factor.

In some embodiments, the synthetic expression system comprises a second transcriptional unit comprising an output promoter. In some embodiments, the synthetic expression system comprises a second transcriptional unit comprising an output promoter and an insertion site. In some embodiments, in a second transcriptional unit, an insertion site is located such that a gene of interest inserted into the insertion site is operably linked to and capable of being expressed from the output promoter. In some embodiments, the transcription factor (a portion or all of which is encoded by the first transcriptional unit) is an activator of the output promoter of the second transcriptional unit. In some embodiments, the output promoter is operably linked to and regulates transcription of a gene of interest, wherein the transcription factor encoded by the first transcriptional unit is an activator of the output promoter of the second transcriptional unit. In some embodiments, the transcription factor and/or the output promoter are synthetic. The present disclosure also pertains to a host cell comprising a synthetic expression system, and methods of using the host cell, a transcriptional unit, or a synthetic expression system.

In some embodiments, a synthetic expression system within a host cell can be used to produce a bioproduct. In some embodiments, design of the synthetic expression system, selection of the host cell, and parameters of culturing conditions can be manipulated to control the timing and level of production of the bioproduct.

### Synthetic Expression Systems (SES)

Aspects of this disclosure provide transcriptional units and synthetic expression systems which may be useful, for example, in the biosynthesis of desired bioproducts.

As used in this disclosure, a “synthetic expression system” refers to a non-naturally occurring expression system that enables expression of genes of interest [for example, endogenous and/or synthetic (e.g., modified, heterologous, or exogenous-to-the-host-cell, etc.) genes of interest] for the purpose of synthesizing desired bioproducts. In some embodiments, the synthetic expression system comprises one or more transcriptional units. In some embodiments, the first and/or second transcriptional units are synthetic.

In some embodiments, a synthetic expression system comprises a first transcriptional unit comprising a polynucleotide encoding a transcription factor (e.g., a transcriptional activator) or at least one component of a transcription factor, and a second transcriptional unit comprising an output promoter which is cognate to the transcription factor. In some embodiments, a synthetic expression system comprises a first transcriptional unit comprising a first insertion site and a polynucleotide encoding a transcription factor (e.g., a transcriptional activator) or at least one component of a transcription factor, and a second transcriptional unit comprising an output promoter which is cognate to the transcription factor and a second insertion site, wherein a promoter (e.g., an input promoter) inserted into the first insertion site is operably linked to and capable of facilitating the expression of the polynucleotide, and wherein a gene of interest inserted into the second insertion site is operably linked to and capable of being expressed by the output promoter. In some embodiments, a synthetic expression system comprises one or more of the following components: (a) a first transcriptional unit, which comprises: an input promoter operably linked to and capable of expressing a polynucleotide encoding a transcription factor or at least one component of a transcription factor; and (b) a second transcriptional unit, which comprises: an output promoter operably linked to and capable of expressing a gene of interest, and, optionally, a transcriptional terminator downstream of the gene of interest. In some embodiments, a transcription factor is a synthetic transcription factor (sTF). In some embodiments, a synthetic transcription factor can be a one-component sTF, two-component sTF, or multi-component sTF. In some embodiments, an input promoter (P(in)) of a first transcriptional unit, the transcriptional activity of which is regulated by certain culture conditions, drives expression of a polynucleotide encoding a transcription factor or at least one component of a transcription factor, which in turn mediates the transcriptional activation of one or more second transcriptional units. In some embodiments, each second transcriptional unit comprises an output promoter (P(out)) that comprises binding sites for the transcription factor or the at least one component of a transcription factor, and a gene of interest. In some embodiments, the output promoter is synthetic. In some embodiments, an output promoter comprises an upstream activating sequence (UAS), which can comprise one or more binding sites for the transcription factor; a core promoter; and a 5′-untranslated region (5′-UTR). In some embodiments, an input promoter comprises a 5′-UTR. The 5′-UTR is the portion of the promoter from the +1 transcriptional start (inclusive) to the ATG translation start (not inclusive).

The Examples, including Examples 1, 3, and 4, illustrate several non-limiting examples of transcriptional units and synthetic expression systems of this disclosure.

It will be appreciated by those of skill in the art that the transcriptional units or synthetic expression systems of this disclosure can be configured in various ways within the host cell genome (e.g., on contiguous or non-contiguous polynucleotide sequences; on the same or different chromosomes; oriented in the same or the opposite direction with respect to the direction of transcription).

In some embodiments, the synthetic expression system (e.g., including a first and second transcriptional unit) is located on a single plasmid or chromosome. In some embodiments, the synthetic expression system is located on two or more plasmids and/or chromosomes. For example, in some embodiments, the first transcriptional unit is located on a first plasmid or chromosome, and the second transcriptional unit is located on a second plasmid or chromosome.

In some embodiments, a synthetic expression system comprises two or more copies of a first transcriptional unit; and/or two or more copies of a second transcriptional unit.

In some embodiments, a synthetic expression system comprises two or more first transcriptional units, which are the same as or different from each other; and/or two or more second transcriptional units, which are the same as or different from each other.

In some embodiments, a synthetic expression system is capable of expressing two or more copies of the same or different genes of interest. In some embodiments, a synthetic expression system is capable of producing two or more different bioproducts.

In some embodiments, the first transcriptional unit is present in a single copy and the second transcriptional unit is present in multiple copies (e.g., two or more copies). In some embodiments, at least two of the multiple second transcriptional units comprise different genes of interest. For example, second transcriptional unit #1 may comprise a gene encoding an enzyme of a heme biosynthesis pathway, and second transcriptional unit #2 may comprise a gene encoding heme or an intermediate in a heme biosynthesis pathway. In some embodiments, the transcription factor (e.g., synthetic transcription factor) of the first transcriptional unit is an activator of each output promoter (e.g., synthetic output promoter) of the multiple second transcriptional units. However, it will be understood that the multiple second transcriptional units need not comprise identical output promoters (or output promoters comprising identical components) in order for the transcription factor (e.g., synthetic transcription factor) of the first transcriptional unit to activate each output promoter (e.g., synthetic output promoter) of the multiple second transcriptional units. For example, the output promoters (e.g., synthetic output promoters) of the multiple second transcriptional units may each comprise different core promoter elements, but may share a common upstream activation sequence (UAS) and thereby each be activated by the transcription factor (e.g., synthetic transcription factor) of the first transcriptional unit.

In some embodiments, a synthetic expression system comprises at least two first transcriptional units, each comprising a different input promoter operably linked to a polynucleotide encoding the same transcription factor or at least one component of a transcription factor; and/or at least two second transcriptional units, each comprising an output promoter activatable by the transcription factor and operably linked to a gene of interest, wherein the output promoters and the genes of interest of the at least two second transcriptional units are the same or different.

In some embodiments, a synthetic expression system comprises a first transcriptional unit capable of expressing a transcription factor or at least one component of a transcription factor; and two or more different second transcriptional units, each comprising a synthetic output promoter activated by the transcription factor and operably linked to a different gene of interest.

In some embodiments, a first transcriptional unit comprises: an input promoter, which is operably linked to two or more polynucleotides, each expressing the same or a different transcription factor or at least one component of a transcription factor (e.g., a polycistronic system or locus). In some embodiments, the same or different transcription factors activate transcription of the same or different genes of interest.

In some embodiments, a synthetic expression system comprises: (a) a first transcriptional unit comprising: an input promoter, which is operably linked to two or more polynucleotides, each expressing the same or a different transcription factor or at least one component of a transcription factor; and (b) one or more second transcriptional units, each of which comprises a synthetic output promoter, which is activated by a transcription factor, and which is operably linked to a gene of interest; wherein the synthetic output promoters and/or genes of interest of the one or more second transcriptional units are the same or different.

In some embodiments, a functioning unit of DNA comprises two or more genes under the control of the same promoter (e.g., a multicistronic or polycistronic unit). In various embodiments, the transcriptional unit comprising the gene encoding the transcription factor or at least one component of a transcription factor is multicistronic or polycistronic [e.g., the transcriptional unit encodes multiple different transcription factors (or components thereof) or multiple copies of the same transcription factor (or component thereof)]; and/or the transcriptional unit comprising the gene of interest is multicistronic or polycistronic (e.g., the transcriptional unit encodes multiple different genes of interest or multiple copies of the same gene of interest). In some embodiments, a first transcriptional unit comprises a single input promoter operably linked to two or more polynucleotides encoding the same or different transcription factors, or components thereof. In some embodiments, a second transcriptional unit comprises a single output promoter operably linked to two or more genes of interest (which can be the same or different).

In some embodiments, a synthetic expression system comprises: (a) a transcriptional unit comprising: an input promoter, which is operably linked to two or more polynucleotides, each expressing a different transcription factor or at least one component of a transcription factor; and (b) two or more second transcriptional units, each comprising a synthetic output promoter activated by a different transcription factor or at least one component of a transcription factor encoded by the first transcriptional unit and operably linked to a different gene of interest.

In some embodiments, a host cell comprises in its genome multiple copies of a transcriptional unit or synthetic expression system, such as any one of the transcriptional units or systems described in this application. These multiple copies may in some embodiments result from multiple introductions of the system or unit into the host cell genome, or from a single introduction followed by self-replication of one or more plasmids comprising the synthetic expression system, unit(s), or components thereof.

In some embodiments, a synthetic expression system comprises one or more of the following components: input promoters, polynucleotides encoding transcription factors or at least one component thereof, synthetic output promoters, genes of interest, and transcriptional terminators (see, e.g., Examples 1, 3, and 4). Examples 1, 3, and 4 describe several non-limiting examples of transcriptional units and synthetic expression systems of this disclosure. In some embodiments, a synthetic expression system of this disclosure comprises or consists of a sequence (e.g., nucleic acid or amino acid sequence) that is at least 90%, at least 91%, at least 92%, at least 93%, at least 94%, at least 95%, at least 96%, at least 97%, at least 98%, at least 99%, or 100% identical, to a sequence in Examples 1 and 3, Tables 21, 28, and 30-36, or to a sequence selected from any one of SEQ ID NOs: 1-166.

In some embodiments, a host cell comprises a synthetic expression system that comprises one or a multitude of different first and/or second transcriptional units. The various transcriptional units can be in any orientation relative to each other.

In some embodiments, a spacer can be placed between any two components of a transcriptional unit or a synthetic expression system. Spacers are typically short polynucleotide or amino acid sequences that can be inserted between components for various purposes, e.g., to decrease or facilitate interactivity. A spacer is an optional part type, and is not considered to be necessary for proper functioning of the transcriptional units and/or synthetic expression systems of this disclosure. A non-limiting example of a spacer is described in Table 20, and the corresponding DNA sequence of this part type is provided in Table 21. In some embodiments, a spacer comprises a polynucleotide having the sequence of SEQ ID NO: 166.

In some embodiments, a synthetic expression system comprises, in order from 5′ to 3′, the DNA sequence of the following parts: (1) P(in), (2) polynucleotide encoding a TF (e.g., a sTF) or at least one component of a transcription factor, and (3) TT of the first transcriptional unit, (4) an optional spacer, (5) P(out), and (6) a gene of interest. In some embodiments, a synthetic expression system comprises, in order from 5′ to 3′, the DNA sequence of the following parts: (1) P(in), (2) polynucleotide encoding a TF (e.g., a sTF) or at least one component of a transcription factor, and (3) TT of the first transcriptional unit, (4) an optional spacer, (5) P(out), (6) a secretion tag, and (7) a gene of interest. In some embodiments, a synthetic expression system comprises, in order from 5′ to 3′, the DNA sequence of the following parts: (1) P(in), (2) polynucleotide encoding a TF (e.g., a sTF) or at least one component of a transcription factor, and (3) TT of the first transcriptional unit, (4) an optional spacer, (5) P(out), (6) a secretion tag, (7) a detection tag, and (8) a gene of interest.

In some embodiments, an expression system is cognate with respect to a production process. In some embodiments, an expression system is cognate with respect to a particular production process (e.g., a process described in this document such as Process 1, Process 2, Process 3, or Process 4) if the expression system is activated under a particular culturing step or condition in that process (e.g., for Process 1, limiting glycerol and added formic acid; for Process 2, limiting glucose and added formic acid; and for Process 3, limiting glucose and depleted thiamine).

Table 2 shows the combinatorial design of non-limiting examples of synthetic expression systems. These are useful for, among other things, a production process according to Process 2, wherein glucose is limiting and formic acid is added.

Table 3 shows the combinatorial design of non-limiting examples of synthetic expression systems. These are useful for, among other things, a production process according to Process 3, wherein glucose is limiting and thiamine is depleted.

In the process of designing, constructing, and/or evaluating a synthetic expression system, a reporter gene can be used as the gene of interest. As shown in the Examples, several synthetic expression systems were constructed using red fluorescent protein (RFP) as a reporter gene. After successful evaluation of a synthetic expression system (for example, those described in the Examples), if a reporter gene was used as the gene of interest, the reporter gene can be replaced by a different gene of interest useful for production of a particular bioproduct.

In some embodiments, a synthetic expression system is methanol-independent. In some embodiments, the methanol-independent synthetic expression system comprises (a) a first transcriptional unit comprising a polynucleotide encoding a transcription factor or at least one component of a transcription factor, and (b) a second transcriptional unit comprising a synthetic output promoter. In some embodiments, one or more components of the transcription factor is an activator of the synthetic output promoter of the second transcriptional unit. In some embodiments, the synthetic, methanol-independent expression system is expressed in a host cell of the genera Pichia, Komagataella, Hansenula, Candida, or any yeast, including but not limited to any methylotrophic yeast.

Certain aspects of the present disclosure encompass synthetic expression systems for use in yeast under fermentation conditions of limiting glycerol and added formic acid (Process 1 as described in the Examples). Such synthetic expression systems comprise an input promoter (P(in)) operably linked to a synthetic transcription factor (sTF), and a synthetic output promoter (P(out)) operably linked to a gene of interest. In some embodiments, the P(in) may be selected from P(GQ6704499), P(HGT1), and P(FDH1) (a non-limiting example of a specific sequence for each of these promoters may be found in Table 21; suitable variants of these promoters are within the skill of one in the art, as detailed above). In some embodiments, the sTF may be selected from a TetR-based one-component system, a VanR_AM-based one-component system, a PhlF-based one component system, or a PhlF-based two component system. Non-limiting examples of specific sequences for each of these sTFs may be found in Tables 30 and 36 (nucleic acid sequences) and 31 (amino acid sequences); suitable variants of these sTFs are within the skill of one in the art, as detailed above). In some embodiments, P(out) is selected from a P(AOX1) or P(HHF2) core promoter modified with 8×tetO, 4×vanO, 8×phlO, 1×tetO, or 2×phlO (a non-limiting example of a specific sequence for each P(out) may be found in Table 33 or Table 36; suitable variants of these promoters are within the skill of one in the art, as detailed above).

Certain aspects of the present disclosure also encompass synthetic expression systems for use in yeast under fermentation conditions of limiting glucose and added formic acid (Process 2 as described in the Examples). Such synthetic expression systems comprise an input promoter (P(in)) operably linked to a synthetic transcription factor (sTF), and a synthetic output promoter (P(out)) operably linked to a gene of interest. In some embodiments, the P(in) may be selected from P(AOX2), P(RGI2), and P(FDH1) (a non-limiting example of a specific sequence for each of these promoters may be found in Table 21; suitable variants of these promoters are within the skill of one in the art, as detailed above). In some embodiments, the sTF may be selected from a Bm3R1-based one-component system, a PhlF-based one component system, or a PhlF-based two component system. Non-limiting examples of specific sequences for each of these sTFs may be found in Tables 30 and 36 (nucleic acid sequences) and 31 (amino acid sequences); suitable variants of these sTFs are within the skill of one in the art, as detailed above). In some embodiments, P(out) is selected from a P(AOX1) or P(PMP20) core promoter modified with 4×bmO, 8×bmO, 8×phlO, or 2×phlO (a non-limiting example of a specific sequence for each P(out) may be found in Table 33 or Table 36; suitable variants of these promoters are within the skill of one in the art, as detailed above).

Certain aspects of the present disclosure also encompass synthetic expression systems for use in yeast under fermentation conditions of limiting glucose and depleted thiamine (Process 3 as described in the Examples). Such synthetic expression systems comprise an input promoter (P(in)) operably linked to a synthetic transcription factor (sTF), and a synthetic output promoter (P(out)) operably linked to a gene of interest. In some embodiments, the P(in) may be selected from P(THI13)_short and P(THI13)_long (a non-limiting example of a specific sequence for each of these promoters may be found in Table 21; suitable variants of these promoters are within the skill of one in the art, as detailed above). In some embodiments, the sTF may be selected from a Bm3R1-based two-component system or a PhlF-based two component system. Non-limiting examples of specific sequences for each of these sTFs may be found in Tables 30 and 36 (nucleic acid sequences) and 31 (amino acid sequences); suitable variants of these sTFs are within the skill of one in the art, as detailed above). In some embodiments, the P(out) promoter is a P(AOX1) core promoter modified with 2×bmO or 2×phlO (a non-limiting example for each P(out) may be found in Table 33 or Table 36; suitable variants of these promoters are within the skill of one in the art, as detailed above).

### Bioproducts

The transcriptional units, synthetic expression systems, host cells, and other methods described in this disclosure can be used for the high-yield, large-scale production of bioproducts, for example, in methanol-independent conditions.

The term “bioproduct” refers to any product that is made by or from biomass and which may be expressed by a transcriptional unit and/or synthetic expression system of this disclosure. “Biomass” refers to any biological material that is available on a renewable basis, including by production in any host cells.

In some embodiments, a bioproduct is a protein expressed from a gene of interest, or a polynucleotide; or any other composition that is, directly or indirectly, synthesized, modified, or otherwise acted upon by a protein or polynucleotide expressed from a gene of interest.

In some embodiments, a bioproduct is a protein, nucleic acid (e.g., mRNA; or polynucleotide), small or large molecule, complex or supramolecular complex (or a component of either), or a compound or composition that is, directly or indirectly, synthesized (in whole or in part), modified, and/or converted into another, a final, or a more useful or stable form by the action of the protein or nucleic acid encoded by a gene of interest.

In some embodiments, where a gene of interest expresses a protein, the protein is an enzyme, a structural protein, a signaling protein, a regulatory protein, a transport protein, a sensory protein, a motor protein, a defense protein, or a storage protein.

In some embodiments, the protein is an enzyme. In some embodiments, a protein expressed by a gene of interest is an enzyme of a heme biosynthesis pathway. In some embodiments, an enzyme expressed by a gene of interest is one or more of cytochrome P450, 9-adenylate cyclase, soluble guanylate cyclase, peroxidase, catalase, or cytochrome oxidase.

In some embodiments, the protein synthesizes, modifies, or converts a molecule. In some embodiments, the molecule is heme.

In some embodiments, synthetic expression systems are used to produce a heme-binding protein. Various classes of heme-binding proteins that can be expressed using the transcriptional units or synthetic expression systems of this disclosure include, without limitation, globins (e.g., hemoglobin, myoglobin, neuroglobin, cytoglobin, leghemoglobin), cytochromes (e.g., a-, b-, and c-types, cdl-nitrite reductase, cytochrome oxidase), transferrins (e.g., lactotransferrin, serotransferrin, melanotransferrin), bacterioferritins, hydroxylamine oxidoreductase, nitrophorins, peroxidases (e.g., lignin peroxidase), cyclooxygenases (e.g., COX-1, COX-2, COX-3, prostaglandin H synthase), catalases, cytochrome P-450s, chloroperoxidases, PAS-domain heme sensors, H-NOX heme sensors (e.g., soluble guanylate cyclase, FixL, DOS, HemAT, and CooA), heme-oxygenases, and nitric oxide synthases. In some embodiments, the recombinant heme-binding protein expressed using the transcriptional units or synthetic expression systems of this disclosure can be of prokaryotic or eukaryotic origin. In some embodiments the heme-binding protein is of mammalian origin. In some embodiments the heme-binding protein is of bovine origin. In some embodiments, the heme-binding protein is of bacterial origin. In some embodiments, the heme-binding protein is of fungal (e.g., yeast) origin. In some embodiments, the heme-binding protein is of plant origin, or of any other origin.

In some embodiments, one or more synthetic expression systems are used to produce heme and a heme binding protein in a host cell.

In some embodiments, the synthetic expression system further comprises in the second transcriptional unit a polynucleotide encoding a secretion tag. In some embodiments, the secretion tag is native to the host cell. In some embodiments, the secretion tag is naturally occurring, but not native to the host cell. In some embodiments, the secretion tag is Pre-OST1-Pro Sc MF alpha 1, murine IgG1, PHA-E, Sc invertase, Sc MEL1, Sc INU, YILip11, YILip2, Dan4, GAS1, MSB2, FRE2, PHO1, PHO5, SOD1, EXG1, BGL2, CPR5, YPS1, ENO1, PEP4, THI4, ILV5, CTR9, PIR3, FLO10, HSP150, NU145, MUC1, ROT1, or MET6. In some embodiments, the secretion tag is an α-amylase secretion tag, an Sc Mf al secretion tag, or a pre-inulinase secretion tag. In some embodiments wherein the second transcriptional unit further comprises a secretion tag and wherein the gene of interest encodes a protein, the protein is capable of being secreted from the host cell. As will be understood, in some embodiments secretion of the protein encoded by the synthetic expression system from a host cell is advantageous, because the host cell does not need to be lysed or otherwise damaged in order to extract and purify the encoded protein. Thus, in some embodiments the host cell is able to continue to produce proteins of interest even after collection of the encoded protein. In some embodiments, the secreted protein is an α-amylase, a β-lactoglobulin, or an ovalbumin.

In some embodiments, a bioproduct is a nucleic acid transcribed from a gene of interest (e.g., an mRNA). In some embodiments, a bioproduct is an mRNA that encodes a viral protein. In some embodiments, a bioproduct is an mRNA that encodes a SARS-CoV-2 viral protein and is useful as a vaccine against COVID-19. In some embodiments, a SARS-CoV-2 viral protein is a spike protein. In some embodiments, a bioproduct is an mRNA that encodes a viral protein and is useful as an mRNA vaccine. In some embodiments, the bioproduct is a vaccinia capping enzyme. In some embodiments, the bioproduct is an O-methyltransferase or T7 polymerase.

In some embodiments, the bioproduct is a small molecule. In some embodiments, the small molecule is heme.

In some embodiments, a bioproduct is a small or large molecule which is synthesized (in whole or in part), modified, and/or converted into another, a final or a more useful or stable form, directly or indirectly, by the action of a protein expressed from a gene of interest.

In some embodiments, a bioproduct is a complex or supramolecular complex comprising any one or more of: RNA(s), protein(s), and/or large or small molecules; or a bioproduct is a component of such a complex or supramolecular complex.

In some embodiments, a bioproduct is a component (e.g., a protein, nucleic acid, small or large molecule, etc.) which is useful in a bioconversion process.

### Measuring Bioproducts

The amount of production of a bioproduct may be evaluated at any one or multiple steps of a pathway, such as a final product or an intermediate product, using metrics familiar to those of skill in the art. Production may be assessed by any metric known in the art, for example, by assessing volumetric productivity, enzyme kinetics/reaction rate, specific productivity, biomass-specific productivity, titer, yield, and total titer of one or more bioproducts.

In some embodiments, the metric used to measure production may depend on whether a continuous process is being monitored or whether a particular end product is being measured. For example, in some embodiments, metrics used to monitor production by a continuous process may include volumetric productivity, enzyme kinetics, and reaction rate. In some embodiments, metrics used to monitor production of a particular product may include specific productivity, biomass-specific productivity, activity, titer, and yield of one or more bioproducts. The term “volumetric productivity” or “production rate” refers to the amount of product formed per volume of medium per unit of time. Volumetric productivity can be reported in grams per liter per hour (g/L/h).

It should be appreciated that bioproducts can be measured by any means known to one of ordinary skill in the art.

In some embodiments, the bioproducts may be determined by, e.g., measuring the amount of bioproduct produced per unit biomass per unit time. For example, the bioproducts may be measured in, e.g., mmol bioproduct produced per liter of fermentation medium per hour. In some embodiments, a transcriptional unit or synthetic expression system of this disclosure, or a host cell comprising a transcriptional unit or synthetic expression system of this disclosure, may produce at least 0.1 mmol, at least 1 mmol, at least 1.5 mmol, at least 2 mmol, at least 2.5 mmol, at least 3, at least 3.5 mmol, at least 4 mmol, at least 4.5 mmol, at least 5 mmol, at least 5.5 mmol, at least 6 mmol, at least 6.5 mmol, at least 7 mmol, at least 7.5 mmol, at least 8 mmol, at least 8.5 mmol, at least 9 mmol, at least 9.5 mmol, or at least 10 mmol of bioproduct. In some embodiments, a transcriptional unit or synthetic expression system of this disclosure, or a host cell comprising a transcriptional unit or synthetic expression system of this disclosure, may produce from about 0.1 to about 0.6 mmol, about 0.5 to about 1 mmol, about 0.9 to about 1.4 mmol, about 1.3 to about 1.8 mmol, about 1.7 to about 2.5 mmol, about 2.4 to about 2.9 mmol, about 2.8 to about 3.3 mmol, about 3.2 to about 3.7 mmol, about 3.6 to about 4.1 mmol, about 4 to about 4.5 mmol, about 4.4 to about 4.9 mmol, about 4.8 to about 5.3 mmol, about 5.2 to about 5.7 mmol, about 5.6 to about 6.1 mmol, about 6 to about 6.5 mmol, about 6.4 to about 6.9 mmol, about 6.8 to about 7.3 mmol, about 7.2 to about 7.7 mmol, about 7.6 to about 8.1 mmol, about 8 to about 8.5 mmol, about 8.4 to about 8.9 mmol, about 8.8 to about 9.3 mmol, about 9.2 to about 9.7 mmol, or about 9.6 to about 10 mmol of bioproduct. In some embodiments, a transcriptional unit or synthetic expression system of this disclosure, or a host cell comprising a transcriptional unit or synthetic expression system of this disclosure, may produce from about 0.1 to about 3 mmol, about 0.5 to about 4 mmol, about 1 to about 4.5 mmol, about 2 to about 5 mmol, about 2.5 to about 5 mmol, about 3 to about 7 mmol, about 3.5 to about 7.5 mmol, about 4 to about 8 mmol, about 4.5 to about 9 mmol, about 5 to about 10 mmol, about 6 to about 10 mmol, about 7 to about 10 mmol, or about 8 to about 10 mmol of bioproduct.

In some embodiments, the bioproducts may be determined by, e.g., measuring the quantity of transcripts of bioproduct produced by the cell per million total transcripts (of any identity) produced by the cell (e.g., “transcripts per million”). In some embodiments, a transcriptional unit or synthetic expression system of this disclosure, or a host cell comprising a transcriptional unit or synthetic expression system of this disclosure, may produce at least 300 transcripts per million, at least 500 transcripts per million, at least 1000 transcripts per million, at least 5000 transcripts per million, at least 10,000 transcripts per million, at least 50,000 transcripts per million, at least 100,000 transcripts per million, at least 300,000 transcripts per million, at least 400,000 transcripts per million, at least 500,000 transcripts per million, or at least 600,000 transcripts per million of bioproduct. In some embodiments, a transcriptional unit or synthetic expression system of this disclosure, or a host cell comprising a transcriptional unit or synthetic expression system of this disclosure, may produce more than 600,000 transcripts per million of bioproduct.

In some embodiments, the bioproducts may be determined by, e.g., comparing the quantity or amount of bioproduct produced by a host cell comprising a synthetic expression system of the disclosure to a control host cell. In some embodiments, the synthetic expression system provides for production of a bioproduct encoded by the gene of interest at a level that that is higher than the level of the bioproduct produced in a control host cell. In some embodiments, the control host cell is a cell that comprises a methanol-inducible promoter, such as P(AOX1) of P. pastoris, operably linked to a gene of interest. In some embodiments, the control host cell is a cell that comprises a methanol-inducible promoter, such as P(AOX1) of P. pastoris, operably linked to a gene of interest. In some embodiments, the gene of interest encoded by the control host cell is the same gene of interest encoded by the methylotrophic host cell. In some embodiments, the methanol-inducible promoter of the control host cell is P(AOX1) of P. pastoris. In some embodiments, the control host cell is cultured in the presence of exogenously-added methanol. In some embodiments, the control host cell is a cell that comprises a P(AOX1) of P. pastoris operably linked to the same gene of interest as the gene of interest of the synthetic expression system, and is cultured in the presence of exogenously-added methanol. In some embodiments, the exogenously-added methanol induces P(AOX1). In some embodiments, the control host cell and the host cell comprising the synthetic expression system are of the same species.

In some embodiments, the control host cell comprises a transcriptional unit or synthetic expression system according to this disclosure, but is cultured in different (e.g., methanol-dependent) conditions than a host cell comprising an identical transcriptional unit or synthetic expression system, wherein the host cells are of the same type. In some embodiments, the control host cell comprises an endogenous transcriptional unit or expression system that is cultured in the same or different conditions as or from a host cell that comprises the transcriptional unit or synthetic expression system, wherein the host cells are of the same type. In some embodiments, the control host cell comprises a transcriptional unit or expression system that is expressed in a methanol-dependent manner. In some embodiments, a control host cell is a wild-type cell, such as a wild-type Pichia pastoris, Komagataella phaffii, Komagataella pastoris, Komagataella pseudopastoris, Hansenula polymorpha, Candida boidinii, or Pichia methanolica cell. In some embodiments, the control host cell comprises a transcriptional unit or expression system that is identical to a transcriptional unit or synthetic expression system expressed in a host cell of a different type. In some embodiments, the concentration (or quantity, amount, etc.) of bioproduct produced by a synthetic expression system of this disclosure, or a host cell comprising a synthetic expression system of this disclosure, is at least 1.1 fold, at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold greater than that of a control host cell. In some embodiments, the concentration (or quantity, amount, etc.) of bioproduct produced by a host cell comprising a synthetic expression system of this disclosure is 100 fold greater than the same host cell that does not comprise the synthetic expression system. In some embodiments, the concentration of bioproduct produced by a synthetic expression system of this disclosure, or a host cell comprising a synthetic expression system of this disclosure, is from about 1.1 to about 4 fold, from about 2 to about 10 fold, from about 5 to about 15 fold, from about 10 to about 20 fold, from about 15 about 30 fold, from about 25 to about 40 fold, from about 35 to about 50 fold, from about 45 to about 60 fold, from about 55 to about 70 fold, from about 70 to about 90 fold, or from about 85 to about 100 fold greater than that of a control host cell comprising a synthetic expression system or the same host cell that does not comprise the synthetic expression system.

In some embodiments, the level (or concentration, quantity, amount, etc.) of bioproduct produced by a host cell comprising a synthetic expression system of this disclosure is at least 100%, at least 200%, at least 300%, at least 400%, at least 500%, at least 600%, at least 700%, at least 800%, at least 900%, at least 1000%, at least 1100%, at least 1200%, at least 1300%, at least 1400%, at least 1500%, at least 1600%, at least 1700%, at least 1800%, at least 1900%, at least 2000%, at least 2100%, at least 2200%, at least 23000%, at least 2400%, at least 2500%, at least 2600%, at least 2700%, at least 2800%, at least 2900%, at least 3000%, at least 3200%, at least 3400%, at least 3600%, at least 3800%, at least 4000%, or at least 5000% higher than that of a control host cell. In some embodiments, the level (or concentration, quantity, amount, etc.) of bioproduct produced by a host cell comprising a synthetic expression system of this disclosure is more than 5000% higher than that of a control host cell.

In some embodiments, the level (or concentration, quantity, amount, etc.) of bioproduct produced by a synthetic expression system of this disclosure, or a host cell comprising a synthetic expression system of this disclosure, is from about 100% to about 500%, from about 300% to about 600%, from about 300% to about 800%, from about 500% to about 1000%, from about 800% to about 1200%, from about 800% to about 1500%, from about 1000% to about 1500%, from about 1000% to about 2000%, from about 1200% to about 2000%, from about 1500% to about 2000%, from about 1800% to about 2500%, from about 2000% to about 2500%, from about 2200% to about 3000%, from about 2500% to about 3000%, from about 3000% to about 3500%, from about 3500% to about 4000%, from about 4000% to about 4500%, or from about 4500% to about 5000% higher than that of a control host cell. In some embodiments, a transcriptional unit or synthetic expression system of this disclosure, or a host cell comprising a transcriptional unit or synthetic expression system of this disclosure, is capable of producing at least 5 g/L, 10 g/L, at least 15 g/L, at least 20 g/L, at least at least 25 g/L, at least 30 g/L, at least 35 g/L, or at least 40 g/L of one or more bioproducts. In some embodiments, a transcriptional unit or synthetic expression system of this disclosure, or a host cell comprising a transcriptional unit or synthetic expression system of this disclosure, is capable of producing more than 40 g/L of one or more bioproducts. In some embodiments, a transcriptional unit or synthetic expression system of this disclosure, or a host cell comprising a transcriptional unit or synthetic expression system of this disclosure, is capable of producing from about 5 g/L to about 11 g/L, from about 9 g/L to about 15 g/L, from about 13 g/L to about 19 g/L, from about 17 g/L to about 23 g/L, from about 21 g/L to about 27 g/L, from about 25 g/L to about 31 g/L, from about 29 g/L to about 35 g/L, or from about 33 g/L to about 40 g/L of one or more bioproducts.

In some embodiments, a transcriptional unit or synthetic expression system of this disclosure, or a host cell comprising a transcriptional unit or synthetic expression system of this disclosure, is capable of producing at least 5 g/L, 10 g/L, 15 g/L, at least 20 g/L, at least 25 g/L, at least 30 g/L, at least 35 g/L, or at least 40 g/L of one or more bioproducts in methanol-independent conditions. In some embodiments, a transcriptional unit or synthetic expression system of this disclosure, or a host cell comprising a transcriptional unit or synthetic expression system of this disclosure, is capable of producing from about 5 g/L to about 11 g/L, from about 9 g/L to about 15 g/L, from about 13 g/L to about 19 g/L, from about 17 g/L to about 23 g/L, from about 21 g/L to about 27 g/L, from about 25 g/L to about 31 g/L, from about 29 g/L to about 35 g/L, or from about 33 g/L to about 40 g/L of one or more bioproducts in methanol-independent conditions. In some embodiments, the potency of a synthetic expression system is evaluated based on the amount of bioproduct generated in specific culture phases (e.g., growth phase, production phase, etc.). Excess bioproduct generated in the growth phase may be an indication of non-specific, or “leaky,” promoter activity, which may be undesirable. In some embodiments, the amount of bioproduct produced using a synthetic expression system of the present disclosure is greater in the production phase than in the growth phase. In some embodiments, the amount of bioproduct produced using a synthetic expression system of the present disclosure in the production phase is greater than that which can be produced in the production phase by a control host cell.

In some embodiments, the amount of bioproduct produced using a synthetic expression system of the present disclosure, or using a host cell comprising a transcriptional unit or synthetic expression system of this disclosure, in the production phase is at least 80%, at least 90%, at least 100%, at least 150%, at least 200%, at least 250%, at least 300%, at least 350%, at least 400%, at least 450%, or at least 500% higher than that which can be produced in the production phase by a control host cell. In some embodiments, the amount of bioproduct produced using a synthetic expression system of the present disclosure, or using a host cell comprising a transcriptional unit or synthetic expression system of this disclosure, in the production phase is more than 500% higher than that which can be produced in the production phase by a control host cell. In some embodiments, the amount of bioproduct produced using a synthetic expression system of the present disclosure, or using a host cell comprising a transcriptional unit or synthetic expression system of this disclosure, in the production phase is from about 80% to about 120%, from about 110% to about 150%, from about 140% to about 180%, from about 170% to about 220%, from about 210% to about 260%, from about 250% to about 300%, from about 290% to about 340%, from about 330% to about 380%, from about 370% to about 420%, from about 410% to about 460%, or from about 450% about 500% higher than that which can be produced in the production phase by a control host cell. In some embodiments, the amount of bioproduct produced using a synthetic expression system of the present disclosure, or using a host cell comprising a transcriptional unit or synthetic expression system of this disclosure, in the production phase is from about 1% to about 100%, from about 50% to about 150%, from about 100% to about 200%, or from about 150% to about 200% greater than that which can be produced in the production phase by a control cell or the same host cell that does not comprise the synthetic expression system. In some embodiments, the amount of bioproduct produced using a synthetic expression system of the present disclosure is less in the growth phase than in the production phase. In some embodiments, the amount of bioproduct produced using a synthetic expression system of the present disclosure in the growth phase is less than that which is produced in the growth phase by a control host cell.

In some embodiments, the amount of bioproduct produced using a synthetic expression system of the present disclosure, or using a host cell comprising a transcriptional unit or synthetic expression system of this disclosure, in the growth phase is at least 80%, at least 90%, at least 100%, at least 150%, at least 200%, at least 250%, at least 300%, at least 350%, at least 400%, at least 450%, or at least 500% less than that which is produced in the growth phase by a control cell or the same host cell that does not comprise the synthetic expression system. In some embodiments, the amount of bioproduct produced using a synthetic expression system of the present disclosure, or using a host cell comprising a transcriptional unit or synthetic expression system of this disclosure, in the growth phase is from about 80% to about 120%, about 110% to about 150%, about 140% to about 180%, about 170% to about 220%, about 210% to about 260%, about 250% to about 300%, about 290% to about 340%, about 330% to about 380%, about 370% to about 420%, about 410% to about 460%, or about 450% about 500% less than that which is produced in the growth phase by a control cell or the same host cell that does not comprise the synthetic expression system.

In some embodiments, the efficiency of a synthetic expression system of the present disclosure, or of a host cell comprising a transcriptional unit or synthetic expression system of this disclosure, may be expressed as a ratio of bioproduct expressed in the growth phase versus the bioproduct expressed in the production phase (e.g., 1:1, 1:2, 1:3, etc.). In some embodiments, the ratio of bioproduct expressed in the growth phase versus the bioproduct expressed in the production phase using a synthetic expression system of the present disclosure, or using a host cell comprising a transcriptional unit or synthetic expression system of this disclosure, is about 1:1.1, about 1:1.2, about 1:1.3, about 1:1.4, about 1:1.5, about 1:1.6, about 1:1.7, about 1:1.8, about 1:1.9, about 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, about 1:110, about 1:130, about 1:130, about 1:140, about 1:150, about 1:160, about 1:170, about 1:180, about 1:190, or about 1:200 (or any ratio included therein). In some embodiments, the ratio of bioproduct expressed in the growth phase versus the bioproduct expressed in the production phase using a synthetic expression system of the present disclosure, or using a host cell comprising a transcriptional unit or synthetic expression system of this disclosure, is from about 1:1.1 to about 1:10, from about 1:9.5 to about 1:20, from about 1:10 to about 1:40, from about 1:30 to about 1:60, from about 1:50 to about 1:80, from about 1:70 to about 1:100, from about 1:100 to about 1:140, from about 1:140 to about 1:170, from about 1:160 to about 1:190, or from about 1:180 to about 1:200. In some embodiments, the ratio of bioproduct expressed in the growth phase versus the bioproduct expressed in the production phase using a synthetic expression system of the present disclosure, or using a host cell comprising a transcriptional unit or synthetic expression system of this disclosure, is from about 1:10 to about 1:50, from about 1:25 to about 1:75, from about 1:50 to about 1:100, from about 1:75 to about 1:125, from about 1:100 to about 1:150, or from about 1:150 to about 1:200.

In some embodiments, any of the methods described in this application may include isolation and/or purification of products of the expression of genes of interest (e.g., proteins and/or nucleic acids). For example, the isolation and/or purification can involve one or more of cell lysis, centrifugation, extraction, column chromatography, distillation, crystallization, and lyophilization.

Products produced by any of the synthetic expression systems, host cells expressing the synthetic expression systems disclosed in this application, or any of the in vitro methods described in this application, may be identified, isolated, extracted, and/or purified using any method known in the art. Mass spectrometry (e.g., LC-MS, GC-MS) is a non-limiting example of a method for identification and may be used to analyze the chemical composition and/or chemical structure and/or concentration of a compound of interest.

### Cell-Free Expression

In some embodiments, a transcriptional unit or synthetic expression system of this disclosure is a component of a cell-free expression system. In some embodiments, a transcriptional unit or synthetic expression system of this disclosure is utilized to produce one or more bioproducts in a cell-free expression system. Exemplary cell-free expressions systems include cell extracts made from E. coli (ECE), rabbit reticulocytes (RRL), wheat germ (WGE), insect cells (ICE) or Yeast Kluyveromyces (the D2P system).

### Host Cells

In some embodiments, the present disclosure provides host cells comprising a transcriptional unit or a synthetic expression system. Any of the transcriptional units or synthetic expression systems of the disclosure may be used in a host cell.

Transcriptional units or synthetic expression systems described in this application may be introduced into a suitable host cell using any methods known in the art.

In some embodiments, a host cell comprises a transcriptional unit or synthetic expression system integrated into the host cell genome. In some embodiments, a synthetic expression system comprises one copy of a first transcriptional unit; and one copy of a second transcriptional unit. The quantity of first and second transcriptional units may be expressed as a ratio of first transcriptional unit to second transcriptional unit, or as a ratio of second transcriptional unit to first transcriptional unit (i.e., “copy number ratio”). In some embodiments, the copy number ratio of the first transcriptional unit to the second transcriptional unit is 1:1.

In some embodiments, a host cell comprises multiple copies of a transcriptional unit or a synthetic expression system. In some embodiments, the first or second transcriptional unit is present in multiple copies. In some embodiments, both the first and second transcriptional units are present in multiple copies. In some embodiments, a synthetic expression system comprises two or more copies of a first transcriptional unit; and/or two or more copies of a second transcriptional unit. In some embodiments, the copy number ratio of the first transcriptional unit to the second transcriptional unit is at least 2:1, at least 3:1, at least 4:1, at least 5:1, at least 10:1, at least 20:1, or at least 30:1. In some embodiments, the copy number ratio of the second transcriptional unit to the first transcriptional unit is at least 2:1, at least 3:1, at least 4:1, at least 5:1, at least 10:1, at least 20:1, or at least 30:1.

In some embodiments, the first transcriptional unit is present in a single copy and the second transcriptional unit is present in multiple copies in the host cell genome.

In some embodiments, the synthetic expression system comprises one or more sequences that are endogenous to the host cell.

A “host cell” refers to a cell that can be used to express a transcriptional unit or synthetic expression system and precursors thereof. In some embodiments, the host cell is a Pichia pastoris, Pichia pseudopastoris, Komagataella phaffii, Pichia stipitis, Pichia membranifaciens, Komagataella pastoris, Komagataella pseudopastoris, Komagataella kurtzmanii, Komagataella mondaviorum, Hansenula polymorpha, Candida boidinii, or Pichia methanolica cell. It is understood that in some embodiments, a host cell refers not only to a particular recombinant host in which a transcriptional unit or synthetic expression system is introduced, but also to the progeny or potential progeny of such a host cell. The term “cell,” as used in this application, may refer to a single cell or a population of cells, such as a population of cells belonging to the same cell line or strain. Use of the singular term “cell” should not be construed to refer explicitly to a single cell rather than a population of cells.

Any suitable host cell may be used to express the transcriptional units or synthetic expression systems disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

In some embodiments, the host cell is a yeast cell. In some embodiments, the host cell is methylotrophic. A “methylotrophic cell” is one that naturally (i.e., prior to any manipulation by a human) has an ability to utilize reduced one-carbon compounds, such as methanol or methane, as the carbon source for its growth, and multi-carbon compounds that contain no carbon-carbon bonds, such as dimethyl ether and dimethylamine. Methylotrophic cells are known in the art, and include, for example, those in the genera Pichia, Komagataella, Hansenula, and Candida. A host cell that is naturally methylotrophic, such as one from among the genera Pichia, Komagataella, Hansenula, or Candida but has been rendered unable to utilize methanol, e.g. by engineering, is still considered to be a methylotrophic host cell for purposes of this disclosure.

In some embodiments, a host cell includes any of: a member of the genera Pichia, Komagataella, Candida, Dipodascus, Galactomyces, Hansenula, Kluyveromyces (e.g., K. lactis), Magnusiomyces, Ogatae, Phaffomyces, Saccharomyces (e.g., S. cerevisiae), Schizosaccharomyces, Starmera, Starmerella, Sugiyamaella, Trichomonascus, Wickerhamomyces, Wickerhamiella, Williopsis, Yarrowia, or Zygoascus; or a member of Komagataella Clade, Phaffomyces Clade, Dipodascaceae, Phaffomycetaceae, or Trichomonascaceae. In some embodiments, the host cell is a member of the genera Pichia or Komagataella. In some embodiments, the host cell is any of: Pichia pastoris, Pichia pseudopastoris, Pichia stipitis, Pichia membranifaciens, Pichia methanolica, Pichia finlandica, Pichia trehalophila, Pichia kodamae, Pichia opuntiae, Pichia thermotolerans, Pichia salictaria, Pichia quercuum, Pichia pijperi, Pichia angusta, Komagataella phaffii, Komagataella pastoris, Komagataella pseudopastoris, Komagataella kurtzmanii, Komagataella mondaviorum, Wickerhamomyces anomalus, Candida albicans, Candida lusitaniae, Ogataea glucozyma, Candida blankii, Candida boidinii, Candida orba, Candida petrohuensis, Candida santjacobensis, Candida sorboxylosa, Candida sp., Dipodascus albidus, Galactomyces geotrichum, Hansenula polymorpha, Kluyveromyces lactis, Magnusiomyces magnusii, Phaffomyces antillensis, Phaffomyces opuntiae, Phaffomyces thermotolerans, Saccharomyces cerevisiae, Saccharomyces carlsbergensis, Saccharomyces diastaticus, Saccharomyces norbensis, Saccharomyces kluyveri, Schizosaccharomyces pombe, Starmerella bombicola, Sugiyamaella smithiae, Trichomonascus petasosporus, Wickerhamiella domercqiae, Yarrowia lipolytica, or Zygoascus hellenicus. In some embodiments, a host cell is an undescribed species of Pichia or Komagataella. In some embodiments, a host cell is a Pichia sp. or Komagataella sp.

In some embodiments, the yeast strain is an industrial yeast strain. In some embodiments, the host cell is a fungal cell. In some embodiments, a fungal cell includes a cell of Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., or Trichoderma spp.

Without wishing to be bound by any particular theory, the present disclosure notes that some reports in the scientific literature reassigned P. pastoris to the genus Komagataella, and various strains of P. pastoris were separated into K. phaffii, K. pastoris, and K. pseudopastoris. In some embodiments, Pichia pastoris is identical to Komagataella phaffii, and Komagataella phaffii is sometimes referred to by its former species name Pichia pastoris. As used in this disclosure, Pichia pseudopastoris is interchangeable with Komagataella pseudopastoris. These various genera and species, and the relationships between them, are described in the scientific literature, for example: Feng et al. 2020 Yeast 37(2):237-245; De Schutter et al. 2009. Nature Biotechnology. 27 (6): 561-566; Heistinger et al. 2018 Molecular and Cellular Biology 38 Issue 2 e00398-17; Kurtzman International Journal of Systematic and Evolutionary Microbiology (2005), 55: 973-976; Kurtzman 2011 Antonie van Leeuwenhoek 99:13-23; Kurtzman 2013 Antonie van Leeuwenhoek 104:339-347; Kurtzman 2012. Antonie van Leeuwenhoek 101: 859-868; Naumov 2018 Antonie van Leeuwenhoek 111:1197-1207; and Yamada et al. 1995 Biosci. Biotech. Biochem. 59: 439-444.

In some embodiments, the host cell is an algal cell such as Chlamydomonas (e.g., C. reinhardtii) and Phormidium (P. sp. ATCC29409).

In some embodiments, the host cell is a prokaryotic cell. Suitable prokaryotic cells include gram positive, gram negative, and gram-variable bacterial cells.

Various strains that may be used as host cells in the practice of the disclosure are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

A host cell may comprise genetic modifications relative to a wild-type counterpart in addition to harboring the transcriptional unit. In some embodiments, a host cell is modified to reduce or inactivate one or more endogenous genes. Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014; 1205:45-78) or gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

It will be appreciated by those of skill in the art that the transcriptional units or synthetic expression systems of this disclosure can be configured in various ways within the host cell genome (e.g., on the same or different polynucleotide sequences; on the same or different chromosomes; oriented in the 5′ or 3′ direction with respect to the primary direction of transcription mediated by the promoters of the first and second transcriptional units).

In some embodiments, the synthetic expression system (e.g., including a first and second transcriptional unit) is located on a single plasmid. In some embodiments, the first and second transcriptional units are located on a single plasmid. In some embodiments, the synthetic expression system is located on two or more (e.g., different) plasmids. In some embodiments, the first and second transcriptional units are located on two or more (e.g., different) plasmids. For example, in some embodiments, the first transcriptional unit is located on a first plasmid, and the second transcriptional unit is located on a second plasmid.

In some embodiments, the synthetic expression system is located on a single chromosome in the host cell genome. In some embodiments, components of the synthetic expression system are located on two or more (e.g., different) chromosomes in the host cell genome. In some embodiments, the first and second transcriptional units are located on the same chromosome in the host cell genome. In some embodiments, the synthetic expression system is located on two or more (e.g., different) chromosomes in the host cell genome. In some embodiments, the first and second transcriptional units are located on two or more (e.g., different) chromosomes in the host cell genome.

In some embodiments, the first and second transcriptional units are oriented in the same direction (e.g., oriented in the same 5′ or 3′ direction with respect to the primary direction of transcription mediated by the promoters of the first and second transcriptional units). In some embodiments, the first and second transcriptional units are oriented in different directions (e.g., the 5′ or 3′ direction with respect to the primary direction of transcription mediated by the promoters of the first and second transcriptional units). In some embodiments, multiple different first and/or second transcriptional units may be present within a host cell, and they can be in any orientation relative to each other. In some embodiments, a host cell may be engineered for synthetic protein expression, wherein such engineering comprises transforming the host cell with one or more polynucleotides comprising a synthetic expression system. Any synthetic expression system of the present disclosure may be used.

Host cells may be cultured under any suitable conditions, including, but not limited to, the culture conditions described in this disclosure. For example, any media, temperature, and incubation conditions known in the art may be used. Example culture conditions are provided in this disclosure and include methanol-independent conditions. For host cells carrying an inducible vector, cells may be cultured with an appropriate inducible agent to promote expression.

### Expression of Genes of Interest in Host Cells

This disclosure encompasses expression of genes of interest by a synthetic expression system in a host cell. In some embodiments, the methods by which genes of interest are expressed in host cells comprise culturing a host cell. The host cell may be any host cell of the present disclosure.

In some embodiments, the expressed genes of interest are synthetic. In some embodiments, a synthetic gene of interest that is introduced into the host cell may be a polynucleotide that comes from a different organism, genus, or species from the host cell; or a synthetic, engineered, or chimeric polynucleotide, or a polynucleotide that is also endogenously expressed in the same organism or species as the host cell but has been altered. For example, a polynucleotide that is endogenously present in a host cell may be considered synthetic when it is altered to be: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide.

In some embodiments, a synthetic gene of interest is a polynucleotide that is endogenously present in a host cell but whose expression is driven by a promoter that does not naturally regulate expression of the polynucleotide. In some embodiments, a synthetic gene of interest is a polynucleotide that is endogenously present in a host cell and whose expression is driven by a promoter that does naturally regulate expression of the polynucleotide, and the promoter is modified. In some embodiments, the promoter is recombinantly activated or repressed. For example, gene-editing-based techniques may be used to regulate expression of a polynucleotide, including an endogenous polynucleotide, from a promoter, including an endogenous promoter. See, e.g., Chavez et al., Nat Methods. 2016 July; 13(7): 563-567. A synthetic gene of interest may comprise a variant sequence as compared with a reference polynucleotide sequence; or may comprise a wild-type sequence but may not be in the wild-type context within a genome (e.g., a wild-type sequence that is expressed in/by a host cell or in a chromosomal location where it is not normally expressed).

In some embodiments, the gene of interest encodes a heme-binding protein or one or more enzymes of a heme biosynthesis pathway. In some embodiments, the heme-binding protein is hemoglobin, myoglobin, neuroglobin, cytoglobin, or leghemoglobin. In some embodiments, the heme-binding protein is myoglobin. In some embodiments, the one or more enzymes of a heme biosynthesis pathway is cytochrome P450, 9-adenylate cyclase, soluble guanylate cyclase, peroxidase, catalase, and/or cytochrome oxidase. In some embodiments, the gene of interest encodes a vaccinia capping enzyme, a T7 polymerase enzyme, or an O-methyltransferase enzyme.

In some embodiments, the coding sequence of the gene of interest may be codon optimized for expression in a particular host cell, including, but not limited to, a Pichia pastoris, Pichia pseudopastoris, Komagataella phaffii, Pichia stipitis, Pichia membranifaciens, Komagataella pastoris, Komagataella pseudopastoris, Komagataella kurtzmanii, Komagataella mondaviorum, Hansenula polymorpha, Candida boidinii, or Pichia methanolica cell.

### Culturing of Host Cells

In some embodiments, the present disclosure pertains to a host cell comprising a transcriptional unit or a synthetic expression system, wherein, when the host cell is cultured, the unit or system within the host cell is capable of producing a bioproduct (e.g., a molecule of interest). In some embodiments, the bioproduct is obtained from biomass or culture. In some embodiments, obtaining the bioproduct comprises extracting the bioproduct from biomass. In some embodiments, obtaining the bioproduct comprises collecting the bioproduct from the culture medium.

In some embodiments, methods of producing a bioproduct are provided comprising steps of expressing a gene of interest by culturing a host cell, purifying an enzyme encoded by the gene of interest, and using the purified enzyme for bioconversion of a substrate to a molecule of interest. In some embodiments, the molecule of interest is heme.

Any of the host cells comprising a synthetic expression system disclosed in this application can be cultured using any method and in media of any type (e.g., rich and/or minimal and/or nutrient-limiting, etc.) known in the art in order to control the timing and/or level of production of a bioproduct.

In some embodiments, culturing can occur over several phases, and it may be desirable to limit expression of a gene of interest until a later phase, e.g., the production phase, as expression or high expression of the gene of interest may cause toxicity and/or otherwise reduce cell growth. Without wishing to be bound by any particular theory, the present disclosure notes that, even in a relatively tightly controlled genetic system, a low or basal level of expression of a gene of interest may occur prior to production phase, but if such expression leads to toxicity, the cells and synthetic expression system can be maintained under conditions to decrease the expression to as low a level as technically feasible.

As non-limiting examples, the culturing conditions of a host cell comprising a synthetic expression system can be altered in production phase, such that the input promoter is induced and, via the action of the transcription factor and synthetic output promoter, a high level of expression of the gene of interest is achieved.

In some embodiments, an input promoter can be activated by limitation of a nutrient or another change in culture conditions during production phase to induce the promoter and increase the expression of the gene of interest. In some embodiments, expression of the gene of interest can be further increased by addition of a second nutrient.

In some embodiments, an input promoter is not inducible and/or cannot be activated by limitation of a nutrient or another change in culture conditions, and is constitutively active.

In some embodiments, host cells comprising a synthetic expression system disclosed in this application are cultured in a methanol-independent medium or using a methanol-independent method. “Methanol-independent” or “methanol-free,” as related to media, culture conditions, transcriptional units, synthetic expression systems, etc., means that exogenous methanol has not been added to the culture medium. Without wishing to be bound by any particular theory, the present disclosure notes that, under some culture conditions, some host cells may endogenously produce small amounts of methanol, but such methanol is disregarded in considering whether a medium is methanol-free or not. “Methanol-independent” means that the synthetic expression system operates in the host cell independently of exogenous methanol added to the culture medium, and addition of exogenous methanol is not required for the functioning of the synthetic expression system. The fact that, under some culture conditions, some host cells may endogenously produce small amounts of methanol is disregarded in considering whether a system is methanol-independent or methanol-dependent.

In some embodiments, methods are provided for expressing a gene of interest or producing a molecule of interest comprising steps of: (a) culturing a host cell according to the methods of this disclosure in a suitable medium for a period of time to allow cell growth, and (b) changing one or more culture conditions to facilitate expression of the gene of interest or production of the molecule of interest. In some embodiments, changing the culture conditions comprises changing the composition of the culture medium. In some embodiments, changing the culture conditions comprises limiting or depleting a nutrient, such as thiamine, glycerol, one or more monosaccharides, and/or formic acid. In some embodiments, changing the culture conditions comprises limiting any of: a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate. In some embodiments, changing the culture conditions comprises the limitation of a combination of any two nutrients. In some embodiments, changing the culture conditions comprises adding formic acid.

In some embodiments, culturing of host cells comprising a synthetic expression system occurs over several phases or stages. The terms “stage” and “phase” are used interchangeably in this application.

In some embodiments, culturing of host cells occurs over the stages of: Stage I, Stage II, and Stage III.

In some embodiments, in Stage I (also known as the batch phase), fresh, sterile medium is initially inoculated with host cells comprising a synthetic expression system. After a period of growth, the culture from Stage I is ready for the subsequent phase.

In some embodiments, in Stage II (also known as a cell growth phase), the cultures grow, and biomass increases. In some embodiments, in at least part of Stage II, cell growth is exponential.

In some embodiments, in Stage III (also known as a production phase or induction phase), the synthetic expression system, if not already induced, is induced to express the gene of interest. In some embodiments, the synthetic expression system is not induced in Stage I or Stage II, but during Stage III, allowing high expression of the gene of interest. In some embodiments, during a production phase, an additional component is added to the culture medium. In some embodiments, the additional component is a nutrient. In some embodiments, the additional component further increases expression from the input promoter. In some embodiments, the additional component is: formic acid or methanol.

The various stages can also occur using the same or different growth media, volumes, duration, temperatures (e.g., 30 C, 35° C., 37° C., or 42° C.), pH levels (e.g., acidic, slightly acidic, neutral, slightly basic, or basic), agitation levels, aeration levels, dissolved oxygen levels, levels and/or concentrations and/or flowrates of the limiting nutrient, additional nutrients, conditions, etc.

As is known in the art, and as appropriate for differences in culture volumes and cell density, the various stages can occur in any vessel and do not need to occur in the same type or size of vessel.

In some embodiments, host cells can be cultured in an industrial-scale process. In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion modes of operation.

In some embodiments, a bioreactor, fermentor, or other vessel includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, nutrient concentrations, metabolite concentrations, etc.), physical/mechanical parameters (e.g., density, conductivity, degree of agitation, pressure, and flow rate, etc.).

The culture medium may comprise any of various components including but not limited to: potassium, potassium phosphate monobasic, ammonium, ammonium sulfate, calcium, calcium sulfate dihydrate, potassium sulfate, magnesium, magnesium sulfate heptahydrate, a trace metal, PTM4 solution, copper, copper (II) sulfate pentahydrate, sodium iodide, manganese, manganese (II) sulfate monohydrate, sodium, molybdenum, sodium molybdate dihydrate, boric acid, cobalt, cobalt (II) chloride (anhydrous), zinc, zinc chloride (anhydrous), iron, iron (II) sulfate heptahydrate, biotin, sulfate, sulfuric acid, water and other optional nutrients (which can be present, present in abundance, present in excess, or limiting (e.g., the nutrient is absent or not exogenously added to the medium). The medium can be sterilized by any method known in the art. In some embodiments, the culture medium comprises a carbon source. In some embodiments, a carbon source(s) during fermentation (e.g., a growth phase such as Stage I and/or Stage II) is: glucose; glycerol and/or sorbitol; or glycerol and/or sorbitol. Various non-limiting examples of culture media are described in the Examples. A variety of culture media suitable for various vessels, purposes, and host cells are described in this document and/or are generally known in the art.

As detailed below, in some embodiments, the input promoter (e.g., in a synthetic expression system) is inducible (e.g., can be induced by depletion of a nutrient). In some embodiments, the nutrient is present in abundance or excess in Stage I and Stage II (e.g., when the input promoter is not induced), but is limited in Stage III (e.g., when the input promoter is induced, and the gene of interest is highly expressed). In some embodiments, the nutrient is added as a bolus in Stage I and/or Stage II (e.g., when the input promoter is not induced), but is limited in Stage III. In some embodiments, a nutrient may be depleted.

“Limitation” means that a nutrient or other culture additive is consumed at a rate equal to or faster than it is added exogenously. “Depletion” means that a nutrient or other culture additive that is added exogenously is consumed either partially or completely.

In some cases, a limiting nutrient comprises carbon; thus “nutrient limitation” (and similar phrases) may also be referenced as “carbon limitation.” In some embodiments, the act of limiting a nutrient (e.g., during a production phase) is referenced as an induction. In some embodiments, a condition of limitation of a nutrient does not require the complete absence of the nutrient.

In some embodiments, a nutrient that may be depleted or limited is: inositol, methionine, phosphate, glucose, glycerol, or thiamine. In some embodiments, in a condition of the limitation or depletion of a nutrient, the nutrient is provided in or fed into a culture medium (e.g., at a low or moderate level), but the host cells consume the nutrient at a rate faster than it is supplied, such that there is no available free (or detectable) level of the nutrient in the culture medium.

In some embodiments, in a condition which is not a limitation of a nutrient, the nutrient is provided in or fed into a culture medium (e.g., at a high level or in excess), and the host cells consume the nutrient at a rate slower than it is supplied, such that there exists available free (or detectable) levels of the nutrient in the culture medium.

At any particular cell density or biomass density, one of ordinary skill in the art, with basic knowledge of the host cells (and the biochemistry and growth patterns thereof) and with an understanding of the synthetic expression system, can calculate, predict, and/or monitor the rates of feeding a particular nutrient into a culture medium, in order to achieve, as desired, a condition wherein the nutrient is limiting or depleted, or a condition wherein the nutrient is not limiting.

In some embodiments, the culturing process includes a batch phase, in which the nutrient is maintained at excess, and a fed-batch phase, wherein the culture is step-fed to maintain excess levels of the nutrient. In some embodiments, dissolved oxygen levels can provide an indication of nutrient levels; for example, depletion of a nutrient can result in an increase in dissolved oxygen, and such an increase can trigger a fed-batch phase, wherein the culture is step-fed to maintain excess levels of the nutrient. In some embodiments, the input promoter is induced by depletion of glucose, and depletion of glucose can trigger a sudden dissolved oxygen spike. In some embodiments, the batch phase can be considered the last part of Stage I, and is followed by the fed-batch phase in Stage II. In some embodiments, the batch phase can be considered to the first part of Stage II, and is followed by the fed-batch phase in the second part of Stage II.

Aspects of the disclosure relate to the production of proteins and/or nucleic acids expressed from a gene of interest in methanol-independent fermentation conditions. In some embodiments, the input promoter of the first transcriptional unit and/or the output promoter of the second transcriptional unit is an inducible promoter. In some embodiments, the inducible promoter is responsive (e.g., inducible) in the absence of methanol. In some embodiments, the inducible promoter is responsive to nutrient limitation, addition, or depletion with respect to a cognate cultivation process.

In some embodiments, the input promoter is responsive to thiamine depletion. In some embodiments, the input promoter is responsive to glycerol depletion. In some embodiments, the input promoter is responsive to glucose limitation. In some embodiments, the input promoter is responsive to formic acid limitation. In some embodiments, the inducible promoter is responsive to monosaccharide limitation. In some embodiments, the inducible promoter is responsive to the limitation of a carbon source, a sugar, a starch, galactose, maltose, glucose, dextrose, sorbitol, inositol, glycerol, methionine, a vitamin, phosphate, a steroid, a nitrogen source, nitrate, nitrite, ammonium, an amino acid, methionine, a metal (e.g., a heavy metal), copper, benzoic acid, hydrogen peroxide, a calcium-containing compound, an alcohol, methanol, tetracycline, a steroid, and/or phosphate. Various inducible promoters are known in the art. In some embodiments, the inducible promoter is responsive to the presence of or addition of (e.g., the addition to the medium of an excess of) any of: a nutrient, an antibiotic, tetracycline, doxycycline, a sugar, a starch, galactose, maltose, glucose, sorbitol, inositol, glycerol, formic acid, a vitamin, a steroid, a nitrogen source, nitrate, nitrite, ammonium, an amino acid, methionine, an ion, sodium, and/or phosphate.

In some embodiments, a single limiting nutrient is used. In some embodiments, the inducible promoter is responsive to limitation of a combination of nutrients (e.g., two nutrients, or more than two nutrients) (e.g., during production phase of the culturing of a host cell comprising a synthetic expression system). In some embodiments, a combination of limiting nutrients is used. In some embodiments, the inducible promoter is responsive to limitation of a combination of nutrients, including but not limited to: glycerol, glucose, and thiamine, or wherein the combination is: glycerol and formic acid; glucose and formic acid; or glucose and thiamine. In some embodiments, the activity of the inducible promoter is increased by the presence of formic acid. In some embodiments, the response of the promoter (e.g., once induced) is activation. In some embodiments, the response of the promoter (e.g., once induced) is repression.

In some embodiments, more than one nutrient can be limiting or depleted, and any method or composition useful for culturing host cells under conditions of controlled limitation or depletion of a single nutrient can be combined, duplicated, and/or altered for use in culturing host cells under conditions of controlled limitation or depletion of two or more nutrients.

In some embodiments, nutrients simultaneously limited during a production phase are thiamine and glucose. In some embodiments, glucose may be limited while thiamine may be depleted.

In some embodiments, the limiting nutrient is inositol; and the carbon source(s) during fermentation (e.g., a growth phase such as Stage I and/or Stage II) is glucose, glycerol or sorbitol; and the input promoter P(in) is P(INO1).

In some embodiments, the limiting nutrient is methionine; and the carbon source(s) during fermentation (e.g., a growth phase such as Stage I and/or Stage II) is glucose, glycerol or sorbitol; and the input promoter P(in) is: P(MET6), P(SAH1), P(SAM2), or P(MXR1).

In some embodiments, the limiting nutrient is phosphate; and the carbon source(s) during fermentation (e.g., a growth phase such as Stage I and/or Stage II) is glucose, glycerol or sorbitol; and the input promoter P(in) is P(PH089).

In some embodiments, the limiting nutrient is glucose; and the carbon source(s) during fermentation (e.g., a growth phase such as Stage I and/or Stage II) is glycerol or sorbitol; and the input promoter P(in) is any of the various promoters inducible by limiting glucose described in this disclosure.

In some embodiments, the limiting nutrient is glycerol; and the carbon source(s) during fermentation (e.g., a growth phase such as Stage I and/or Stage II) is glucose or sorbitol; and the input promoter P(in) is any of various promoters inducible by limiting glycerol described in this disclosure.

In some embodiments, the inducible promoter is a chemically regulated promoter.

In some embodiments, an inducible promoter is a physically regulated promoter, e.g., wherein transcriptional activity is regulated by a change in culture conditions, including but not limited to: a change in light (e.g., frequency of light, wavelength of light, brightness of light, duration of light, light/dark cycle, etc.), temperature (e.g., a heat shock or cold shock promoter), pressure, gravity, pH (acidic or basic conditions), salinity, or a change in any other physical condition. In some embodiments, during the production phase of the culturing of a host cell comprising a synthetic expression system, if the input promoter is a physically regulated promoter, then culture conditions (e.g., light or temperature) can be altered during production phase to activate the input promoter, resulting in high expression of the gene of interest.

Example 1 shows various non-limiting examples of synthetic expression systems, and various culture conditions (Processes 1, 2, and 3) useful for culturing host cells comprising these systems and expressing a bioproduct.

Table 1 shows the combinatorial design of non-limiting examples of synthetic expression systems. These are useful for, among other things, a production process according to Process 1, wherein glycerol is limiting and formic acid is added.

In some embodiments, a promoter is cognate with respect to a production process. In some embodiments, an input promoter is cognate with respect to a particular production process (e.g., a process described in this document such as Process 1, Process 2, or Process 3) if the input promoter is activated under a particular culturing step or condition in that process (e.g., for Process 1, limiting glycerol+added formic acid; for Process 2, limiting glucose+added formic acid; and for Process 3, limiting glucose+depleted thiamine).

In some embodiments, an output promoter is cognate with respect to a transcription factor. In some embodiments, a particular output promoter is cognate with respect to a particular transcription factor [e.g. a transcription factor (TF) or synthetic transcription factor (sTF)] in that the transcription factor activates transcription from the output promoter.

In some embodiments, a synthetic transcription factor (sTF) is described as being based on a wild-type transcription factor (e.g., a sTF may be a Bm3R1-based sTF), in that a portion (e.g., a DNA binding domain or a TAD) of the sTF may be derived from, or may be a variant of, the corresponding portion of the wild-type transcription factor. In some embodiments, a synthetic output promoter is cognate to (e.g., cognate with respect to) a particular sTF in that the output promoter can be activated by the sTF; in a non-limiting example, a synthetic output promoter cognate to a sTF can comprise an operator which is bound by a DNA binding domain of the sTF.

In some embodiments, the synthetic expression system comprises: an input promoter cognate with respect to Process 1, for example, as listed in Table 4; a one-component Bm3R1-based sTF, Table 7; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 1, for example, as listed in Table 4; a one-component PhlF_AM-based sTF, Table 8; an optional transcriptional terminator, Table 19; an optional spacer, Table 20; a synthetic output promoter cognate with respect to the PhlF_AM-based sTF, Table 16; and a gene of interest. phIF_AM refers to phIF^{AM}, as described in Meyer et al. 2019 Nat. Chem. Biol. 15: 196, or a variant or derivative thereof.

In some embodiments, the synthetic expression system comprises: an input promoter cognate with respect to Process 1, for example, as listed in Table 4; a one-component TetR-based sTF, Table 9; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 1, for example, as listed in Table 4; a one-component VanR_AM-based sTF, Table 10; an optional transcriptional terminator, Table 19; an optional spacer, Table 20; a synthetic output promoter cognate with respect to the VanR_AM-based sTF, Table 18; and a gene of interest. VanR_AM refers to VanR^{AM}, as described in Meyer et al. 2019 Nat. Chem. Biol. 15: 196, or a variant or derivative thereof.

In some embodiments, the synthetic expression system comprises: an input promoter cognate with respect to Process 1, for example, one listed in Table 4; a two-component Bm3R1-based sTF, Table 11; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 1, for example, one listed in Table 4; a two-component PhlF_AM-based sTF, Table 12; an optional transcriptional terminator, Table 19; an optional spacer, Table 20; a synthetic output promoter cognate with respect to the PhlF_AM-based sTF, Table 16; and a gene of interest.

In some embodiments, the synthetic expression system comprises: an input promoter cognate with respect to Process 1, for example, one listed in Table 4; a two-component TetR-based sTF, Table 13; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 1, for example, one listed in Table 4; a two-component VanR_AM-based sTF, Table 14; an optional transcriptional terminator, Table 19; an optional spacer, Table 20; a synthetic output promoter cognate with respect to the VanR_AM-based sTF, Table 18; and a gene of interest.

Table 2 shows the combinatorial design of non-limiting examples of synthetic expression systems. These are useful for, among other things, a production process according to Process 2, wherein glucose is limiting and formic acid is added.

In some embodiments, the synthetic expression system comprises: an input promoter cognate with respect to Process 2, for example, one listed in Table 5; a one-component Bm3R1-based sTF, Table 7; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 2, for example, one listed in Table 5; a one-component PhlF_AM-based sTF, Table 8; an optional transcriptional terminator, Table 19; an optional spacer, Table 20; a synthetic output promoter cognate with respect to the PhlF_AM-based sTF, Table 16; and a gene of interest.

In some embodiments, the synthetic expression system comprises: an input promoter cognate with respect to Process 2, for example, one listed in Table 5; a one-component TetR-based sTF, Table 9; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 2, for example, one listed in Table 5; a one-component VanR_AM-based sTF, Table 10; an optional transcriptional terminator, Table 19; an optional spacer, Table 20; a synthetic output promoter cognate with respect to the VanR_AM-based sTF, Table 18; and a gene of interest.

In some embodiments, the synthetic expression system comprises: an input promoter cognate with respect to Process 2, for example, one listed in Table 5; a two-component Bm3R1-based sTF, Table 11; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 2, for example, one listed in Table 5; a two-component PhlF_AM-based sTF, Table 12; an optional transcriptional terminator, Table 19; an optional spacer, Table 20; a synthetic output promoter cognate with respect to the PhlF_AM-based sTF, Table 16; and a gene of interest.

In some embodiments, the synthetic expression system comprises: an input promoter cognate with respect to Process 2, for example, one listed in Table 5; a two-component TetR-based sTF, Table 13; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 2, for example, one listed in Table 5; a two-component VanR_AM-based sTF, Table 14; an optional transcriptional terminator, Table 19; an optional spacer, Table 20; a synthetic output promoter cognate with respect to the VanR_AM-based sTF, Table 18; and a gene of interest.

Table 3 shows the combinatorial design of non-limiting examples of synthetic expression systems. These are useful for, among other things, a production process according to Process 3, wherein glucose is limiting and thiamine is depleted.

In some embodiments, the synthetic expression system comprises: an input promoter cognate with respect to Process 3, for example, one listed in Table 6; a one-component Bm3R1-based sTF, Table 7; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 3, for example, one listed in Table 6; a one-component PhlF_AM-based sTF, Table 8; an optional transcriptional terminator, Table 19; an optional spacer, Table 20; a synthetic output promoter cognate with respect to the PhlF_AM-based sTF, Table 16; and a gene of interest.

In some embodiments, the synthetic expression system comprises: an input promoter cognate with respect to Process 3, for example, one listed in Table 6; a one-component TetR-based sTF, Table 9; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 3, for example, one listed in Table 6; a one-component VanR_AM-based sTF, Table 10; an optional transcriptional terminator, Table 19; an optional spacer, Table 20; a synthetic output promoter cognate with respect to the VanR_AM-based sTF, Table 18; and a gene of interest.

In some embodiments, the synthetic expression system comprises: an input promoter cognate with respect to Process 3, for example, one listed in Table 6; a two-component Bm3R1-based sTF, Table 11; an optional transcriptional terminator, Table 19; an optional spacer, Table 20; a synthetic output promoter cognate with respect to the Bm3R1-based sTF, Table 15; and a gene of interest.

In some embodiments, the synthetic expression system comprises: an input promoter cognate with respect to Process 3, for example, one listed in Table 6; a two-component PhlF_AM-based sTF, Table 12; an optional transcriptional terminator, Table 19; an optional spacer, Table 20; a synthetic output promoter cognate with respect to the PhlF_AM-based sTF, Table 16; and a gene of interest.

In some embodiments, the synthetic expression system comprises: an input promoter cognate with respect to Process 3, for example, one listed in Table 6; a two-component TetR-based sTF, Table 13; an optional transcriptional terminator, Table 19; an optional spacer, Table 20; a synthetic output promoter cognate with respect to the TetR-based sTF, Table 17; and a gene of interest.

In some embodiments, the synthetic expression system comprises: an input promoter cognate with respect to Process 3, for example, one listed in Table 6; a two-component VanR_AM-based sTF, Table 14; an optional transcriptional terminator, Table 19; an optional spacer, Table 20; a synthetic output promoter cognate with respect to the VanR_AM-based sTF, Table 18; and a gene of interest.

In some embodiments, the disclosure pertains to compositions and methods related to a synthetic expression system selected from: P96.sTF.Tet.13.102.4; P96.sTF.Van.9.103.4; P96.sTF.Phl.12.99.6; P96.sTF.Tet.1.106.4; P96.sTF.Phl.7.11.7; and P96.sTF.Phl.5.107.4; and the synthetic expression system further comprises a gene of interest.

In some embodiments, the disclosure pertains to a method of producing a bioproduct from host cells cultured under Process 1 and comprising a synthetic expression system selected from: P96.sTF.Tet.13.102.4; P96.sTF.Van.9.103.4; P96.sTF.Phl.12.99.6; P96.sTF.Tet.1.106.4; P96.sTF.Phl.7.11.7; and P96.sTF.Phl.5.107.4; and the synthetic expression system further comprises a gene of interest. The various components of these synthetic expression systems are described in detail.

In some embodiments, the disclosure pertains to compositions and methods related to a synthetic expression system selected from: P96.sTF.Phl.5.40.8; P96.sTF.Bm.9.118.8; P96.sTF.Phl.12.25.7; P96.sTF.Phl.5.109.8; P96.sTF.Bm.13.100.7; P96.sTF.Phl.12.17.9; and P96.sTF.Phl.9.107.7; and the synthetic expression system further comprises a gene of interest.

In some embodiments, the disclosure pertains to a method of producing a bioproduct from host cells cultured under Process 2 and comprising a synthetic expression system selected from: P96.sTF.Phl.5.40.8; P96.sTF.Bm.9.118.8; P96.sTF.Phl.12.25.7; P96.sTF.Phl.5.109.8; P96.sTF.Bm.13.100.7; P96.sTF.Phl.12.17.9; and P96.sTF.Phl.9.107.7; and the synthetic expression system further comprises a gene of interest. The various components of these synthetic expression systems are described in detail in this document.

In some embodiments, the disclosure pertains to compositions and methods related to a synthetic expression system selected from: P96.sTF.Phl.5.41.10; and P96.sTF.Bm.5.23.11; and the synthetic expression system further comprises a gene of interest.

In some embodiments, the disclosure pertains to a method of producing a bioproduct from host cells cultured under Process 3 and comprising a synthetic expression system selected from: P96.sTF.Phl.5.41.10; and P96.sTF.Bm.5.23.11; and the synthetic expression system further comprises a gene of interest. The various components of these synthetic expression systems are described in detail.

In some embodiments, a synthetic expression system comprises or consists of a polynucleotide having at least 90%, at least 95%, or at least 99% identity to the nucleic acid sequence of any one of SEQ ID NOs: 1-15. Within these synthetic expression systems, in some embodiments the input promoter comprises or consists of a polynucleotide having at least 90%, at least 95%, or at least 99% identity to the nucleic acid sequence of any one of SEQ ID NOs: 16-25, the transcription factor or a component of a transcription factor is encoded by a polynucleotide having at least 90%, at least 95%, or at least 99% identity to the nucleic acid sequence of any one of SEQ ID NOs: 26-40 or 182-185, the transcription factor or a component of a transcription factor comprises a polypeptide having at least 90%, at least 95%, or at least 99% identity to the amino acid sequence of any one of SEQ ID NOs: 41-55, and/or the output promoter comprises a polynucleotide having at least 90%, at least 95%, or at least 99% identity to the nucleic acid sequence of any one of SEQ ID NOs: 56-70 or 186-193.

In some embodiments, the disclosure provides individual components (transcriptional unit, input promoter, transcription factor or components thereof, synthetic output promoter, gene of interest, transcriptional terminator, spacer, etc.) that can be used in a transcriptional unit or an expression system.

In some embodiments, a synthetic expression system comprises an input promoter comprising or consisting of a polynucleotide having at least 90%, at least 95%, or at least 99% identity to the nucleic acid sequence of any one of SEQ ID NOs: 16-25. In some embodiments, a synthetic expression system comprises an output promoter comprising or consisting of a polynucleotide having at least 90%, at least 95%, or at least 99% identity to the nucleic acid sequence of any one of SEQ ID NOs: 56-70 or 186-193. In some embodiments, a synthetic expression system comprises a transcription factor encoded by a polynucleotide having at least 90%, at least 95%, or at least 99% identity to the nucleic acid sequence of any one of SEQ ID NOs: 26-40 or 182-185, or comprising or consisting of a polypeptide having at least 90%, at least 95%, or at least 99% identity to the amino acid sequence of any one of SEQ ID NOs: 41-55.

### Transcriptional Units

In some embodiments, the disclosure provides a transcriptional unit, or a synthetic expression system comprising a first and a second transcriptional unit. In some embodiments, a synthetic expression system comprises a first transcriptional unit comprising a transcription factor (or at least one component thereof), and a second transcriptional unit comprising a synthetic output promoter. In some embodiments, the transcription factor is an activator of the synthetic output promoter of the second transcriptional unit.

In some embodiments, a first transcriptional unit comprises an insertion site (e.g., a site suitable for insertion of a promoter) upstream of a polynucleotide encoding a transcription factor or component thereof.

In some embodiments, a promoter can be inserted into an insertion site of a first transcriptional unit such that the promoter is operably linked to and capable of expressing a polynucleotide encoding a transcription factor or component thereof.

In some embodiments, a second transcriptional unit comprises an output promoter upstream of an insertion site (e.g., a site suitable for insertion of a gene of interest).

In some embodiments, a gene of interest can be inserted into an insertion site of a second transcriptional unit, such that the output promoter is operably linked to and capable of expressing the gene of interest.

In some embodiments, the disclosure provides an expression vector comprising a synthetic expression system or transcriptional unit. In some embodiments, an expression vector comprises an insertion site. In some embodiments, a gene of interest encoding the protein of interest is inserted into the insertion site. In some embodiments, an expression vector facilitates expression of a protein of interest.

In some embodiments, an insertion site is a site in a nucleic acid which is suitable for directed insertion of a polynucleotide (e.g., a synthetic or exogenous polynucleotide), including but not limited to: a promoter or a gene of interest. In some embodiments, an insertion site comprises one or more restriction enzyme sites. In some embodiments, an insertion site is a multi-cloning site. In some embodiments, a multi-cloning site is a short span of a nucleic acid which comprises two or more restriction sites (e.g., EcoRI, SalI, XmaI, BamHI, SwaI, AsiSI, NotI, SaclI, NheI, AccI, etc.). In some embodiments, an insertion site is a landing pad. In some embodiments, an insertion site is a landing pad, wherein the landing pad is suitable for recombinase-mediated insertion of a synthetic or exogenous polynucleotide (e.g., a promoter or a gene of interest). In some embodiments, an insertion site is a multi-landing pad site. Various landing pads and multi-landing pads are known in the art, e.g., Leonid Gaidukov et al. 2018 Nucleic Acids Res. 46(8): 4072-4086; Chi et al. 2019 PLOS ONE, Published: Jul. 25, 2019, A system for site-specific integration of transgenes in mammalian cells; and Phan et al. 2017 Nature Scientific Rep. 7:17771.

In some embodiments, a synthetic expression system comprises one or more of the following components: (a) a first transcriptional unit, which comprises: an input promoter operably linked to and capable of expressing a polynucleotide encoding a transcription factor or at least one component of a transcription factor; and (b) a second transcriptional unit, which comprises: a synthetic output promoter operably linked to and capable of expressing a genes of interest, and, optionally, a transcriptional terminator downstream of the gene of interest.

Various terms used in this disclosure, pertaining to transcriptional units and other components of synthetic expression systems and other aspects of the invention, are further explicated below.

As used in this disclosure, in some embodiments, a “transcriptional unit” refers to a sequence of nucleotides that codes for at least one RNA molecule (e.g., a polynucleotide encoding a transcription factor or at least one component of a transcription factor in a first transcriptional unit; or a gene of interest in a second transcriptional unit), along with the sequences necessary for its instantiation, such as a promoter; and the transcriptional unit optionally comprises a transcriptional terminator and/or other regulatory sequences. A “transcriptional unit” refers to a sequence of nucleotides that codes for at least one RNA molecule (e.g., a polynucleotide encoding a transcription factor or at least one component of a transcription factor), along with a site suitable for insertion (e.g., an insertion site) of the sequences necessary for its instantiation, such as a promoter; and the transcriptional unit optionally comprises a transcriptional terminator and/or other regulatory sequences. In some embodiments, a “transcriptional unit” refers to a sequence of nucleotides that comprises a promoter (e.g., an output promoter) and a site suitable for insertion (e.g., an insertion site) of a gene of interest, along with sequences necessary for its instantiation, such as, optionally, a transcriptional terminator and/or other regulatory sequences. In some embodiments, a transcriptional unit further comprises a spacer. In some embodiments, a promoter and/or a polynucleotide encoding a transcription factor, at least one component of a transcription factor, or a gene of interest comprises additional sequences for expression, transcription, and/or translation of a protein encoded thereby, e.g., a 5′-UTR (5′-untranslated region), a leader sequence, and/or a 3′-UTR (3′-untranslated region), and/or one or more introns.

A “synthetic transcriptional unit” refers to a transcriptional unit that does not occur in nature. A “synthetic expression system” refers to an expression system that does not occur in nature. In some embodiments, a synthetic transcriptional unit or synthetic expression system is one in which one or more modifications have been made to one or more sequences found in nature, including but not limited to: rearranging the sequences; creating a chimera between two sequences of different origins (e.g., from different species, or from different arrangements within a single genome); changing the spacing between sequences (e.g., such that proteins that bind to different sequences are better aligned rotationally on the DNA helix to improve their interaction); changing DNA binding sequences to improve binding of proteins to those sequences; introducing point mutations to increase expression or control of expression; substituting or rearranging different domains of a transcription factor or other polypeptide; rearranging components of or introducing components (e.g., an operator, enhancer, upstream activating sequence, etc.) into a promoter; replacing a promoter which is normally upstream of a particular gene of interest with a different promoter; etc. In some embodiments, a synthetic expression system of the present disclosure is comprised of two or more transcriptional units.

In some embodiments, a synthetic expression system comprises a second transcriptional unit comprising a gene of interest, wherein the gene of interest is a gene [operably linked to various cis-acting components such as 5′-UTR, coding segment, 3′-UTR, optional intron(s), an optional translation enhancer, an optional translational terminator, etc.] that is desirable to be expressed in a host cell. A gene of interest may be expressed, for example, to produce an mRNA or a protein of interest. In some embodiments, a bioproduct is an mRNA expressed from a gene of interest. In some embodiments, a bioproduct is a compound or other composition that is, directly or indirectly, synthesized, modified, or otherwise acted upon by a protein or polynucleotide expressed from a gene of interest.

In some embodiments, a first transcriptional unit comprises a transcription factor. In some embodiments, the first transcriptional unit further comprises an input promoter. In some embodiments, the first transcriptional unit comprises an input promoter that is operably linked to and regulates transcription of a polynucleotide encoding a transcription factor or component thereof. In some embodiments, the first transcriptional unit further comprises a transcriptional terminator.

In some embodiments, a first transcriptional unit is integrated into the genome of a host cell. In some embodiments, a first transcriptional unit is present on a plasmid.

In some embodiments, a first transcriptional unit comprises a transcriptional terminator.

In some embodiments, a first transcriptional unit comprises an input promoter (P(in)) that is operably linked to and regulates transcription of a polynucleotide encoding a transcription factor or component thereof, and a transcriptional terminator. In some embodiments, the first transcriptional unit comprises or consists of a polynucleotide sequence that is at least 70%, at least 80%, at least 90%, at least 91%, at least 92%, at least 93%, at least 94%, at least 95%, at least 96%, at least 97%, at least 98%, at least 99%, or 100% identical to a the nucleic acid sequence in Example 1, Example 3, Tables 21, 28, and 30-36, or to the nucleic acid sequence of any of SEQ ID NOs: 71-85, and is capable of encoding a transcription factor (e.g., a transcriptional activator) or at least one component thereof.

In some embodiments a first transcriptional unit is integrated into the genome of a host cell or is present on a plasmid in combination with a second transcriptional unit, thereby comprising a synthetic expression system.

In some embodiments, the first and second transcriptional units are separated by a spacer. In some embodiments, the spacer is a polynucleotide sequence having from about 2 to about 30 base pairs, from about 2 to about 25 base pairs, from about 2 to about 20 base pairs, from about 2 to about 10 base pairs, or from about 5 to about 10 base pairs. In some embodiments, the spacer is a polynucleotide having at least 7 base pairs. In some embodiments, the spacer comprises a polynucleotide having the sequence GCTTACA (SEQ ID NO: 166).

In some embodiments, a second transcriptional unit comprises a synthetic output promoter. In some embodiments, the synthetic output promoter is operably linked to a gene of interest. In some embodiments, a gene of interest is endogenous. In some embodiments, a gene of interest is exogenous to the host cell. In some embodiments, a gene of interest is synthetic. In some embodiments, a second transcriptional unit further comprises a transcriptional terminator. In some embodiments, the second transcriptional unit comprises a polynucleotide that is at least 70%, at least 80%, at least 90%, at least 91%, at least 92%, at least 93%, at least 94%, at least 95%, at least 96%, at least 97%, at least 98%, at least 99%, or 100% identical to a nucleic acid sequence in Example 1, Example 3, or Tables 21, 28, and 30-36.

In some embodiments, induction of the input promoter by user-controlled cultivation conditions activates transcription of a first transcriptional unit. In some embodiments, the transcription factor of a first transcriptional unit activates the synthetic output promoter of a second transcriptional unit. In some embodiments, activation of the synthetic output promoter activates transcription of a second transcriptional unit.

### Promoters

As used in this application, a “promoter” (e.g., an input promoter or an output promoter) refers to a regulatory region of DNA which directs the transcription of a sequence of DNA into RNA. In some embodiments, a promoter (e.g., an input promoter or an output promoter) comprises a TATA box, or similar sequence, which is capable of directing RNA polymerase II to initiate RNA synthesis at the appropriate transcription initiation site for a particular polynucleotide sequence. In some embodiments, a promoter (e.g., an input promoter or an output promoter) may additionally comprise other sequences, generally but not always positioned upstream of the TATA box, referred to as upstream promoter elements, which influence the transcription initiation rate.

In some embodiments, a promoter (e.g., an input promoter or an output promoter) comprises an upstream activating sequence (UAS) and a core promoter element. In some embodiments, a promoter (e.g., an input promoter or an output promoter) comprises a core promoter element, and does not comprise an upstream activating sequence (UAS).

In certain organisms (e.g., yeasts), a promoter (e.g., an input promoter or an output promoter) may be understood to encompass a sequence spanning from up to 1500 bp upstream of the start codon of the gene to the base abutting the first base of the start codon of the gene. In some embodiments, the 5′-UTR region is the region of an mRNA that begins at the transcription start site and ends directly upstream from the start codon. In some embodiments, a promoter (e.g., an input promoter or an output promoter) comprises a 5′-UTR, which comprises the region from the +1 position of the transcriptional start to the base abutting (immediately upstream of) the start codon (e.g., ATG) of the gene. In some embodiments, a promoter (e.g., an input promoter or an output promoter) comprises the core promoter and the 5′ untranslated region (5′-UTR). In some embodiments, for any particular promoter (e.g., an input promoter or an output promoter), the exact 5′ and 3′ ends of the promoter sequence may be defined differently by different sources, scientific references, etc. In some embodiments, the present disclosure pertains to any sequence of any promoter (e.g., an input promoter or an output promoter) as defined in this document (e.g., any sequence in the appended sequence listing, or to those shown in Tables 21, 28, and 30-36).

In some embodiments, a promoter (e.g., an input promoter or an output promoter) comprises or consists of a polynucleotide having the nucleic acid sequence of any one of SEQ ID NOs: 16-25, 56-70, or 186-193, or a functional fragment thereof. In some embodiments, the promoter (e.g., an input promoter or an output promoter) comprises or consists of a polynucleotide having at least at least 70%, at least 80%, at least 90%, at least 91%, at least 92%, at least 93%, at least 94%, at least 95%, at least 96%, at least 97%, at least 98%, at least 99%, or 100% identity to the nucleic acid sequence of any one of SEQ ID NOs: 16-25, 56-70, or 186-193, or a functional fragment thereof.

A “fragment” of a promoter (e.g., an input promoter or an output promoter) refers to a portion less than the full-length promoter sequence. A “functional fragment” of a promoter (e.g., an input promoter or an output promoter) of the disclosure refers to a biologically active portion of a promoter sequence. A “biologically active portion” of a genetic regulatory element such as a promoter (e.g., an input promoter or an output promoter) may comprise a portion or fragment of a full-length genetic regulatory element and have the same or similar type of activity as the full-length genetic regulatory element, although the level of activity of the biologically active portion of the genetic regulatory element may vary compared to the level of activity of the full-length genetic regulatory element.

### Input Promoters

In some embodiments, this disclosure provides the expression of a polynucleotide encoding a transcription factor or at least one component of a transcription factor under the control of an input promoter, as part of a first transcriptional unit. As used in this application, an “input promoter” refers to a promoter that is operably linked to and capable of activating transcription of a polynucleotide encoding a transcription factor or at least one component of a transcription factor. In some embodiments, the input promoter drives expression of (e.g., is operatively coupled to) the transcription factor or a component of the transcription factor.

In some embodiments, an input promoter comprises an upstream activating sequence (UAS) and a core promoter element. In some embodiments, an input promoter comprises a core promoter element, and does not comprise an upstream activating sequence (UAS).

In some embodiments, the input promoter is naturally occurring. In some embodiments, the input promoter has at least 90% sequence identity to a naturally occurring promoter. In some embodiments, the input promoter is endogenous to the host cell. In some embodiments, the input promoter is exogenous to the host cell. In some embodiments, the input promoter is synthetic.

In some embodiments, the input promoter of the first transcriptional unit is a regulatable input promoter. As used in this application, a “regulatable input promoter” is an input promoter controlled by the presence or absence of a molecule, nutrient, or compound, or by certain physical conditions. In some embodiments, the regulatable input promoter is inducible. In some embodiments, the regulatable input promoter is repressible. A regulatable input promoter may be used, for example, to controllably activate (e.g., induce) or repress the expression of a transcription factor or at least one component of a transcription factor, and the transcription factor activates a synthetic output promoter to express a gene of interest. As will be understood, “repression” of the expression of a transcription factor or at least one component of a transcription factor may in some embodiments comprise reducing the level of expression of a transcription factor or at least one component of a transcription factor. In some embodiments, the expression of a transcription factor or at least one component of a transcription factor may be eliminated completely, and still be considered “repressed,” as the term is used in this disclosure.

Non-limiting examples of regulatable input promoters include chemically regulated input promoters and physically regulated input promoters. For chemically regulated input promoters, transcriptional activity can be regulated by one or more compounds, such as an alcohol (e.g., methanol), tetracycline, galactose, glycerol, glucose, maltose, dextrose, sorbitol, inositol, methionine, formic acid, phosphate, a steroid, a metal, a nutrient, and combinations thereof; and in some embodiments, transcriptional activity is regulated by the addition of or by the limitation or depletion of the compound or combination thereof. For physically regulated input promoters, transcriptional activity can be regulated by a change in light, pressure, temperature, or other factors.

Non-limiting examples of tetracycline-regulated promoters include anhydrotetracycline (aTc)-responsive promoters and other tetracycline-responsive promoter systems (e.g., a tetracycline repressor protein (TetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells.

In some embodiments, a regulatable input promoter is a methanol-inducible input promoter. As used in this disclosure, a “methanol-inducible promoter” is a promoter (e.g., an input promoter or an output promoter) whose activity is substantially increased by the presence of methanol in a culture medium. In some embodiments, wherein a methanol-inducible promoter drives expression of a gene of interest, a “substantial increase of activity” is when at least 20× more transcripts per million of the gene of interest are produced when exogenously added methanol is present in the culture medium, compared to when exogenously added methanol is not present in the culture medium.

Conversely, a promoter that is “not a methanol-inducible promoter” is a promoter (e.g., an input promoter or an output promoter) whose activity is not substantially increased by the presence of methanol in a culture medium. In some embodiments, wherein a methanol-inducible promoter drives expression of a gene of interest, a “non-substantial increase of activity” is when the difference between the transcripts per million of the gene of interest produced when exogenously added methanol is present in the culture medium compared to when exogenously added methanol is not present in the culture medium is less than 2×.

In some embodiments, a regulatable input promoter is regulatable in methanol-independent conditions. In some embodiments, the regulatable input promoter is regulatable in the absence of exogenously provided methanol. In some embodiments, the input promoter is not methanol inducible. In some embodiments, the inducible input promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, polynucleotides, protein transcriptional activators and repressors, cytokines, toxins, petroleum-based compounds, metal containing compounds, salts, ions, enzyme substrate analogs, hormones, or any combination thereof.

Aspects of the disclosure relate to the production of proteins and/or nucleic acids expressed from a gene of interest in methanol-independent fermentation conditions. In some embodiments, the input promoter of the first transcriptional unit and/or the output promoter of the second transcriptional unit is a regulatable input promoter. In some embodiments, the regulatable input promoter is responsive (e.g., inducible) in the absence of methanol. In some embodiments, the regulatable input promoter is responsive to nutrient addition, limitation, or depletion with respect to a cognate cultivation process. In some embodiments, the regulatable input promoter is responsive to thiamine depletion. In some embodiments, the regulatable input promoter is responsive to glycerol depletion. In some embodiments, the regulatable input promoter is responsive to glucose limitation. In some embodiments, the regulatable input promoter is responsive to formic acid limitation. In some embodiments, the regulatable inducible promoter is responsive to monosaccharide limitation. In some embodiments, the regulatable inducible promoter is responsive to the limitation of a carbon source, a sugar, a starch, galactose, maltose, glucose, dextrose, sorbitol, inositol, glycerol, methionine, a vitamin, phosphate, a steroid, a nitrogen source, nitrate, nitrite, ammonium, an amino acid, methionine, a metal (e.g., a heavy metal), copper, benzoic acid, hydrogen peroxide, a calcium-containing compound, an alcohol, methanol, tetracycline, a steroid, and/or phosphate. Various regulatable input promoters are known in the art. In some embodiments, the regulatable input promoter is responsive to the presence of or addition of (e.g., the addition to the medium of an excess of) any of: a nutrient, an antibiotic, tetracycline, doxycycline, a sugar, a starch, galactose, maltose, glucose, sorbitol, inositol, glycerol, formic acid, a vitamin, a steroid, a nitrogen source, nitrate, nitrite, ammonium, an amino acid, methionine, an ion, sodium, and/or phosphate.

In some embodiments, a single limiting nutrient is used. In some embodiments, the regulatable input promoter is responsive to limitation of a combination of nutrients (e.g., two nutrients, or more than two nutrients). In some embodiments, a combination of limiting nutrients is used. In some embodiments, the regulatable input promoter is responsive to limitation of a combination of nutrients, including but not limited to: glycerol, glucose, and thiamine, or wherein the combination is: glycerol and formic acid; glucose and formic acid; or glucose and thiamine. In some embodiments, the activity of the regulatable input promoter is increased by the presence of exogenously provided formic acid. The activity of the regulatable input promoter is considered “increased” by the presence of exogenously provided formic acid when the level of expression of e.g. a transcription factor is elevated compared the level of expression of the transcription factor prior to the exogenous provision of formic acid. In some embodiments, the response of the regulatable input promoter (e.g., once induced) is activation. In some embodiments, the response of the regulatable input promoter is repression.

In some embodiments, an input promoter is cognate with respect to a particular production process (e.g., a process described in this document such as Process 1, Process 2, or Process 3) if the input promoter is activated under a particular culturing step or condition in that process (e.g., for Process 1, limiting glycerol+added formic acid; for Process 2, limiting glucose+added formic acid; and for Process 3, limiting glucose+depleted thiamine).

In some embodiments, an input promoter is activated under the culture conditions of glycerol and added formic acid. In some embodiments, an input promoter is activated under the culture conditions of glucose and added formic acid. In some embodiments, an input promoter is activated under the culture conditions of glucose and depleted thiamine. In some embodiments, an input promoter is activated in a methanol-dependent process. In some embodiments, an input promoter is cognate with respect to Process 1, which comprises the culture conditions of glycerol and added formic acid. In some embodiments, an input promoter is cognate with respect to Process 2, which comprises the culture conditions of glucose and added formic acid. In some embodiments, an input promoter is cognate with respect to Process 3, which comprises the culture conditions of glucose and depleted thiamine. In some embodiments, an input promoter is cognate with respect to Process 4, which is a methanol-dependent process. In some experiments described herein, Process 4 is used as a control.

In some embodiments, an input promoter is cognate with respect to Process 1, for example, one listed in Table 4. In some embodiments, an input promoter is cognate with respect to Process 2, for example, one listed in Table 5. In some embodiments, an input promoter is cognate with respect to Process 3, for example, one listed in Table 6. In some embodiments, an input promoter is one listed in Table 4. In some embodiments, an input promoter is one listed in Table 5. In some embodiments, an input promoter is one listed in Table 6.

In some embodiments, an input promoter of the first transcriptional unit and/or an output promoter of the second transcriptional unit is a constitutive promoter. As used in this application, a “constitutive promoter” is a promoter that, when operably linked to a DNA sequence in the context of a given host genome, leads to continuous transcription of the DNA sequence. Non-limiting examples of constitutive promoters include P(GAP), P(ENO1), P(GPM1), P(HSP82), P(ILV5), P(KAR2), P(KEX2), P(PET9), P(PGK1), P(SSA4), P(TEF1), P(TPI1), and P(YPT1).

In some embodiments, the input promoter of the first transcriptional unit comprises or consists of a polynucleotide that is at least 90%, at least 91%, at least 92%, at least 93%, at least 94%, at least 95%, at least 96%, at least 97%, at least 98%, at least 99%, or 100% identical to a nucleic acid sequence in Examples 1 and 3, Table 21, or to the nucleic acid sequence of any one of SEQ ID NOs: 16-25. In some embodiments, the input promoter comprises a polynucleotide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 100, 150, 200, 250, or 300 nucleotide substitutions, insertions, additions, or deletions relative to the nucleic acid sequence of any one of SEQ ID NOs: 16-25. In some embodiments, the input promoter is capable of initiating transcription of a polynucleotide encoding a transcription factor or at least one component thereof. In some embodiments, the transcription factor is a transcriptional activator. In some embodiments, the input promoter of the first transcriptional unit comprises or consists of a polynucleotide having the nucleic acid sequence of any one of SEQ ID NOs: 16-25.

In some embodiments, the input promoter is any one of P(CMC1), P(JEN1), P(GQ6704499), P(GQ700926), P(HGT1), P(FDH1), P(AOX2), P(RGI2), P(PIH1), P(THI4a), or P(THI4b). In some embodiments, the input promoter is P(AT249_GQ6704499). In some embodiments, the input promoter does not comprise P(AOX1), or a promoter having more than 90%, 80%, or 70% sequence identity to P(AOX1).

Non-limiting examples of P(in)s are described in Table 4, Table 5, and Table 6.

DNA sequences (by SEQ ID NO) of P(in)s in Table 4, Table 5, or Table 6 can be found in Table 21.

Transcription Factors [TF or sTF]

In some embodiments, the disclosure pertains to a transcriptional unit that expresses at least one component of a transcription factor. In some embodiments a synthetic expression system comprises a first and a second transcriptional unit, wherein the first transcriptional unit expresses at least one component of a transcription factor, and the second transcriptional unit comprises a synthetic output promoter activated by the transcription factor, wherein the synthetic output promoter promotes expression of a gene of interest.

In some embodiments, a synthetic expression system is provided, wherein the input promoter drives the expression of at least one component of a transcription factor, which is encoded by a polynucleotide present in the first transcriptional unit. In some embodiments, the synthetic transcription factor is not an activator of the input promoter. In some embodiments, the synthetic transcription factor is an activator of the synthetic output promoter. In some embodiments, a component of the transcription factor binds to the synthetic output promoter of the second transcriptional unit and drives expression of a gene of interest. In some embodiments, a transcription factor (TF) is a synthetic transcription factor (sTF).

A “transcription factor” is a protein that controls the rate of transcription from a cognate promoter by binding to one or more specific DNA sequences in or around the promoter.

In some embodiments, a transcription factor increases the rate of transcription of a gene of interest by binding to the synthetic output promoter operably linked to the gene of interest. A transcription factor can work alone, or can work with other proteins in a complex by recruiting components of and/or stabilizing a complex comprising an RNA polymerase at the synthetic output promoter. In some embodiments, a transcription factor comprises at least one of: (1) a DNA-binding domain, which binds to a specific DNA sequence, and/or (2) a transcriptional activation domain (e.g., a trans-acting domain; TAD), which can interact with another protein such as a RNA polymerase, another protein, or another component in a complex comprising the RNA polymerase.

Without wishing to be bound by any particular theory, it is noted that a transcription factor can increase expression from a synthetic output promoter by various mechanisms, including but not limited to: stabilizing the binding of RNA polymerase to the promoter; catalyzing the acetylation of histone proteins via histone acetyltransferase (HAT) activity; weakening the association of DNA with histones and making the DNA more accessible to transcription; and/or recruiting coactivator or corepressor proteins to the transcription complex. In some embodiments, a transcription factor comprises a signal-sensing domain (SSD) (e.g., a ligand binding domain), which senses external signals and, in response, transmits these signals to the rest of the transcription complex, resulting in up-regulation of expression of the gene of interest.

Various transcription factors, and their structures and functions, are described in the literature, including: Latchman 1997 Int. J. Biochem. Cell Biology. 29 (12): 1305-12; Karin 1990 The New Biologist. 2 (2): 126-31; Babu et al. 2004 Current Opinion in Structural Biology. 14 (3): 283-91; Roeder 1996 Trends in Biochemical Sciences. 21 (9): 327-35; Nikolov et al. 1997 Proc. Nat. Acad. Sci. United States of America. 94 (1): 15-22; Lee et al. 2000 Annual Review of Genetics. 34: 77-137; Mitchell et al. 1989 Science. 245 (4916): 371-8; Ptashne et al. 1997 Nature. 386 (6625): 569-77; Jin et al. 2014 Nucleic Acids Research. 42 (Database issue): D1182-7; and Matys et al. 2006 Nucleic Acids Research. 34 (Database issue): D108-10.

In some embodiments, the transcription factor comprises one or more of the following components: (a) a DNA-binding domain, which binds to the synthetic output promoter (e.g., binds to an operator within the synthetic output promoter), and/or (b) a transcriptional activation domain, which binds to another factor which facilitates transcription from the synthetic output promoter (e.g., a RNA polymerase), (c) optionally, a nuclear localization signal, (d) optionally, an oligomerization domain, and (e), optionally, one or more linkers between any of components (a) to (d), if present. In some embodiments, the transcription factor further comprises one or more of the following components: (f) optionally, one or more additional domains, and (g) optionally, if one or more components (f) is present, one or more linkers between component (f) and any of components (a) to (d), and/or, if more than one component (f) is present, one or more linkers between any of the components (f). In some embodiments, the one or more components (f) can perform any of various functions, including, but not limited to: binding ATP; directly or indirectly catalyzing the acetylation or deacetylation of one or more histones; binding to another protein; recruiting a coactivator; binding to another transcription factor; binding to a component of the transcription preinitiation complex; binding to a ligand or signal compound; acting as a signal-sensing domain; performing a function in a signaling cascade; performing a function related to regulation of the cell cycle; performing a function related to regulation of development; acting as a site for phosphorylation; and/or binding to a membrane. As used in this disclosure, a “component” or “component part” of a transcription factor refers to part-types such as those provided in (a)-(f) above. In some embodiments, the transcription factor is chimeric, in that any two or more of: the DNA-binding domain, transcriptional activation domain, nuclear localization signal (NLS), and/or any other component are derived from different sources (e.g., different species).

In some embodiments, a synthetic expression system comprises a transcription factor or at least one component of a transcription factor, wherein the transcription factor comprises or consists of a sequence (e.g., nucleic acid or amino acid sequence) that is at least 90%, at least 91%, at least 92%, at least 93%, at least 94%, at least 95%, at least 96%, at least 97%, at least 98%, at least 99%, or 100% identical to a sequence selected from any one of SEQ ID NOs: 26-55. In some embodiments, a synthetic expression system comprises a transcription factor or at least one component of a transcription factor, wherein the transcription factor does not comprise methanol expression regulator 1 (mxr1), or a transcription factor having 90%, 80% or 70% sequence identity to mxr1. In some embodiments, a synthetic expression system comprises a transcription factor or at least one component of a transcription factor, wherein the transcription factor does not comprise human estrogen receptor alpha (hERα) or a transcription factor having 90%, 80% or 70% sequence identity to hERα. In some embodiments, a synthetic expression system comprises a transcription factor or at least one component of a transcription factor, wherein the transcription factor does not comprise pheromone-regulated membrane protein 1 (prm1) or a transcription factor having 90%, 80% or 70% sequence identity to prm1.

In some embodiments, a transcription factor comprises a DNA-binding domain (DBD). A “DNA binding domain” is an independently folded protein domain that contains at least one structural motif that recognizes double- or single-stranded DNA. A DNA binding domain can recognize a specific DNA sequence (a recognition sequence) or have a general affinity to DNA. In some embodiments, the DNA-binding domain is or is derived from that of Bm3R1, TetR, PhlF_AM, or VanR_AM.

In some embodiments, the DNA binding domain of Bm3R1 is a DNA binding domain that is based on (e.g., a portion of, derived from, a variant of, etc.) the full-length Bm3R1 repressor. In some embodiments, Bm3R1 encodes the full-length sequence of the transcriptional repressor Bm3R1 from Bacillus megaterium (NCBI accession #WP_013083972.1).

In some embodiments, the DNA binding domain of TetR is a DNA binding domain that is based on the full-length TetR repressor. In some embodiments, TetR encodes the full-length sequence of the transcriptional repressor TetR from Tn10 (NCBI accession #WP_000088605.1).

In some embodiments, the DNA binding domain of PhlF_AM is a DNA binding domain that is based on full-length PhlF_AM. In some embodiments, PhlF_AM encodes a variant of the full-length sequence of transcriptional repressor PhlF from Pseudomonas fluorescens (NCBI accession #AYJ72227.1).

In some embodiments, the DNA binding domain of VanR_AM is a DNA binding domain based on full-length VanR_AM. In some embodiments, VanR_AM encodes a variant of the full-length sequence of the transcriptional repressor VanR from Caulobacter (NCBI accession #AYJ72236.1).

In some embodiments, the DNA binding domain comprises or consists of a polynucleotide having the nucleic acid sequence of any one of SEQ ID NOs: 86-89, or a functional fragment thereof. In some embodiments, the DNA binding domain comprises an amino acid having the nucleic acid sequence of any of SEQ ID NOs: 90-93, or a functional fragment thereof.

In some embodiments, a transcription factor is a one-component, two component, or multi-component transcription factor.

In some embodiments, a transcription factor is any one of eight different types of sTFs: (1) one-component Bm3R1-based sTFs, (2) one-component PhlF_AM-based sTFs, (3) one-component TetR-based sTFs, (4) one-component VanR_AM-based sTFs, (5) two-component Bm3R1-based sTFs, (6) two-component PhlF_AM-based sTFs, (7) two-component TetR-based sTFs, and (8) two-component VanR_AM-based sTFs. As used here, “one-component,” “two-component,” and “multi-component” refers to a number of subunits present in the sTF. An sTF “subunit” may comprise component parts (such as, for example, DNA binding domains, transcriptional activation domains, BPP1s, BPP2s, nuclear localization signals, spacers, etc.). In some embodiments, a one-component sTF is a synthetic transcription factor comprising one or more monomers of a polypeptide chain bearing a DBD, an NLS, and a TAD, wherein the polypeptide chain is encoded by a single DNA coding sequence.

In some embodiments, a transcription factor is a Bm3R1-based sTF. In some embodiments, a transcription factor is a PhlF_AM-based sTF. In some embodiments, a transcription factor is a TetR-based sTF. In some embodiments, a transcription factor is a VanR_AM-based sTF. In some embodiments, a transcription factor is a Bm3R1-based sTF. In some embodiments, a transcription factor is a PhlF_AM-based sTF. In some embodiments, a transcription factor is a TetR-based sTF. In some embodiments, a transcription factor is a VanR_AM-based sTF. In some embodiments, a transcription factor is a one-component Bm3R1-based sTF. In some embodiments, a transcription factor is a one-component PhlF_AM-based sTF. In some embodiments, a transcription factor is a one-component TetR-based sTF. In some embodiments, a transcription factor is a one-component VanR_AM-based sTF. In some embodiments, a transcription factor is a one-component Bm3R1-based sTF, for example, one listed in Table 7. In some embodiments, a transcription factor is a one-component PhlF_AM-based sTF, for example, one listed in Table 8. In some embodiments, a transcription factor is a one-component TetR-based sTF, for example, one listed in Table 9. In some embodiments, a transcription factor is a one-component VanR_AM-based sTF, for example, one listed in Table 10.

In some embodiments, a transcription factor is a two-component Bm3R1-based sTF. In some embodiments, a transcription factor is a two-component PhlF_AM-based sTF. In some embodiments, a transcription factor is a two-component TetR-based sTF. In some embodiments, a transcription factor is a two-component VanR_AM-based sTF. In some embodiments, a transcription factor is a two-component Bm3R1-based sTF, for example, one listed in Table 11. In some embodiments, a transcription factor is a two-component PhlF_AM-based sTF, for example, one listed in Table 12. In some embodiments, a transcription factor is a two-component TetR-based sTF, for example, one listed in Table 13. In some embodiments, a transcription factor is a two-component VanR_AM-based sTF, for example, one listed in Table 14.

In some embodiments, one-component sTFs are designed to bring within molecular proximity a DBD and a TAD that, in conjugation with a cognate synthetic output promoter and RNA polymerase complex, mediate transcriptional activation of the synthetic output promoter. In some embodiments, the DBD and TAD are essential components with respect to the functionality of synthetic expression systems mediated by one-component sTFs.

### Transcription Factors Comprising One or More Components

In some embodiments, the present disclosure pertains to a synthetic expression system comprising any transcription factor or at least one component of a transcription factor as described in the present disclosure, or a method of use thereof. In some embodiments, the present disclosure pertains to any transcription factor or at least one component of a transcription factor as described in the present disclosure, or a method of use thereof. In some embodiments, the present disclosure pertains to any transcription factor or at least one component of a transcription factor as described in the present disclosure, for use in combination of a cognate output promoter, or a method of use thereof.

In some embodiments, a transcription factor comprises one component, two components, three components, four components, five components, or more than five components. Transcription factors having more than two components are referred to as “multi-component” transcription factors. In some embodiments, a transcription factor comprises or consists of one component. In some embodiments, a transcription factor comprises two components. In some embodiments, a transcription factor comprises or consists of two or more components.

In some embodiments, at least one component of a transcription factor comprises a DNA-binding domain (DBD) or a portion thereof. In some embodiments, at least one component of a transcription factor comprises a transcriptional activation domain (TAD) or a portion thereof. In some embodiments, at least one component of a transcription factor comprises a portion which binds to a different component of the transcription factor. In various embodiments, two or more components of a transcription factor can be derived from different sources (e.g., different genera, different species, etc.).

In some embodiments, two or more components of a transcription factor join (e.g., bind one to the other, or bind each other) to form the transcription factor. In some embodiments, two or more components of a transcription factor join, forming a heterodimer, chimera, or fusion. In some embodiments, two components of a transcription factor join to form a heterodimer, chimera, or fusion through any biochemical mechanism in which a portion of one component binds to the other, or vice versa, or wherein portions of the two components bind to each other.

In some embodiments, a two-component sTF is a synthetic transcription factor which comprises a complex comprising one or more monomers of two-component sTF component polypeptide #1 and one or more monomers of two-component sTF component polypeptide #2. The inter-molecular complexation between or among two-component sTF component polypeptide #1 and two-component sTF component polypeptide #2 can be mediated by either non-covalent interactions or covalent bonding (e.g., using bioconjugate proteins). In some embodiments, a two-component or multi-component transcription factor further comprises a bioconjugate protein.

In some embodiments of non-covalent complexation, two-component sTF component polypeptide #1 and two-component sTF component polypeptide #2 can be brought together via specific, high-affinity, non-covalent interactions between a protein domain or other sub-sequence (e.g., a short epitope or tag) on two-component sTF component polypeptide #1 and a cognate protein domain or other sub-sequence (e.g., a short epitope or tag) on two-component sTF component polypeptide #2. An example of such a system is embodied in the ALFA-tag/NbALFA system, wherein the ALFA-tag comprises a short peptide sequence tightly bound by a cognate nanobody (NbALFA).

In some embodiments of covalent complexation, two-component sTF component polypeptide #1 and two-component sTF component polypeptide #2 can be brought together via a specific covalent bond formation event between a protein domain or other sub-sequence (e.g., a short epitope or tag) on two-component sTF component polypeptide #1 and a cognate protein domain or other sub-sequence (e.g., a short epitope or tag) on two-component sTF component polypeptide #2. In some embodiments, the covalent bond formed is an isopeptide bond. An example of such a system is embodied in the SpyTag/SpyCatcher system, wherein the SpyTag comprises a short peptide sequence that forms an isopeptide bond with a cognate SpyCatcher domain.

In some embodiments, two-component sTF component polypeptide #1 harbors a DBD and a first NLS (NLS1), while two-component sTF component polypeptide #2 harbors a TAD and a second NLS (NLS2). Two-component sTFs are thus designed to bring within molecular proximity one or more DBDs and one or more TADs that, in conjugation with a cognate synthetic output promoter, mediate transcriptional activation of the synthetic output promoter.

In some embodiments, the inter-molecular complexation between or among two-component sTF component polypeptide #1 and two-component sTF component polypeptide #2 is mediated by the formation of a covalent isopeptide bond. In some embodiments, the two-component or multi-component transcription factor further comprises SpyTag and/or SpyCatcher. In some embodiments, two-component sTF component polypeptide #1 bears one or more copies of the SpyTag variant and two-component sTF component polypeptide #2 bears one copy of the SpyCatcher variant. Other examples of such systems include variants of SpyTag/SpyCatcher, SnoopTag/SnoopCatcher, SdyTag/SdyCatcher, and/or any other bioconjugate proteins known in the art. As used in this document, a short protein sequence equivalent in functionality to the SpyTag variant is referred to as “Bioconjugate Protein Part 1 (BPP1)”, and a relatively large cognate protein sequence equivalent in functionality to the SpyCatcher variant is referred to as “Bioconjugate Protein Part 2 (BPP2).”

In some embodiments, a transcription factor comprises a single copy of a BPP1. In some embodiments, the single copy of a BPP1 comprises or consists of a polynucleotide having the nucleic acid sequence of SEQ ID NO: 148. In some embodiments, the single copy of a BPP1 comprises or consists of a polypeptide having the amino acid sequence of SEQ ID NO: 151.

In some embodiments, a transcription factor comprises multiple copies of a BPP1, for example 2, 3, 4, 5, 6, 7, 8, 9, 10, or more copies. In some embodiments, a transcription factor comprises 2 copies of a BPP1. In some embodiments, the 2 copies of a BPP1 comprise or consist of a polynucleotide having the sequence of SEQ ID NO: 149. In some embodiments, the 2 copies of a BPP1 comprise or consist of a polypeptide having the amino acid sequence of SEQ ID NO: 152. In some embodiments, a transcription factor comprises 6 copies of a BPP1. In some embodiments, the 6 copies of a BPP1 comprise or consist of a polynucleotide having the nucleic acid sequence of SEQ ID NO: 150. In some embodiments, the six copies of a BPP1 comprise or consist of a polypeptide having the amino acid sequence of SEQ ID NO: 153.

In some embodiments, a transcription factor comprises one or more copies of a BPP1 (e.g., 2, 3, 4, 5, 6, 7, 8, 9, 10, or more than 10 copies) and/or a single copy of a BPP2. In some embodiments, the single copy of a BPP2 comprises or consists of a polynucleotide having the sequence of SEQ ID NO: 154. In some embodiments, the single copy of a BPP2 comprises or consists of a polypeptide having the amino acid sequence of SEQ ID NO: 155. In some embodiments, a transcription factor comprises one or more copies of a BPP1 (e.g., 1, 2, 3, 4, 5, 6, 7, 8, 9, 10, or more than 10 copies) and/or one or more copies (e.g., 1, 2, 3, 4, 5, 6, 7, 8, 9, 10, or more than 10 copies) of a BPP2.

In some embodiments, the transcription factor further comprises a self-cleaving polypeptide. In some embodiments, the self-cleaving polypeptide is a 2A peptide. In some embodiments, the self-cleaving polypeptide is ERBV_1_P2A. In some embodiments, the self-cleaving polypeptide is E2A, F2A, or T2A.

In some embodiments, the protein sequence of the two-component sTF component polypeptide #1 and the protein sequence of two-component sTF component polypeptide #2 have been encoded in the same transcriptional unit, driven by a single promoter, wherein the two different polypeptide chains are generated from a single coding sequence via a “ribosome skipping” event mediated by an intervening encoded 2A peptide sequence. In some embodiments, the protein sequence of two-component sTF component polypeptide #1 and the protein sequence of two-component sTF component polypeptide #2 are encoded in separate transcriptional units, each driven by a separate promoter.

In some embodiments, different components of a transcription factor are encoded by different genes. In some embodiments of a synthetic expression system, a transcription factor comprises or consists of two or more components, wherein each component is encoded by a different gene.

In some embodiments of a synthetic expression system, a transcription factor comprises or consists of two or more components, wherein each component is encoded by a different gene, and wherein the different genes encoding the two or more components are polycistronic (e.g., controlled by an input promoter, and/or controlled by the same promoter).

In some embodiments of a synthetic expression system, a transcription factor comprises or consists of three or more components, wherein each component is encoded by a different gene, and wherein two or more of the different genes are polycistronic (e.g., controlled by an input promoter, and/or controlled by the same promoter, which can be regulatable or not regulatable).

In various embodiments, a synthetic expression system can comprise a transcription factor comprising two or more components, wherein the components are expressed as parts of the same or different transcriptional units.

In some embodiments, a first transcriptional unit comprises an input promoter which controls expression of two genes, each encoding a component of the transcription factor, wherein the input promoter and the two genes are parts of a polycistronic (or bicistronic) unit (e.g., a polycistronic or bicistronic locus, system, etc.).

In some embodiments, a transcription factor comprises two components, each encoded by a separate gene, wherein both of the genes are expressed as a part of a first transcriptional unit, and when the genes encoding the two components are expressed, the two components are capable of joining to form the transcription factor, and the transcription factor is capable of activating the output promoter, which is operably linked to and expresses a gene of interest.

In some embodiments, a synthetic expression system comprises: (a) a first transcriptional unit, which comprises: a first input promoter operably linked to and capable of expressing: (i) a gene encoding a first component of a transcription factor, and (ii) a gene encoding a second component of the transcription factor; and (b) a second transcriptional unit, which comprises: an output promoter operably linked to and capable of expressing a gene of interest; wherein the first and second components are capable of joining to form the transcription factor, and the transcription factor is capable of activating the output promoter to express a gene of interest.

In some embodiments, a transcription factor comprises at least two components, wherein each component is expressed as a part of the same or a different transcriptional unit, and when the genes encoding the at least two components are expressed, the at least two components are capable of joining to form the transcription factor, and the transcription factor is capable of activating the output promoter, which is operably linked to and expresses a gene of interest.

In some embodiments, a synthetic expression system comprises: (a) a first transcriptional unit, which comprises: a first input promoter operably linked to and capable of expressing a gene encoding a first component of a transcription factor; (b) a second transcriptional unit, which comprises: an output promoter operably linked to and capable of expressing a gene of interest; and (c) a third transcriptional unit, which comprises: a second input promoter operably linked to and capable of expressing a gene encoding a second component of the transcription factor; wherein the first and second input promoters are the same or different; wherein neither, either, or both of the first and second input promoters are inducible; and wherein the first and second components are capable of joining to form the transcription factor, and the transcription factor is capable of activating the output promoter to express a gene of interest.

In some embodiments, a transcription factor comprises at least three components, wherein each component is expressed as a part of a different transcriptional unit, and when the genes encoding the at least three components are expressed, the at least three components are capable of joining to form the transcription factor, and the transcription factor is capable of activating the output promoter, which is operably linked to and expresses a gene of interest.

In some embodiments, a synthetic expression system comprises: (a) a first transcriptional unit, which comprises: a first input promoter operably linked to and capable of expressing a gene encoding a first component of a transcription factor; (b) a second transcriptional unit, which comprises: an output promoter operably linked to and capable of expressing a gene of interest; and (c) a third transcriptional unit, which comprises: a second input promoter operably linked to and capable of expressing a gene encoding a second component of the transcription factor; and (d) a fourth transcriptional unit, which comprises: a third input promoter operably linked to and capable of expressing a gene encoding a third component of the transcription factor; wherein the first, second, and third input promoters are the same or different; wherein none, any, or all of the first, second, or third input promoters are inducible; and wherein the first, second, and third components are capable of joining to form the transcription factor, and the transcription factor is capable of activating the output promoter to express the gene of interest.

In some embodiments, a transcription factor comprises at least three components, wherein each component is expressed as a part of the same or a different transcriptional unit, and, when the genes encoding the at least three components are expressed, the at least three components are capable of joining to form the transcription factor, and the transcription factor is capable of activating the output promoter, which is operably linked to and expresses the gene of interest.

In some embodiments, a synthetic expression system comprises: (a) a first transcriptional unit, which comprises: a first input promoter operably linked to and capable of expressing a gene encoding a first component of a transcription factor; (b) a second transcriptional unit, which comprises: an output promoter operably linked to and capable of expressing a gene of interest; and (c) a third transcriptional unit, which comprises: a second input promoter operably linked to and capable of expressing: (i) a gene encoding a second component of the transcription factor, and (ii) a gene encoding a third component of the transcription factor; wherein the first and second input promoters are the same or different; wherein neither, either, or both of the input promoters are inducible; and wherein the first, second, and third components are capable of joining to form the transcription factor, and the transcription factor is capable of activating the output promoter to express a gene of interest.

In some embodiments, a transcription factor comprises n components, wherein n is two or more, wherein two or more of the n components are expressed as a part of the same or different transcriptional units, and when the genes encoding the n components are expressed, the n components are capable of joining to form the transcription factor, and the transcription factor is capable of activating the output promoter, which is operably linked to and expresses a gene of interest.

In some embodiments, a synthetic expression system comprising a transcription factor comprising n components, wherein each of the n components is encoded by a different gene, wherein the system comprises: (a) a first transcriptional unit, which comprises: a first input promoter operably linked to and capable of expressing the genes encoding the n components of the transcription factor; (b) a second transcriptional unit, which comprises: an output promoter operably linked to and capable of expressing a gene of interest; and wherein the n components are capable of joining to form the transcription factor, and the transcription factor is capable of activating the output promoter to express the gene of interest; wherein n is two or more.

In some embodiments, a synthetic expression system comprises a transcription factor comprising multiple components, wherein each of the components is encoded by a different gene, and wherein the system comprises: (a) one or more first transcriptional units, each of which comprises: an input promoter operably linked to and capable of expressing at least one gene encoding a component of the transcription factor, wherein together all of the transcriptional units express all of the components of the transcription factor; and (b) a second transcriptional unit, which comprises: an output promoter operably linked to and capable of expressing a gene of interest; and wherein the multiple components are capable of joining to form the transcription factor, and the transcription factor is capable of activating the output promoter to express the gene of interest.

In some embodiments, a synthetic expression system comprising a transcription factor comprising multiple components, wherein each of the components is encoded by a different gene, and wherein the system comprises: (a) one or more first transcriptional units, each of which comprises: an input promoter operably linked to and capable of expressing at least one gene encoding a component of the transcription factor, wherein the input promoters on the one or more first transcriptional units are the same or different, and the number of the one or more first transcriptional units is equal to or less than the number of the components, and wherein together all of the transcriptional units express all of the components of the transcription factor; and (b) a second transcriptional unit, which comprises: an output promoter operably linked to and capable of expressing a gene of interest; and wherein the multiple components are capable of joining to form the transcription factor, and the transcription factor is capable of activating the output promoter to express the gene of interest.

In some embodiments, the transcription factor comprises a transcriptional activation domain (TAD). A “transcriptional activation domain” is a region of a transcription factor which, in conjunction with a DNA binding domain, can activate transcription from a promoter. In some embodiments, the transcriptional activation domain is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD (e.g., a transcriptional activation domain of B112, a transcriptional activation domain of B42, a transcriptional activation domain of GAL4, a transcriptional activation domain of miniVPR, a transcriptional activation domain of Mxr1, a transcriptional activation domain of PH, a transcriptional activation domain of VP16, a transcriptional activation domain of VP64, a transcriptional activation domain of VP64v2, a transcriptional activation domain of VPH, or a transcriptional activation domain of VPR, respectively). In some constructs described in this document, for example, in some controls, “No_TAD” indicates that there is no TAD present in that particular construct. In some constructs described in this document, for example, in some controls, the location of a transcriptional activation domain is described in this document as No_TAD, indicating that the transcriptional activation domain is absent. In some constructs, for example, in some controls, a component (e.g., a TAD, an operator, etc.) can be absent and can be replaced by a spacer.

In some embodiments, the DNA-binding domain is, or is derived from that of Bm3R1, TetR, PhlF_AM, or VanR_AM, and the transcriptional activation domain any one of B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD (as described in this disclosure).

In some embodiments, the transcriptional activation domain comprises a polynucleotide having at least 90%, at least 95%, or at least 99% identity to the nucleic acid sequence of any one of SEQ ID NOs: 94-104, or a functional fragment thereof. In some embodiments, the transcriptional activation domain comprises a polypeptide having at least 90%, at least 95%, or at least 99% identity to the nucleic acid sequence of any one of SEQ ID NOs: 105-115, or a functional fragment thereof.

In some embodiments, the transcription factor optionally includes a nuclear localization signal. A “nuclear localization signal” is an amino acid sequence that mediates the transport of nuclear proteins into the cell nucleus. In some embodiments, the nuclear localization signal is from simian virus 40 (SV40). In some embodiments, a polynucleotide sequence encoding a nuclear localization signal from SV40 is GAGTTCCCACCAAAAAAAAAGAGGAAAGTC (SEQ ID NO: 116). In some embodiments, a polynucleotide sequence encoding a nuclear localization signal from SV40 is GAGTTCCCCCCCAAGAAAAAGAGGAAAGTT (SEQ ID NO: 117). In some embodiments, a polynucleotide sequence of a nuclear localization signal from SV40 encodes a protein (e.g., a polypeptide) having the amino acid sequence EFPPKKKRKV (SEQ ID NO: 118). In some embodiments, a polynucleotide sequence of a nuclear localization signal from SV40 encodes a protein having the amino acid sequence PKKKRKV (SEQ ID NO: 119).

Many different nuclear localization signals have been described in the art, for example, as part of a protein, including but not limited to: the homeodomain of yeast repressor alpha 2; cytosolic proteins; the maize regulatory protein Opaque-2; Ras; Rho family small GTPases; agrobacterium VirD2 protein; VirE2 and VirD2; the hsp56 immunophilin component of steroid receptor heterocomplexes; cytoplasmic anchoring proteins; various signal transducers; the glucocorticoid receptor; the UL84 protein of human cytomegalovirus; proteins in the pore complex or in the cytoplasm; ErbB3 13; ErbB4 1; ErbB2; retinoblastoma gene product; a Ty1 integrase; or SV40. See for example: Nguyen et al. BMC Bioinformatics volume 10, Article number: 202 (2009); Lin et al. PLoS One, Oct. 29, 2013, https://doi.org/10.1371/journal.pone.0076864; Hawkins et al. J. Proteome Res. 2007, 6, 4, 1402-1409; Nair et al. Nucleic Acids Research, Volume 31, Issue 1, 1 Jan. 2003, Pages 397-399.

In some embodiments, the transcription factor further comprises an oligomerization domain (OD). In some embodiments, the oligomerization domain is Linker_only_for_oligomerization (e.g., a linker for oligomerization; SEQ ID NO: 157); Trimerization_domain (e.g., a trimerization domain; SEQ ID NO: 158); or Heptamerization_domain (e.g., a heptamerization domain; SEQ ID NO: 157). In some embodiments, the transcription factor comprises an oligomerization domain comprising a polynucleotide having the sequence of any one of SEQ ID NOs: 156-158. In some embodiments, the transcription factor comprises an oligomerization domain comprising a polypeptide having the amino acid sequence of any one of SEQ ID NOs: 159-161.

As used in this disclosure, “Linker_only_for_oligomerization” refers to a polynucleotide that encodes “Linker 1” in the Supplementary Information of Kim D et al. (2014) Biomaterials, 35:6026. In some embodiments, this linker is used for two-part transcription factors lacking oligomerization domains.

In some embodiments, Trimerization_Domain encodes an oligomerization domain flanked by linkers on each side. In some embodiments, Trimerization_domain comprises a linker followed by a human Collagen Xv Trimerization Domain and a second linker. In some embodiments, Trimerization_Domain encodes a concatenation of the following three coding sub-parts, in the indicated order: (i) “Linker 1” in the Supplementary Information of Kim D et al. (2014); (ii) the trimerization domain sourced from Wirz J A et al. (2011) Matrix Biol., 30:9, and the associated TDB structure 3N3F; and (iii) “Linker 2” in the Supplementary Information of Kim D et al. (2014).

In some embodiments, Heptamerization_domain encodes a heptamerization domain flanked by linkers on each side. In some embodiments, Heptamerization_domain encodes a linker followed by an Archaeoglobus fulgidus Sm1 heptamerization domain and second linker. In some embodiments, Heptamerization_domain encodes a concatenation of the following three coding sub-parts, in the indicated order: (i) “Linker 1” in the Supplementary Information of Kim D et al. (2014); (ii) “Heptamerization domain” sourced from Table 1 of Kim D et al. (2012) PLoS One., 7:e43077; and (iii) “Linker 2” in the Supplementary Information of Kim D et al. (2014).

In some embodiments, the transcription factor comprises one or more linkers. In some embodiments, the linker comprises a polynucleotide having the nucleic acid sequence of SEQ ID NO: 120. In some embodiments, the linker comprises or consists of a polynucleotide having the nucleic acid sequence of SEQ ID NO: 121. In some embodiments, the linker comprises or consists of an amino acid having the nucleic acid sequence of SEQ ID NO: 122. In some embodiments, the linker comprises or consists of a polypeptide having the amino acid sequence of SEQ ID NO: 123.

It will be appreciated by those of skill in the art that a transcription factor may be oligomeric (e.g., containing multiple monomers or subunits) or monomeric (e.g., containing a single monomer or subunit). In some embodiments, a transcription factor which is oligomeric can comprise two or more subunits which are the same or different. In some embodiments, when expressed in a host cell, the transcription factor is translated as a single polypeptide chain. In some embodiments, the transcription factor is translated as multiple polypeptide chains. In some embodiments, the transcription factor comprises at least two polypeptide chains associated post-translationally. In some embodiments, the polypeptide chains are encoded by a polynucleotide that encodes a self-cleaving polypeptide. In some embodiments, the self-cleaving polypeptide is a 2A peptide. In some embodiments, the self-cleaving polypeptide is P2A. In some embodiments, the self-cleaving polypeptide is E2A, F2A, or T2A. In some embodiments, the self-cleaving polypeptide comprises a polynucleotide having the nucleic acid sequence of SEQ ID NO: 124. In some embodiments, the self-cleaving polypeptide comprises a polypeptide having the amino acid sequence of SEQ ID NO: 125.

In some embodiments, the transcription factor comprises one or more linkers.

Non-limiting examples of one-component sTFs (used for all three processes) are described in Table 7, Table 8, Table 9, and Table 10, and comprise 4 subparts: DBD, NLS, linker, and TAD. Part types of a sTF that are necessary for synthetic expression system functionality include the DBD and TAD.

Non-limiting examples of two-component sTFs are described in Table 11, Table 12, Table 13, Table 14, and Table 36, and comprise 9 possible subparts: DBD (required), NLS1, linker, BPP1, 2A, BPP2, NLS2, OD, and TAD (required).

Non-limiting examples of sTF variants are described in Table 7, Table 8, Table 9, Table 10, Table 11, Table 12, Table 13, Table 14, and Table 36.

In some embodiments, the DNA sequence of the gene encoding the sTF variant can be obtained by referring to the corresponding row of Table 7, Table 8, Table 9, Table 10, Table 11, Table 12, Table 13, Table 14, or Table 36, and then by generation of the full sTF gene sequence via concatenation of the DNA sequences of corresponding component part-type variants as noted in Table 21. The full DNA and amino acid sequences of certain sTF variants of this disclosure can be found (by SEQ ID NO) in Tables 30, 31, and 36.

In some embodiments, the DNA sequence of the transcriptional terminator of a first transcriptional unit (e.g., as used in Example 1) appears in Table 21 (by SEQ ID NO).

In some embodiments, the polynucleotide encoding a transcription factor comprises or consists of a sequence that is at least 70%, at least 80%, at least 90%, at least 91%, at least 92%, at least 93%, at least 94%, at least 95%, at least 96%, at least 97%, at least 98%, at least 99%, or 100% identical to a nucleic acid sequence in Example 2, Example 3, Tables 21, 30, and 36, or to the nucleic acid sequence of any one of SEQ ID NOs: 26-40 or 182-185. In some embodiments, the polynucleotide encoding a transcription factor comprises the nucleic acid sequence of any one of SEQ ID NOs: 26-40 or 182-185. In some embodiments, the polynucleotide encoding a transcription factor comprises or consists of not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 100, 150, 200, 250, or 300 nucleotide substitutions, insertions, additions, or deletions relative to the nucleic acid sequence of any one of SEQ ID NOs: 26-40 or 182-185.

In some embodiments, the transcription factor comprises or consists of a polypeptide that is at least 70%, at least 80%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs: 41-55. In some embodiments, the transcription factor comprises a polypeptide comprising or consisting of the sequence of the amino acid sequence of any one of SEQ ID NOs: 41-55. In some embodiments, an encoded transcription factor comprises or consists of a polypeptide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, or 100 amino acid substitutions, insertions, additions, or deletions relative to the amino acid sequence of any one of SEQ ID NOs: 41-55.

### Synthetic Output Promoters [P(Out)]

In some embodiments, this disclosure provides a second transcriptional unit comprising a gene of interest under the control of a synthetic output promoter. In some embodiments, this disclosure provides a second transcriptional unit comprising a synthetic output promoter and an insertion site, wherein the insertion site is located such that a gene of interest inserted into the insertion site is operably linked to and under the control of the synthetic output promoter. As used in this application, for example, a “synthetic output promoter” or “P(out)” refers to a synthetic promoter that is driven by (e.g., is cognate with respect to) a transcription factor of a first transcriptional unit and is operably linked to and capable of activating transcription of a polynucleotide encoding a gene of interest. In some embodiments, when expressed in a host cell genome, a gene of interest may or may not be endogenous to the host cell.

A coding sequence and a regulatory sequence (e.g., a promoter sequence) are said to be “operably joined” or “operably linked” when the coding sequence and the regulatory sequence are covalently linked and/or the expression or transcription of the coding sequence is under the influence or control of the regulatory sequence.

In some embodiments, P(out) is operably linked to a gene of interest that encodes an RNA. In some embodiments, P(out) is operably linked to a gene of interest that encodes a protein. In some embodiments, the gene of interest encodes an enzyme. In some embodiments, the gene of interest encodes a protein involved in the biosynthesis of an organic molecule.

If the coding sequence is to be translated into a functional bioproduct, the coding sequence and the regulatory sequence are said to be operably joined or linked if induction of a promoter in the 5′ regulatory sequence permits the coding sequence to be transcribed and if the nature of the link between the coding sequence and the regulatory sequence does not (1) result in a frameshift event that changes the reading frame of the coding sequence, (2) interfere with the ability of the promoter region to direct the transcription of the coding sequence, or (3) interfere with the ability of the corresponding RNA transcript to be translated into a protein.

In some embodiments, an output promoter is a synthetic output promoter cognate with respect to the Bm3R1-based sTF. In some embodiments, an output promoter is a synthetic output promoter cognate with respect to the PhlF_AM-based sTF. In some embodiments, an output promoter is a synthetic output promoter cognate with respect to the TetR-based sTF. In some embodiments, an output promoter is a synthetic output promoter cognate with respect to the VanR_AM-based sTF. In some embodiments, an output promoter is a synthetic output promoter cognate with respect to the Bm3R1-based sTF, for example, one listed in Table 15. In some embodiments, an output promoter is a synthetic output promoter cognate with respect to the PhlF_AM-based sTF, for example, one listed in Table 16. In some embodiments, an output promoter is a synthetic output promoter cognate with respect to the TetR-based sTF, for example, one listed in Table 17. In some embodiments, an output promoter is a synthetic output promoter cognate with respect to the VanR_AM-based sTF, for example, one listed in Table 18.

In some embodiments, a synthetic expression system comprises an output promoter, wherein the output promoter comprises or consists of a polynucleotide that is at least 90%, at least 91%, at least 92%, at least 93%, at least 94%, at least 95%, at least 96%, at least 97%, at least 98%, at least 99%, or 100% identical to a nucleic acid sequence in Examples 2 and 3, Table 33, Table 36, or to the nucleic acid sequence of any one of SEQ ID NOs: 56-70 or 186-193. In some embodiments, the output promoter comprises or consists of a polynucleotide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, nucleotide substitutions, insertions, additions, or deletions relative to the nucleic acid sequence of any one of SEQ ID NOs: 56-70 or 186-193. In some embodiments, the synthetic output promoter comprises or consists of a polynucleotide having the nucleic acid sequence of any one of SEQ ID NOs: 56-70 or 186-193.

In some embodiments, a transcription factor binds to an output promoter. In some embodiments, a synthetic output promoter comprises a DNA sequence directly bound by an RNA polymerase and a DNA sequence bound by the DNA-binding domain component of the transcription factor. In some embodiments, a DNA sequence bound by a transcription factor is or comprises an operator and/or an enhancer. In some embodiments, an upstream activating sequence is or comprises an operator and/or an enhancer. In some embodiments, an operator is a DNA sequence directly bound by a transcription factor, and an enhancer is a larger region of DNA comprising an operator. In some embodiments, a core promoter or core promoter sequence is the polynucleotide segment or sequence directly bound by an RNA polymerase.

As used in this application, a “core promoter” refers to the minimal portion of a promoter that is required to initiate transcription and that includes the transcriptional start site. Typically, a core promoter extends from approximately 15-20 bases upstream of a TATA box to a translation start site.

In some embodiments, a core promoter of an output promoter refers to a polynucleotide comprising nucleotide sequences which are directly bound by an RNA polymerase and are the minimal nucleotide sequences necessary for initiation of transcription of an operably linked coding sequence.

In some embodiments, a promoter (e.g., an input promoter or an output promoter) comprises (a) a core promoter and (b) a polynucleotide or sequence bound by a transcription factor which is or comprises one or more copies of any one or more of: an upstream activating sequence, operator, and/or enhancer. In some embodiments, an enhancer can comprise multiple operators. In some embodiments, a synthetic output promoter can comprise one or more operators and/or enhancers.

In some embodiments, the synthetic output promoter comprises an upstream activating sequence and a core promoter. In some embodiments, the synthetic output promoter comprises a core promoter element, and does not comprise an upstream activating sequence (UAS). In some embodiments, the upstream activating sequence is operably linked to the core promoter. In some embodiments, the upstream activating sequence is synthetic. In some embodiments, the upstream activating sequence is chimeric. In some embodiments, the upstream activating sequence comprises one or more operators. In some embodiments, the one or more operators may include bmO, tetO, phlO, or vanO. In some embodiments, the designation or description of an operator (or other component of a transcriptional unit or an expression system) comprises a prefix indicating the number of copies of the operator (or other component), for example: 1× indicates 1 copy, 2× indicates two copies, etc.

In some constructs, for example, controls, the designation or description of an operator (or other component of a transcriptional unit or expression system) comprises a prefix indicating the number of copies of the operator (or other component), wherein Ox indicates no (zero) copies (e.g., the operator or component is absent). In some constructs, for example controls, the designation or description of a TAD (or other component of a transcriptional unit or an expression system) comprises the prefix “No_”, indicating that this component is absent; for example, “No_TAD” indicates that there is no TAD present.

In some embodiments, one copy of bmO lies within an upstream activating sequence. bmO is created by the concatenation of the following three non-coding sub-parts, in the indicated order: (i) a non-repeated-sequence spacer, (ii) the Bm3R1 operator CGGAATGAACTTTCATTCCG (SEQ ID NO: 130), and (iii) a non-repeated-sequence spacer. In some embodiments, one copy of bmO comprises SEQ ID NO: 126.

In some embodiments, one copy of tetO lies within an upstream activating sequence comprising one copy of a TetR operator. tetO is created by the concatenation of the following three non-coding sub-parts, in the indicated order: (i) a non-repeated-sequence spacer, (ii) the TetR operator TCCCTATCAGTGATAGAGA (SEQ ID NO: 131), and (iii) a non-repeated-sequence spacer. In some embodiments, one copy of tetO comprises SEQ ID NO: 128.

In some embodiments, one copy of a phlO lies within an upstream activating sequence. phlO is created by the concatenation of the following three non-coding sub-parts, in the indicated order: (i) a non-repeated-sequence spacer, (ii) the PhlF operator ATGATACGAAACGTACCGTATCGTTAAGGT (SEQ ID NO: 132), and (iii) a non-repeated-sequence spacer. In some embodiments, one copy of phlO comprises SEQ ID NO: 127.

In some embodiments, one copy of vanO lies within an upstream activating sequence comprising one copy of a VanR operator. vanO is created by the concatenation of the following three non-coding sub-parts, in the indicated order: (i) a non-repeated-sequence spacer, (ii) the VanR operator ATTGGATCCAAT (SEQ ID NO: 133), and (iii) a non-repeated-sequence spacer. In some embodiments, one copy of vanO comprises SEQ ID NO: 129.

In some embodiments, the upstream activating sequence comprises no operators (“0xoperator”).

In some embodiments, the one or more operators is bound by a transcription factor, wherein the transcription factor or a component thereof is encoded a first transcriptional unit of this disclosure. In some embodiments, the one or more bound operators activates the core promoter sequence.

In some embodiments, the core promoter comprises a core promoter sequence that is naturally occurring. In some embodiments, the core promoter sequence comprises or consists of a sequence that is at least 90%, at least 95%, or is 100% identical to a naturally occurring core promoter sequence. In some embodiments, the core promoter sequence is synthetic. In some embodiments, the core promoter sequence is endogenous to the host cell. In some embodiments, the core promoter sequence is exogenous to the host cell. In some embodiments, the core promoter sequence comprises a homologous sequence to the endogenous core promoter sequence of the host cell. In some embodiments, the core promoter sequence comprises or consists of a sequence that is at least 90%, at least 95%, or is 100% identical to an endogenous core promoter sequence of the host cell. In some embodiments, the core promoter sequence comprises or consists of a sequence that is at least 90%, is at least 95%, or is 100% identical to a core promoter sequence from P(AOX1) (SEQ ID NO: 162), P(DAS2) (SEQ ID NO: 163), P(HHF2) (SEQ ID NO: 164), or P(PMP20) (SEQ ID NO: 165).

Non-limiting examples of synthetic output promoters are described in Table 15, Table 16, Table 17, Table 18, and Table 36, based on the four different DBD types used, and comprise 2 components: an upstream activating sequence (UAS) and a core promoter.

The DNA sequences of synthetic output promoters used in Example 1 can be obtained by referring to the corresponding row of Table 15, Table 16, Table 17, Table 18, or Table 36. The full DNA sequences of the synthetic output promoters used in Example 1 appear in Table 33 and Table 36 (by SEQ ID NO).

In some embodiments, the transcription factor comprises the DNA-binding domain of Bm3R1 and the upstream activating sequence of the synthetic output promoter comprises no, one, two, four, or eight copies, or other multiple copies of bmO. In some embodiments, two copies of bmO comprise or consist of SEQ ID NO: 134. In some embodiments, four copies of bmO comprise or consist of SEQ ID NO: 135. In some embodiments, eight copies of bmO comprise or consist of SEQ ID NO: 136.

In some embodiments, the transcription factor comprises the DNA-binding domain of PhlF_AM and the upstream activating sequence of the synthetic output promoter comprises no, one, two, four, or eight copies, or other multiple copies of phlO. In some embodiments, two copies of phlO comprise or consist of SEQ ID NO: 137. In some embodiments, four copies of phlO comprise or consist of SEQ ID NO: 138. In some embodiments, eight copies of phlO comprise or consist of SEQ ID NO: 139.

In some embodiments, the transcription factor comprises the DNA-binding domain of TetR and the upstream activating sequence of the synthetic output promoter comprises no, one, two, four, or eight copies, or other multiple copies of tetO. In some embodiments, two copies of tetO comprise or consist of SEQ ID NO: 140. In some embodiments, four copies of tetO comprise or consist of SEQ ID NO: 141. In some embodiments, eight copies of tetO comprise or consist of SEQ ID NO: 142.

In some embodiments, the transcription factor comprises the DNA-binding domain of VanR_AM and the upstream activating sequence of the synthetic output promoter comprises no, one, two, four, or eight copies, or other multiple copies of vanO. In some embodiments, two copies of vanO comprise or consist of SEQ ID NO: 143. In some embodiments, four copies of vanO comprise or consist of SEQ ID NO: 144. In some embodiments, eight copies of vanO comprise or consist of SEQ ID NO: 145.

### Transcriptional Terminators [TT]

In some embodiments, transcriptional units optionally comprise a transcriptional terminator.

In some embodiments, a transcriptional terminator is capable of terminating transcription (e.g., transcription of a transcription factor, a transcriptional activator, or a bioproduct). In some embodiments, the transcriptional terminator is a forward terminator. When located downstream of a polynucleotide sequence primed for transcription, a forward transcriptional terminator will cause transcription to terminate following transcription of the polynucleotide.

In some embodiments, either or both of a first transcriptional unit and/or a second transcriptional unit optionally comprise a transcriptional terminator. In some embodiments, a first transcriptional unit may comprise an optional first transcriptional terminator downstream of the polynucleotide encoding the transcriptional factor. In some embodiments, a second transcriptional unit may comprise an optional transcriptional terminator downstream of the gene of interest. In various embodiments, the first and second transcriptional terminators are the same or different.

In some embodiments, the first and/or second transcriptional unit comprises a transcriptional terminator that is naturally occurring. In some embodiments, the first and/or second transcriptional unit comprises a synthetic transcriptional terminator. In some embodiments, when expressed in a host cell, the first and/or second transcriptional unit comprises a transcriptional terminator that is endogenous to the host cell.

In some embodiments, a transcriptional terminator comprises or consists of a polynucleotide that is at least 70%, at least 80%, at least 90%, at least 91%, at least 92%, at least 93%, at least 94%, at least 95%, at least 96%, at least 97%, at least 98%, at least 99%, or 100% identical to a nucleic acid sequence in Example 1, Example 3, Tables 21 and 32, or to the nucleic acid sequence of either of SEQ ID NOs: 146 and 147. In some embodiments, the transcriptional terminator comprises or consists of a polynucleotide having the nucleic acid sequence of either of SEQ ID NOs: 146 and 147.

In some embodiments, the transcriptional terminators of a first and second transcriptional unit comprise the same polynucleotide sequence. In some embodiments, the first and/or second transcriptional unit comprise a transcriptional terminator from a gene encoding a ribosomal protein. In some embodiments, the first and/or second transcriptional unit comprise a transcriptional terminator from a gene encoding ribosomal protein S2 (RPS2) (SEQ ID NO: 146). In some embodiments, the first and/or second transcriptional unit comprises a transcriptional terminator from a gene encoding aldehyde oxidase 1 (AOX1) (SEQ ID NO: 147).

A non-limiting example of a transcriptional terminator (TT) is described in Tables 19, 21 (the corresponding DNA sequence of the transcriptional terminator from RPS2), and 32 (the corresponding DNA sequence of the transcriptional terminator from AOX1).

Various transcriptional terminators are described in this document and/or in the scientific literature. See, for example: Matsuyama et al. 2019 J. Biosci. Bioeng. 128: 655-661; Candelli et al. 2018 EMBO J. 37: e97490; Fox et al. 2016 WIREs RNA 7: 91-104; LaRochelle et al. 2018 Nat. Comm. 9: Article 4364; and Karbalaei et al. 202 J. Cell. Phys. 9: 5867. Any suitable transcriptional terminator described in this document and/or in the scientific literature can be incorporated as a component in a transcriptional unit or in a synthetic expression system.

### Variants

In some embodiments, the disclosure provides variants of a transcriptional unit or a synthetic expression system.

Aspects of the disclosure relate to polynucleotides, including a polynucleotide encoding a transcription factor (e.g., expressed from the input promoter) and a gene of interest encoding a bioproducts (e.g., expressed from a synthetic output promoter activated by the transcription factor). Variants of polynucleotides, transcription factors, and bioproducts described in this application are also encompassed by this disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., polynucleotide or amino acid residues). Identity measures the percent of identical matches between the smaller of two or more sequences with gap alignments (if any) addressed by a particular mathematical model, algorithms, or computer program.

Identity of related polynucleotide sequences, transcription factors, and/or bioproducts can be readily calculated by any of the methods known to one of ordinary skill in the art. In preferred embodiments, the “percent identity” of two sequences (e.g., polynucleotide or amino acid sequences) is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two polynucleotides is determined by aligning the two nucleotide sequences and calculating the number of identical nucleotide and dividing by the length of one of the polynucleotides.

For multiple sequence alignments, computer programs including Clustal Omega (Sievers et al., Mol Syst Biol. 2011 Oct. 11; 7:539) may be used. In some embodiments, a sequence, including a polynucleotide or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence X is at the counterpart position of Z in sequence Y when sequences X and Y are aligned using amino acid sequence alignment tools known in the art.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, variant sequences include homologous sequences. As used in this application, homologous sequences are sequences (e.g., polynucleotide or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 95%, at least 96%, at least 97%, at least 98%, at least 99%, or 100% percent identity. Homologous sequences include but are not limited to paralogous sequences, orthologous sequences, or sequences arising from convergent evolution. In some embodiments, paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant comprises a domain that shares a secondary structure (e.g., alpha helix, beta sheet) with a reference polypeptide. In some embodiments, a polypeptide variant shares a tertiary structure with a reference polypeptide. As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of the proteins, enzymes, or other bioproducts disclosed in this application are also encompassed by this disclosure. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

The skilled artisan will also realize that mutations in a bioproduct coding sequence may result in conservative amino acid substitutions to provide functionally equivalent variants of the foregoing bioproducts, e.g., variants that retain the activities of the bioproducts. As used in this application, a “conservative amino acid substitution” refers to an amino acid substitution that does not alter the relative charge or size characteristics or functional activity of the bioproduct in which the amino acid substitution is made.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 29). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) T; (f) Q, N; and (g) E, D. Additional non-limiting examples of conservative amino acid substitutions are provided in Table 29.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 29
Non-limiting Examples of conservative amino acid substitutions.

| | | Conservative |
|---|---|---|
| Original | | Amino Acid |
| Residue | R Group Type | Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

In some embodiments, a polynucleotide encoding any of the bioproducts described in this application is under the control of one or more regulatory sequences. In some embodiments, a polynucleotide is expressed under the control of a promoter. In some embodiments, the promoter is a native promoter. As used herein, a “native” promoter refers to a promoter for which at least one copy naturally occurs in a host cell. A native promoter may include but is not limited to the original copy or copies in the host cell; a promoter at a different locus from its native locus in a cell is nonetheless considered a promoter that is native to the cell. In some embodiments, the promoter is synthetic.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

This invention is further illustrated by the following Examples. Specific details of any particular method, process, medium or condition in the Examples are examples only and not intended to be limiting.

### Enumerated Embodiments

Certain embodiments are set forth in the enumerated clauses below.

1. A methylotrophic host cell comprising a synthetic expression system that comprises:
(a) a first transcriptional unit comprising:
   (i) an input promoter comprising an upstream activating sequence (UAS) and a core promoter element, and
   (ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
      wherein the input promoter drives expression of the at least one component of the synthetic transcription factor; and
(b) a second transcriptional unit comprising a synthetic output promoter operably linked to a gene of interest, wherein the synthetic transcription factor is an activator of the synthetic output promoter, and
   wherein the gene of interest is expressed in the absence of exogenously provided methanol.
      2. The methylotrophic host cell of clause 1, wherein the polynucleotide of the first transcriptional unit encodes all components of the synthetic transcription factor.
      3. The methylotrophic host cell of any of clauses 1-2, wherein the input promoter is synthetic.
      4. The methylotrophic host cell of clause 3, wherein the input promoter has at least 90% sequence identity to a naturally occurring promoter.
      5. The methylotrophic host cell of clause 4, wherein the input promoter is naturally occurring.
      6. The methylotrophic host cell of clause 5, wherein the input promoter is native to the cell.
      7. The methylotrophic host cell of any of clauses 1-5, wherein the input promoter is a regulatable input promoter.
      8. The methylotrophic host cell of clause 7, wherein the regulatable input promoter is inducible.
      9. The methylotrophic host cell of clause 7, wherein the regulatable input promoter is repressible.
      10. The methylotrophic host cell of clause 7, wherein the regulatable input promoter is responsive to nutrient addition, limitation, or depletion with respect to a cognate cultivation process.
      11. The methylotrophic host cell of clause 10, wherein the regulatable input promoter is responsive to thiamine depletion, glycerol limitation, monosaccharide limitation, or to the limitation of a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate.
      12. The methylotrophic host cell of clause 10, wherein the regulatable input promoter is responsive to the limitation or depletion of a combination of any two or more nutrients.
      13. The methylotrophic host cell of clause 10, wherein activity of the regulatable input promoter is increased by the presence of exogenously provided formic acid.
      14. The methylotrophic host cell of clause 7, wherein the regulatable input promoter is regulatable in the absence of exogenously provided methanol.
      15. The methylotrophic host cell of any of clauses 1-14, wherein the input promoter is not methanol inducible.
      16. The methylotrophic host cell of any of clauses 1-9, wherein the input promoter is a constitutive input promoter.
      17. The methylotrophic host cell of any of clauses 1-16, wherein the upstream activating sequence (UAS) and/or the core promoter element of the input promoter is not native to the methylotrophic host cell.
      18. The methylotrophic host cell of any of clauses 1-15, wherein the input promoter is P(JEN1), P(GQ6704499), P(GQ6700926), P(HGT1), P(FDH1), P(AOX2), P(RGI2), P(THI13)_short, P(THI13)_long, or P(THI4).
      19. The methylotrophic host cell of any of clauses 1-18, wherein the input promoter is polynucleotide having at least 90%, at least 95%, or at least 99% identity to a nucleic acid sequence of any one of SEQ ID NOs: 16-25.
      20. The methylotrophic host cell of any of clauses 1-18, wherein the input promoter is a polynucleotide having the nucleic acid sequence of any one of SEQ ID NOs: 16-25.
      21. The methylotrophic host cell of any of clauses 1-20, wherein the DNA binding domain (DBD) of the synthetic transcription factor is Bm3R1, TetR, PhlF_AM, or VanR_AM.
      22. The methylotrophic host cell of any of clauses 1-21, wherein the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD.
      23. The methylotrophic host cell of any of clauses 1-22, wherein the DNA-binding domain (DBD) of the synthetic transcription factor is Bm3R1, TetR, PhlF_AM, or VanR_AM, and the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD.
      24. The methylotrophic host cell of any of clauses 1-23, wherein the synthetic transcription factor is not an activator of the input promoter.
      25. The methylotrophic host cell of any of clauses 1-23, wherein the synthetic transcription factor is a one-component synthetic transcription factor.
      26. The methylotrophic host cell of any of clauses 1-23, wherein the synthetic transcription factor is a two-component or multi-component synthetic transcription factor.
      27. The methylotrophic host cell of clause 26, wherein the two-component or multi-component synthetic transcription factor comprises at least two bioconjugate protein products.
      28. The methylotrophic host cell of clause 27, wherein a first bioconjugate protein product (BPP1) is SpyTag002 and a second bioconjugate protein product (BPP2) is SpyCatcher002.
      29. The methylotrophic host cell of any of clauses 1-28, wherein the synthetic transcription factor comprises a nuclear localization signal (NLS).
      30. The methylotrophic host cell of clause 29, wherein the nuclear localization signal is an SV40 nuclear localization signal.
      31. The methylotrophic host cell of any of clauses 1-30, wherein the synthetic transcription factor comprises a linker.
      32. The methylotrophic host cell of any of clauses 1-31, wherein the synthetic transcription factor comprises a self-cleaving polypeptide.
      33. The methylotrophic host cell of clause 32, wherein the self-cleaving polypeptide is a 2A peptide.
      34. The methylotrophic host cell of clause 32, wherein the self-cleaving polypeptide is ERBV_1_P2A.
      35. The methylotrophic host cell of any of clauses 1-34, wherein the synthetic transcription factor comprises an oligomerization domain.
      36. The methylotrophic host cell of clause 35, wherein the oligomerization domain is Linker_only_for_oligomerization, Trimerization_domain, or Heptamerization_domain.
      37. The methylotrophic host cell of any of clauses 1-36, wherein the synthetic transcription factor comprises a polypeptide having the amino acid sequence of any one of SEQ ID NOs: 41-55.
      38. The methylotrophic host cell of any of clauses 1-37, wherein the first transcriptional unit comprises a polynucleotide having the nucleic acid sequence of any one of SEQ ID NOs: 26-40 or 182-185.
      39. The methylotrophic host cell of any of clauses 1-38, wherein the synthetic output promoter is not methanol inducible.
      40. The methylotrophic host cell of any of clauses 1-39, wherein the synthetic output promoter comprises an upstream activating sequence (UAS) and a core promoter element.
      41. The methylotrophic host cell of clause 40, wherein the upstream activating sequence (UAS) of the synthetic output promoter is not native to the methylotrophic host cell.
      42. The methylotrophic host cell of clause 40, wherein the core promoter element of the synthetic output promoter has a nucleic acid sequence that is no more than 300 base pairs in length.
      43. The methylotrophic host cell of clause 40, wherein the core promoter element of the synthetic output promoter has a nucleic acid sequence that is from about 6 base pairs to about 300 base pairs, from about 25 base pairs to about 250 base pairs, from about 75 to about 225 base pairs, or from about 100 base pairs to about 175 base pairs in length.
      44. The methylotrophic host cell of clause 40, wherein the distance between the 3′ end of the upstream activating sequence (UAS) and the 5′ end of the core promoter element of the synthetic output promoter is from 0 to about 200 base pairs in length.
      45. The methylotrophic host cell of clause 40, wherein the distance between the upstream activating sequence (UAS) and core promoter element of the synthetic output promoter is a nucleic acid sequence having from about 6 base pairs to about 200 base pairs, from about 6 base pairs to about 53 base pairs, from about 20 base pairs to about 150 base pairs, from about 50 base pairs to about 125 base pairs, or from about 50 base pairs to about 100 base pairs in length.
      46. The methylotrophic host cell of clause 40, wherein the core promoter element of the synthetic output promoter comprises a core promoter sequence that is at least 90%, at least 95%, or 100% identical to a naturally occurring core promoter sequence.
      47. The methylotrophic host cell of clause 40, wherein the core promoter element of the synthetic output promoter comprises a core promoter sequence that is at least 90%, at least 95%, or 100% identical to a core promoter sequence from P(AOX1), P(DAS2), P(HHF2), or P(PMP20).
      48. The methylotrophic host cell of clause 40, wherein the upstream activating sequence (UAS) of the synthetic output promoter is bmO, tetO, phlO, or vanO.
      49. The methylotrophic host cell of clause 40, wherein the synthetic output promoter further comprises one or more operators.
      50. The methylotrophic host cell of clause 49, wherein the one or more operators of the synthetic output promoter are not native to the methylotrophic host cell.
      51. The methylotrophic host cell of clause 40, wherein the synthetic transcription factor comprises the DNA-binding domain (DBD) Bm3R1, and the upstream activating sequence (UAS) of the synthetic output promoter comprises one or more copies of bmO.
      52. The methylotrophic host cell of clause 40, wherein the synthetic transcription factor comprises the DNA-binding domain (DBD) PhlF_AM and the upstream activating sequence (UAS) of the synthetic output promoter comprises one or more copies of phlO.
      53. The methylotrophic host cell of clause 40, wherein the synthetic transcription factor comprises the DNA-binding domain (DBD) TetR and the upstream activating sequence (UAS) of the synthetic output promoter comprises one or more copies of tetO.
      54. The methylotrophic host cell of clause 40, wherein the synthetic transcription factor comprises the DNA-binding domain (DBD) VanR_AM and the upstream activating sequence (UAS) of the synthetic output promoter comprises one or more copies of vanO.
      55. The methylotrophic host cell of any of clauses 1-54, wherein the synthetic output promoter comprises a polynucleotide having the nucleic acid sequence of any one of SEQ ID NOs: 56-70 or 186-193.
      56. The methylotrophic host cell of any of clauses 1-55, wherein the gene of interest is expressed as an RNA.
      57. The methylotrophic host cell of any of clauses 1-55, wherein the gene of interest encodes a protein.
      58. The methylotrophic host cell of clause 57, wherein the gene of interest encodes an enzyme, a structural protein, a signaling protein, a regulatory protein, a transport protein, a sensory protein, a motor protein, a defense protein, or a storage protein.
      59. The methylotrophic host cell of clause 57, wherein the protein synthesizes, modifies, or converts a molecule.
      60. The methylotrophic host cell of clause 59, wherein the molecule is heme or an intermediate in a heme biosynthesis pathway.
      61. The methylotrophic host cell of clause 57, wherein the protein is a heme-binding protein.
      62. The methylotrophic host cell of clause 61, wherein the heme-binding protein is hemoglobin, neuroglobin, cytoglobin, leghemoglobin, or myoglobin.
      63. The methylotrophic host cell of clause 57, wherein the protein is vaccinia capping enzyme, T7 polymerase, or O-methyltransferase.
      64. The methylotrophic host cell of clause 57, wherein the protein is an enzyme of a heme biosynthesis pathway.
      65. The methylotrophic host cell of clause 64, wherein the enzyme of a heme biosynthesis pathway is cytochrome P450, 9-adenylate cyclase, soluble guanylate cyclase, peroxidase, catalase, and/or cytochrome oxidase.
      66. The methylotrophic host cell of clause 1, further comprising in the second transcriptional unit a polynucleotide encoding a secretion tag.
      67. The methylotrophic host cell of clause 66, wherein the secretion tag is an α-amylase secretion tag, an Sc Mf al secretion tag, or a pre-inulinase secretion tag.
      68. The methylotrophic host cell of clause 66, wherein the gene of interest encodes a protein, and wherein the protein is secreted from the methylotrophic host cell.
      69. The methylotrophic host cell of clause 68, wherein the secreted protein is an α-amylase, a β-lactoglobulin, or an ovalbumin.
      70. The methylotrophic host cell of clause 1, wherein the first transcriptional unit and/or the second transcriptional unit further comprises a transcriptional terminator.
      71. The methylotrophic host cell of clause 70, wherein the transcriptional terminator of the first and/or second transcriptional unit is naturally occurring.
      72. The methylotrophic host cell of clause 70, wherein the transcriptional terminator of the first and/or second transcriptional unit is synthetic.
      73. The methylotrophic host cell of clause 70, wherein the transcriptional terminator of the first and/or second transcriptional unit is from a gene encoding a ribosomal protein.
      74. The methylotrophic host cell of clause 73, wherein the gene encodes the ribosomal protein S2 (RPS2).
      75. The methylotrophic host cell of clause 73, wherein the transcriptional terminator comprises a polynucleotide having the nucleic acid sequence of either SEQ ID NO: 146 or SEQ ID NO: 147.
      76. The methylotrophic host cell of any of clauses 1-75, wherein the first transcriptional unit and the second transcriptional unit are separated by a spacer.
      77. The methylotrophic host cell of any of clauses 1-76, wherein the first and/or second transcriptional unit is present in multiple copies.
      78. The methylotrophic host cell of clause 77, wherein the copy number ratio of the first transcriptional unit to the second transcriptional unit is 1:1.
      79. The methylotrophic host cell of clause 77, wherein the copy number ratio of the first transcriptional unit to the second transcriptional unit is at least 2:1, at least 4:1, or at least 10:1.
      80. The methylotrophic host cell of clause 77, wherein the copy number ratio of the second transcriptional unit to the first transcriptional unit is at least 2:1, at least 4:1, or at least 10:1.
      81. The methylotrophic host cell of clause 77, wherein the first transcriptional unit is present in a single copy and the second transcriptional unit is present in multiple copies.
      82. The methylotrophic host cell of clause 81, wherein at least two of the multiple second transcriptional units comprise different genes of interest.
      83. The methylotrophic host cell of clause 81, wherein the synthetic transcription factor of the first transcriptional unit is an activator of each synthetic output promoter of the multiple second transcriptional units.
      84. The methylotrophic host cell of any of clauses 1-83, wherein the synthetic expression system comprises one or more sequences that are endogenous to the methylotrophic host cell.
      85. The methylotrophic host cell of any of clauses 1-84, wherein the first and second transcriptional units are located on a single plasmid.
      86. The methylotrophic host cell of any of clauses 1-84, wherein the first and second transcriptional units are located on different plasmids.
      87. The methylotrophic host cell of any of clauses 1-84, wherein the first and/or second transcriptional units are integrated into the genome of the methylotrophic host cell.
      88. The methylotrophic host cell of clause 87, wherein the first and second transcriptional units are located on the same chromosome in the methylotrophic host cell genome.
      89. The methylotrophic host cell of any of clauses 87-88, wherein the first and second transcriptional units are oriented in the same direction.
      90. The methylotrophic host cell of any of clauses 87-88, wherein the first and second transcriptional units are oriented in different directions.
      91. The methylotrophic host cell of clause 87, wherein the first and second transcriptional units are located on different chromosomes in the methylotrophic host cell genome.
      92. The methylotrophic host cell of any of clauses 1-91, wherein the methylotrophic host cell is a methylotrophic yeast cell.
      93. The methylotrophic host cell of any of clauses 1-92, wherein the methylotrophic host cell is from a genus selected from: Pichia, Komagataella, Hansenula, or Candida.
      94. The methylotrophic host cell of clause 93, wherein the methylotrophic host cell is Pichia pastoris, Pichia pseudopastoris, Komagataella phaffii, Pichia stipitis, Pichia membranifaciens, Komagataella pseudopastoris, Komagataella pastoris, Komagataella kurtzmanii, Komagataella mondaviorum, Hansenula polymorpha, Candida boidinii, or Pichia methanolica.
      95. The methylotrophic host cell of clause 93, wherein the methylotrophic host cell is Pichia pastoris.
      96. The methylotrophic host cell of any of clauses 1-95, wherein the synthetic expression system provides for production of a bioproduct encoded by the gene of interest at a level that is higher than the level of the bioproduct produced in a control host cell.
      97. The methylotrophic host cell of clause 96, wherein the control host cell is of the same species as the methylotrophic host cell.
      98. The methylotrophic host cell of clause 97, wherein the control host cell comprises a methanol-inducible promoter operably linked to a gene of interest.
      99. The methylotrophic host cell of clause 98, wherein the gene of interest encoded by the control host cell is the same gene of interest encoded by the methylotrophic host cell.
      100. The methylotrophic host cell of clause 98, wherein the methanol-inducible promoter is P(AOX1) of P. pastoris.
      101. The methylotrophic host cell of clause 100, wherein the control cell is cultured in the presence of exogenously-added methanol.
      102. The methylotrophic host cell of any of clauses 1-101, wherein the methylotrophic host cell is cultured under conditions comprising a growth phase and a production phase.
      103. The methylotrophic host cell of clause 102, wherein the quantity of transcripts of the gene of interest produced in the methylotrophic host cell in the production phase is at least 100% higher than the quantity of transcripts of the gene of interest produced in the methylotrophic host cell in the growth phase.
      104. The methylotrophic host cell of clause 102, wherein the quantity of transcripts of the gene of interest produced in the methylotrophic host cell in the production phase is at least 200%, at least 300%, at least 400%, or at least 500% higher than the quantity of transcripts of the gene of interest produced in the methylotrophic host cell in the growth phase.
      105. The methylotrophic host cell of any of clauses 1-104, wherein the synthetic expression system provides for production of a bioproduct encoded by the gene of interest at a level that is at least 200% higher than the level of the bioproduct produced in a control host cell.
      106. The methylotrophic host cell of clause 105, wherein the synthetic expression system provides for production of a bioproduct encoded by the gene of interest at a level that is at least 600%, at least 900%, at least 1200%, at least 1500%, at least 1800%, at least 2100%, at least 2400%, at least 2700%, or at least 3000% higher than the level of the bioproduct produced in a control host cell.
      107. The methylotrophic host cell of clause 105, wherein the synthetic expression system provides for production of a bioproduct encoded by the gene of interest at a level that is from about 300% to about 600%, from about 500% to about 1000%, from about 800% to about 1500%, from about 1000% to about 2000%, from about 1200% to about 2000%, from about 1800% to about 2500%, from about 2000% to about 2500%, or from about 2200% to about 3000% higher than the level of the bioproduct produced in a control host cell.
      108. A methylotrophic host cell comprising a synthetic expression system that comprises:
(a) a first transcriptional unit comprising:
   (i) an input promoter comprising an upstream activating sequence (UAS) and a core promoter element, and
   (ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
      wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter,

wherein the gene of interest is expressed in the absence of exogenously provided methanol,

wherein methylotrophic host cell is cultured under conditions comprising a growth phase and a production phase, and

wherein the quantity of transcripts of the gene of interest produced by methylotrophic host cell in the production phase is at least 100% higher than in the growth phase.

109. A methylotrophic host cell comprising a synthetic expression system that comprises:
(a) a first transcriptional unit comprising:
   (i) an input promoter comprising an upstream activating sequence (UAS) and a core promoter element, and
   (ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
      wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter, and a polynucleotide encoding a secretion tag;

wherein the gene of interest is expressed in the absence of exogenously provided methanol.

110. A methylotrophic host cell comprising a synthetic expression system that comprises:
(a) a first transcriptional unit comprising:
   (i) an input promoter comprising an upstream activating sequence (UAS) and a core promoter element, and
   (ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
      wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter,

wherein the gene of interest is expressed in the absence of exogenously provided methanol, and

wherein the synthetic expression system provides for production of a bioproduct encoded by the gene of interest at a level that is at least 300% higher than the level of the bioproduct produced in a control host cell.

111. A method of expressing a gene of interest comprising culturing the methylotrophic host cell according to any one of clauses 1-110.
112. The method of clause 111, wherein the gene of interest encodes a heme-binding protein or one or more enzymes of a heme biosynthesis pathway.
113. The method of clause 112, wherein the heme-binding protein is hemoglobin, myoglobin, neuroglobin, cytoglobin, or leghemoglobin.
114. The method of clause 112, wherein the one or more enzymes of a heme biosynthesis pathway is cytochrome P450, 9-adenylate cyclase, soluble guanylate cyclase, peroxidase, catalase, and/or cytochrome oxidase.
115. The method of clause 111, wherein the gene of interest encodes a vaccinia capping enzyme, T7 polymerase enzyme, or O-methyltransferase enzyme.
116. A method of manufacturing a molecule of interest comprising culturing the methylotrophic host cell according to any one of clauses 1-110 and obtaining the molecule of interest from biomass or culture.
117. The method of clause 116, wherein the obtaining comprises extracting the molecule of interest from biomass.
118. The method of clause 116, wherein the obtaining comprises collecting the molecule from culture, culture medium, cell-free spent culture medium, and/or cell-containing culture medium.
119. A method of producing a molecule of interest comprising expressing a gene of interest according to any one of clauses 111-115, wherein the gene of interest encodes an enzyme, the method comprising:
(a) purifying the enzyme encoded by the gene of interest; and
(b) using the purified enzyme for bioconversion of a substrate to the molecule of interest.
   120. The method of any one of clauses 116-119, wherein the molecule of interest is heme.
   121. A method of expressing a gene of interest or producing a molecule of interest comprising steps of:
(a) culturing a methylotrophic host cell according to any one of clauses 1-110 in a suitable medium for a period of time to allow cell growth, and
(b) changing one or more culture conditions to facilitate expression of the gene of interest or production of the molecule of interest.
   122. The method of clause 121, wherein changing one or more culture conditions comprises changing the composition of the culture medium.
   123. The method of any one of clauses 121-122, wherein step (b) comprises limiting, adding, and/or depleting a nutrient.
   124. The method of any one of clauses 121-123, wherein step (b) comprises thiamine depletion, glycerol limitation, monosaccharide limitation, or formic acid addition.
   125. The method of any one of clauses 121-124, wherein step (b) comprises limitation of any a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate.
   126. The method of any one of clauses 121-125, wherein step (b) comprises the limitation of a combination of any two nutrients.
   127. The method of any one of clauses 121-125, wherein step (b) comprises limitation of glucose and depletion of thiamine.
   128. A synthetic expression system comprising a polynucleotide having at least 90%, at least 95%, or at least 99% identity to the nucleic acid sequence of any one of SEQ ID NOs: 1-15.
   129. The synthetic expression of clause 128, wherein the synthetic expression system comprises an input promoter comprising a polynucleotide having at least 90%, at least 95%, or at least 99% identity to the nucleic acid sequence of any one of SEQ ID NOs: 16-25.
   130. The synthetic expression of any of clauses 128-129, wherein the synthetic expression system comprises a polynucleotide encoding at least one component of a synthetic transcription factor.
   131. The synthetic expression system of clause 130, wherein the polynucleotide encoding at least one component of a synthetic transcription factor comprises a polynucleotide having at least 90%, at least 95%, or at least 99% identity to the nucleic acid sequence of any one of SEQ ID NOs: 26-40 or 182-185.
   132. The synthetic expression system of clause 131, wherein the encoded synthetic transcription factor comprises a polypeptide having at least 90%, at least 95%, or at least 99% identity to the amino acid sequence of any one of SEQ ID NOs: 41-55.
   133. The synthetic expression of any one of clauses 128-132, wherein the synthetic expression system comprises a synthetic output promoter comprising a polynucleotide having at least 90%, at least 95%, or at least 99% identity to the nucleic acid sequence of any one of SEQ ID NOs: 56-70 or 186-193.
   134. The methylotrophic host cell according to any one of clauses 1-110 comprising a polynucleotide having at least 90%, at least 95%, or at least 99% identity to the nucleic acid sequence of any one of SEQ ID NOs: 16-25.
   135. The methylotrophic host cell according to any one of clauses 1-110 comprising a polynucleotide having at least 90%, at least 95%, or at least 99% identity to the nucleic acid sequence of any one of SEQ ID NOs: 56-70 or 186-193.
   136. The methylotrophic host cell according to any one of clauses 1-110, wherein the synthetic transcription factor is encoded by a polynucleotide having at least 90%, at least 95%, or at least 99% identity to the nucleic acid sequence of any one of SEQ ID NOs: 26-40 or 182-185.
   137. The methylotrophic host cell according to any one of clauses 1-110, wherein the synthetic transcription factor comprises a polypeptide having at least 90%, at least 95%, or at least 99% identity to the amino acid sequence of any one of SEQ ID NOs: 41-55.
   138. A method of engineering a host cell for protein expression comprising: transforming the host cell with the synthetic expression system according to any one of clauses 128-133.
   139. A plurality of nucleic acid molecules, each nucleic acid molecule in the plurality comprising:
(a) a first transcriptional unit comprising a nucleic acid sequence encoding a transcription factor or component thereof;
(b) a second transcriptional unit comprising a nucleic acid sequence of a synthetic output promoter operably linked to a nucleic acid sequence encoding a fluorescent protein, and
(c) a nucleic acid sequence of a distinct barcode,
   wherein the transcription factor is an activator of the synthetic output promoter.
   140. The plurality of nucleic acid molecules of clause 139, wherein the plurality of nucleic acid molecules comprises at least 1000, at least 5000, at least 10,000, at least 25,000, at least 30,000, at least 40,000 or at least 50,000 distinct nucleic acid molecules.
   141. A method of screening a plurality of host cells comprising nucleic acid molecules according to any one of clauses 139-140 for a property of interest, the method comprising:
(a) expressing the plurality of nucleic acid molecules in individual host cells; and
(b) isolating host cells having the property of interest.
   142. The method of clause 141, wherein the nucleic acid molecules in (b) are identified by next generation sequencing.

### EXAMPLES

### Example 1. Library Design and Construction

This non-limiting example describes the design and construction of host cells comprising example synthetic expression systems. Other embodiments of synthetic expression systems according to this disclosure are not excluded in this example.

Synthetic expression system (SES) libraries were designed to increase expression of a gene of interest using three methanol-independent cultivation processes of interest, namely Process 1 (limiting glycerol and added formic acid), Process 2 (limiting glucose and added formic acid), and Process 3 (limiting glucose and depleted thiamine). The design principle was the same in all cases.

Specifically, a first transcriptional unit was designed to express a synthetic transcription factor (sTF) under the transcriptional control of an input promoter. The input promoters for this experiment were selected on the basis of being induced during the production phase of a methanol-independent process (e.g., Process 1, Process 2, or Process 3, although other processes, with corresponding input promoters, could have been chosen). A second transcriptional unit was designed to express a reporter gene encoding red fluorescent protein (RFP) (i.e., a gene of interest) under the transcriptional control of a synthetic output promoter designed to be cognate to the DNA-binding domain of the sTF expressed by the first transcriptional unit. Together, a particular first transcriptional unit and a particular second transcriptional unit comprise an SES.

For each of Processes 1-3, the DNA-based SES library comprised 8 SES sub-libraries, wherein each sub-library corresponded to one of 8 different types of sTFs: (1) one-component Bm3R1-based sTFs, (2) one-component PhlF_AM-based sTFs, (3) one-component TetR-based sTFs, (4) one-component VanR_AM-based sTFs, (5) two-component Bm3R1-based sTFs, (6) two-component PhlF_AM-based sTFs, (7) two-component TetR-based sTFs, and (8) two-component VanR_AM-based sTFs. Each SES library for each given Process # was made by pooling together 8 SES sub-libraries. Full details can be found in Tables 1, 2, and 3.

The two-component sTFs that have been used in this Example were of the type wherein the inter-molecular complexation between or among two-component sTF component polypeptide #1 and two-component sTF component polypeptide #2 are mediated by covalent isopeptide bond formation using a version of the SpyTag/SpyCatcher system, but any short protein sequence equivalent in functionality to a SpyTag variant may be referred to as a “Bioconjugate Protein Part 1 (BPP1),” and any cognate protein sequence equivalent in functionality to a SpyCatcher variant may be referred to as a “Bioconjugate Protein Part 2 (BPP2).”

Furthermore, in the two-component sTFs used in this Example, the protein sequence of two-component sTF component polypeptide #1 and the protein sequence of two-component sTF component polypeptide #2 were encoded in the same transcriptional unit, driven by a single promoter, and the two different polypeptide chains were generated from a single coding sequence via a “ribosome skipping” event mediated by an intervening encoded P2A sequence. Another possible configuration would have been one where the protein sequence of two-component sTF component polypeptide #1 and the protein sequence of two-component sTF component polypeptide #2 were encoded in separate transcriptional units, each driven by a separate promoter.

Each of the 8 SES sub-libraries comprised a combinatorial assembly of 5 part types, wherein each of these 5 part types comprised one or more variant DNA sequences, and wherein there was a functional interdependence between the part types to maintain the cognate relationship between the DNA-binding domain of the sTF (i.e., Bm3R1, PhlF_AM, TetR, or VanR_AM) and the upstream activating sequence (UAS) of the synthetic output promoter.

More specifically, the part types for the combinatorial assembly of each sub-library were: (1) input promoter [P(in)], (2) sTF (one of the eight types set forth above), (3) transcriptional terminator (TT) of the first transcriptional unit, (4) spacer, and (5) synthetic output promoter [P(out)]. Optional parts (3) and (4) were included because they were presumed to impart efficiency to the SES. Endogenous sequences could also be used in situ for some of these parts, or fragments of parts. The theoretical combinatorial size of a given SES sub-library was the product of the number of variant parts across the 5 part types. For example, if there were 5, 24, 1, 1, and 20 variants of part types 1, 2, 3, 4, and 5, respectively, then the theoretical sub-library size would be 2400, or 5×24×1×1×20. The theoretical size of a full SES library is the sum of the theoretical sizes of each of the 8 SES sub-libraries. For example, if there were 2400, 2300, 2300, 2200, 9500, 9100, 9100, and 9200 variants across the 8 SES sub-libraries, then the theoretical full SES library size would be 46, 100, or 2400+2300+2300+2200+9500+9100+9100+9200.

Table 1 describes the part types, and by reference to Tables 4 through 21, the corresponding part-type variants and their sequences, that were used to design the full DNA-based SES library for fermentation Process 1. The theoretical full SES library size of Process 1 was 46,100 variants. Table 2 describes the SES library for fermentation Process 2, the theoretical full SES library size being 55,320 variants. Table 3 describes the SES library for fermentation Process 3, the theoretical full SES library size being 27,660 variants.

TABLE 1
Design of synthetic expression system (SES) library for Process 1 (limiting glycerol +
added formic acid).

| | | . . . wherein | | | . . . that, when |
|---|---|---|---|---|---|
| | | each | . . . wherein | . . . wherein | pooled with all |
| The Process | | indicated | each set of | the theoretical | other SES sub- |
| 1 SES | . . . wherein the | part type | indicated | combinatorial | libraries, |
| library | indicated SES sub- | had the | part-type | size of the | yielded a |
| comprised | library comprised a | indicated | variants are | resulting | theoretical |
| SES sub- | combinatorial | number of | listed in the | SES sub- | combinatorial |
| libraries of | assembly of the | possible | indicated | library was | size of the SES |
| type . . . | indicated part types . . . | variants . . . | Table . . . | . . . | library of . . . |
| One- | P(in) cognate w.r.t. | 5 | Table 4 | 2,400 | 46,100 |
| component | Process 1 | | | (5 × 24 × 1 × | (2,400 + |
| Bm3R1- | One-component | 24 | Table 7 | 1 × 20) | 2,300 + |
| based sTFs | Bm3R1-based sTF | | | | 2,300 + |
| | TT | 1 | Table 19 | | 2,200 + |
| | Spacer | 1 | Table 20 | | 9,500 + |
| | P(out) cognate w.r.t. | 20 | Table 15 | | 9,100 + |
| | Bm3R1-based sTF | | | | 9,100 + |
| One- | P(in) cognate w.r.t. | 5 | Table 4 | 2,300 | 9,200) |
| component | Process 1 | | | (5 × 23 × 1 × | |
| PhlF_AM- | One-component | 23 | Table 8 | 1 × 20) | |
| based sTFs | PhlF_AM-based sTF | | | | |
| | TT | 1 | Table 19 | | |
| | Spacer | 1 | Table 20 | | |
| | P(out) cognate w.r.t. | 20 | Table 16 | | |
| | PhlF_AM | | | | |
| One- | P(in) cognate w.r.t. | 5 | Table 4 | 2,300 | |
| component | Process 1 | | | (5 × 23 × 1 × | |
| TetR-based | One-component TetR- | 23 | Table 9 | 1 × 20) | |
| sTFs | based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| One- | P(in) cognate w.r.t. | 5 | Table 4 | 2,200 | |
| component | Process 1 | | | (5 × 22 × 1 × | |
| VanR_AM- | One-component | 22 | Table 10 | 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |
| Two- | P(in) cognate w.r.t. | 5 | Table 4 | 9,500 | |
| component | Process 1 | | | (5 × 95 × 1 × | |
| Bm3R1- | Two-component | 95 | Table 11 | 1 × 20) | |
| based sTFs | Bm3R1-based sTF | | | | |
| | TT | 1 | Table 19 | | |
| | Spacer | 1 | Table 20 | | |
| | P(out) cognate w.r.t. | 20 | Table 15 | | |
| | Bm3R1 | | | | |
| Two- | P(in) cognate w.r.t. | 5 | Table 4 | 9,100 | |
| component | Process 1 | | | (5 × 91 × 1 × | |
| PhlF_AM- | Two-component | 91 | Table 12 | 1 × 20) | |
| based sTFs | PhlF_AM-based sTF | | | | |
| | TT | 1 | Table 19 | | |
| | Spacer | 1 | Table 20 | | |
| | P(out) cognate w.r.t. | 20 | Table 16 | | |
| | PhlF_AM-based sTF | | | | |
| Two- | P(in) cognate w.r.t. | 5 | Table 4 | 9,100 | |
| component | Process 1 | | | (5 × 91 × 1 × | |
| TetR-based | Two-component TetR- | 91 | Table 13 | 1 × 20) | |
| sTFs | based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| Two- | P(in) cognate w.r.t. | 5 | Table 4 | 9,200 | |
| component | Process 1 | | | (5 × 92 × 1 | |
| VanR_AM- | Two-component | 92 | Table 14 | 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |

Abbreviation: w.r.t., with respect to.

TABLE 2
Design of synthetic expression system (SES) library for Process 2
(limiting glucose + added formic acid).

| | | . . . wherein | | | . . . that, when |
|---|---|---|---|---|---|
| | | each | . . . wherein | . . . wherein | pooled with |
| | . . . wherein the | indicated | each set of | the theoretical | all other SES |
| The Process 2 | indicated SES sub- | part-type | indicated | combinatorial | sub-libraries, |
| SES library | library comprised a | had the | part-type | size of the | yielded a |
| comprised | combinatorial | indicated | variants are | resulting | theoretical |
| SES sub- | assembly of the | number of | listed in the | SES sub- | combinatorial |
| libraries of | indicated part types | possible | indicated | library was | size of the SES |
| type . . . | . . . | variants . . . | Table . . . | . . . | library of . . . |
| One- | P(in) cognate w.r.t. | 6 | Table 5 | 2,880 | 55,320 |
| component | Process 2 | | | (6 × 24 × 1 × | (2,880 + |
| Bm3R1-based | One-component | 24 | Table 7 | 1 × 20) | 2,760 + |
| sTFs | Bm3R1-based sTF | | | | 2,760 + |
| | TT | 1 | Table 19 | | 2,640 + |
| | Spacer | 1 | Table 20 | | 11,400 + |
| | P(out) cognate w.r.t. | 20 | Table 15 | | 10,920 + |
| | Bm3R1-based sTF | | | | 10,920 + |
| One- | P(in) cognate w.r.t. | 6 | Table 5 | 2,760 | 11,040) |
| component | Process 2 | | | (6 × 23 × 1 × | |
| PhlF_AM- | One-component | 23 | Table 8 | 1 × 20) | |
| based sTFs | PhlF_AM-based sTF | | | | |
| | TT | 1 | Table 19 | | |
| | Spacer | 1 | Table 20 | | |
| | P(out) cognate w.r.t. | 20 | Table 16 | | |
| | PhlF_AM-based sTF | | | | |
| One- | P(in) cognate w.r.t. | 6 | Table 5 | 2,760 | |
| component | Process 2 | | | (6 × 23 × 1 × | |
| TetR-based | One-component TetR- | 23 | Table 9 | 1 × 20) | |
| sTFs | based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| One- | P(in) cognate w.r.t. | 6 | Table 5 | 2,640 | |
| component | Process 2 | | | (6 × 22 × 1 × | |
| VanR_AM- | One-component | 22 | Table 10 | 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |
| Two- | P(in) cognate w.r.t. | 6 | Table 5 | 11,400 | |
| component | Process 2 | | | (6 × 95 × 1 × | |
| Bm3R1-based | Two-component | 95 | Table 11 | 1 × 20) | |
| sTFs | Bm3R1-based sTF | | | | |
| | TT | 1 | Table 19 | | |
| | Spacer | 1 | Table 20 | | |
| | P(out) cognate w.r.t. | 20 | Table 15 | | |
| | Bm3R1-based sTF | | | | |
| Two- | P(in) cognate w.r.t. | 6 | Table 5 | 10,920 | |
| component | Process 2 | | | (6 × 91 × 1 × | |
| PhlF_AM- | Two-component | 91 | Table 12 | 1 × 20) | |
| based sTFs | PhlF_AM-based sTF- | | | | |
| | based sTF | | | | |
| | TT | 1 | Table 19 | | |
| | Spacer | 1 | Table 20 | | |
| | P(out) cognate w.r.t. | 20 | Table 16 | | |
| | PhlF_AM-based sTF | | | | |
| Two- | P(in) cognate w.r.t. | 6 | Table 5 | 10,920 | |
| component | Process 2 | | | (6 × 91 × 1 × | |
| TetR-based | Two-component TetR- | 91 | Table 13 | 1 × 20) | |
| sTFs | based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| Two- | P(in) cognate w.r.t. | 6 | Table 5 | 11,040 | |
| component | Process 2 | | | (6 × 92 × 1 × | |
| VanR_AM- | Two-component | 92 | Table 14 | 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |

TABLE 3
Design of synthetic expression system (SES) library for Process 3 (limiting glucose + depleted thiamine).

| | | | | | . . . that, when |
|---|---|---|---|---|---|
| | . . . wherein the | . . . wherein | . . . wherein | . . . wherein | pooled with all |
| | indicated SES sub- | each indicated | each set of | the theoretical | other SES sub- |
| The Process 3 | library comprised a | part type had | indicated part- | combinatorial | libraries, yielded |
| SES library | combinatorial | the indicated | type variants | size of the | a theoretical |
| comprised SES | assembly of | number | are listed in | resulting SES | combinatorial |
| sub-libraries | the indicated part | of possible | the indicated | sub-library | size of the SES |
| of type . . . | types . . . | variants . . . | Table . . . | was . . . | library of . . . |
| One- | P(in) cognate w.r.t. | 3 | Table 6 | 1,440 | 27,660 |
| component | Process 3 | | | (3 × 24 × | (1,440 + |
| Bm3R1-based | One-component | 24 | Table 7 | 1 × 1 × 20) | 1,380 + |
| sTFs | Bm3R1-based sTF | | | | 1,380 + |
| | TT | 1 | Table 19 | | 1,320 + |
| | Spacer | 1 | Table 20 | | 5,700 + |
| | P(out) cognate w.r.t. | 20 | Table 15 | | 5,460 + |
| | Bm3R1-based sTF | | | | 5,460 + |
| One- | P(in) cognate w.r.t. | 3 | Table 6 | 1,380 | 5,520) |
| component | Process 3 | | | (3 × 23 × | |
| PhlF_AM- | One-component | 23 | Table 8 | 1 × 1 × 20) | |
| based sTFs | PhlF_AM-based sTF | | | | |
| | TT | 1 | Table 19 | | |
| | Spacer | 1 | Table 20 | | |
| | P(out) cognate w.r.t. | 20 | Table 16 | | |
| | PhlF_AM-based sTF | | | | |
| One- | P(in) cognate w.r.t. | 3 | Table 6 | 1,380 | |
| component | Process 3 | | | (3 × 23 × | |
| TetR-based | One-component | 23 | Table 9 | 1 × 1 × 20) | |
| sTFs | TetR-based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| One- | P(in) cognate w.r.t. | 3 | Table 6 | 1,320 | |
| component | Process 3 | | | (3 × 22 × | |
| VanR_AM- | One-component | 22 | Table 10 | 1 × 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |
| Two- | P(in) cognate w.r.t. | 3 | Table 6 | 5,700 | |
| component | Process 3 | | | (3 × 95 × | |
| Bm3R1-based | Two-component | 95 | Table 11 | 1 × 1 × 20) | |
| sTFs | Bm3R1-based sTF | | | | |
| | TT | 1 | Table 19 | | |
| | Spacer | 1 | Table 20 | | |
| | P(out) cognate w.r.t. | 20 | Table 15 | | |
| | Bm3R1-based sTF | | | | |
| Two- | P(in) cognate w.r.t. | 3 | Table 6 | 5,460 | |
| component | Process 3 | | | (3 × 91 × | |
| PhlF_AM- | Two-component | 91 | Table 12 | 1 × 1 × 20) | |
| based sTFs | PhlF_AM-based sTF | | | | |
| | TT | 1 | Table 19 | | |
| | Spacer | 1 | Table 20 | | |
| | P(out) cognate w.r.t. | 20 | Table 16 | | |
| | PhlF_AM | | | | |
| Two- | P(in) cognate w.r.t. | 3 | Table 6 | 5,460 | |
| component | Process 3 | | | (3 × 91 × | |
| TetR-based | Two-component | 91 | Table 13 | 1 × 1 × 20) | |
| sTFs | TetR-based sTF | | | | |
| | TT | 1 | Table 19 | | |
| | Spacer | 1 | Table 20 | | |
| | P(out) cognate w.r.t. | 20 | Table 17 | | |
| | TetR-based sTF | | | | |
| Two- | P(in) cognate w.r.t. | 3 | Table 6 | 5,520 | |
| component | Process 3 | | | (3 × 92 × | |
| VanR_AM- | Two-component | 92 | Table 14 | 1 × 1 × 20) | |
| based sTFs | VanR_AM-based sTF | | | | |
| | TT | 1 | Table 19 | | |
| | Spacer | 1 | Table 20 | | |
| | P(out) cognate w.r.t. | 20 | Table 18 | | |
| | VanR_AM-based sTF | | | | |

For each SES sub-library, parts (1) input promoter (P(in)), (2) sTF (one of the eight types set forth above), and (5) synthetic output promoter (P(out)) had multiple variants. P(in)s are described in Table 4, Table 5, and Table 6. There is overlap between the P(in)s used for Process 1 and those used for Process 2. For all input promoter variants described in Table 4, Table 5, and Table 6, the corresponding DNA sequences are noted in Table 21.

TABLE 4
Input promoters for use with Process 1 (limiting glycerol + added formic
acid). DNA sequences of part-type variants are noted in Table 21.
Part types that are considered necessary for SES functionality are
labeled ″Necessary part type.″ While the input promoter part type is
necessary, other specific input promoters could have been chosen.
P(in)
Necessary part type
P(JEN1)
P(GQ6704499)
P(GQ6700926)
P(HGT1)
P(FDH1)

TABLE 5
Input promoters for use with Process 2 (limiting glucose + added formic
acid). DNA sequences of part-type variants are noted in Table 21.
Part types that are considered necessary for SES functionality are
labeled ″Necessary part type.″ While the input promoter part type is
necessary, other specific input promoters could have been chosen.
P(in)
Necessary part type
P(GQ6704499)
P(GQ6700926)
P(HGT1)
P(FDH1)
P(AOX2)
P(RGI2)

TABLE 6
Input promoters for use with Process 3 (limiting glucose + depleted
thiamine). DNA sequences of part-type variants are noted in Table 21.
Part types that are considered necessary for SES functionality are
labeled ″Necessary part type.″ While the input promoter part type is
necessary, other specific input promoters could have been chosen.
P(in)
Necessary part type
P(THI13)_short
P(THI13)_long
P(THI4)

One-component sTFs (used for all three processes) are described in Table 7, Table 8, Table 9, and Table 10, and comprise 4 subparts: DBD, NLS (optional), Linker (optional), and TAD. For a given DBD, up to 24 possible one-component sTFs were used for SES library assembly, based on a combinatorial assembly of 1 DBD variant×1 NLS variant×2 Linker variants×12 TAD variants.

TABLE 7
One-component sTFs based on DNA-binding domain Bm3R1. The ″Full name″ entry
on a given row is the name of both the gene encoding that row's sTF protein as well as that of
this sTF protein. The DNA sequence of this sTF-encoding gene is a concatenation of the DNA
sequences of the indicated part-type variants on the corresponding row in left-to-right order.
The amino acid sequence of this sTF protein is a concatenation of the amino acid sequences
of the indicated part-type variants on the corresponding row in left-to-right order. DNA and
amino acid sequences of component part-type variants are in Table 21. The ″Used?″ entry on
a given row is ″Yes″ if that row's sTF-encoding gene was actually used for SES library
construction, and ″No″ if it was not. Part types that are considered necessary for SES
functionality are labeled ″Necessary part type,″ while those that are considered optional for
SES functionality are labeled ″Optional part type.″

| DBD | NLS | Linker | TAD | | |
|---|---|---|---|---|---|
| Necessary | Necessary | Optional | Necessary | | |
| part type | part type | part type | part type | Full name | Used? |
| Bm3R1 | SV40_NLS | Linker1_ | B112_TAD | Bm3R1-SV40_NLS-Linker1_long- | Yes |
| | | long | | B112_TAD | |
| Bm3R1 | SV40_NLS | Linker1_ | B112_TAD | Bm3R1-SV40_NLS-Linker1_short- | Yes |
| | | short | | B112_TAD | |
| Bm3R1 | SV40_NLS | Linker1_ | B42_TAD | Bm3R1-SV40_NLS-Linker1_long- | Yes |
| | | long | | B42_TAD | |
| Bm3R1 | SV40_NLS | Linker1_ | B42_TAD | Bm3R1-SV40_NLS-Linker1_short- | Yes |
| | | short | | B42_TAD | |
| Bm3R1 | SV40_NLS | Linker1_ | GAL4_TAD | Bm3R1-SV40_NLS-Linker1_long- | Yes |
| | | long | | GAL4_TAD | |
| Bm3R1 | SV40_NLS | Linker1_ | GAL4_TAD | Bm3R1-SV40_NLS-Linker1_short- | Yes |
| | | short | | GAL4_TAD | |
| Bm3R1 | SV40_NLS | Linker1_ | miniVPR_TAD | Bm3R1-SV40_NLS-Linker1_long- | Yes |
| | | long | | miniVPR_TAD | |
| Bm3R1 | SV40_NLS | Linker1_ | miniVPR_TAD | Bm3R1-SV40_NLS-Linker1_short- | Yes |
| | | short | | miniVPR_TAD | |
| Bm3R1 | SV40_NLS | Linker1_ | Mxr1_TAD | Bm3R1-SV40_NLS-Linker1_long- | Yes |
| | | long | | Mxr1_TAD | |
| Bm3R1 | SV40_NLS | Linker1_ | Mxr1_TAD | Bm3R1-SV40_NLS-Linker1_short- | Yes |
| | | short | | Mxr1_TAD | |
| Bm3R1 | SV40_NLS | Linker1_ | No_TAD | Bm3R1-SV40_NLS-Linker1_long- | Yes |
| | | long | | No_TAD | |
| Bm3R1 | SV40_NLS | Linker1_ | No_TAD | Bm3R1-SV40_NLS-Linker1_short- | Yes |
| | | short | | No_TAD | |
| Bm3R1 | SV40_NLS | Linker1_ | PH_TAD | Bm3R1-SV40_NLS-Linker1_long- | Yes |
| | | long | | PH_TAD | |
| Bm3R1 | SV40_NLS | Linker1_ | PH_TAD | Bm3R1-SV40_NLS-Linker1_short- | Yes |
| | | short | | PH_TAD | |
| Bm3R1 | SV40_NLS | Linker1_ | VP16_TAD | Bm3R1-SV40_NLS-Linker1_long- | Yes |
| | | long | | VP16_TAD | |
| Bm3R1 | SV40_NLS | Linker1_ | VP16_TAD | Bm3R1-SV40_NLS-Linker1_short- | Yes |
| | | short | | VP16_TAD | |
| Bm3R1 | SV40_NLS | Linker1_ | VP64_TAD | Bm3R1-SV40_NLS-Linker1_long- | Yes |
| | | long | | VP64_TAD | |
| Bm3R1 | SV40_NLS | Linker1_ | VP64_TAD | Bm3R1-SV40_NLS-Linker1_short- | Yes |
| | | short | | VP64_TAD | |
| Bm3R1 | SV40_NLS | Linker1_ | VP64v2_TAD | Bm3R1-SV40_NLS-Linker1_long- | Yes |
| | | long | | VP64v2_TAD | |
| Bm3R1 | SV40_NLS | Linker1_ | VP64v2_TAD | Bm3R1-SV40_NLS-Linker1_short- | Yes |
| | | short | | VP64v2_TAD | |
| Bm3R1 | SV40_NLS | Linker1_ | VPH_TAD | Bm3R1-SV40_NLS-Linker1_long- | Yes |
| | | long | | VPH_TAD | |
| Bm3R1 | SV40_NLS | Linker1_ | VPH_TAD | Bm3R1-SV40_NLS-Linker1_short- | Yes |
| | | short | | VPH_TAD | |
| Bm3R1 | SV40_NLS | Linker1_ | VPR_TAD | Bm3R1-SV40_NLS-Linker1_long- | Yes |
| | | long | | VPR_TAD | |
| Bm3R1 | SV40_NLS | Linker1_ | VPR_TAD | Bm3R1-SV40_NLS-Linker1_short- | Yes |
| | | short | | VPR_TAD | |

TABLE 8
One-component sTFs based on DNA-binding domain PhlF_AM. The ″Full name″
entry on a given row is the name of both the gene encoding that row's sTF protein as well as
that of this sTF protein. The DNA sequence of this sTF-encoding gene is a concatenation of
the DNA sequences of the indicated part-type variants on the corresponding row in left-to-
right order. The amino acid sequence of this sTF protein is a concatenation of the amino acid
sequences of the indicated part-type variants on the corresponding row in left-to-right order.
DNA and amino acid sequences of component part-type variants are in Table 21. The
″Used?″ entry on a given row is ″Yes″ if that row's sTF-encoding gene was actually used for
SES library construction, and ″No″ if it was not. Part types that are considered necessary for
SES functionality are labeled ″Necessary part type,″ while those that are considered optional
for SES functionality are labeled ″Optional part type.″

| DBD | NLS | Linker | TAD | | |
|---|---|---|---|---|---|
| Necessary | Necessary | Optional | Necessary | | |
| part type | part type | part type | part type | Full name | Used? |
| PhlF_AM | SV40_NLS | Linker1_ | B112_TAD | PhlF_AM-SV40_NLS-Linker1_long- | Yes |
| | | long | | B112_TAD | |
| PhlF_AM | SV40_NLS | Linker1_ | B112_TAD | PhlF_AM-SV40_NLS-Linker1_short- | Yes |
| | | short | | B112_TAD | |
| PhlF_AM | SV40_NLS | Linker1_ | B42_TAD | PhlF_AM-SV40_NLS-Linker1_long- | Yes |
| | | long | | B42_TAD | |
| PhlF_AM | SV40_NLS | Linker1_ | B42_TAD | PhlF_AM-SV40_NLS-Linker1_short- | Yes |
| | | short | | B42_TAD | |
| PhlF_AM | SV40_NLS | Linker1_ | GAL4_TAD | PhlF_AM-SV40_NLS-Linker1_long- | Yes |
| | | long | | GAL4_TAD | |
| PhlF_AM | SV40_NLS | Linker1_ | GAL4_TAD | PhlF_AM-SV40_NLS-Linker1_short- | Yes |
| | | short | | GAL4_TAD | |
| PhlF_AM | SV40_NLS | Linker1_ | miniVPR_TAD | PhlF_AM-SV40_NLS-Linker1_long- | Yes |
| | | long | | miniVPR_TAD | |
| PhlF_AM | SV40_NLS | Linker1_ | miniVPR_TAD | PhlF_AM-SV40_NLS-Linker1_short- | Yes |
| | | short | | miniVPR_TAD | |
| PhlF_AM | SV40_NLS | Linker1_ | Mxr1_TAD | PhlF_AM-SV40_NLS-Linker1_long- | Yes |
| | | long | | Mxr1_TAD | |
| PhlF_AM | SV40_NLS | Linker1_ | Mxr1 TAD | PhlF_AM-SV40_NLS-Linker1_short- | Yes |
| | | short | | Mxr1_TAD | |
| PhlF_AM | SV40_NLS | Linker1_ | No_TAD | PhlF_AM-SV40_NLS-Linker1_long- | Yes |
| | | long | | No_TAD | |
| PhlF_AM | SV40_NLS | Linker1_ | No_TAD | PhlF_AM-SV40_NLS-Linker1_short- | Yes |
| | | short | | No_TAD | |
| PhlF_AM | SV40_NLS | Linker1_ | PH_TAD | PhlF_AM-SV40_NLS-Linker1_long- | Yes |
| | | long | | PH_TAD | |
| PhlF_AM | SV40_NLS | Linker1_ | PH_TAD | PhlF_AM-SV40_NLS-Linker1_short- | Yes |
| | | short | | PH_TAD | |
| PhlF_AM | SV40_NLS | Linker1_ | VP16_TAD | PhlF_AM-SV40_NLS-Linker1_long- | Yes |
| | | long | | VP16_TAD | |
| PhlF_AM | SV40_NLS | Linker1_ | VP16_TAD | PhlF_AM-SV40_NLS-Linker1_short- | Yes |
| | | short | | VP16_TAD | |
| PhlF_AM | SV40_NLS | Linker1_ | VP64_TAD | PhlF_AM-SV40_NLS-Linker1_long- | Yes |
| | | long | | VP64_TAD | |
| PhlF_AM | SV40_NLS | Linker1_ | VP64_TAD | PhlF_AM-SV40_NLS-Linker1_short- | No |
| | | short | | VP64_TAD | |
| PhlF_AM | SV40_NLS | Linker1_ | VP64v2_TAD | PhlF_AM-SV40_NLS-Linker1_long- | Yes |
| | | long | | VP64v2_TAD | |
| PhlF_AM | SV40_NLS | Linker1_ | VP64v2_TAD | PhlF_AM-SV40_NLS-Linker1_short- | Yes |
| | | short | | VP64v2_TAD | |
| PhlF_AM | SV40_NLS | Linker1_ | VPH_TAD | PhlF_AM-SV40_NLS-Linker1_long- | Yes |
| | | long | | VPH_TAD | |
| PhlF_AM | SV40_NLS | Linker1_ | VPH_TAD | PhlF_AM-SV40_NLS-Linker1_short- | Yes |
| | | short | | VPH_TAD | |
| PhlF_AM | SV40_NLS | Linker1_ | VPR_TAD | PhlF_AM-SV40_NLS-Linker1_long- | Yes |
| | | long | | VPR_TAD | |
| PhlF_AM | SV40_NLS | Linker1_ | VPR_TAD | PhlF_AM-SV40_NLS-Linker1_short- | Yes |
| | | short | | VPR_TAD | |

TABLE 9
One-component sTFs based on DNA-binding domain TetR. The ″Full name″ entry
on a given row is the name of both the gene encoding that row's sTF protein as well as that of
this sTF protein. The DNA sequence of this sTF-encoding gene is a concatenation of the DNA
sequences of the indicated part-type variants on the corresponding row in left-to-right order.
The amino acid sequence of this sTF protein is a concatenation of the amino acid sequences
of the indicated part-type variants on the corresponding row in left-to-right order. DNA and
amino acid sequences of component part-type variants are in Table 21. The ″Used?″ entry on
a given row is ″Yes″ if that row's sTF-encoding gene was actually used for SES library
construction, and ″No″ if it was not. Part types that are considered necessary for SES
functionality are labeled ″Necessary part type,″ while those that are considered optional for
SES functionality are labeled ″Optional part type.″

| DBD | NLS | Linker | TAD | | |
|---|---|---|---|---|---|
| Necessary | Necessary | Optional | Necessary | | |
| part type | part type | part type | part type | Full name | Used? |
| TetR | SV40_NLS | Linker1_ | B112_TAD | TetR-SV40_NLS-Linker1_long- | Yes |
| | | long | | B112_TAD | |
| TetR | SV40_NLS | Linker1_ | B112_TAD | TetR-SV40_NLS-Linker1_short- | Yes |
| | | short | | B112_TAD | |
| TetR | SV40_NLS | Linker1_ | B42_TAD | TetR-SV40_NLS-Linker1_long- | Yes |
| | | long | | B42_TAD | |
| TetR | SV40_NLS | Linker1_ | B42_TAD | TetR-SV40_NLS-Linker1_short- | Yes |
| | | short | | B42_TAD | |
| TetR | SV40_NLS | Linker1_ | GAL4_TAD | TetR-SV40_NLS-Linker1_long- | Yes |
| | | long | | GAL4_TAD | |
| TetR | SV40_NLS | Linker1_ | GAL4_TAD | TetR-SV40_NLS-Linker1_short- | Yes |
| | | short | | GAL4_TAD | |
| TetR | SV40_NLS | Linker1_ | miniVPR_TAD | TetR-SV40_NLS-Linker1_long- | Yes |
| | | long | | miniVPR_TAD | |
| TetR | SV40_NLS | Linker1_ | miniVPR_TAD | TetR-SV40_NLS-Linker1_short- | Yes |
| | | short | | miniVPR_TAD | |
| TetR | SV40_NLS | Linker1_ | Mxr1_TAD | TetR-SV40_NLS-Linker1_long- | Yes |
| | | long | | Mxr1_TAD | |
| TetR | SV40_NLS | Linker1_ | Mxr1_TAD | TetR-SV40_NLS-Linker1_short- | Yes |
| | | short | | Mxr1_TAD | |
| TetR | SV40_NLS | Linker1_ | No_TAD | TetR-SV40_NLS-Linker1_long- | Yes |
| | | long | | No_TAD | |
| TetR | SV40_NLS | Linker1_ | No_TAD | TetR-SV40_NLS-Linker1_short- | Yes |
| | | short | | No_TAD | |
| TetR | SV40_NLS | Linker1_ | PH_TAD | TetR-SV40_NLS-Linker1_long- | Yes |
| | | long | | PH_TAD | |
| TetR | SV40_NLS | Linker1_ | PH_TAD | TetR-SV40_NLS-Linker1_short- | Yes |
| | | short | | PH_TAD | |
| TetR | SV40_NLS | Linker1_ | VP16_TAD | TetR-SV40_NLS-Linker1_long- | Yes |
| | | long | | VP16_TAD | |
| TetR | SV40_NLS | Linker1_ | VP16_TAD | TetR-SV40_NLS-Linker1_short- | Yes |
| | | short | | VP16_TAD | |
| TetR | SV40_NLS | Linker1_ | VP64_TAD | TetR-SV40_NLS-Linker1_long- | No |
| | | long | | VP64_TAD | |
| TetR | SV40_NLS | Linker1_ | VP64_TAD | TetR-SV40_NLS-Linker1_short- | Yes |
| | | short | | VP64_TAD | |
| TetR | SV40_NLS | Linker1_ | VP64v2_TAD | TetR-SV40_NLS-Linker1_long- | Yes |
| | | long | | VP64v2_TAD | |
| TetR | SV40_NLS | Linker1_ | VP64v2_TAD | TetR-SV40_NLS-Linker1_short- | Yes |
| | | short | | VP64v2_TAD | |
| TetR | SV40_NLS | Linker1_ | VPH_TAD | TetR-SV40_NLS-Linker1_long- | Yes |
| | | long | | VPH_TAD | |
| TetR | SV40_NLS | Linker1_ | VPH_TAD | TetR-SV40_NLS-Linker1_short- | Yes |
| | | short | | VPH_TAD | |
| TetR | SV40_NLS | Linker1_ | VPR_TAD | TetR-SV40_NLS-Linker1_long- | Yes |
| | | long | | VPR_TAD | |
| TetR | SV40_NLS | Linker1_ | VPR_TAD | TetR-SV40_NLS-Linker1_short- | Yes |
| | | short | | VPR_TAD | |

TABLE 10
One-component sTFs based on DNA-binding domain VanR_AM. The ″Full name″
entry on a given row is the name of both the gene encoding that row's sTF protein as well as
that of this sTF protein. The DNA sequence of this sTF-encoding gene is a concatenation of
the DNA sequences of the indicated part-type variants on the corresponding row in left-to-
right order. The amino acid sequence of this sTF protein is a concatenation of the amino acid
sequences of the indicated part-type variants on the corresponding row in left-to-right order.
DNA and amino acid sequences of component part-type variants are in Table 21. The
″Used?″ entry on a given row is ″Yes″ if that row's sTF-encoding gene was actually used for
SES library construction, and ″No″ if it was not. Part types that are considered necessary for
SES functionality are labeled ″Necessary part type,″ while those that are considered optional
for SES functionality are labeled ″Optional part type.″

| DBD | NLS | Linker | TAD | | |
|---|---|---|---|---|---|
| Necessary | Necessary | Optional | Necessary | | |
| part type | part type | part type | part type | Full name | Used? |
| VanR_AM | SV40_NLS | Linker1_ | B112_TAD | VanR_AM-SV40_NLS- | Yes |
| | | long | | Linker1_long-B112_TAD | |
| VanR_AM | SV40_NLS | Linker1_ | B112_TAD | VanR_AM-SV40_NLS- | Yes |
| | | short | | Linker1_short-B112_TAD | |
| VanR_AM | SV40 NLS | Linker1_ | B42_TAD | VanR_AM-SV40_NLS- | Yes |
| | | long | | Linker1_long-B42_TAD | |
| VanR_AM | SV40_NLS | Linker1_ | B42_TAD | VanR_AM-SV40_NLS- | Yes |
| | | short | | Linker1_short-B42_TAD | |
| VanR_AM | SV40_NLS | Linker1_ | GAL4_TAD | VanR_AM-SV40_NLS- | Yes |
| | | long | | Linker1_long-GAL4_TAD | |
| VanR_AM | SV40_NLS | Linker1_ | GAL4_TAD | VanR_AM-SV40_NLS- | Yes |
| | | short | | Linker1_short-GAL4_TAD | |
| VanR_AM | SV40_NLS | Linker1_ | miniVPR_TAD | VanR_AM-SV40_NLS- | Yes |
| | | long | | Linker1_long-miniVPR_TAD | |
| VanR_AM | SV40_NLS | Linker1_ | miniVPR_TAD | VanR_AM-SV40_NLS- | Yes |
| | | short | | Linker1_short-miniVPR_TAD | |
| VanR_AM | SV40_NLS | Linker1_ | Mxr1_TAD | VanR_AM-SV40_NLS- | Yes |
| | | long | | Linker1_long-Mxr1_TAD | |
| VanR_AM | SV40_NLS | Linker1_ | Mxr1_TAD | VanR_AM-SV40_NLS- | Yes |
| | | short | | Linker1_short-Mxr1_TAD | |
| VanR_AM | SV40_NLS | Linker1_ | No_TAD | VanR_AM-SV40_NLS- | Yes |
| | | long | | Linker1_long-No_TAD | |
| VanR_AM | SV40_NLS | Linker1_ | No_TAD | VanR_AM-SV40_NLS- | Yes |
| | | short | | Linker1_short-No_TAD | |
| VanR_AM | SV40_NLS | Linker1_ | PH_TAD | VanR_AM-SV40_NLS- | Yes |
| | | long | | Linker1_long-PH_TAD | |
| VanR_AM | SV40_NLS | Linker1_ | PH_TAD | VanR_AM-SV40_NLS- | Yes |
| | | short | | Linker1_short-PH_TAD | |
| VanR_AM | SV40_NLS | Linker1_ | VP16_TAD | VanR_AM-SV40_NLS- | Yes |
| | | long | | Linker1_long-VP16_TAD | |
| VanR_AM | SV40_NLS | Linker1_ | VP16_TAD | VanR_AM-SV40_NLS- | Yes |
| | | short | | Linker1_short-VP16_TAD | |
| VanR_AM | SV40_NLS | Linker1_ | VP64_TAD | VanR_AM-SV40_NLS- | No |
| | | long | | Linker1_long-VP64_TAD | |
| VanR_AM | SV40_NLS | Linker1_ | VP64_TAD | VanR_AM-SV40_NLS- | No |
| | | short | | Linker1_short-VP64_TAD | |
| VanR_AM | SV40_NLS | Linker1_ | VP64v2_TAD | VanR_AM-SV40_NLS- | Yes |
| | | long | | Linker1_long-VP64v2_TAD | |
| VanR_AM | SV40_NLS | Linker1_ | VP64v2_TAD | VanR_AM-SV40_NLS- | Yes |
| | | short | | Linker1_short-VP64v2_TAD | |
| VanR_AM | SV40_NLS | Linker1_ | VPH_TAD | VanR_AM-SV40_NLS- | Yes |
| | | long | | Linker1_long-VPH_TAD | |
| VanR_AM | SV40_NLS | Linker1_ | VPH_TAD | VanR_AM-SV40_NLS- | Yes |
| | | short | | Linker1_short-VPH_TAD | |
| VanR_AM | SV40_NLS | Linker1_ | VPR_TAD | VanR_AM-SV40_NLS- | Yes |
| | | long | | Linker1_long-VPR_TAD | |
| VanR_AM | SV40_NLS | Linker1_ | VPR_TAD | VanR_AM-SV40_NLS- | Yes |
| | | short | | Linker1_short-VPR_TAD | |

Two-component sTFs (used for all three processes) are described in Table 11, Table 12, Table 13, and Table 14, and comprise 9 subparts: DBD, NLS1, Linker, BPP1, 2A, BPP2, NLS2, GD, and TAD. Part types that are considered necessary for SES functionality are the DBD and TAD. For a given DBD, up to 108 possible two-component sTFs were used for SES library assembly, based on a combinatorial assembly of 1 DBD variant×1 NLS 1 variant×1 Linker variant×3 BPP1 variants×1 2A variant×1 BPP2 variant×1 NLS2 variant×3 GD variants×12 TAD variants.

TABLE 11
Two-component sTFs based on DNA-binding domain Bm3R1. The “Full name” entry on a given row is the name of both the gene encoding that row's sTF protein as well as
the name of the corresponding sTF protein. The DNA sequence of each sTF-encoding gene is a concatenation of the DNA sequences of the indicated part-type variants on the
corresponding row in left-to-right order. The amino acid sequence of each sTF protein is a concatenation of the amino acid sequences of the indicated part-type variants on the
corresponding row in left-to-right order. DNA and amino acid sequences of component part-type variants are in Table 21. The “Used?” entry on a given row is “Yes” if that
row's sTF-encoding gene was actually used for SES library construction, and “No” if it was not.

| DBD | NLS1 | Linker | BPP1 | 2A | BPP2 | NLS2 | OD | TAD | | |
|---|---|---|---|---|---|---|---|---|---|---|
| Necessary | Necessary | Optional | Optional | Optional | Optional | Necessary | Optional | Necessary | | |
| part | part | part | part | part | part | part | part | part | | |
| type | type | type | type | type | type | type | type | type | Full Name | Used? |
| Bm3R1 | SV40_NLS_1 | Linker1_long | 1 × SpyTag002 | ERBV_1_P2A | SpyCatcher002 | SV40_NLS_2 | Linker_only_for_oligomerization | B112_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B112_TAD | |
| Bm3R1 | SV40_NLS_1 | Linker1_long | 1 × SpyTag002 | ERBV_1_P2A | SpyCatcher002 | SV40_NLS_2 | Trimerization_domain | B112_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-B112_TAD | |
| Bm3R1 | SV40_NLS_1 | Linker1_long | 1 × SpyTag002 | ERBV_1_P2A | SpyCatcher002 | SV40_NLS_2 | Heptamerization_domain | B112_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain-B112_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | B42_TAD | Bm3R1-SV40_NLS_1-Linker1_long- | No |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B42_TAD | |
| Bm3R1 | SV40_NLS_1 | Linker1_long | 1 × SpyTag002 | ERBV_1_P2A | SpyCatcher002 | SV40_NLS_2 | Trimerization_domain | B42_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-B42_TAD | |
| Bm3R1 | SV40_NLS_1 | Linker1_long | 1 × SpyTag002 | ERBV_1_P2A | SpyCatcher002 | SV40_NLS_2 | Heptamerization_domain | B42_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain-B42_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | GAL4_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | GAL4_TAD | |
| Bm3R1 | SV40_NLS_1 | Linker1_long | 1 × SpyTag002 | ERBV_1_P2A | SpyCatcher002 | SV40_NLS_2 | Trimerization_domain | GAL4_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain- | |
| | | | | | | | | | GAL4_TAD | |
| Bm3R1 | SV40_NLS_1 | Linker1_long | 1 × SpyTag002 | ERBV_1_P2A | SpyCatcher002 | SV40_NLS_2 | Heptamerization_domain | GAL4_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | GAL4_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | miniVPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | miniVPR_TAD | |
| Bm3R1 | SV40_NLS_1 | Linker1_long | 1 × SpyTag002 | ERBV_1_P2A | SpyCatcher002 | SV40_NLS_2 | Trimerization_domain | miniVPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain- | |
| | | | | | | | | | miniVPR_TAD | |
| Bm3R1 | SV40_NLS_1 | Linker1_long | 1 × SpyTag002 | ERBV_1_P2A | SpyCatcher002 | SV40_NLS_2 | Heptamerization_domain | miniVPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | miniVPR_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | Mxr1_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | Mxr1_TAD | |
| Bm3R1 | SV40_NLS_1 | Linker1_long | 1 × SpyTag002 | ERBV_1_P2A | SpyCatcher002 | SV40_NLS_2 | Trimerization_domain | Mxr1_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-Mxr1_TAD | |
| Bm3R1 | SV40_NLS_1 | Linker1_long | 1 × SpyTag002 | ERBV_1_P2A | SpyCatcher002 | SV40_NLS_2 | Heptamerization_domain | Mxr1_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | Mxr1_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | No_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | No_TAD | |
| Bm3R1 | SV40_NLS_1 | Linker1_long | 1 × SpyTag002 | ERBV_1_P2A | SpyCatcher002 | SV40_NLS_2 | Trimerization_domain | No_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-No_TAD | |
| Bm3R1 | SV40_NLS_1 | Linker1_long | 1 × SpyTag002 | ERBV_1_P2A | SpyCatcher002 | SV40_NLS_2 | Heptamerization_domain | No_TAD | Bm3R1-SV40_NLS_1-Linker1_long- | No |
| | | | | | | | | | 1 × SpyTag002-ERBV_1_P2A- | |
| | | | | | | | | | SpyCatcher002-SV40_NLS_2- | |
| | | | | | | | | | Heptamerization_domain-No_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | PH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | PH_TAD | |
| Bm3R1 | SV40_NLS_1 | Linker1_long | 1 × SpyTag002 | ERBV_1_P2A | SpyCatcher002 | SV40_NLS_2 | Trimerization_domain | PH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-PH_TAD | |
| Bm3R1 | SV40_NLS_1 | Linker1_long | 1 × SpyTag002 | ERBV_1_P2A | SpyCatcher002 | SV40_NLS_2 | Heptamerization_domain | PH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain-PH_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | VP16_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP16_TAD | |
| Bm3R1 | SV40_NLS_1 | Linker1_long | 1 × SpyTag002 | ERBV_1_P2A | SpyCatcher002 | SV40_NLS_2 | Trimerization_domain | VP16_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-VP16_TAD | |
| Bm3R1 | SV40_NLS_1 | Linker1_long | 1 × SpyTag002 | ERBV_1_P2A | SpyCatcher002 | SV40_NLS_2 | Heptamerization_domain | VP16_TAD | Bm3R1-SV40_NLS_1-Linker1_long- | No |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP16_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | VP64_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64_TAD | |
| Bm3R1 | SV40_NLS_1 | Linker1_long | 1 × SpyTag002 | ERBV_1_P2A | SpyCatcher002 | SV40_NLS_2 | Trimerization_domain | VP64_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-VP64_TAD | |
| Bm3R1 | SV40_NLS_1 | Linker1_long | 1 × SpyTag002 | ERBV_1_P2A | SpyCatcher002 | SV40_NLS_2 | Heptamerization_domain | VP64_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | VP64v2_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64v2_TAD | |
| Bm3R1 | SV40_NLS_1 | Linker1_long | 1 × SpyTag002 | ERBV_1_P2A | SpyCatcher002 | SV40_NLS_2 | Trimerization_domain | VP64v2_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-VP64v2_TAD | |
| Bm3R1 | SV40_NLS_1 | Linker1_long | 1 × SpyTag002 | ERBV_1_P2A | SpyCatcher002 | SV40_NLS_2 | Heptamerization_domain | VP64v2_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64v2_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | VPH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPH_TAD | |
| Bm3R1 | SV40_NLS_1 | Linker1_long | 1 × SpyTag002 | ERBV_1_P2A | SpyCatcher002 | SV40_NLS_2 | Trimerization_domain | VPH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-VPH_TAD | |
| Bm3R1 | SV40_NLS_1 | Linker1_long | 1 × SpyTag002 | ERBV_1_P2A | SpyCatcher002 | SV40_NLS_2 | Heptamerization_domain | VPH_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain-VPH_TAD | |
| Bm3R1 | SV40_NLS_1 | Linker1_long | 1 × SpyTag002 | ERBV_1_P2A | SpyCatcher002 | SV40_NLS_2 | Linker_only_for_oligomerization | VPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPR_TAD | |
| Bm3R1 | SV40_NLS_1 | Linker1_long | 1 × SpyTag002 | ERBV_1_P2A | SpyCatcher002 | SV40_NLS_2 | Trimerization_domain | VPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Trimerization_domain-VPR_TAD | |
| Bm3R1 | SV40_NLS_1 | Linker1_long | 1 × SpyTag002 | ERBV_1_P2A | SpyCatcher002 | SV40_NLS_2 | Heptamerization_domain | VPR_TAD | Bm3R1-SV40_NLS_1-Linker1_long- | Yes |
| | | | | | | | | | 1 × SpyTag002-ERBV_1_P2A- | |
| | | | | | | | | | SpyCatcher002-SV40_NLS_2- | |
| | | | | | | | | | Heptamerization_domain-VPR_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | B112_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B112_TAD | |
| Bm3R1 | SV40_NLS_1 | Linker1_long | 2 × SpyTag002 | ERBV_1_P2A | SpyCatcher002 | SV40_NLS_2 | Trimerization_domain | B112_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-B112_TAD | |
| Bm3R1 | SV40_NLS_1 | Linker1_long | 2 × SpyTag002 | ERBV_1_P2A | SpyCatcher002 | SV40_NLS_2 | Heptamerization_domain | B112_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain-B112_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | B42_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B42_TAD | |
| Bm3R1 | SV40_NLS_1 | Linker1_long | 2 × SpyTag002 | ERBV_1_P2A | SpyCatcher002 | SV40_NLS_2 | Trimerization_domain | B42_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-B42_TAD | |
| Bm3R1 | SV40_NLS_1 | Linker1_long | 2 × SpyTag002 | ERBV_1_P2A | SpyCatcher002 | SV40_NLS_2 | Heptamerization_domain | B42_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain-B42_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | GAL4_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | GAL4_TAD | |
| Bm3R1 | SV40_NLS_1 | Linker1_long | 2 × SpyTag002 | ERBV_1_P2A | SpyCatcher002 | SV40_NLS_2 | Trimerization_domain | GAL4_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain- | |
| | | | | | | | | | GAL4_TAD | |
| Bm3R1 | SV40_NLS_1 | Linker1_long | 2 × SpyTag002 | ERBV_1_P2A | SpyCatcher002 | SV40_NLS_2 | Heptamerization_domain | GAL4_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | GAL4_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | miniVPR_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | miniVPR_TAD | |
| Bm3R1 | SV40_NLS_1 | Linker1_long | 2 × SpyTag002 | ERBV_1_P2A | SpyCatcher002 | SV40_NLS_2 | Trimerization_domain | miniVPR_TAD | Bm3R1-SV40_NLS_1-Linker1_long- | No |
| | | | | | | | | | 2 × SpyTag002-ERBV_1_P2A- | |
| | | | | | | | | | SpyCatcher002-SV40_NLS_2- | |
| | | | | | | | | | Trimerization_domain- | |
| | | | | | | | | | miniVPR_TAD | |
| Bm3R1 | SV40_NLS_1 | Linker1_long | 2 × SpyTag002 | ERBV_1_P2A | SpyCatcher002 | SV40_NLS_2 | Heptamerization_domain | miniVPR_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | miniVPR_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | Mxr1_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | Mxr1_TAD | |
| Bm3R1 | SV40_NLS_1 | Linker1_long | 2 × SpyTag002 | ERBV_1_P2A | SpyCatcher002 | SV40_NLS_2 | Trimerization_domain | Mxr1_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-Mxr1_TAD | |
| Bm3R1 | SV40_NLS_1 | Linker1_long | 2 × SpyTag002 | ERBV_1_P2A | SpyCatcher002 | SV40_NLS_2 | Heptamerization_domain | Mxr1_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | Mxr1_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | No_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | No_TAD | |
| Bm3R1 | SV40_NLS_1 | Linker1_long | 2 × SpyTag002 | ERBV_1_P2A | SpyCatcher002 | SV40_NLS_2 | Trimerization_domain | No_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-No_TAD | |
| Bm3R1 | SV40_NLS_1 | Linker1_long | 2 × SpyTag002 | ERBV_1_P2A | SpyCatcher002 | SV40_NLS_2 | Heptamerization_domain | No_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain-No_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | PH_TAD | Bm3R1-SV40_NLS_1-Linker1_long- | No |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | PH_TAD | |
| Bm3R1 | SV40_NLS_1 | Linker1_long | 2 × SpyTag002 | ERBV_1_P2A | SpyCatcher002 | SV40_NLS_2 | Trimerization_domain | PH_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-PH_TAD | |
| Bm3R1 | SV40_NLS_1 | Linker1_long | 2 × SpyTag002 | ERBV_1_P2A | SpyCatcher002 | SV40_NLS_2 | Heptamerization_domain | PH_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain-PH_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | VP16_TAD | Bm3R1-SV40_NLS_1-Linker1_long- | No |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP16_TAD | |
| Bm3R1 | SV40_NLS_1 | Linker1_long | 2 × SpyTag002 | ERBV_1_P2A | SpyCatcher002 | SV40_NLS_2 | Trimerization_domain | VP16_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-VP16_TAD | |
| Bm3R1 | SV40_NLS_1 | Linker1_long | 2 × SpyTag002 | ERBV_1_P2A | SpyCatcher002 | SV40_NLS_2 | Heptamerization_domain | VP16_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP16_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | VP64_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64_TAD | |
| Bm3R1 | SV40_NLS_1 | Linker1_long | 2 × SpyTag002 | ERBV_1_P2A | SpyCatcher002 | SV40_NLS_2 | Trimerization_domain | VP64_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-VP64_TAD | |
| Bm3R1 | SV40_NLS_1 | Linker1_long | 2 × SpyTag002 | ERBV_1_P2A | SpyCatcher002 | SV40_NLS_2 | Heptamerization_domain | VP64_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | VP64v2_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64v2_TAD | |
| Bm3R1 | SV40_NLS_1 | Linker1_long | 2 × SpyTag002 | ERBV_1_P2A | SpyCatcher002 | SV40_NLS_2 | Trimerization_domain | VP64v2_TAD | Bm3R1-SV40_NLS_1-Linker1_long- | No |
| | | | | | | | | | 2 × SpyTag002-ERBV_1_P2A- | |
| | | | | | | | | | SpyCatcher002-SV40_NLS_2- | |
| | | | | | | | | | Trimerization_domain-VP64v2_TAD | |
| Bm3R1 | SV40_NLS_1 | Linker1_long | 2 × SpyTag002 | ERBV_1_P2A | SpyCatcher002 | SV40_NLS_2 | Heptamerization_domain | VP64v2_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64v2_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | VPH_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPH_TAD | |
| Bm3R1 | SV40_NLS_1 | Linker1_long | 2 × SpyTag002 | ERBV_1_P2A | SpyCatcher002 | SV40_NLS_2 | Trimerization_domain | VPH_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-VPH_TAD | |
| Bm3R1 | SV40_NLS_1 | Linker1_long | 2 × SpyTag002 | ERBV_1_P2A | SpyCatcher002 | SV40_NLS_2 | Heptamerization_domain | VPH_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Heptamerization_domain-VPH_TAD | |
| Bm3R1 | SV40_NLS_1 | Linker1_long | 2 × SpyTag002 | ERBV_1_P2A | SpyCatcher002 | SV40_NLS_2 | Linker_only_for_oligomerization | VPR_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPR_TAD | |
| Bm3R1 | SV40_NLS_1 | Linker1_long | 2 × SpyTag002 | ERBV_1_P2A | SpyCatcher002 | SV40_NLS_2 | Trimerization_domain | VPR_TAD | Bm3R1-SV40_NLS_1-Linker1_long- | Yes |
| | | | | | | | | | 2 × SpyTag002-ERBV_1_P2A- | |
| | | | | | | | | | SpyCatcher002-SV40_NLS_2- | |
| | | | | | | | | | Trimerization_domain-VPR_TAD | |
| Bm3R1 | SV40_NLS_1 | Linker1_long | 2 × SpyTag002 | ERBV_1_P2A | SpyCatcher002 | SV40_NLS_2 | Heptamerization_domain | VPR_TAD | Bm3R1-SV40_NLS_1-Linker1_long- | No |
| | | | | | | | | | 2 × SpyTag002-ERBV_1_P2A- | |
| | | | | | | | | | SpyCatcher002-SV40_NLS_2- | |
| | | | | | | | | | Heptamerization_domain-VPR_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | B112_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B112_TAD | |
| Bm3R1 | SV40_NLS_1 | Linker1_long | 6 × SpyTag002 | ERBV_1_P2A | SpyCatcher002 | SV40_NLS_2 | Trimerization_domain | B112_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-B112_TAD | |
| Bm3R1 | SV40_NLS_1 | Linker1_long | 6 × SpyTag002 | ERBV_1_P2A | SpyCatcher002 | SV40_NLS_2 | Heptamerization_domain | B112_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-B112_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | B42_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | B42_TAD | |
| Bm3R1 | SV40_NLS_1 | Linker1_long | 6 × SpyTag002 | ERBV_1_P2A | SpyCatcher002 | SV40_NLS_2 | Trimerization_domain | B42_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-B42_TAD | |
| Bm3R1 | SV40_NLS_1 | Linker1_long | 6 × SpyTag002 | ERBV_1_P2A | SpyCatcher002 | SV40_NLS_2 | Heptamerization_domain | B42_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-B42_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | GAL4_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | GAL4_TAD | |
| Bm3R1 | SV40_NLS_1 | Linker1_long | 6 × SpyTag002 | ERBV_1_P2A | SpyCatcher002 | SV40_NLS_2 | Trimerization_domain | GAL4_TAD | Bm3R1-SV40_NLS_1-Linker1_long- | No |
| | | | | | | | | | 6 × SpyTag002-ERBV_1_P2A- | |
| | | | | | | | | | SpyCatcher002-SV40_NLS_2- | |
| | | | | | | | | | Trimerization_domain- | |
| | | | | | | | | | GAL4_TAD | |
| Bm3R1 | SV40_NLS_1 | Linker1_long | 6 × SpyTag002 | ERBV_1_P2A | SpyCatcher002 | SV40_NLS_2 | Heptamerization_domain | GAL4_TAD | Bm3R1-SV40_NLS_1-Linker1_long- | No |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | GAL4_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | miniVPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | miniVPR_TAD | |
| Bm3R1 | SV40_NLS_1 | Linker1_long | 6 × SpyTag002 | ERBV_1_P2A | SpyCatcher002 | SV40_NLS_2 | Trimerization_domain | miniVPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain- | |
| | | | | | | | | | miniVPR_TAD | |
| Bm3R1 | SV40_NLS_1 | Linker1_long | 6 × SpyTag002 | ERBV_1_P2A | SpyCatcher002 | SV40_NLS_2 | Heptamerization_domain | miniVPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | miniVPR_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | Mxr1_TAD | Bm3R1-SV40_NLS_1-Linker1_long- | No |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | Mxr1_TAD | |
| Bm3R1 | SV40_NLS_1 | Linker1_long | 6 × SpyTag002 | ERBV_1_P2A | SpyCatcher002 | SV40_NLS_2 | Trimerization_domain | Mxr1_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-Mxr1_TAD | |
| Bm3R1 | SV40_NLS_1 | Linker1_long | 6 × SpyTag002 | ERBV_1_P2A | SpyCatcher002 | SV40_NLS_2 | Heptamerization_domain | Mxr1_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | Mxr1_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | No_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | No_TAD | |
| Bm3R1 | SV40_NLS_1 | Linker1_long | 6 × SpyTag002 | ERBV_1_P2A | SpyCatcher002 | SV40_NLS_2 | Trimerization_domain | No_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-No_TAD | |
| Bm3R1 | SV40_NLS_1 | Linker1_long | 6 × SpyTag002 | ERBV_1_P2A | SpyCatcher002 | SV40_NLS_2 | Heptamerization_domain | No_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-No_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | PH_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | PH_TAD | |
| Bm3R1 | SV40_NLS_1 | Linker1_long | 6 × SpyTag002 | ERBV_1_P2A | SpyCatcher002 | SV40_NLS_2 | Trimerization_domain | PH_TAD | Bm3R1-SV40_NLS_1-Linker1_long- | No |
| | | | | | | | | | 6 × SpyTag002-ERBV_1_P2A- | |
| | | | | | | | | | SpyCatcher002-SV40_NLS_2- | |
| | | | | | | | | | Trimerization_domain-PH_TAD | |
| Bm3R1 | SV40_NLS_1 | Linker1_long | 6 × SpyTag002 | ERBV_1_P2A | SpyCatcher002 | SV40_NLS_2 | Heptamerization_domain | PH_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-PH_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | VP16_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP16_TAD | |
| Bm3R1 | SV40_NLS_1 | Linker1_long | 6 × SpyTag002 | ERBV_1_P2A | SpyCatcher002 | SV40_NLS_2 | Trimerization_domain | VP16_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-VP16_TAD | |
| Bm3R1 | SV40_NLS_1 | Linker1_long | 6 × SpyTag002 | ERBV_1_P2A | SpyCatcher002 | SV40_NLS_2 | Heptamerization_domain | VP16_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP16_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | VP64_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64_TAD | |
| Bm3R1 | SV40_NLS_1 | Linker1_long | 6 × SpyTag002 | ERBV_1_P2A | SpyCatcher002 | SV40_NLS_2 | Trimerization_domain | VP64_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-VP64_TAD | |
| Bm3R1 | SV40_NLS_1 | Linker1_long | 6 × SpyTag002 | ERBV_1_P2A | SpyCatcher002 | SV40_NLS_2 | Heptamerization_domain | VP64_TAD | Bm3R1-SV40_NLS_1-Linker1_long- | No |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | VP64v2_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VP64v2_TAD | |
| Bm3R1 | SV40_NLS_1 | Linker1_long | 6 × SpyTag002 | ERBV_1_P2A | SpyCatcher002 | SV40_NLS_2 | Trimerization_domain | VP64v2_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-VP64v2_TAD | |
| Bm3R1 | SV40_NLS_1 | Linker1_long | 6 × SpyTag002 | ERBV_1_P2A | SpyCatcher002 | SV40_NLS_2 | Heptamerization_domain | VP64v2_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain- | |
| | | | | | | | | | VP64v2_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | VPH_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPH_TAD | |
| Bm3R1 | SV40_NLS_1 | Linker1_long | 6 × SpyTag002 | ERBV_1_P2A | SpyCatcher002 | SV40_NLS_2 | Trimerization_domain | VPH_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-VPH_TAD | |
| Bm3R1 | SV40_NLS_1 | Linker1_long | 6 × SpyTag002 | ERBV_1_P2A | SpyCatcher002 | SV40_NLS_2 | Heptamerization_domain | VPH_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-VPH_TAD | |
| Bm3R1 | SV40_NLS_1 | Linker1_long | 6 × SpyTag002 | ERBV_1_P2A | SpyCatcher002 | SV40_NLS_2 | Linker_only_for_oligomerization | VPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Linker_only_for_oligomerization- | |
| | | | | | | | | | VPR_TAD | |
| Bm3R1 | SV40_NLS_1 | Linker1_long | 6 × SpyTag002 | ERBV_1_P2A | SpyCatcher002 | SV40_NLS_2 | Trimerization_domain | VPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Trimerization_domain-VPR_TAD | |
| Bm3R1 | SV40_NLS_1 | Linker1_long | 6 × SpyTag002 | ERBV_1_P2A | SpyCatcher002 | SV40_NLS_2 | Heptamerization_domain | VPR_TAD | Bm3R1-SV40_NLS_1-Linker1_long- | Yes |
| | | | | | | | | | 6 × SpyTag002-ERBV_1_P2A- | |
| | | | | | | | | | SpyCatcher002-SV40_NLS_2- | |
| | | | | | | | | | Heptamerization_domain-VPR_TAD | |

TABLE 12
Two-component sTFs based on DNA-binding domain PhlF_AM. The “Full name” entry on a given row is the name of both the gene encoding that row's sTF protein as well as that of
this sTF protein. The DNA sequence of this sTF-encoding gene is a concatenation of the DNA sequences of the indicated part-type variants on the corresponding row in left-to-right
order. The amino acid sequence of this sTF protein is a concatenation of the amino acid sequences of the indicated part-type variants on the corresponding row in left-to-right order.
DNA and amino acid sequences of component part-type variants are in Table 21. The “Used?” entry on a given row is “Yes” if that row's sTF-encoding gene was actually used for
SES library construction, and “No” if it was not. Part types that are considered necessary for SES functionality are labeled “Necassary part type,” while those that are considered
optional for SES functionality are labeled “Optional part type.”

| DBD | NLS1 | Linker | BPP1 | 2A | BPP2 | NLS2 | OD | TAD | | |
|---|---|---|---|---|---|---|---|---|---|---|
| Necessary | Necessary | Optional | Optional | Optional | Optional | Necessary | Optional | Necessary | | |
| part | part | part | part | part | part | part | part | part | | |
| type | type | type | type | type | type | type | type | type | Full name | Used? |
| PhlF_AM | SV40_NLS_1 | Linker1_long | 1 × SpyTag002 | ERBV_1_P2A | SpyCatcher002 | SV40_NLS_2 | Linker_only_for_oligomerization | B112_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| PhlF_AM | SV40_NLS_1 | Linker1_long | 1 × SpyTag002 | ERBV_1_P2A | SpyCatcher002 | SV40_NLS_2 | Trimerization_domain | B112_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| PhlF_AM | SV40_NLS_1 | Linker1_long | 1 × SpyTag002 | ERBV_1_P2A | SpyCatcher002 | SV40_NLS_2 | Heptamerization_domain | B112_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | B42_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| PhlF_AM | SV40_NLS_1 | Linker1_long | 1 × SpyTag002 | ERBV_1_P2A | SpyCatcher002 | SV40_NLS_2 | Trimerization_domain | B42_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| PhlF_AM | SV40_NLS_1 | Linker1_long | 1 × SpyTag002 | ERBV_1_P2A | SpyCatcher002 | SV40_NLS_2 | Heptamerization_domain | B42_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | GAL4_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| PhlF_AM | SV40_NLS_1 | Linker1_long | 1 × SpyTag002 | ERBV_1_P2A | SpyCatcher002 | SV40_NLS_2 | Trimerization_domain | GAL4_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| PhlF_AM | SV40_NLS_1 | Linker1_long | 1 × SpyTag002 | ERBV_1_P2A | SpyCatcher002 | SV40_NLS_2 | Heptamerization_domain | GAL4_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | miniVPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| PhlF_AM | SV40_NLS_1 | Linker1_long | 1 × SpyTag002 | ERBV_1_P2A | SpyCatcher002 | SV40_NLS_2 | Trimerization_domain | miniVPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| PhlF_AM | SV40_NLS_1 | Linker1_long | 1 × SpyTag002 | ERBV_1_P2A | SpyCatcher002 | SV40_NLS_2 | Heptamerization_domain | miniVPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | Mxr1_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| PhlF_AM | SV40_NLS_1 | Linker1_long | 1 × SpyTag002 | ERBV_1_P2A | SpyCatcher002 | SV40_NLS_2 | Trimerization_domain | Mxr1_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| PhlF_AM | SV40_NLS_1 | Linker1_long | 1 × SpyTag002 | ERBV_1_P2A | SpyCatcher002 | SV40_NLS_2 | Heptamerization_domain | Mxr1_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | No_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| PhlF_AM | SV40_NLS_1 | Linker1_long | 1 × SpyTag002 | ERBV_1_P2A | SpyCatcher002 | SV40_NLS_2 | Trimerization_domain | No_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| PhlF_AM | SV40_NLS_1 | Linker1_long | 1 × SpyTag002 | ERBV_1_P2A | SpyCatcher002 | SV40_NLS_2 | Heptamerization_domain | No_TAD | PhlF_AM-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | PH_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| PhlF_AM | SV40_NLS_1 | Linker1_long | 1 × SpyTag002 | ERBV_1_P2A | SpyCatcher002 | SV40_NLS_2 | Trimerization_domain | PH_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| PhlF_AM | SV40_NLS_1 | Linker1_long | 1 × SpyTag002 | ERBV_1_P2A | SpyCatcher002 | SV40_NLS_2 | Heptamerization_domain | PH_TAD | PhlF_AM-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | VP16_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| PhlF_AM | SV40_NLS_1 | Linker1_long | 1 × SpyTag002 | ERBV_1_P2A | SpyCatcher002 | SV40_NLS_2 | Trimerization_domain | VP16_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| PhlF_AM | SV40_NLS_1 | Linker1_long | 1 × SpyTag002 | ERBV_1_P2A | SpyCatcher002 | SV40_NLS_2 | Heptamerization_domain | VP16_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | VP64_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| PhlF_AM | SV40_NLS_1 | Linker1_long | 1 × SpyTag002 | ERBV_1_P2A | SpyCatcher002 | SV40_NLS_2 | Trimerization_domain | VP64_TAD | PhlF_AM-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64_TAD | |
| PhlF_AM | SV40_NLS_1 | Linker1_long | 1 × SpyTag002 | ERBV_1_P2A | SpyCatcher002 | SV40_NLS_2 | Heptamerization_domain | VP64_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | VP64v2_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization | |
| | | | | | | | | | VP64v2_TAD | |
| PhlF_AM | SV40_NLS_1 | Linker1_long | 1 × SpyTag002 | ERBV_1_P2A | SpyCatcher002 | SV40_NLS_2 | Trimerization_domain | VP64v2_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| PhlF_AM | SV40_NLS_1 | Linker1_long | 1 × SpyTag002 | ERBV_1_P2A | SpyCatcher002 | SV40_NLS_2 | Heptamerization_domain | VP64v2_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain | |
| | | | | | | | | | VP64v2_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | VPH_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| PhlF_AM | SV40_NLS_1 | Linker1_long | 1 × SpyTag002 | ERBV_1_P2A | SpyCatcher002 | SV40_NLS_2 | Trimerization_domain | VPH_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| PhlF_AM | SV40_NLS_1 | Linker1_long | 1 × SpyTag002 | ERBV_1_P2A | SpyCatcher002 | SV40_NLS_2 | Heptamerization_domain | VPH_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| PhlF_AM | SV40_NLS_1 | Linker1_long | 1 × SpyTag002 | ERBV_1_P2A | SpyCatcher002 | SV40_NLS_2 | Linker_only_for_oligomerization | VPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| PhlF_AM | SV40_NLS_1 | Linker1_long | 1 × SpyTag002 | ERBV_1_P2A | SpyCatcher002 | SV40_NLS_2 | Trimerization_domain | VPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| PhlF_AM | SV40_NLS_1 | Linker1_long | 1 × SpyTag002 | ERBV_1_P2A | SpyCatcher002 | SV40_NLS_2 | Heptamerization_domain | VPR_TAD | PhlF_AM-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | B112_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| PhlF_AM | SV40_NLS_1 | Linker1_long | 2 × SpyTag002 | ERBV_1_P2A | SpyCatcher002 | SV40_NLS_2 | Trimerization_domain | B112_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| PhlF_AM | SV40_NLS_1 | Linker1_long | 2 × SpyTag002 | ERBV_1_P2A | SpyCatcher002 | SV40_NLS_2 | Heptamerization_domain | B112_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | B42_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| PhlF_AM | SV40_NLS_1 | Linker1_long | 2 × SpyTag002 | ERBV_1_P2A | SpyCatcher002 | SV40_NLS_2 | Trimerization_domain | B42_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| PhlF_AM | SV40_NLS_1 | Linker1_long | 2 × SpyTag002 | ERBV_1_P2A | SpyCatcher002 | SV40_NLS_2 | Heptamerization_domain | B42_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | GAL4_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| PhlF_AM | SV40_NLS_1 | Linker1_long | 2 × SpyTag002 | ERBV_1_P2A | SpyCatcher002 | SV40_NLS_2 | Trimerization_domain | GAL4_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| PhlF_AM | SV40_NLS_1 | Linker1_long | 2 × SpyTag002 | ERBV_1_P2A | SpyCatcher002 | SV40_NLS_2 | Heptamerization_domain | GAL4_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | miniVPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| PhlF_AM | SV40_NLS_1 | Linker1_long | 2 × SpyTag002 | ERBV_1_P2A | SpyCatcher002 | SV40_NLS_2 | Trimerization_domain | miniVPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| PhlF_AM | SV40_NLS_1 | Linker1_long | 2 × SpyTag002 | ERBV_1_P2A | SpyCatcher002 | SV40_NLS_2 | Heptamerization_domain | miniVPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | Mxr1_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| PhlF_AM | SV40_NLS_1 | Linker1_long | 2 × SpyTag002 | ERBV_1_P2A | SpyCatcher002 | SV40_NLS_2 | Trimerization_domain | Mxr1_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| PhlF_AM | SV40_NLS_1 | Linker1_long | 2 × SpyTag002 | ERBV_1_P2A | SpyCatcher002 | SV40_NLS_2 | Heptamerization_domain | Mxr1_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | No_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| PhlF_AM | SV40_NLS_1 | Linker1_long | 2 × SpyTag002 | ERBV_1_P2A | SpyCatcher002 | SV40_NLS_2 | Trimerization_domain | No_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| PhlF_AM | SV40_NLS_1 | Linker1_long | 2 × SpyTag002 | ERBV_1_P2A | SpyCatcher002 | SV40_NLS_2 | Heptamerization_domain | No_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | PH_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| PhlF_AM | SV40_NLS_1 | Linker1_long | 2 × SpyTag002 | ERBV_1_P2A | SpyCatcher002 | SV40_NLS_2 | Trimerization_domain | PH_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| PhlF_AM | SV40_NLS_1 | Linker1_long | 2 × SpyTag002 | ERBV_1_P2A | SpyCatcher002 | SV40_NLS_2 | Heptamerization_domain | PH_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | VP16_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| PhlF_AM | SV40_NLS_1 | Linker1_long | 2 × SpyTag002 | ERBV_1_P2A | SpyCatcher002 | SV40_NLS_2 | Trimerization_domain | VP16_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| PhlF_AM | SV40_NLS_1 | Linker1_long | 2 × SpyTag002 | ERBV_1_P2A | SpyCatcher002 | SV40_NLS_2 | Heptamerization_domain | VP16_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | V64_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | V64_TAD | |
| PhlF_AM | SV40_NLS_1 | Linker1_long | 2 × SpyTag002 | ERBV_1_P2A | SpyCatcher002 | SV40_NLS_2 | Trimerization_domain | V64_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | V64_TAD | |
| PhlF_AM | SV40_NLS_1 | Linker1_long | 2 × SpyTag002 | ERBV_1_P2A | SpyCatcher002 | SV40_NLS_2 | Heptamerization_domain | VP64_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | VP64v2_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| PhlF_AM | SV40_NLS_1 | Linker1_long | 2 × SpyTag002 | ERBV_1_P2A | SpyCatcher002 | SV40_NLS_2 | Trimerization_domain | VP64v2_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| PhlF_AM | SV40_NLS_1 | Linker1_long | 2 × SpyTag002 | ERBV_1_P2A | SpyCatcher002 | SV40_NLS_2 | Heptamerization_domain | VP64v2_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | VPH_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| PhlF_AM | SV40_NLS_1 | Linker1_long | 2 × SpyTag002 | ERBV_1_P2A | SpyCatcher002 | SV40_NLS_2 | Trimerization_domain | VPH_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| PhlF_AM | SV40_NLS_1 | Linker1_long | 2 × SpyTag002 | ERBV_1_P2A | SpyCatcher002 | SV40_NLS_2 | Heptamerization_domain | VPH_TAD | PhlF_AM-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| PhlF_AM | SV40_NLS_1 | Linker1_long | 2 × SpyTag002 | ERBV_1_P2A | SpyCatcher002 | SV40_NLS_2 | Linker_only_for_oligomerization | VPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| PhlF_AM | SV40_NLS_1 | Linker1_long | 2 × SpyTag002 | ERBV_1_P2A | SpyCatcher002 | SV40_NLS_2 | Trimerization_domain | VPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| PhlF_AM | SV40_NLS_1 | Linker1_long | 2 × SpyTag002 | ERBV_1_P2A | SpyCatcher002 | SV40_NLS_2 | Heptamerization_domain | VPR_TAD | PhlF_AM-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | B112_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| PhlF_AM | SV40_NLS_1 | Linker1_long | 6 × SpyTag002 | ERBV_1_P2A | SpyCatcher002 | SV40_NLS_2 | Trimerization_domain | B112_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| PhlF_AM | SV40_NLS_1 | Linker1_long | 6 × SpyTag002 | ERBV_1_P2A | SpyCatcher002 | SV40_NLS_2 | Heptamerization_domain | B112_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | B42_TAD | PhlF_AM-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| PhlF_AM | SV40_NLS_1 | Linker1_long | 6 × SpyTag002 | ERBV_1_P2A | SpyCatcher002 | SV40_NLS_2 | Trimerization_domain | B42_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| PhlF_AM | SV40_NLS_1 | Linker1_long | 6 × SpyTag002 | ERBV_1_P2A | SpyCatcher002 | SV40_NLS_2 | Heptamerization_domain | B42_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | GAL4_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| PhlF_AM | SV40_NLS_1 | Linker1_long | 6 × SpyTag002 | ERBV_1_P2A | SpyCatcher002 | SV40_NLS_2 | Trimerization_domain | GAL4_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| PhlF_AM | SV40_NLS_1 | Linker1_long | 6 × SpyTag002 | ERBV_1_P2A | SpyCatcher002 | SV40_NLS_2 | Heptamerization_domain | GAL4_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | miniVPR_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| PhlF_AM | SV40_NLS_1 | Linker1_long | 6 × SpyTag002 | ERBV_1_P2A | SpyCatcher002 | SV40_NLS_2 | Trimerization_domain | miniVPR_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| PhlF_AM | SV40_NLS_1 | Linker1_long | 6 × SpyTag002 | ERBV_1_P2A | SpyCatcher002 | SV40_NLS_2 | Heptamerization_domain | miniVPR_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | Mxr1_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| PhlF_AM | SV40_NLS_1 | Linker1_long | 6 × SpyTag002 | ERBV_1_P2A | SpyCatcher002 | SV40_NLS_2 | Trimerization_domain | Mxr1_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| PhlF_AM | SV40_NLS_1 | Linker1_long | 6 × SpyTag002 | ERBV_1_P2A | SpyCatcher002 | SV40_NLS_2 | Heptamerization_domain | Mxr1_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | No_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| PhlF_AM | SV40_NLS_1 | Linker1_long | 6 × SpyTag002 | ERBV_1_P2A | SpyCatcher002 | SV40_NLS_2 | Trimerization_domain | No_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| PhlF_AM | SV40_NLS_1 | Linker1_long | 6 × SpyTag002 | ERBV_1_P2A | SpyCatcher002 | SV40_NLS_2 | Heptamerization_domain | No_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | PH_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| PhlF_AM | SV40_NLS_1 | Linker1_long | 6 × SpyTag002 | ERBV_1_P2A | SpyCatcher002 | SV40_NLS_2 | Trimerization_domain | PH_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| PhlF_AM | SV40_NLS_1 | Linker1_long | 6 × SpyTag002 | ERBV_1_P2A | SpyCatcher002 | SV40_NLS_2 | Heptamerization_domain | PH_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | VP16_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| PhlF_AM | SV40_NLS_1 | Linker1_long | 6 × SpyTag002 | ERBV_1_P2A | SpyCatcher002 | SV40_NLS_2 | Trimerization_domain | VP16_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| PhlF_AM | SV40_NLS_1 | Linker1_long | 6 × SpyTag002 | ERBV_1_P2A | SpyCatcher002 | SV40_NLS_2 | Heptamerization_domain | VP16_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | VP64_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| PhlF_AM | SV40_NLS_1 | Linker1_long | 6 × SpyTag002 | ERBV_1_P2A | SpyCatcher002 | SV40_NLS_2 | Trimerization_domain | VP64_TAD | PhlF_AM-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64_TAD | |
| PhlF_AM | SV40_NLS_1 | Linker1_long | 6 × SpyTag002 | ERBV_1_P2A | SpyCatcher002 | SV40_NLS_2 | Heptamerization_domain | VP64_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | VP64v2_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| PhlF_AM | SV40_NLS_1 | Linker1_long | 6 × SpyTag002 | ERBV_1_P2A | SpyCatcher002 | SV40_NLS_2 | Trimerization_domain | VP64v2_TAD | PhlF_AM-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| PhlF_AM | SV40_NLS_1 | Linker1_long | 6 × SpyTag002 | ERBV_1_P2A | SpyCatcher002 | SV40_NLS_2 | Heptamerization_domain | VP64v2_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | VPH_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| PhlF_AM | SV40_NLS_1 | Linker1_long | 6 × SpyTag002 | ERBV_1_P2A | SpyCatcher002 | SV40_NLS_2 | Trimerization_domain | VPH_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| PhlF_AM | SV40_NLS_1 | Linker1_long | 6 × SpyTag002 | ERBV_1_P2A | SpyCatcher002 | SV40_NLS_2 | Heptamerization_domain | VPH_TAD | PhlF_AM-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| PhlF_AM | SV40_NLS_1 | Linker1_long | 6 × SpyTag002 | ERBV_1_P2A | SpyCatcher002 | SV40_NLS_2 | Linker_only_for_oligomerization | VPR_TAD | PhlF_AM-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| PhlF_AM | SV40_NLS_1 | Linker1_long | 6 × SpyTag002 | ERBV_1_P2A | SpyCatcher002 | SV40_NLS_2 | Trimerization_domain | VPR_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| PhlF_AM | SV40_NLS_1 | Linker1_long | 6 × SpyTag002 | ERBV_1_P2A | SpyCatcher002 | SV40_NLS_2 | Heptamerization_domain | VPR_TAD | PhlF_AM-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |

TABLE 13
Two-component sTFs based on DNA-binding domain tETr. The “Full name” entry on a given row is the name of both the gene encoding that row's sTF protein as well as that of
this sTF protein. The DNA sequence of this sTF-encoding gene is a concatenation of the DNA sequences of the indicated part-type variants on the corresponding row in left-to-right
order. The amino acid sequence of this sTF protein is a concatenation of the amino acid sequences of the indicated part-type variants on the corresponding row in left-to-right order.
DNA and amino acid sequences of component part-type variants are in Table 21. The “Used?” entry on a given row is “Yes” if that row's sTF-encoding gene was actually used for
SES library construction, and “No” if it was not. Part types that are considered necessary for SES functionality are labeled “Necassary part type,” while those that are considered
optional for SES functionality are labeled “Optional part type.”

| DBD | NLS1 | Linker | BPP1 | 2A | BPP2 | NLS2 | OD | TAD | | |
|---|---|---|---|---|---|---|---|---|---|---|
| Necessary | Necessary | Optional | Optional | Optional | Optional | Necessary | Optional | Necessary | | |
| part | part | part | part | part | part | part | part | part | | |
| type | type | type | type | type | type | type | type | type | Full name | Used? |
| TetR | SV40_NLS_1 | Linker1_long | 1 × SpyTag002 | ERBV_1_P2A | SpyCatcher002 | SV40_NLS_2 | Linker_only_for_oligomerization | B112_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| TetR | SV40_NLS_1 | Linker1_long | 1 × SpyTag002 | ERBV_1_P2A | SpyCatcher002 | SV40_NLS_2 | Trimerization_domain | B112_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| TetR | SV40_NLS_1 | Linker1_long | 1 × SpyTag002 | ERBV_1_P2A | SpyCatcher002 | SV40_NLS_2 | Heptamerization_domain | B112_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| TetR | SV40_NLS_1 | Linker1_long | 1 × SpyTag002 | ERBV_1_P2A | SpyCatcher002 | SV40_NLS_2 | Linker_only_for_oligomerization | B42_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| TetR | SV40_NLS_1 | Linker1_long | 1 × SpyTag002 | ERBV_1_P2A | SpyCatcher002 | SV40_NLS_2 | Trimerization_domain | B42_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| TetR | SV40_NLS_1 | Linker1_long | 1 × SpyTag002 | ERBV_1_P2A | SpyCatcher002 | SV40_NLS_2 | Heptamerization_domain | B42_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| TetR | SV40_NLS_1 | Linker1_long | 1 × SpyTag002 | ERBV_1_P2A | SpyCatcher002 | SV40_NLS_2 | Linker_only_for_oligomerization | GAL4_TAD | TetR-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| TetR | SV40_NLS_1 | Linker1_long | 1 × SpyTag002 | ERBV_1_P2A | SpyCatcher002 | SV40_NLS_2 | Trimerization_domain | GAL4_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| TetR | SV40_NLS_1 | Linker1_long | 1 × SpyTag002 | ERBV_1_P2A | SpyCatcher002 | SV40_NLS_2 | Heptamerization_domain | GAL4_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| TetR | SV40_NLS_1 | Linker1_long | 1 × SpyTag002 | ERBV_1_P2A | SpyCatcher002 | SV40_NLS_2 | Linker_only_for_oligomerization | miniVPR_TAD | TetR-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| TetR | SV40_NLS_1 | Linker1_long | 1 × SpyTag002 | ERBV_1_P2A | SpyCatcher002 | SV40_NLS_2 | Trimerization_domain | miniVPR_TAD | TetR-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| TetR | SV40_NLS_1 | Linker1_long | 1 × SpyTag002 | ERBV_1_P2A | SpyCatcher002 | SV40_NLS_2 | Heptamerization_domain | miniVPR_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| TetR | SV40_NLS_1 | Linker1_long | 1 × SpyTag002 | ERBV_1_P2A | SpyCatcher002 | SV40_NLS_2 | Linker_only_for_oligomerization | Mxr1_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| TetR | SV40_NLS_1 | Linker1_long | 1 × SpyTag002 | ERBV_1_P2A | SpyCatcher002 | SV40_NLS_2 | Trimerization_domain | Mxr1_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| TetR | SV40_NLS_1 | Linker1_long | 1 × SpyTag002 | ERBV_1_P2A | SpyCatcher002 | SV40_NLS_2 | Heptamerization_domain | Mxr1_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| TetR | SV40_NLS_1 | Linker1_long | 1 × SpyTag002 | ERBV_1_P2A | SpyCatcher002 | SV40_NLS_2 | Linker_only_for_oligomerization | No_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| TetR | SV40_NLS_1 | Linker1_long | 1 × SpyTag002 | ERBV_1_P2A | SpyCatcher002 | SV40_NLS_2 | Trimerization_domain | No_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| TetR | SV40_NLS_1 | Linker1_long | 1 × SpyTag002 | ERBV_1_P2A | SpyCatcher002 | SV40_NLS_2 | Heptamerization_domain | No_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| TetR | SV40_NLS_1 | Linker1_long | 1 × SpyTag002 | ERBV_1_P2A | SpyCatcher002 | SV40_NLS_2 | Linker_only_for_oligomerization | PH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| TetR | SV40_NLS_1 | Linker1_long | 1 × SpyTag002 | ERBV_1_P2A | SpyCatcher002 | SV40_NLS_2 | Trimerization_domain | PH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| TetR | SV40_NLS_1 | Linker1_long | 1 × SpyTag002 | ERBV_1_P2A | SpyCatcher002 | SV40_NLS_2 | Heptamerization_domain | PH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| TetR | SV40_NLS_1 | Linker1_long | 1 × SpyTag002 | ERBV_1_P2A | SpyCatcher002 | SV40_NLS_2 | Linker_only_for_oligomerization | VP16_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| TetR | SV40_NLS_1 | Linker1_long | 1 × SpyTag002 | ERBV_1_P2A | SpyCatcher002 | SV40_NLS_2 | Trimerization_domain | VP16_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| TetR | SV40_NLS_1 | Linker1_long | 1 × SpyTag002 | ERBV_1_P2A | SpyCatcher002 | SV40_NLS_2 | Heptamerization_domain | VP16_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| TetR | SV40_NLS_1 | Linker1_long | 1 × SpyTag002 | ERBV_1_P2A | SpyCatcher002 | SV40_NLS_2 | Linker_only_for_oligomerization | VP64_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| TetR | SV40_NLS_1 | Linker1_long | 1 × SpyTag002 | ERBV_1_P2A | SpyCatcher002 | SV40_NLS_2 | Trimerization_domain | VP64_TAD | TetR-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64_TAD | |
| TetR | SV40_NLS_1 | Linker1_long | 1 × SpyTag002 | ERBV_1_P2A | SpyCatcher002 | SV40_NLS_2 | Heptamerization_domain | VP64_TAD | TetR-SV40_NLS_1- | No |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| TetR | SV40_NLS_1 | Linker1_long | 1 × SpyTag002 | ERBV_1_P2A | SpyCatcher002 | SV40_NLS_2 | Linker_only_for_oligomerization | VP64v2_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization | |
| | | | | | | | | | VP64v2_TAD | |
| TetR | SV40_NLS_1 | Linker1_long | 1 × SpyTag002 | ERBV_1_P2A | SpyCatcher002 | SV40_NLS_2 | Trimerization_domain | VP64v2_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| TetR | SV40_NLS_1 | Linker1_long | 1 × SpyTag002 | ERBV_1_P2A | SpyCatcher002 | SV40_NLS_2 | Heptamerization_domain | VP64v2_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain | |
| | | | | | | | | | VP64v2_TAD | |
| TetR | SV40_NLS_1 | Linker1_long | 1 × SpyTag002 | ERBV_1_P2A | SpyCatcher002 | SV40_NLS_2 | Linker_only_for_oligomerization | VPH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| TetR | SV40_NLS_1 | Linker1_long | 1 × SpyTag002 | ERBV_1_P2A | SpyCatcher002 | SV40_NLS_2 | Trimerization_domain | VPH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| TetR | SV40_NLS_1 | Linker1_long | 1 × SpyTag002 | ERBV_1_P2A | SpyCatcher002 | SV40_NLS_2 | Heptamerization_domain | VPH_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| TetR | SV40_NLS_1 | Linker1_long | 1 × SpyTag002 | ERBV_1_P2A | SpyCatcher002 | SV40_NLS_2 | Linker_only_for_oligomerization | VPR_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| TetR | SV40_NLS_1 | Linker1_long | 1 × SpyTag002 | ERBV_1_P2A | SpyCatcher002 | SV40_NLS_2 | Trimerization_domain | VPR_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| TetR | SV40_NLS_1 | Linker1_long | 1 × SpyTag002 | ERBV_1_P2A | SpyCatcher002 | SV40_NLS_2 | Heptamerization_domain | VPR_TAD | TetR-SV40_NLS_1- | Yes |
| | | | | | | | | | Linker1_long-1 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |
| TetR | SV40_NLS_1 | Linker1_long | 2 × SpyTag002 | ERBV_1_P2A | SpyCatcher002 | SV40_NLS_2 | Linker_only_for_oligomerization | B112_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| TetR | SV40_NLS_1 | Linker1_long | 2 × SpyTag002 | ERBV_1_P2A | SpyCatcher002 | SV40_NLS_2 | Trimerization_domain | B112_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| TetR | SV40_NLS_1 | Linker1_long | 2 × SpyTag002 | ERBV_1_P2A | SpyCatcher002 | SV40_NLS_2 | Heptamerization_domain | B112_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| TetR | SV40_NLS_1 | Linker1_long | 2 × SpyTag002 | ERBV_1_P2A | SpyCatcher002 | SV40_NLS_2 | Linker_only_for_oligomerization | B42_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| TetR | SV40_NLS_1 | Linker1_long | 2 × SpyTag002 | ERBV_1_P2A | SpyCatcher002 | SV40_NLS_2 | Trimerization_domain | B42_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| TetR | SV40_NLS_1 | Linker1_long | 2 × SpyTag002 | ERBV_1_P2A | SpyCatcher002 | SV40_NLS_2 | Heptamerization_domain | B42_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| TetR | SV40_NLS_1 | Linker1_long | 2 × SpyTag002 | ERBV_1_P2A | SpyCatcher002 | SV40_NLS_2 | Linker_only_for_oligomerization | GAL4_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| TetR | SV40_NLS_1 | Linker1_long | 2 × SpyTag002 | ERBV_1_P2A | SpyCatcher002 | SV40_NLS_2 | Trimerization_domain | GAL4_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| TetR | SV40_NLS_1 | Linker1_long | 2 × SpyTag002 | ERBV_1_P2A | SpyCatcher002 | SV40_NLS_2 | Heptamerization_domain | GAL4_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| TetR | SV40_NLS_1 | Linker1_long | 2 × SpyTag002 | ERBV_1_P2A | SpyCatcher002 | SV40_NLS_2 | Linker_only_for_oligomerization | miniVPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| TetR | SV40_NLS_1 | Linker1_long | 2 × SpyTag002 | ERBV_1_P2A | SpyCatcher002 | SV40_NLS_2 | Trimerization_domain | miniVPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| TetR | SV40_NLS_1 | Linker1_long | 2 × SpyTag002 | ERBV_1_P2A | SpyCatcher002 | SV40_NLS_2 | Heptamerization_domain | miniVPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| TetR | SV40_NLS_1 | Linker1_long | 2 × SpyTag002 | ERBV_1_P2A | SpyCatcher002 | SV40_NLS_2 | Linker_only_for_oligomerization | Mxr1_TAD | TetR-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| TetR | SV40_NLS_1 | Linker1_long | 2 × SpyTag002 | ERBV_1_P2A | SpyCatcher002 | SV40_NLS_2 | Trimerization_domain | Mxr1_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| TetR | SV40_NLS_1 | Linker1_long | 2 × SpyTag002 | ERBV_1_P2A | SpyCatcher002 | SV40_NLS_2 | Heptamerization_domain | Mxr1_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| TetR | SV40_NLS_1 | Linker1_long | 2 × SpyTag002 | ERBV_1_P2A | SpyCatcher002 | SV40_NLS_2 | Linker_only_for_oligomerization | No_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| TetR | SV40_NLS_1 | Linker1_long | 2 × SpyTag002 | ERBV_1_P2A | SpyCatcher002 | SV40_NLS_2 | Trimerization_domain | No_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| TetR | SV40_NLS_1 | Linker1_long | 2 × SpyTag002 | ERBV_1_P2A | SpyCatcher002 | SV40_NLS_2 | Heptamerization_domain | No_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| TetR | SV40_NLS_1 | Linker1_long | 2 × SpyTag002 | ERBV_1_P2A | SpyCatcher002 | SV40_NLS_2 | Linker_only_for_oligomerization | PH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| TetR | SV40_NLS_1 | Linker1_long | 2 × SpyTag002 | ERBV_1_P2A | SpyCatcher002 | SV40_NLS_2 | Trimerization_domain | PH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| TetR | SV40_NLS_1 | Linker1_long | 2 × SpyTag002 | ERBV_1_P2A | SpyCatcher002 | SV40_NLS_2 | Heptamerization_domain | PH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| TetR | SV40_NLS_1 | Linker1_long | 2 × SpyTag002 | ERBV_1_P2A | SpyCatcher002 | SV40_NLS_2 | Linker_only_for_oligomerization | VP16_TAD | TetR-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| TetR | SV40_NLS_1 | Linker1_long | 2 × SpyTag002 | ERBV_1_P2A | SpyCatcher002 | SV40_NLS_2 | Trimerization_domain | VP16_TAD | TetR-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| TetR | SV40_NLS_1 | Linker1_long | 2 × SpyTag002 | ERBV_1_P2A | SpyCatcher002 | SV40_NLS_2 | Heptamerization_domain | VP16_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| TetR | SV40_NLS_1 | Linker1_long | 2 × SpyTag002 | ERBV_1_P2A | SpyCatcher002 | SV40_NLS_2 | Linker_only_for_oligomerization | V64_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | V64_TAD | |
| TetR | SV40_NLS_1 | Linker1_long | 2 × SpyTag002 | ERBV_1_P2A | SpyCatcher002 | SV40_NLS_2 | Trimerization_domain | V64_TAD | TetR-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | V64_TAD | |
| TetR | SV40_NLS_1 | Linker1_long | 2 × SpyTag002 | ERBV_1_P2A | SpyCatcher002 | SV40_NLS_2 | Heptamerization_domain | VP64_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| TetR | SV40_NLS_1 | Linker1_long | 2 × SpyTag002 | ERBV_1_P2A | SpyCatcher002 | SV40_NLS_2 | Linker_only_for_oligomerization | VP64v2_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| TetR | SV40_NLS_1 | Linker1_long | 2 × SpyTag002 | ERBV_1_P2A | SpyCatcher002 | SV40_NLS_2 | Trimerization_domain | VP64v2_TAD | TetR-SV40_NLS_1- | No |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| TetR | SV40_NLS_1 | Linker1_long | 2 × SpyTag002 | ERBV_1_P2A | SpyCatcher002 | SV40_NLS_2 | Heptamerization_domain | VP64v2_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| TetR | SV40_NLS_1 | Linker1_long | 2 × SpyTag002 | ERBV_1_P2A | SpyCatcher002 | SV40_NLS_2 | Linker_only_for_oligomerization | VPH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| TetR | SV40_NLS_1 | Linker1_long | 2 × SpyTag002 | ERBV_1_P2A | SpyCatcher002 | SV40_NLS_2 | Trimerization_domain | VPH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| TetR | SV40_NLS_1 | Linker1_long | 2 × SpyTag002 | ERBV_1_P2A | SpyCatcher002 | SV40_NLS_2 | Heptamerization_domain | VPH_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| TetR | SV40_NLS_1 | Linker1_long | 2 × SpyTag002 | ERBV_1_P2A | SpyCatcher002 | SV40_NLS_2 | Linker_only_for_oligomerization | VPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| TetR | SV40_NLS_1 | Linker1_long | 2 × SpyTag002 | ERBV_1_P2A | SpyCatcher002 | SV40_NLS_2 | Trimerization_domain | VPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| TetR | SV40_NLS_1 | Linker1_long | 2 × SpyTag002 | ERBV_1_P2A | SpyCatcher002 | SV40_NLS_2 | Heptamerization_domain | VPR_TAD | TetR-SV40_NLS_1- | Yes |
| | | | | | | | | | Linker1_long-2 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |
| TetR | SV40_NLS_1 | Linker1_long | 6 × SpyTag002 | ERBV_1_P2A | SpyCatcher002 | SV40_NLS_2 | Linker_only_for_oligomerization | B112_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| TetR | SV40_NLS_1 | Linker1_long | 6 × SpyTag002 | ERBV_1_P2A | SpyCatcher002 | SV40_NLS_2 | Trimerization_domain | B112_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B112_TAD | |
| TetR | SV40_NLS_1 | Linker1_long | 6 × SpyTag002 | ERBV_1_P2A | SpyCatcher002 | SV40_NLS_2 | Heptamerization_domain | B112_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B112_TAD | |
| TetR | SV40_NLS_1 | Linker1_long | 6 × SpyTag002 | ERBV_1_P2A | SpyCatcher002 | SV40_NLS_2 | Linker_only_for_oligomerization | B42_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| TetR | SV40_NLS_1 | Linker1_long | 6 × SpyTag002 | ERBV_1_P2A | SpyCatcher002 | SV40_NLS_2 | Trimerization_domain | B42_TAD | TetR-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | B42_TAD | |
| TetR | SV40_NLS_1 | Linker1_long | 6 × SpyTag002 | ERBV_1_P2A | SpyCatcher002 | SV40_NLS_2 | Heptamerization_domain | B42_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | B42_TAD | |
| TetR | SV40_NLS_1 | Linker1_long | 6 × SpyTag002 | ERBV_1_P2A | SpyCatcher002 | SV40_NLS_2 | Linker_only_for_oligomerization | GAL4_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| TetR | SV40_NLS_1 | Linker1_long | 6 × SpyTag002 | ERBV_1_P2A | SpyCatcher002 | SV40_NLS_2 | Trimerization_domain | GAL4_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | GAL4_TAD | |
| TetR | SV40_NLS_1 | Linker1_long | 6 × SpyTag002 | ERBV_1_P2A | SpyCatcher002 | SV40_NLS_2 | Heptamerization_domain | GAL4_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| TetR | SV40_NLS_1 | Linker1_long | 6 × SpyTag002 | ERBV_1_P2A | SpyCatcher002 | SV40_NLS_2 | Linker_only_for_oligomerization | miniVPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| TetR | SV40_NLS_1 | Linker1_long | 6 × SpyTag002 | ERBV_1_P2A | SpyCatcher002 | SV40_NLS_2 | Trimerization_domain | miniVPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| TetR | SV40_NLS_1 | Linker1_long | 6 × SpyTag002 | ERBV_1_P2A | SpyCatcher002 | SV40_NLS_2 | Heptamerization_domain | miniVPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| TetR | SV40_NLS_1 | Linker1_long | 6 × SpyTag002 | ERBV_1_P2A | SpyCatcher002 | SV40_NLS_2 | Linker_only_for_oligomerization | Mxr1_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| TetR | SV40_NLS_1 | Linker1_long | 6 × SpyTag002 | ERBV_1_P2A | SpyCatcher002 | SV40_NLS_2 | Trimerization_domain | Mxr1_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | Mxr1_TAD | |
| TetR | SV40_NLS_1 | Linker1_long | 6 × SpyTag002 | ERBV_1_P2A | SpyCatcher002 | SV40_NLS_2 | Heptamerization_domain | Mxr1_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| TetR | SV40_NLS_1 | Linker1_long | 6 × SpyTag002 | ERBV_1_P2A | SpyCatcher002 | SV40_NLS_2 | Linker_only_for_oligomerization | No_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| TetR | SV40_NLS_1 | Linker1_long | 6 × SpyTag002 | ERBV_1_P2A | SpyCatcher002 | SV40_NLS_2 | Trimerization_domain | No_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | No_TAD | |
| TetR | SV40_NLS_1 | Linker1_long | 6 × SpyTag002 | ERBV_1_P2A | SpyCatcher002 | SV40_NLS_2 | Heptamerization_domain | No_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | No_TAD | |
| TetR | SV40_NLS_1 | Linker1_long | 6 × SpyTag002 | ERBV_1_P2A | SpyCatcher002 | SV40_NLS_2 | Linker_only_for_oligomerization | PH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| TetR | SV40_NLS_1 | Linker1_long | 6 × SpyTag002 | ERBV_1_P2A | SpyCatcher002 | SV40_NLS_2 | Trimerization_domain | PH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | PH_TAD | |
| TetR | SV40_NLS_1 | Linker1_long | 6 × SpyTag002 | ERBV_1_P2A | SpyCatcher002 | SV40_NLS_2 | Heptamerization_domain | PH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | PH_TAD | |
| TetR | SV40_NLS_1 | Linker1_long | 6 × SpyTag002 | ERBV_1_P2A | SpyCatcher002 | SV40_NLS_2 | Linker_only_for_oligomerization | VP16_TAD | TetR-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| TetR | SV40_NLS_1 | Linker1_long | 6 × SpyTag002 | ERBV_1_P2A | SpyCatcher002 | SV40_NLS_2 | Trimerization_domain | VP16_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP16_TAD | |
| TetR | SV40_NLS_1 | Linker1_long | 6 × SpyTag002 | ERBV_1_P2A | SpyCatcher002 | SV40_NLS_2 | Heptamerization_domain | VP16_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| TetR | SV40_NLS_1 | Linker1_long | 6 × SpyTag002 | ERBV_1_P2A | SpyCatcher002 | SV40_NLS_2 | Linker_only_for_oligomerization | VP64_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| TetR | SV40_NLS_1 | Linker1_long | 6 × SpyTag002 | ERBV_1_P2A | SpyCatcher002 | SV40_NLS_2 | Trimerization_domain | VP64_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64_TAD | |
| TetR | SV40_NLS_1 | Linker1_long | 6 × SpyTag002 | ERBV_1_P2A | SpyCatcher002 | SV40_NLS_2 | Heptamerization_domain | VP64_TAD | TetR-SV40_NLS_1- | No |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| TetR | SV40_NLS_1 | Linker1_long | 6 × SpyTag002 | ERBV_1_P2A | SpyCatcher002 | SV40_NLS_2 | Linker_only_for_oligomerization | VP64v2_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| TetR | SV40_NLS_1 | Linker1_long | 6 × SpyTag002 | ERBV_1_P2A | SpyCatcher002 | SV40_NLS_2 | Trimerization_domain | VP64v2_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VP64v2_TAD | |
| TetR | SV40_NLS_1 | Linker1_long | 6 × SpyTag002 | ERBV_1_P2A | SpyCatcher002 | SV40_NLS_2 | Heptamerization_domain | VP64v2_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| TetR | SV40_NLS_1 | Linker1_long | 6 × SpyTag002 | ERBV_1_P2A | SpyCatcher002 | SV40_NLS_2 | Linker_only_for_oligomerization | VPH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| TetR | SV40_NLS_1 | Linker1_long | 6 × SpyTag002 | ERBV_1_P2A | SpyCatcher002 | SV40_NLS_2 | Trimerization_domain | VPH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPH_TAD | |
| TetR | SV40_NLS_1 | Linker1_long | 6 × SpyTag002 | ERBV_1_P2A | SpyCatcher002 | SV40_NLS_2 | Heptamerization_domain | VPH_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPH_TAD | |
| TetR | SV40_NLS_1 | Linker1_long | 6 × SpyTag002 | ERBV_1_P2A | SpyCatcher002 | SV40_NLS_2 | Linker_only_for_oligomerization | VPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| TetR | SV40_NLS_1 | Linker1_long | 6 × SpyTag002 | ERBV_1_P2A | SpyCatcher002 | SV40_NLS_2 | Trimerization_domain | VPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | VPR_TAD | |
| TetR | SV40_NLS_1 | Linker1_long | 6 × SpyTag002 | ERBV_1_P2A | SpyCatcher002 | SV40_NLS_2 | Heptamerization_domain | VPR_TAD | TetR-SV40_NLS_1- | Yes |
| | | | | | | | | | Linker1_long-6 × SpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VPR_TAD | |

TABLE 14
Two-component sTFs based on DNA-binding domain VanR_AM. The “Full name”
entry on a given row is the name of both the gene encoding that row's sTF protein as well as
that of this sTF protein. The DNA sequence of this sTF-encoding gene is a concatenation of
the DNA sequences of the indicated part-type variants on the corresponding row in left-to-
right order. The amino acid sequence of this sTF protein is a concatenation of the amino acid
sequences of the indicated part-type variants on the corresponding row in left-to-right order.
DNA and amino acid sequences of component part-type variants are in Table 21. The
“Used?” entry on a given row is “Yes” if that row's sTF-encoding gene was actually used for
SES library construction, and “No” if it was not. Part types that are considered necessary for
SES functionality are labeled “Necessary part type,” while those that are considered optional
for SES functionality are labeled “Optional part type.”

| DBD | NLS1 | Linker | BPP1 | 2A | BPP2 | NLS2 | OD | TAD | | |
|---|---|---|---|---|---|---|---|---|---|---|
| Necessary | Necessary | Optional | Optional | Optional | Optional | Necessary | Optional | Necessary | | |
| part | part | part | part | part | part | part | part | part | | |
| type | type | type | type | type | type | type | type | type | Full name | Used? |
| VanR_AM | SV40_NLS_1 | Linker1_long | 1xSpyTag002 | ERBV_1P2A | SpyCatcher002 | SV40_NLS_2 | Linker_only_for_oligomerization | B112_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| VanR_AM | SV40_NLS_1 | Linker1_long | 1xSpyTag002 | ERBV_1P2A | SpyCatcher002 | SV40_NLS_2 | Trimerization_domain | B112_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-B112_TAD | |
| VanR_AM | SV40_NLS_1 | Linker1_long | 1xSpyTag002 | ERBV_1P2A | SpyCatcher002 | SV40_NLS_2 | Heptamerization_domain | B112_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-B112_TAD | |
| VanR_AM | SV40_NLS_1 | Linker1_long | 1xSpyTag002 | ERBV_1P2A | SpyCatcher002 | SV40_NLS_2 | Linker_only_for_oligomerization | B42_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| VanR_AM | SV40_NLS_1 | Linker1_long | 1xSpyTag002 | ERBV_1P2A | SpyCatcher002 | SV40_NLS_2 | Trimerization_domain | B42_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-B42_TAD | |
| VanR_AM | SV40_NLS_1 | Linker1_long | 1xSpyTag002 | ERBV_1P2A | SpyCatcher002 | SV40_NLS_2 | Heptamerization_domain | B42_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-B42_TAD | |
| VanR_AM | SV40_NLS_1 | Linker1_long | 1xSpyTag002 | ERBV_1P2A | SpyCatcher002 | SV40_NLS_2 | Linker_only_for_oligomerization | GAL4_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| VanR_AM | SV40_NLS_1 | Linker1_long | 1xSpyTag002 | ERBV_1P2A | SpyCatcher002 | SV40_NLS_2 | Trimerization_domain | GAL4_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-GAL4_TAD | |
| VanR_AM | SV40_NLS_1 | Linker1_long | 1xSpyTag002 | ERBV_1P2A | SpyCatcher002 | SV40_NLS_2 | Heptamerization_domain | GAL4_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| VanR_AM | SV40_NLS_1 | Linker1_long | 1xSpyTag002 | ERBV_1P2A | SpyCatcher002 | SV40_NLS_2 | Linker_only_for_oligomerization | mini | VanR_AM-SV40_NLS_1- | Yes |
| | | | | | | | | VPR_TAD | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| VanR_AM | SV40_NLS_1 | Linker1_long | 1xSpyTag002 | ERBV_1P2A | SpyCatcher002 | SV40_NLS_2 | Trimerization_domain | mini | VanR_AM-SV40_NLS_1- | Yes |
| | | | | | | | | VPR_TAD | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| VanR_AM | SV40_NLS_1 | Linker1_long | 1xSpyTag002 | ERBV_1P2A | SpyCatcher002 | SV40_NLS_2 | Heptamerization_domain | mini | VanR_AM-SV40_NLS_1- | Yes |
| | | | | | | | | VPR_TAD | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| VanR_AM | SV40_NLS_1 | Linker1_long | 1xSpyTag002 | ERBV_1P2A | SpyCatcher002 | SV40_NLS_2 | Linker_only_for_oligomerization | Mxr1_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| VanR_AM | SV40_NLS_1 | Linker1_long | 1xSpyTag002 | ERBV_1P2A | SpyCatcher002 | SV40_NLS_2 | Trimerization_domain | Mxr1_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-Mxr1_TAD | |
| VanR_AM | SV40_NLS_1 | Linker1_long | 1xSpyTag002 | ERBV_1P2A | SpyCatcher002 | SV40_NLS_2 | Heptamerization_domain | Mxr1_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| VanR_AM | SV40_NLS_1 | Linker1_long | 1xSpyTag002 | ERBV_1P2A | SpyCatcher002 | SV40_NLS_2 | Linker_only_for_oligomerization | No_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| VanR_AM | SV40_NLS_1 | Linker1_long | 1xSpyTag002 | ERBV_1P2A | SpyCatcher002 | SV40_NLS_2 | Trimerization_domain | No_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-No_TAD | |
| VanR_AM | SV40_NLS_1 | Linker1_long | 1xSpyTag002 | ERBV_1P2A | SpyCatcher002 | SV40_NLS_2 | Heptamerization_domain | No_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-No_TAD | |
| VanR_AM | SV40_NLS_1 | Linker1_long | 1xSpyTag002 | ERBV_1P2A | SpyCatcher002 | SV40_NLS_2 | Linker_only_for_oligomerization | PH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| VanR_AM | SV40_NLS_1 | Linker1_long | 1xSpyTag002 | ERBV_1P2A | SpyCatcher002 | SV40_NLS_2 | Trimerization_domain | PH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-PH_TAD | |
| VanR_AM | SV40_NLS_1 | Linker1_long | 1xSpyTag002 | ERBV_1P2A | SpyCatcher002 | SV40_NLS_2 | Heptamerization_domain | PH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-PH_TAD | |
| VanR_AM | SV40_NLS_1 | Linker1_long | 1xSpyTag002 | ERBV_1P2A | SpyCatcher002 | SV40_NLS_2 | Linker_only_for_oligomerization | VP16_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| VanR_AM | SV40_NLS_1 | Linker1_long | 1xSpyTag002 | ERBV_1P2A | SpyCatcher002 | SV40_NLS_2 | Trimerization_domain | VP16_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-VP16_TAD | |
| VanR_AM | SV40_NLS_1 | Linker1_long | 1xSpyTag002 | ERBV_1P2A | SpyCatcher002 | SV40_NLS_2 | Heptamerization_domain | VP16_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| VanR_AM | SV40_NLS_1 | Linker1_long | 1xSpyTag002 | ERBV_1P2A | SpyCatcher002 | SV40_NLS_2 | Linker_only_for_oligomerization | VP64_TAD | VanR_AM-SV40_NLS_1- | No |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| VanR_AM | SV40_NLS_1 | Linker1_long | 1xSpyTag002 | ERBV_1P2A | SpyCatcher002 | SV40_NLS_2 | Trimerization_domain | VP64_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-VP64_TAD | |
| VanR_AM | SV40_NLS_1 | Linker1_long | 1xSpyTag002 | ERBV_1P2A | SpyCatcher002 | SV40_NLS_2 | Heptamerization_domain | VP64_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| VanR_AM | SV40_NLS_1 | Linker1_long | 1xSpyTag002 | ERBV_1P2A | SpyCatcher002 | SV40_NLS_2 | Linker_only_for_oligomerization | VP64v2_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| VanR_AM | SV40_NLS_1 | Linker1_long | 1xSpyTag002 | ERBV_1P2A | SpyCatcher002 | SV40_NLS_2 | Trimerization_domain | VP64v2_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-VP64v2_TAD | |
| VanR_AM | SV40_NLS_1 | Linker1_long | 1xSpyTag002 | ERBV_1P2A | SpyCatcher002 | SV40_NLS_2 | Heptamerization_domain | VP64v2_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| VanR_AM | SV40_NLS_1 | Linker1_long | 1xSpyTag002 | ERBV_1P2A | SpyCatcher002 | SV40_NLS_2 | Linker_only_for_oligomerization | VPH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| VanR_AM | SV40_NLS_1 | Linker1_long | 1xSpyTag002 | ERBV_1P2A | SpyCatcher002 | SV40_NLS_2 | Trimerization_domain | VPH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-VPH_TAD | |
| VanR_AM | SV40_NLS_1 | Linker1_long | 1xSpyTag002 | ERBV_1P2A | SpyCatcher002 | SV40_NLS_2 | Heptamerization_domain | VPH_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-VPH_TAD | |
| VanR_AM | SV40_NLS_1 | Linker1_long | 1xSpyTag002 | ERBV_1P2A | SpyCatcher002 | SV40_NLS_2 | Linker_only_for_oligomerization | VPR_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| VanR_AM | SV40_NLS_1 | Linker1_long | 1xSpyTag002 | ERBV_1P2A | SpyCatcher002 | SV40_NLS_2 | Trimerization_domain | VPR_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Trimerization_domain-VPR_TAD | |
| VanR_AM | SV40_NLS_1 | Linker1_long | 1xSpyTag002 | ERBV_1P2A | SpyCatcher002 | SV40_NLS_2 | Heptamerization_domain | VPR_TAD | VanR_AM-SV40_NLS_1- | Yes |
| | | | | | | | | | Linker1_long-1xSpyTag002- | |
| | | | | | | | | | ERBV_1_P2A-SpyCatcher002- | |
| | | | | | | | | | SV40_NLS_2- | |
| | | | | | | | | | Heptamerization_domain-VPR_TAD | |
| VanR_AM | SV40_NLS_1 | Linker1_long | 2xSpyTag002 | ERBV_1P2A | SpyCatcher002 | SV40_NLS_2 | Linker_only_for_oligomerization | B112_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| VanR_AM | SV40_NLS_1 | Linker1_long | 2xSpyTag002 | ERBV_1P2A | SpyCatcher002 | SV40_NLS_2 | Trimerization_domain | B112_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-B112_TAD | |
| VanR_AM | SV40_NLS_1 | Linker1_long | 2xSpyTag002 | ERBV_1P2A | SpyCatcher002 | SV40_NLS_2 | Heptamerization_domain | B112_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-B112_TAD | |
| VanR_AM | SV40_NLS_1 | Linker1_long | 2xSpyTag002 | ERBV_1P2A | SpyCatcher002 | SV40_NLS_2 | Linker_only_for_oligomerization | B42_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| VanR_AM | SV40_NLS_1 | Linker1_long | 2xSpyTag002 | ERBV_1P2A | SpyCatcher002 | SV40_NLS_2 | Trimerization_domain | B42_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-B42_TAD | |
| | | | 2xSpyTag002 | ERBV_1P2A | SpyCatcher002 | SV40_NLS_2 | Heptamerization_domain | B42_TAD | VanR_AM-SV40_NLS_1- | Yes |
| VanR_AM | SV40_NLS_1 | Linker1_long | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-B42_TAD | |
| VanR_AM | SV40_NLS_1 | Linker1_long | 2xSpyTag002 | ERBV_1P2A | SpyCatcher002 | SV40_NLS_2 | Linker_only_for_oligomerization | GAL4_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| VanR_AM | SV40_NLS_1 | Linker1_long | 2xSpyTag002 | ERBV_1P2A | SpyCatcher002 | SV40_NLS_2 | Trimerization_domain | GAL4_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-GAL4_TAD | |
| VanR_AM | SV40_NLS_1 | Linker1_long | 2xSpyTag002 | ERBV_1P2A | SpyCatcher002 | SV40_NLS_2 | Heptamerization_domain | GAL4_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| VanR_AM | SV40_NLS_1 | Linker1_long | 2xSpyTag002 | ERBV_1P2A | SpyCatcher002 | SV40_NLS_2 | Linker_only_for_oligomerization | mini | VanR_AM-SV40_NLS_1- | No |
| | | | | | | | | VPR_TAD | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| VanR_AM | SV40_NLS_1 | Linker1_long | 2xSpyTag002 | ERBV_1P2A | SpyCatcher002 | SV40_NLS_2 | Trimerization_domain | mini | VanR_AM-SV40_NLS_1- | Yes |
| | | | | | | | | VPR_TAD | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| VanR_AM | SV40_NLS_1 | Linker1_long | 2xSpyTag002 | ERBV_1P2A | SpyCatcher002 | SV40_NLS_2 | Heptamerization_domain | mini | VanR_AM-SV40_NLS_1- | Yes |
| | | | | | | | | VPR_TAD | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| VanR_AM | SV40_NLS_1 | Linker1_long | 2xSpyTag002 | ERBV_1P2A | SpyCatcher002 | SV40_NLS_2 | Linker_only_for_oligomerization | Mxr1_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| VanR_AM | SV40_NLS_1 | Linker1_long | 2xSpyTag002 | ERBV_1P2A | SpyCatcher002 | SV40_NLS_2 | Trimerization_domain | Mxr1_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-Mxr1_TAD | |
| VanR_AM | SV40_NLS_1 | Linker1_long | 2xSpyTag002 | ERBV_1P2A | SpyCatcher002 | SV40_NLS_2 | Heptamerization_domain | Mxr1_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| VanR_AM | SV40_NLS_1 | Linker1_long | 2xSpyTag002 | ERBV_1P2A | SpyCatcher002 | SV40_NLS_2 | Linker_only_for_oligomerization | No_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| VanR_AM | SV40_NLS_1 | Linker1_long | 2xSpyTag002 | ERBV_1P2A | SpyCatcher002 | SV40_NLS_2 | Trimerization_domain | No_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-No_TAD | |
| VanR_AM | SV40_NLS_1 | Linker1_long | 2xSpyTag002 | ERBV_1P2A | SpyCatcher002 | SV40_NLS_2 | Heptamerization_domain | No_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-No_TAD | |
| VanR_AM | SV40_NLS_1 | Linker1_long | 2xSpyTag002 | ERBV_1P2A | SpyCatcher002 | SV40_NLS_2 | Linker_only_for_oligomerization | PH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| VanR_AM | SV40_NLS_1 | Linker1_long | 2xSpyTag002 | ERBV_1P2A | SpyCatcher002 | SV40_NLS_2 | Trimerization_domain | PH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-PH_TAD | |
| VanR_AM | SV40_NLS_1 | Linker1_long | 2xSpyTag002 | ERBV_1P2A | SpyCatcher002 | SV40_NLS_2 | Heptamerization_domain | PH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-PH_TAD | |
| VanR_AM | SV40_NLS_1 | Linker1_long | 2xSpyTag002 | ERBV_1P2A | SpyCatcher002 | SV40_NLS_2 | Linker_only_for_oligomerization | VP16_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| VanR_AM | SV40_NLS_1 | Linker1_long | 2xSpyTag002 | ERBV_1P2A | SpyCatcher002 | SV40_NLS_2 | Trimerization_domain | VP16_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-VP16_TAD | |
| VanR_AM | SV40_NLS_1 | Linker1_long | 2xSpyTag002 | ERBV_1P2A | SpyCatcher002 | SV40_NLS_2 | Heptamerization_domain | VP16_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| VanR_AM | SV40_NLS_1 | Linker1_long | 2xSpyTag002 | ERBV_1P2A | SpyCatcher002 | SV40_NLS_2 | Linker_only_for_oligomerization | VP64_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| VanR_AM | SV40_NLS_1 | Linker1_long | 2xSpyTag002 | ERBV_1P2A | SpyCatcher002 | SV40_NLS_2 | Trimerization_domain | VP64_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-VP64_TAD | |
| VanR_AM | SV40_NLS_1 | Linker1_long | 2xSpyTag002 | ERBV_1P2A | SpyCatcher002 | SV40_NLS_2 | Heptamerization_domain | VP64_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| VanR_AM | SV40_NLS_1 | Linker1_long | 2xSpyTag002 | ERBV_1P2A | SpyCatcher002 | SV40_NLS_2 | Linker_only_for_oligomerization | VP64v2_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| VanR_AM | SV40_NLS_1 | Linker1_long | 2xSpyTag002 | ERBV_1P2A | SpyCatcher002 | SV40_NLS_2 | Trimerization_domain | VP64v2_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-VP64v2_TAD | |
| VanR_AM | SV40_NLS_1 | Linker1_long | 2xSpyTag002 | ERBV_1P2A | SpyCatcher002 | SV40_NLS_2 | Heptamerization_domain | VP64v2_TAD | VanR_AM-SV40_NLS_1- | No |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| VanR_AM | SV40_NLS_1 | Linker1_long | 2xSpyTag002 | ERBV_1P2A | SpyCatcher002 | SV40_NLS_2 | Linker_only_for_oligomerization | VPH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| VanR_AM | SV40_NLS_1 | Linker1_long | 2xSpyTag002 | ERBV_1P2A | SpyCatcher002 | SV40_NLS_2 | Trimerization_domain | VPH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-VPH_TAD | |
| VanR_AM | SV40_NLS_1 | Linker1_long | 2xSpyTag002 | ERBV_1P2A | SpyCatcher002 | SV40_NLS_2 | Heptamerization_domain | VPH_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-VPH_TAD | |
| VanR_AM | SV40_NLS_1 | Linker1_long | 2xSpyTag002 | ERBV_1P2A | SpyCatcher002 | SV40_NLS_2 | Linker_only_for_oligomerization | VPR_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| VanR_AM | SV40_NLS_1 | Linker1_long | 2xSpyTag002 | ERBV_1P2A | SpyCatcher002 | SV40_NLS_2 | Trimerization_domain | VPR_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Trimerization_domain-VPR_TAD | |
| VanR_AM | SV40_NLS_1 | Linker1_long | 2xSpyTag002 | ERBV_1P2A | SpyCatcher002 | SV40_NLS_2 | Heptamerization_domain | VPR_TAD | VanR_AM-SV40_NLS_1- | Yes |
| | | | | | | | | | Linker1_long-2xSpyTag002- | |
| | | | | | | | | | ERBV_1_P2A-SpyCatcher002- | |
| | | | | | | | | | SV40_NLS_2- | |
| | | | | | | | | | Heptamerization_domain-VPR_TAD | |
| VanR_AM | SV40_NLS_1 | Linker1_long | 6xSpyTag002 | ERBV_1P2A | SpyCatcher002 | SV40_NLS_2 | Linker_only_for_oligomerization | B112_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B112_TAD | |
| VanR_AM | SV40_NLS_1 | Linker1_long | 6xSpyTag002 | ERBV_1P2A | SpyCatcher002 | SV40_NLS_2 | Trimerization_domain | B112_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-B112_TAD | |
| VanR_AM | SV40_NLS_1 | Linker1_long | 6xSpyTag002 | ERBV_1P2A | SpyCatcher002 | SV40_NLS_2 | Heptamerization_domain | B112_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-B112_TAD | |
| VanR_AM | SV40_NLS_1 | Linker1_long | 6xSpyTag002 | ERBV_1P2A | SpyCatcher002 | SV40_NLS_2 | Linker_only_for_oligomerization | B42_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | B42_TAD | |
| VanR_AM | SV40_NLS_1 | Linker1_long | 6xSpyTag002 | ERBV_1P2A | SpyCatcher002 | SV40_NLS_2 | Trimerization_domain | B42_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-B42_TAD | |
| VanR_AM | SV40_NLS_1 | Linker1_long | 6xSpyTag002 | ERBV_1P2A | SpyCatcher002 | SV40_NLS_2 | Heptamerization_domain | B42_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-B42_TAD | |
| VanR_AM | SV40_NLS_1 | Linker1_long | 6xSpyTag002 | ERBV_1P2A | SpyCatcher002 | SV40_NLS_2 | Linker_only_for_oligomerization | GAL4_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | GAL4_TAD | |
| VanR_AM | SV40_NLS_1 | Linker1_long | 6xSpyTag002 | ERBV_1P2A | SpyCatcher002 | SV40_NLS_2 | Trimerization_domain | GAL4_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-GAL4_TAD | |
| VanR_AM | SV40_NLS_1 | Linker1_long | 6xSpyTag002 | ERBV_1P2A | SpyCatcher002 | SV40_NLS_2 | Heptamerization_domain | GAL4_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | GAL4_TAD | |
| VanR_AM | SV40_NLS_1 | Linker1_long | 6xSpyTag002 | ERBV_1P2A | SpyCatcher002 | SV40_NLS_2 | Linker_only_for_oligomerization | mini | VanR_AM-SV40_NLS_1- | Yes |
| | | | | | | | | VPR_TAD | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | miniVPR_TAD | |
| VanR_AM | SV40_NLS_1 | Linker1_long | 6xSpyTag002 | ERBV_1P2A | SpyCatcher002 | SV40_NLS_2 | Trimerization_domain | mini | VanR_AM-SV40_NLS_1- | Yes |
| | | | | | | | | VPR_TAD | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain- | |
| | | | | | | | | | miniVPR_TAD | |
| VanR_AM | SV40_NLS_1 | Linker1_long | 6xSpyTag002 | ERBV_1P2A | SpyCatcher002 | SV40_NLS_2 | Heptamerization_domain | mini | VanR_AM-SV40_NLS_1- | Yes |
| | | | | | | | | VPR_TAD | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | miniVPR_TAD | |
| VanR_AM | SV40_NLS_1 | Linker1_long | 6xSpyTag002 | ERBV_1P2A | SpyCatcher002 | SV40_NLS_2 | Linker_only_for_oligomerization | Mxr1_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | Mxr1_TAD | |
| VanR_AM | SV40_NLS_1 | Linker1_long | 6xSpyTag002 | ERBV_1P2A | SpyCatcher002 | SV40_NLS_2 | Trimerization_domain | Mxr1_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | 1 | | | | | | | | Trimerization_domain-Mxr1_TAD | |
| VanR_AM | SV40_NLS_1 | Linker1_long | 6xSpyTag002 | ERBV_1P2A | SpyCatcher002 | SV40_NLS_2 | Heptamerization_domain | Mxr1_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | Mxr1_TAD | |
| VanR_AM | SV40_NLS_1 | Linker1_long | 6xSpyTag002 | ERBV_1P2A | SpyCatcher002 | SV40_NLS_2 | Linker_only_for_oligomerization | No_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | No_TAD | |
| VanR_AM | SV40_NLS_1 | Linker1_long | 6xSpyTag002 | ERBV_1P2A | SpyCatcher002 | SV40_NLS_2 | Trimerization_domain | No_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-No_TAD | |
| VanR_AM | SV40_NLS_1 | Linker1_long | 6xSpyTag002 | ERBV_1P2A | SpyCatcher002 | SV40_NLS_2 | Heptamerization_domain | No_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-No_TAD | |
| VanR_AM | SV40_NLS_1 | Linker1_long | 6xSpyTag002 | ERBV_1P2A | SpyCatcher002 | SV40_NLS_2 | Linker_only_for_oligomerization | PH_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | PH_TAD | |
| VanR_AM | SV40_NLS_1 | Linker1_long | 6xSpyTag002 | ERBV_1P2A | SpyCatcher002 | SV40_NLS_2 | Trimerization_domain | PH_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-PH_TAD | |
| VanR_AM | SV40_NLS_1 | Linker1_long | 6xSpyTag002 | ERBV_1P2A | SpyCatcher002 | SV40_NLS_2 | Heptamerization_domain | PH_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-PH_TAD | |
| VanR_AM | SV40_NLS_1 | Linker1_long | 6xSpyTag002 | ERBV_1P2A | SpyCatcher002 | SV40_NLS_2 | Linker_only_for_oligomerization | VP16_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP16_TAD | |
| VanR_AM | SV40_NLS_1 | Linker1_long | 6xSpyTag002 | ERBV_1P2A | SpyCatcher002 | SV40_NLS_2 | Trimerization_domain | VP16_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-VP16_TAD | |
| VanR_AM | SV40_NLS_1 | Linker1_long | 6xSpyTag002 | ERBV_1P2A | SpyCatcher002 | SV40_NLS_2 | Heptamerization_domain | VP16_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP16_TAD | |
| VanR_AM | SV40_NLS_1 | Linker1_long | 6xSpyTag002 | ERBV_1P2A | SpyCatcher002 | SV40_NLS_2 | Linker_only_for_oligomerization | VP64_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64_TAD | |
| VanR_AM | SV40_NLS_1 | Linker1_long | 6xSpyTag002 | ERBV_1P2A | SpyCatcher002 | SV40_NLS_2 | Trimerization_domain | VP64_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-VP64_TAD | |
| VanR_AM | SV40_NLS_1 | Linker1_long | 6xSpyTag002 | ERBV_1P2A | SpyCatcher002 | SV40_NLS_2 | Heptamerization_domain | VP64_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64_TAD | |
| VanR_AM | SV40_NLS_1 | Linker1_long | 6xSpyTag002 | ERBV_1P2A | SpyCatcher002 | SV40_NLS_2 | Linker_only_for_oligomerization | VP64v2_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VP64v2_TAD | |
| VanR_AM | SV40_NLS_1 | Linker1_long | 6xSpyTag002 | ERBV_1P2A | SpyCatcher002 | SV40_NLS_2 | Trimerization_domain | VP64v2_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-VP64v2_TAD | |
| VanR_AM | SV40_NLS_1 | Linker1_long | 6xSpyTag002 | ERBV_1P2A | SpyCatcher002 | SV40_NLS_2 | Heptamerization_domain | VP64v2_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain- | |
| | | | | | | | | | VP64v2_TAD | |
| VanR_AM | SV40_NLS_1 | Linker1_long | 6xSpyTag002 | ERBV_1P2A | SpyCatcher002 | SV40_NLS_2 | Linker_only_for_oligomerization | VPH_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPH_TAD | |
| VanR_AM | SV40_NLS_1 | Linker1_long | 6xSpyTag002 | ERBV_1P2A | SpyCatcher002 | SV40_NLS_2 | Trimerization_domain | VPH_TAD | VanR_AM-SV40_NLS_1- | No |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-VPH_TAD | |
| VanR_AM | SV40_NLS_1 | Linker1_long | 6xSpyTag002 | ERBV_1P2A | SpyCatcher002 | SV40_NLS_2 | Heptamerization_domain | VPH_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-VPH_TAD | |
| VanR_AM | SV40_NLS_1 | Linker1_long | 6xSpyTag002 | ERBV_1P2A | SpyCatcher002 | SV40_NLS_2 | Linker_only_for_oligomerization | VPR_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Linker_only_for_oligomerization- | |
| | | | | | | | | | VPR_TAD | |
| VanR_AM | SV40_NLS_1 | Linker1_long | 6xSpyTag002 | ERBV_1P2A | SpyCatcher002 | SV40_NLS_2 | Trimerization_domain | VPR_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Trimerization_domain-VPR_TAD | |
| VanR_AM | SV40_NLS_1 | Linker1_long | 6xSpyTag002 | ERBV_1P2A | SpyCatcher002 | SV40_NLS_2 | Heptamerization_domain | VPR_TAD | VanR_AM-SV40_NLS_1- | Yes |
| | | | | | | | | | Linker1_long-6xSpyTag002- | |
| | | | | | | | | | ERBV_1_P2A-SpyCatcher002- | |
| | | | | | | | | | SV40_NLS_2- | |
| | | | | | | | | | Heptamerization_domain-VPR_TAD | |

Not all possible combinatorial sTF variants of a given type (e.g., one-component TetR-based sTFs) were used for DNA-based SES library assembly. This is because all sTF-encoding genes were submitted for de novo DNA synthesis, and while most were successfully synthesized, not all were.

For all sTF variants described in Table 7, Table 8, Table 9, Table 10, Table 11, Table 12, Table 13, and Table 14, the DNA sequence of the corresponding sTF coding sequence is a concatenation of the DNA sequences of the part-type variants on the corresponding row in left-to-right order. Similarly, the amino acid sequence of the corresponding sTF protein is a concatenation of the amino acid sequences of the part-type variants on the corresponding row in left-to-right order. DNA and amino acid sequences of sTF variants are noted in Table 21.

Synthetic output promoters are described in Table 15, Table 16, Table 17, and Table 18, based on the four different DBD types used, and comprise 2 components: UAS and core promoter. For a given DBD, 20 possible core promoters were used for SES library assembly, based on a combinatorial assembly of 5 UAS variants×4 core promoters. For all synthetic output promoter variants described in Table 15, Table 16, Table 17, and Table 18, the DNA sequence of the corresponding synthetic output promoter variant is a concatenation of the DNA sequences of the UAS and core promoter on the corresponding row in left-to-right order. DNA sequences of component part-type variants are noted in Table 21.

TABLE 15
Synthetic output promoters cognate with respect to the DNA-binding domain
Bm3R1. The ″Full name″ entry on a given row is the name of that row's synthetic output
promoter. The DNA sequence of this synthetic output promoter is a concatenation of the DNA
sequences of the indicated part-type variants on the corresponding row in left-to-right order.
DNA sequences of component part-type variants are in Table 21. The ″Used?″ entry on a
given row is ″Yes″ if that row's synthetic output promoter was actually used for SES library
construction, and ″No″ if it was not. 1×, 2×, etc. indicate the number of copies of a sequence
such as an operator. ″0×operator″ indicates that there is no (0×) operator. Part types that
are considered necessary for SES functionality are labeled ″Necessary part type,″ while those
that are considered optional for SES functionality are labeled ″Optional part type.″ While the
output promoter part type is necessary, other specific output promoters could have been
chosen.

| UAS | | | |
|---|---|---|---|
| Necessary | Core promoter | | |
| part type | Necessary part type | Full name | Used? |
| 0×operator | P(AOX1)_core_promoter | 0×operator-P(AOX1)_core_promoter | Yes |
| 0×operator | P(DAS2)_core_promoter | 0×operator-P(DAS2)_core_promoter | Yes |
| 0×operator | P(HHF2)_core_promoter | 0×operator-P(HHF2)_core_promoter | Yes |
| 0×operator | P(PMP20)_core_promoter | 0×operator-P(PMP20)_core_promoter | Yes |
| 1×bmO | P(AOX1)_core_promoter | 1×bmO-P(AOX1)_core_promoter | Yes |
| 1×bmO | P(DAS2)_core_promoter | 1×bmO-P(DAS2)_core_promoter | Yes |
| 1×bmO | P(HHF2)_core_promoter | 1×bmO-P(HHF2)_core_promoter | Yes |
| 1×bmO | P(PMP20)_core_promoter | 1×bmO-P(PMP20)_core_promoter | Yes |
| 2×bmO | P(AOX1)_core_promoter | 2×bmO-P(AOX1)_core_promoter | Yes |
| 2×bmO | P(DAS2)_core_promoter | 2×bmO-P(DAS2)_core_promoter | Yes |
| 2×bmO | P(HHF2)_core_promoter | 2×bmO-P(HHF2)_core_promoter | Yes |
| 2×bmO | P(PMP20)_core_promoter | 2×bmO-P(PMP20)_core_promoter | Yes |
| 4×bmO | P(AOX1)_core_promoter | 4×bmO-P(AOX1)_core_promoter | Yes |
| 4×bmO | P(DAS2)_core_promoter | 4×bmO-P(DAS2)_core_promoter | Yes |
| 4×bmO | P(HHF2)_core_promoter | 4×bmO-P(HHF2)_core_promoter | Yes |
| 4×bmO | P(PMP20)_core_promoter | 4×bmO-P(PMP20)_core_promoter | Yes |
| 8×bmO | P(AOX1)_core_promoter | 8×bmO-P(AOX1)_core_promoter | Yes |
| 8×bmO | P(DAS2)_core_promoter | 8×bmO-P(DAS2)_core_promoter | Yes |
| 8×bmO | P(HHF2)_core_promoter | 8×bmO-P(HHF2)_core_promoter | Yes |
| 8×bmO | P(PMP20)_core_promoter | 8×bmO-P(PMP20)_core_promoter | Yes |

TABLE 16
Synthetic output promoters cognate with respect to the DNA-binding domain
PhlF_AM. The ″Full name″ entry on a given row is the name of that row's synthetic output
promoter. The DNA sequence of this synthetic output promoter is a concatenation of the DNA
sequences of the indicated part-type variants on the corresponding row in left-to-right order.
DNA sequences of component part-type variants are in Table 21. The ″Used?″ entry on a
given row is ″Yes″ if that row's synthetic output promoter was actually used for SES library
construction, and ″No″ if it was not. Part types that are considered necessary for SES
functionality are labeled ″Necessary part type,″ while those that are considered optional for
SES functionality are labeled ″Optional part type.″ While the output promoter part type is
necessary, other specific output promoters could have been chosen.

| UAS | | | |
|---|---|---|---|
| Necessary | Core promoter | | |
| part type | Necessary part type | Full name | Used? |
| 0×operator | P(AOX1)_core_promoter | 0×operator-P(AOX1)_core_promoter | Yes |
| 0×operator | P(DAS2)_core_promoter | 0×operator-P(DAS2)_core_promoter | Yes |
| 0×operator | P(HHF2)_core_promoter | 0×operator-P(HHF2)_core_promoter | Yes |
| 0×operator | P(PMP20)_core_promoter | 0×operator-P(PMP20)_core_promoter | Yes |
| 1×phlO | P(AOX1)_core_promoter | 1×phlO-P(AOX1)_core_promoter | Yes |
| 1×phlO | P(DAS2)_core_promoter | 1×phlO-P(DAS2)_core_promoter | Yes |
| 1×phlO | P(HHF2)_core_promoter | 1×phlO-P(HHF2)_core_promoter | Yes |
| 1×phlO | P(PMP20)_core_promoter | 1×phlO-P(PMP20)_core_promoter | Yes |
| 2×phlO | P(AOX1)_core_promoter | 2×phlO-P(AOX1)_core_promoter | Yes |
| 2×phlO | P(DAS2)_core_promoter | 2×phlO-P(DAS2)_core_promoter | Yes |
| 2×phlO | P(HHF2)_core_promoter | 2×phlO-P(HHF2)_core_promoter | Yes |
| 2×phlO | P(PMP20)_core_promoter | 2×phlO-P(PMP20)_core_promoter | Yes |
| 4×phlO | P(AOX1)_core_promoter | 4×phlO-P(AOX1)_core_promoter | Yes |
| 4×phlO | P(DAS2)_core_promoter | 4×phlO-P(DAS2)_core_promoter | Yes |
| 4×phlO | P(HHF2)_core_promoter | 4×phlO-P(HHF2)_core_promoter | Yes |
| 4×phlO | P(PMP20)_core_promoter | 4×phlO-P(PMP20)_core_promoter | Yes |
| 8×phlO | P(AOX1)_core_promoter | 8×phlO-P(AOX1)_core_promoter | Yes |
| 8×phlO | P(DAS2)_core_promoter | 8×phlO-P(DAS2)_core_promoter | Yes |
| 8×phlO | P(HHF2)_core_promoter | 8×phlO-P(HHF2)_core_promoter | Yes |
| 8×phlO | P(PMP20)_core_promoter | 8×phlO-P(PMP20)_core_promoter | Yes |

TABLE 17
Synthetic output promoters cognate with respect to the DNA-binding domain TetR.
The ″Full name″ entry on a given row is the name of that row's synthetic output promoter.
The DNA sequence of this synthetic output promoter is a concatenation of the DNA sequences
of the indicated part-type variants on the corresponding row in left-to-right order. DNA
sequences of component part-type variants are in Table 21. The ″Used?″ entry on a given
row is ″Yes″ if that row's synthetic output promoter was actually used for SES library
construction, and ″No″ if it was not. Part types that are considered necessary for SES
functionality are labeled ″Necessary part type,″ while those that are considered optional for
SES functionality are labeled ″Optional part type.″ While the output promoter part type is
necessary, other specific output promoters could have been chosen.

| UAS | | | |
|---|---|---|---|
| Necessary | Core promoter | | |
| part type | Necessary part type | Full name | Used? |
| 0×operator | P(AOX1)_core_promoter | 0×operator-P(AOX1)_core_promoter | Yes |
| 0×operator | P(DAS2)_core_promoter | 0×operator-P(DAS2)_core_promoter | Yes |
| 0×operator | P(HHF2)_core_promoter | 0×operator-P(HHF2)_core_promoter | Yes |
| 0×operator | P(PMP20)_core_promoter | 0×operator-P(PMP20)_core_promoter | Yes |
| 1×tetO | P(AOX1)_core_promoter | 1×tetO-P(AOX1)_core_promoter | Yes |
| 1×tetO | P(DAS2)_core_promoter | 1×tetO-P(DAS2)_core_promoter | Yes |
| 1×tetO | P(HHF2)_core_promoter | 1×tetO-P(HHF2)_core_promoter | Yes |
| 1×tetO | P(PMP20)_core_promoter | 1×tetO-P(PMP20)_core_promoter | Yes |
| 2×tetO | P(AOX1)_core_promoter | 2×tetO-P(AOX1)_core_promoter | Yes |
| 2×tetO | P(DAS2)_core_promoter | 2×tetO-P(DAS2)_core_promoter | Yes |
| 2×tetO | P(HHF2)_core_promoter | 2×tetO-P(HHF2)_core_promoter | Yes |
| 2×tetO | P(PMP20)_core_promoter | 2×tetO-P(PMP20)_core_promoter | Yes |
| 4×tetO | P(AOX1)_core_promoter | 4×tetO-P(AOX1)_core_promoter | Yes |
| 4×tetO | P(DAS2)_core_promoter | 4×tetO-P(DAS2)_core_promoter | Yes |
| 4×tetO | P(HHF2)_core_promoter | 4×tetO-P(HHF2)_core_promoter | Yes |
| 4×tetO | P(PMP20)_core_promoter | 4×tetO-P(PMP20)_core_promoter | Yes |
| 8×tetO | P(AOX1)_core_promoter | 8×tetO-P(AOX1)_core_promoter | Yes |
| 8×tetO | P(DAS2)_core_promoter | 8×tetO-P(DAS2)_core_promoter | Yes |
| 8×tetO | P(HHF2)_core_promoter | 8×tetO-P(HHF2)_core_promoter | Yes |
| 8×tetO | P(PMP20)_core_promoter | 8×tetO-P(PMP20)_core_promoter | Yes |

TABLE 18
Synthetic output promoters cognate with respect to the DNA-binding domain
VanR_AM. The ″Full name″ entry on a given row is the name of that row's synthetic output
promoter. The DNA sequence of this synthetic output promoter is a concatenation of the DNA
sequences of the indicated part-type variants on the corresponding row in left-to-right order.
DNA sequences of component part-type variants are in Table 21. The ″Used?″ entry on a
given row is ″Yes″ if that row's synthetic output promoter was actually used for SES library
construction, and ″No″ if it was not. Part types that are considered necessary for SES
functionality are labeled ″Necessary part type,″ while those that are considered optional for
SES functionality are labeled ″Optional part type.″ While the output promoter part type is
necessary, other specific output promoters could have been chosen.

| UAS | | | |
|---|---|---|---|
| Necessary | Core promoter | | |
| part type | Necessary part type | Full name | Used? |
| 0×operator | P(AOX1)_core_promoter | 0×operator-P(AOX1)_core_promoter | Yes |
| 0×operator | P(DAS2)_core_promoter | 0×operator-P(DAS2)_core_promoter | Yes |
| 0×operator | P(HHF2)_core_promoter | 0×operator-P(HHF2)_core_promoter | Yes |
| 0×operator | P(PMP20)_core_promoter | 0×operator-P(PMP20)_core_promoter | Yes |
| 1×vanO | P(AOX1)_core_promoter | 1×vanO-P(AOX1)_core_promoter | Yes |
| 1×vanO | P(DAS2)_core_promoter | 1×vanO-P(DAS2)_core_promoter | Yes |
| 1×vanO | P(HHF2)_core_promoter | 1×vanO-P(HHF2)_core_promoter | Yes |
| 1×vanO | P(PMP20)_core_promoter | 1×vanO-P(PMP20)_core_promoter | Yes |
| 2×vanO | P(AOX1)_core_promoter | 2×vanO-P(AOX1)_core_promoter | Yes |
| 2×vanO | P(DAS2)_core_promoter | 2×vanO-P(DAS2)_core_promoter | Yes |
| 2×vanO | P(HHF2)_core_promoter | 2×vanO-P(HHF2)_core_promoter | Yes |
| 2×vanO | P(PMP20)_core_promoter | 2×vanO-P(PMP20)_core_promoter | Yes |
| 4×vanO | P(AOX1)_core_promoter | 4×vanO-P(AOX1)_core_promoter | Yes |
| 4×vanO | P(DAS2)_core_promoter | 4×vanO-P(DAS2)_core_promoter | Yes |
| 4×vanO | P(HHF2)_core_promoter | 4×vanO-P(HHF2)_core_promoter | Yes |
| 4×vanO | P(PMP20)_core_promoter | 4×vanO-P(PMP20)_core_promoter | Yes |
| 8×vanO | P(AOX1)_core_promoter | 8×vanO-P(AOX1)_core_promoter | Yes |
| 8×vanO | P(DAS2)_core_promoter | 8×vanO-P(DAS2)_core_promoter | Yes |
| 8×vanO | P(HHF2)_core_promoter | 8×vanO-P(HHF2)_core_promoter | Yes |
| 8×vanO | P(PMP20)_core_promoter | 8×vanO-P(PMP20)_core_promoter | Yes |

For each process, for each SES sub-library, parts (3) (transcriptional terminator (TT) of the first transcriptional unit) and (4) (spacer)—each an optional part, but included in this example as a design choice—have only a single variant, described in Table 19 and Table 20, respectively. The corresponding DNA sequences of these two parts are noted in Table 21.

TABLE 19
Transcriptional terminator for sTF-expressing transcriptional unit. The ″Full
name″ entry on a given row is the name of that row's terminator. In this case, the terminator
is wholly one of the part-type variants in Table 21. The ″Used?″ entry is ″Yes″ if that row's
terminator was actually used for SES library construction, and ″No″ if it was not. Part types
that are considered necessary for SES functionality are labeled ″Necessary part type,″ while
those that are considered optional for SES functionality are labeled ″Optional part type.″

| TT | | |
|---|---|---|
| Optional part type | Full name | Used? |
| T(RPS2) | T(RPS2) | Yes |

TABLE 20
Spacer following sTF-expressing transcriptional unit. The ″Full name″ entry on a
given row is the name of that row's terminator. In this case, the terminator is wholly one of
the part-type variants in Table 21. The ″Used?″ entry is ″Yes″ if that row's terminator was
actually used for SES library construction, and ″No″ if it was not. Part types that are
considered necessary for SES functionality are labeled ″Necessary part type, ″ while those
that are considered optional for SES functionality are labeled ″Optional part type.″

| Spacer | | |
|---|---|---|
| Optional part type | Full name | Used? |
| Spacer | Spacer | Yes |

Thus, each SES library variant, excluding the RFP and transcriptional terminator components of the second transcriptional unit, comprises, in the following order, the DNA sequence of the following parts: (1) P(in), (2) sTF, (3) TT of the first transcriptional unit, (4) Spacer, and (5) P(out). The DNA sequence of the P(in) variant can be obtained by referring to the corresponding row of Table 4, Table 5, or Table 6, and then to the corresponding row of Table 21. Table 21 notes the DNA and, if applicable, the amino acid sequences of all 62 atomic part-type variants referenced in the above tables.

The DNA sequence of the gene encoding the sTF variant can be obtained by referring to the corresponding row of Table 7, Table 8, Table 9, Table 10, Table 11, Table 12, Table 13, or Table 14, and then by generation of the full sTF gene sequence via concatenation of the DNA sequences of corresponding component part-type variants as listed in Table 21. The DNA transcriptional terminator of the first transcriptional unit that was used in library construction appears in Table 21. The DNA sequence of the spacer used for library construction for this assay appears in Table 21. The DNA sequence of the synthetic output promoter variant appears in the row of Table 15, Table 16, Table 17, or Table 18 corresponding to the sTF by concatenation of the DNA sequences of corresponding component part-type variants as listed in Table 21.

TABLE 21
Name, DNA sequence (by SEQ ID NO), and, if applicable, amino acid sequence
(by SEQ ID NO) of all 62 atomic part-type variants used for design of SES libraries for
Processes 1, 2, and 3.

| | Name of part- | DNA sequence of part-type | Amino acid sequence |
|---|---|---|---|
| Part type | type variant | variant | of part-type variant |
| P(in) | P(THI13)_short | (SEQ ID NO: 16) | — |
| P(in) | P(THI13)_long | (SEQ ID NO: 17) | — |
| P(in) | P(THI4) | (SEQ ID NO: 18) | — |
| P(in) | P(JEN1) | (SEQ ID NO: 19) | — |
| P(in) | P(GQ6704499) | (SEQ ID NO: 20) | — |
| P(in) | P(GQ6700926) | (SEQ ID NO: 21) | — |
| P(in) | P(HGT1) | (SEQ ID NO: 22) | — |
| P(in) | P(FDH1) | (SEQ ID NO: 23) | — |
| P(in) | P(AOX2) | (SEQ ID NO: 24) | — |
| P(in) | P(RGI2) | (SEQ ID NO: 25) | — |
| DBD | Bm3R1 | (SEQ ID NO: 86) | (SEQ ID NO: 90) |
| DBD | PhIF_AM | (SEQ ID NO: 87) | (SEQ ID NO: 91) |
| DBD | TetR | (SEQ ID NO: 88) | (SEQ ID NO: 92) |
| DBD | VanR_AM | (SEQ ID NO: 89) | (SEQ ID NO: 93) |
| NLS | SV40_NLS | (SEQ ID NO: 116) | (SEQ ID NO: 118) |
| NLS | SV40_NLS_1 | (SEQ ID NO: 116) | (SEQ ID NO: 118) |
| NLS | SV40_NLS_2 | (SEQ ID NO: 117) | (SEQ ID NO: 119) |
| Linker | Linker1_long | (SEQ ID NO: 120) | (SEQ ID NO: 122) |
| Linker | Linker1_short | (SEQ ID NO: 121) | (SEQ ID NO: 123) |
| Bioconjugate-Tag | 1xSpyTag002 | (SEQ ID NO: 148) | (SEQ ID NO: 151) |
| Bioconjugate-Tag | 2xSpyTag002 | (SEQ ID NO: 149) | (SEQ ID NO: 152) |
| Bioconjugate-Tag | 6xSpyTag002 | (SEQ ID NO: 150) | (SEQ ID NO: 153) |
| 2A | ERBV_1_P2A | (SEQ ID NO: 124) | (SEQ ID NO: 125) |
| Bioconjugate-Mitt | SpyCatcher002 | (SEQ ID NO: 154) | (SEQ ID NO: 155) |
| Oligomerization | Heptamerization | (SEQ ID NO: 156) | (SEQ ID NO: 159) |
| Domain | domain | | |
| Oligomerization | Linker_only_for | (SEQ ID NO: 157) | (SEQ ID NO: 160) |
| Domain | oligomerization | | |
| Oligomerization | Trimerization_do | (SEQ ID NO: 158) | (SEQ ID NO: 161) |
| Domain | main | | |
| TAD | No_TAD | — | — |
| TAD | B112_TAD | (SEQ ID NO: 94) | (SEQ ID NO: 105) |
| TAD | B42_TAD | (SEQ ID NO: 95) | (SEQ ID NO: 106) |
| TAD | GAL4_TAD | (SEQ ID NO: 96) | (SEQ ID NO: 107) |
| TAD | miniVPR_TAD | (SEQ ID NO: 97) | (SEQ ID NO: 108) |
| TAD | Mxr1_TAD | (SEQ ID NO: 98) | (SEQ ID NO: 109) |
| TAD | PH_TAD | (SEQ ID NO: 99) | (SEQ ID NO: 110) |
| TAD | VP16_TAD | (SEQ ID NO: 100) | (SEQ ID NO: 111) |
| TAD | VP64_TAD | (SEQ ID NO: 101) | (SEQ ID NO: 112) |
| TAD | VP64v2_TAD | (SEQ ID NO: 102) | (SEQ ID NO: 113) |
| TAD | VPH_TAD | (SEQ ID NO: 103) | (SEQ ID NO: 114) |
| TAD | VPR_TAD | (SEQ ID NO: 104) | (SEQ ID NO: 115) |
| UAS | 0xoperator | | |
| UAS | 1xbmO | (SEQ ID NO: 126) | — |
| UAS | 1xphlO | (SEQ ID NO: 127) | — |
| UAS | 1xtetO | (SEQ ID NO: 128) | — |
| UAS | 1xvanO | (SEQ ID NO: 129) | — |
| UAS | 2xbmO | (SEQ ID NO: 134) | — |
| UAS | 2xphlO | (SEQ ID NO: 137) | — |
| UAS | 2xtetO | (SEQ ID NO: 140) | — |
| UAS | 2xvanO | (SEQ ID NO: 143) | — |
| UAS | 4xbmO | (SEQ ID NO: 135) | — |
| UAS | 4xphlO | (SEQ ID NO: 138) | — |
| UAS | 4xtetO | (SEQ ID NO: 141) | — |
| UAS | 4xvanO | (SEQ ID NO: 144) | — |
| UAS | 8xbmO | (SEQ ID NO: 136) | — |
| UAS | 8xphlO | (SEQ ID NO: 139) | — |
| UAS | 8xtetO | (SEQ ID NO: 142) | — |
| UAS | 8xvanO | (SEQ ID NO: 145) | — |
| Core Promoter | P(AOX1)_core_pr | (SEQ ID NO: 162) | — |
| | omoter | | |
| Core Promoter | P(DAS2)_core_pr | (SEQ ID NO: 163) | — |
| | omoter | | |
| Core Promoter | P(HHF2)_core_pr | (SEQ ID NO: 164) | — |
| | omoter | | |
| Core Promoter | P(PMP20)_core_ | (SEQ ID NO: 165) | — |
| | promoter | | |
| TT | T(RPS2) | (SEQ ID NO: 146) | — |
| Spacer | Spacer | (SEQ ID NO: 166) | — |

SES libraries were designed in such a way that the RFP expression profile of individual SES variants could be assayed in a multiplex fashion using FACS coupled to next generation sequencing (NGS). More specifically, each DNA-based SES sub-library in Table 1, Table 2, and Table 3 was assembled in a manner that permitted insertion of a short designed DNA-based barcode close to the DNA sequence corresponding to the SES variant. The sequence of each such barcode was uniquely indicative of the full sequence of the much longer (multi-kilobase) SES variant. This design schema enabled the determination, using PCR amplification and short-read NGS, of the population fraction of each SES variant within cell-based SES library sub-pools fractionated, using FACS, based on their level of RFP abundance. This thereby enabled the parallel measurement of the time-course of RFP expression of many cell-based SES variants under each given process condition.

### Example 2. Assay 1

This Example demonstrates that the synthetic expression systems of this disclosure can be built to accommodate a variety of cell culture processes. An equimolar mixture of each of the 8 SES sub-libraries for each library (each designed to be cognate to one of the 3 processes used in Assay 1) was used to transform P. pastoris host cells. The variant SESs were integrated into cells at single copy at the AOX1 locus. SESs were integrated at a position allowing the native AOX1 transcriptional terminator to serve as the transcriptional terminator of the second transcriptional unit, i.e., the transcriptional unit expressing RFP. Not all library members were successfully synthesized, and not all transformed successfully. Thus, as reported in Table 22 below, Assay 1 did not test every possible cell-based SES library variant from the corresponding DNA-based SES library.

Assay 1 assayed RFP abundance of library variants in each of the three processes, using P(AOX1)-driven expression of RFP in a conventional methanol-dependent process as a control. Assay 1 also measured the extent to which an SES tightly regulated expression in pre-production phases of fermentation (which is relevant where the gene product to be expressed, or its metabolites, may be toxic to host cells).

A glycerol stock containing the library of transformed P. pastoris strains was subjected to a lab scale fermentation using a process associated with each input promoter, P(in). Samples were drawn 20 h and 90 h after the start of fermentation and stored at 4° C. after a 100-fold dilution in PBS. Each sample was subjected to fluorescence-activated cell sorting followed by sequencing to confirm the identity of each synthetic expression system and its activity. Table 22 summarizes the performance of library members in comparison to the P(AOX1) control strain evaluated in the conventional methanol-dependent process.

TABLE 22
Summary Results for Assay 1.

| Process number | 1 | 2 | 3 |
|---|---|---|---|
| Inducer during production stage | Limiting | Limiting | Limiting |
| | Glycerol + | Glucose + | Glucose + |
| | Added | Added | Depleted |
| | Formic acid | Formic acid | Thiamine |
| Row 1: Total theoretical number of DNA- | 46,100 | 55,320 | 27,660 |
| based SES library variants | | | |
| Row 2: Number of cell-based SES library | 1,497 | 1,624 | 3,013 |
| variants actually assessed in Assay 1 | | | |
| Row 3: Number of synthetic expression | 683 | 1,005 | 1,448 |
| systems more tightly regulated in fed-batch | | | |
| phase than P(AOX1) in MeOH | | | |
| Row 4: Number of synthetic expression | 281 | 351 | 50 |
| systems stronger (i.e., driving more abundant | | | |
| expression of RFP) in production phase than | | | |
| P(AOX1) in MeOH | | | |
| Row 5: Number of synthetic expression | 26 | 109 | 5 |
| systems more tightly regulated AND stronger | | | |
| than P(AOX1) in MeOH | | | |

A selection of strains harboring synthetic expression systems (Table 22, Row 5) were isolated and subjected to a second assay (Assay 2, described below). As shown in Table 22, above, the strains that were high performing in Assay 1 were assessed based on two criteria—tight regulation in fed-batch phase and strength of expression in production phase—in each case compared to a P(AOX1) control cultured in methanol-dependent fermentation conditions. Surprisingly, more than 100 synthetic expression systems constructed according to this disclosure met those very stringent criteria. Many of the other strains in addition to those listed in Row 5 of Table 22 would be suitable for a high-performing, methanol-independent synthetic expression system based on the criteria used for Assay 1.

The skilled artisan will also appreciate that the designer of a synthetic expression system according to this disclosure could employ entirely different criteria from those chosen for Assay 1. For example, to express some bioproducts, a designer might emphasize overall abundance of production in the fed-batch phase, irrespective of production in the growth phase. As set forth in Table 22, Row 4, almost 700 strains containing synthetic expression systems constructed according to this disclosure outperformed a conventional P(AOX1) control according to that criterion. And for yet other bioproducts, a designer might construct a synthetic expression system according to this disclosure that would express the bioproduct at a less abundant level. Thus the scope of this disclosure is not limited to the SESs identified in Assay 1 and described in Example 3. Any combination of SES component part types as shown in Example 1 may be combined and assessed according to any number of criteria in order to arrive at SESs suitable for conditions of interest.

### Example 3. Assay 2

Pichia pastoris host cells comprising a selection of the top-performing synthetic expression systems from Assay 1 (Example 2) were streaked onto a YEP+4% dextrose agar plate and allowed to grow at 30° C. for 48 hours.

These colonies were subjected to lab-scale fermentation as detailed in Table 27 using a process associated with the P(in) of each synthetic expression system (Processes 1-3). Samples were drawn at various time points during fermentation and stored at 4° C. after a 100-fold dilution in PBS. Intracellular fluorescence of each diluted sample was measured using flow cytometry as a median of fluorescence values from 100,000 cells. A control strain expressing RFP under the control of P(AOX1) was similarly evaluated after a lab scale fermentation using a glycerol to methanol process (Process 4). Results are shown in Tables 23, 24 and 25, below.

TABLE 23
Expression of RFP in various synthetic expression systems in Pichia pastoris
subjected to Process 1, as compared to P(AOX1) in Process 4 (control methanol-dependent
process). Values are median fluorescence measurements taken via clonal cytometry of 100,000 cells.

| | Process | | | | | | |
|---|---|---|---|---|---|---|---|
| | 1 | | | | | | |
| | P96.sTF.- | | P96.sTF.- | | P96.sTF.- | | 4 |
| Time | Tet.13.102.4 | P96.sTF.Van.9.103.4 | Phl.12.99.6 | P96.sTF.Tet.1.106.4 | Phl.7.11.7 | P96.sTF.Phl.5.107.4 | PAOX1 |
| 0 hours | 24972 | 2051 | 294 | 7516 | 9582 | 39641 | 371 |
| 12 hours | 1911 | 483 | 117 | 764 | 3519 | 20124 | 128 |
| 24 hours | 5529 | 4323 | 6978 | 3013 | 5642 | 17883 | 585 |
| 36 hours | 51224 | 38586 | 47560 | 23108 | 27078 | 85821 | 1114 |
| 48 hours | 99888 | 74737 | 86694 | 44759 | 21311 | 164554 | 1823 |
| 60 hours | 133502 | 97557 | 112784 | 61250 | 10782 | 221419 | 13380 |
| 72 hours | 148718 | 104365 | 124793 | 68693 | 441000 | 367233 | 24390 |
| 90 hours | 173679 | 118637 | 139957 | 77562 | 583000 | 445000 | 23343 |

TABLE 24
Expression of RFP in various synthetic expression systems in Pichia pastoris
subjected to Process 2, as compared to P(AOX1) in Process 4 (control). Values are median
fluorescence measurements taken via clonal cytometry of 100,000 cells.

| | Process | | | | | | | |
|---|---|---|---|---|---|---|---|---|
| | 2 | | | | | | | |
| | P96.sTF.- | | P96.sTF.- | | P96.sTF.- | | | 4 |
| Time | Phl.5.40.8 | P96.sTF.Bm.9.118.8 | Phl.12.25.7 | P96.sTF.Phl.5.109.8 | Bm.13.100.7 | P96.sTF.Phl.12.17.9 | P96.sTF.Phl.9.107.7 | pAOX1 |
| 0 hours | 249 | 977 | 2732 | 1829 | 13157 | 2606 | 7669 | 371 |
| 12 hours | 243 | 736 | 836 | 714 | 22799 | 1745 | 6264 | 128 |
| 24 hours | 1484 | 987 | 365 | 556 | 26446 | 4848 | 9811 | 585 |
| 36 hours | 22267 | 26896 | 5797 | 11419 | 25141 | 8807 | 28968 | 1114 |
| 48 hours | 97557 | 106140 | 42694 | 72747 | 28197 | 32269 | 105071 | 1823 |
| 60 hours | 143301 | 173094 | 67773 | 93057 | 25569 | 40588 | 61044 | 13380 |
| 72 hours | 155909 | 194127 | 85821 | 99216 | 29164 | 44910 | 213354 | 24390 |
| 90 hours | 188959 | 250850 | 102274 | 107945 | 40451 | 39242 | 243350 | 23343 |

TABLE 25
Expression of RFP in various synthetic expression systems in Pichia pastoris
subjected to Process 3, as compared to P(AOX1) in Process 4 (control). Values are median
fluorescence measurements taken via clonal cytometry of 100,000 cells.

| Process | 3 | | 4 |
|---|---|---|---|
| Time | P96.STF.Phl.5.41.10 | P96.sTF.Bm.5.23.11 | pAOX1 |
| 0 hours | 21747 | 7774 | 371 |
| 12 hours | 5364 | 2277 | 128 |
| 24 hours | 685 | 1048 | 585 |
| 36 hours | 35346 | 30574 | 1114 |
| 48 hours | 149220 | 146231 | 1823 |
| 60 hours | 231344 | 233697 | 13380 |
| 72 hours | 280384 | 306083 | 24390 |
| 90 hours | 324147 | 372221 | 23343 |

Table 26 describes in detail the composition of SES variants identified by Assay 2 in Table 23, Table 24, and Table 25. Based on Table 26 as well as the tables cited therein as well as the foregoing text, the full DNA sequence of each of these SES variants can be derived in terms of a specific concatenation of part-type variant DNA sequences noted in Table 21. The full DNA sequences of each of these SES variants are noted in Table 28.

TABLE 26
Detailed annotation of SES variants identified by Assay 2 in Table 23, Table 24,
and Table 25. The DNA sequence of a given SES is a concatenation of the DNA sequences
under fields (i) Input promoter variant (see Table 4, Table 5, or Table 6 as well as main text
regarding how to extract sequence), (ii) Synthetic transcription factor variant (see Table 7,
Table 8, Table 9, Table 10, Table 11, Table 12, Table 13, or Table 14 as well as main text
regarding how to extract sequence), (iii) TT of first transcriptional unit (invariant; see Table
19 as well as main text regarding how to extract sequence), (iv) Spacer (invariant; see Table
20 as well as main text regarding how to extract sequence), and (v) synthetic output promoter
variant (see Table 15, Table 16, Table 17, or Table 18 as well as main text regarding how to
extract sequence), on the corresponding row in left-to-right order. The full DNA sequence of
each SES variant is noted in Table 28.

| Process | SES | | | | | | | |
|---|---|---|---|---|---|---|---|---|
| in | variant | | | | | | | |
| which | identifier | | | | | TT of | | |
| SES | in | SES | | Synthetic | | first | | synthetic |
| variant | Tables | variant | Input | transcription | | transcriptional | | output |
| was | 23, 24, | plasmid | promoter | factor | | unit | Spacer | promoter |
| identified | and 25 | identifier | variant | type | Synthetic transcription factor variant | (invariant) | (invariant) | variant |
| 1 | P96.sTF.Tet.13.102.4 | m3921287 | P(GQ6704499) | TetR- | TetR-SV40_NLS-Linker1_short- | T(RPS2) | Spacer | 8xtetO-P(AOX1)_core_promoter |
| | | | | based one- | PH_TAD | | | |
| | | | | component | | | | |
| 1 | P96.sTF.Van.9.103.4 | m3921319 | P(GQ6704499) | VanR_AM- | VanR_AM-SV40_NLS-Linker1_short- | T(RPS2) | Spacer | 4xvanO-P(AOX1)_core_promoter |
| | | | | based | VPR_TAD | | | |
| | | | | one- | | | | |
| | | | | component | | | | |
| 1 | P96.sTF.Phl.12.99.6 | m3921724 | P(HGT1) | PhlF- | PhlF_AM-SV40_NLS-Linker1_short- | T(RPS2) | Spacer | 8xphlO-P(AOX1)_core_promoter |
| | | | | based one- | VP16_TAD | | | |
| | | | | component | | | | |
| 1 | P96.sTF.Tet.1.106.4 | m3920550 | P(GQ6704499) | TetR- | TetR-SV40_NLS-Linker1_short- | T(RPS2) | Spacer | 1xtetO-(AOX1)_core_promoter |
| | | | | based one- | VPH_TAD | | | |
| | | | | component | | | | |
| 1 | P96.sTF.Phl.7.11.7 | m3921850 | P(FDH1) | PhlF- | PhlF_AM-SV40_NLS_1- | T(RPS2) | Spacer | 2xphlO-P(HHF2)_core_promoter |
| | | | | based two- | Linker1_long-1xSpyTag002- | | | |
| | | | | component | ERBV_1_P2A-SpyCatcher002- | | | |
| | | | | | SV40_NLS_2- | | | |
| | | | | | Linker_only_for_oligomerization- | | | |
| | | | | | VPH_TAD | | | |
| 1 | P96.sTF.Phl.5.107.4 | m3921830 | P(GQ6704499) | PhlF- | PhlF_AM-SV40_NLS-Linker1_long- | T(RPS2) | Spacer | 2xph1O-P(AOX1)_core_promoter |
| | | | | based one- | miniVPR_TAD | | | |
| | | | | component | | | | |
| 2 | P96.sTF.Phl.5.40.8 | m3920469 | P(AOX2) | PhlF- | PhlF_AM-SV40_NLS_1- | T(RPS2) | Spacer | 2xphlO-P(AOX1)_core_promoter |
| | | | | based two- | Linker1_long-2xSpyTag002- | | | |
| | | | | component | ERBV_1_P2A-SpyCatcher002- | | | |
| | | | | | SV40_NLS_2- | | | |
| | | | | | Linker_only_for_oligomerization- | | | |
| | | | | | VP64v2_TAD | | | |
| 2 | P96.sTF.Bm.9.118.8 | m3920152 | P(AOX2) | Bm3R1- | Bm3R1-SV40_NLS-Linker1_long- | T(RPS2) | Spacer | 4xbmO-P(AOX1)_core_promoter |
| | | | | based one- | VPH_TAD | | | |
| | | | | component | | | | |
| 2 | P96.sTF.Phl.12.25.7 | m3920290 | P(FDH1) | PhlF- | PhlF_AM-SV40_NLS_1- | T(RPS2) | Spacer | 8xph1O-P(AOX1)_core_promoter |
| | | | | based two- | Linker1_long-1xSpyTag002- | | | |
| | | | | component | ERBV_1_P2A-SpyCatcher002- | | | |
| | | | | | SV40_NLS_2- | | | |
| | | | | | Heptamerization_domain-B42_TAD | | | |
| 2 | P96.sTF.Phl.5.109.8 | m3920459 | P(AOX2) | PhlF- | PhlF_AM-SV40_NLS-Linker1_long- | T(RPS2) | Spacer | 2xphlO-P(AOX1)_core_promoter |
| | | | | based one- | PH_TAD | | | |
| | | | | component | | | | |
| 2 | P96.sTF.Bm.13.100.7 | m3919863 | P(FDH1) | Bm3R1- | Bm3R1-SV40_NLS-Linker1_short- | T(RPS2) | Spacer | 8xbmO-P(AOX1)_core_promoter |
| | | | | based one- | miniVPR_TAD | | | |
| | | | | component | | | | |
| 2 | P96.sTF.Phl.12.17.9 | m3921327 | P(RGI2) | PhlF- | PhlF_AM-SV40_NLS_1- | T(RPS2) | Spacer | 8xph1O-P(AOX1)_core_promoter |
| | | | | based two- | Linker1_long-1xSpyTag002- | | | |
| | | | | component | ERBV_1_P2A-SpyCatcher002- | | | |
| | | | | | SV40_NLS_2-Trimerization_domain- | | | |
| | | | | | miniVPR_TAD | | | |
| 2 | P96.sTF.Phl.9.107.7 | m3920513 | P(FDH1) | PhlF- | PhlF_AM-SV40_NLS-Linker1_long- | T(RPS2) | Spacer | 8xphlO-P(PMP20)_core_promoter |
| | | | | based one- | miniVPR_TAD | | | |
| | | | | component | | | | |
| 3 | P96.sTF.Phl.5.41.10 | m3946808 | P(THI13)_short | PhlF- | PhlF_AM-SV40_NLS_1- | T(RPS2) | Spacer | 2xph1O-P(AOX1)_core_promoter |
| | | | | based two- | Linker1_long-2xSpyTag002- | | | |
| | | | | component | ERBV_1_P2A-SpyCatcher002- | | | |
| | | | | | SV40_NLS_2- | | | |
| | | | | | Linker_only_for_oligomerization- | | | |
| | | | | | VPH_TAD | | | |
| 3 | P96.sTF.Bm.5.23.11 | m3950560 | P(THI13)_long | Bm3R1- | Bm3R1-SV40_NLS_1-Linker1_long- | T(RPS2) | Spacer | 2xbmO-P(AOX1)_core_promoter |
| | | | | based two- | 1xSpyTag002-ERBV_1_P2A- | | | |
| | | | | component | SpyCatcher002-SV40_NLS_2- | | | |
| | | | | | Trimerization_domain-VPR_TAD | | | |

Lab scale fermentation processes (comprising Stage I, Stage II and Stage III) that were used in Assays 1 and 2 and can be used for industrial scale production are described below.

TABLE 27
Lab scale fermentation processes used in Assay 2, including parameters of media
conditions during various phases of Processes 1, 2, 3 and 4. Batch phase (Stage I) is
followed, in order, by fed-batch phase (Stage II), and production phase (Stage III).

| Process | Carbon | Carbon Feed Rate | | | Additions | | |
|---|---|---|---|---|---|---|---|
| Number | source | Batch | Fed-batch | Production | Batch | Fed-batch | Production |
| 1 | Glycerol | N/A | Excess | Limiting | None | None | Formic acid |
| 2 | Glucose | N/A | Excess | Limiting | None | None | Formic acid |
| 3 | Glucose | N/A | Excess | Limiting | Thiamine | None | None |
| 4 | Glycerol | N/A | Limiting | Fixed | Vitamin | None | None |
| | (Batch, Fed- | | | | solution | | |
| | batch) | | | | | | |
| | Methanol | | | | | | |
| | (Production) | | | | | | |

### Lab Scale Fermentation Processes

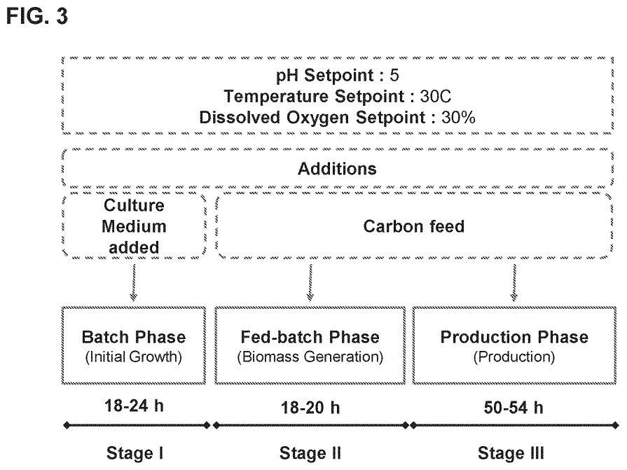

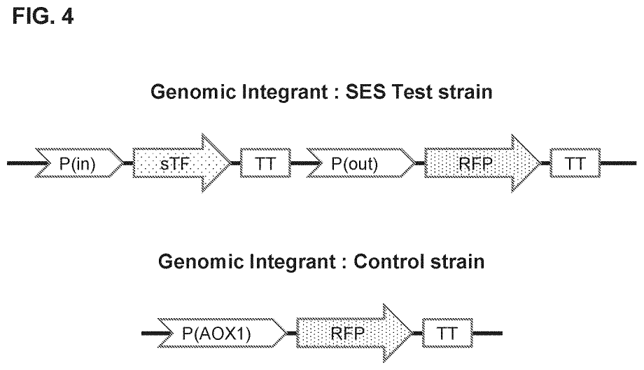

Freshly-grown colonies of the strain(s) of interest were scraped from a solid culture medium plate and used to inoculate an erlenmeyer shake flask with culture medium supplemented with a carbon source corresponding to the process (Table 27). Alternately, the shake flask could be directly inoculated with a thawed glycerol stock of the strain(s). The culture was allowed to grow for 18-20 hours at 30° C., 250 rpm to an optical density (OD) at 600 nm of 20±5. This served as an inoculum for a bioreactor, prefilled with fresh culture medium supplemented with a carbon source and additions as indicated in Table 27. The carbon source was added to a final concentration of 40 g/L. The bioreactor operated continuously while maintaining constant pH, temperature and dissolved oxygen levels (FIG. 3), with no additional carbon being fed during the batch phase. The end of the batch phase was marked by a complete consumption of added carbon and a carbon feed was initiated to mark the fed-batch phase. The rate of carbon feeding was regulated as indicated in Table 27. An excess feed (Processes 1, 2 and 3) is one in which carbon is added to the bioreactor at a rate faster than it can be consumed by the cells. A limiting feed (Process 4) is one in which carbon is added to the bioreactor at a rate equal to or slower than it can be consumed by the cells. The fed-batch phase ended when sufficient biomass was achieved and the production phase was initiated by transitioning to a new carbon source (Process 4) and/or a new carbon feed rate, along with the additions indicated in Table 27. The production phase of Process 3 was additionally characterized by thiamine depletion, during which exogenous/added thiamine was consumed during or diluted by growth to a level that induced expression of Process 3-dependent input promoters (P(in)). Fermentation ended 86 to 96 hours after the start of fermentation.

Composition of Culture medium: Potassium phosphate monobasic, Ammonium sulfate, Calcium sulfate dihydrate, Potassium sulfate, Magnesium sulfate heptahydrate, Copper (II) Sulfate Pentahydrate, Sodium Iodide, Manganese (II) Sulfate Monohydrate, Sodium Molybdate Dihydrate, Boric Acid, Calcium Sulfate Dihydrate, Cobalt (II) Chloride Zinc Chloride, Iron (II) Sulfate Heptahydrate, Biotin and Sulfuric Acid.

Composition of Vitamin solution: Biotin, Calcium pantothenate, Folic Acid, Myo-Inositol, Nicotinic acid, 4-aminobenzoic acid, Pyridoxine hydrochloride, Riboflavin, Thiamine.

TABLE 28
Full DNA sequence (by SEQ ID NO) of SES variants identified by
Assay 2 in Table 23, Table 24, and Table 25.

| Process in | | Full DNA sequence of SES |
|---|---|---|
| which SES | | variant comprising P(in), the |
| variant was | SES variant identifier in | STF coding sequence, |
| identified | Tables 23, 24, and 25 | T(RPS2), Spacer, and P(out) |
| 1 | P96.sTF.Tet.13.102.4 | (SEQ ID NO: 1) |
| 1 | P96.sTF.Van.9.103.4 | (SEQ ID NO: 2) |
| 1 | P96.sTF.Phl.12.99.6 | (SEQ ID NO: 3) |
| 1 | P96.sTF.Tet.1.106.4 | (SEQ ID NO: 4) |
| 1 | P96.sTF.Phl.7.11.7 | (SEQ ID NO: 5) |
| 1 | P96.sTF.Phl.5.107.4 | (SEQ ID NO: 6) |
| 2 | P96.sTF.Phl.5.40.8 | (SEQ ID NO: 7) |
| 2 | P96.sTF.Bm.9.118.8 | (SEQ ID NO: 8) |
| 2 | P96.sTF.Phl.12.25.7 | (SEQ ID NO: 9) |
| 2 | P96.sTF.Phl.5.109.8 | (SEQ ID NO: 10) |
| 2 | P96.sTF.Bm.13.100.7 | (SEQ ID NO: 11) |
| 2 | P96.sTF.Phl.12.17.9 | (SEQ ID NO: 12) |
| 2 | P96.sTF.Phl.9.107.7 | (SEQ ID NO: 13) |
| 3 | P96.sTF.Phl.5.41.10 | (SEQ ID NO: 14) |
| 3 | P96.sTF.Bm.5.23.11 | (SEQ ID NO: 15) |

TABLE 30
DNA coding sequences (by SEQ ID NO) of the STF part types used in
the SES variants identified by Assay 2 in Table 23, Table 24, and Table 25.

| Process in | | |
|---|---|---|
| which SES | | |
| variant was | SES variant identifier in | DNA sequence of sTF |
| identified | Tables 23, 24, and 25 | coding sequence |
| 1 | P96.sTF.Tet.13.102.4 | (SEQ ID NO: 26) |
| 1 | P96.sTF.Van.9.103.4 | (SEQ ID NO: 27) |
| 1 | P96.STF.Phl.12.99.6 | (SEQ ID NO: 28) |
| 1 | P96.sTF.Tet.1.106.4 | (SEQ ID NO: 29) |
| 1 | P96.sTF.Phl.7.11.7 | (SEQ ID NO: 30) |
| 1 | P96.sTF.Phl.5.107.4 | (SEQ ID NO: 31) |
| 2 | P96.sTF.Phl.5.40.8 | (SEQ ID NO: 32) |
| 2 | P96.sTF.Bm.9.118.8 | (SEQ ID NO: 33) |
| 2 | P96.sTF.Phl.12.25.7 | (SEQ ID NO: 34) |
| 2 | P96.sTF.Phl.5.109.8 | (SEQ ID NO: 35) |
| 2 | P96.sTF.Bm.13.100.7 | (SEQ ID NO: 36) |
| 2 | P96.sTF.Phl.12.17.9 | (SEQ ID NO: 37) |
| 2 | P96.sTF.Phl.9.107.7 | (SEQ ID NO: 38) |
| 3 | P96.sTF.Phl.5.41.10 | (SEQ ID NO: 39) |
| 3 | P96.sTF.Bm.5.23.11 | (SEQ ID NO: 40) |

TABLE 31
Amino acid sequences (by SEQ ID NO) of the encoded sTF part
types used in the SES variants identified by Assay 2 in
Table 23, Table 24, and Table 25.

| Process in | | Amino acid sequence |
|---|---|---|
| which SES | | encoded by sTF coding |
| variant was | SES variant identifier in | sequence (coding sequences |
| identified | Tables 23, 24, and 25 | shown in Table 30) |
| 1 | P96.sTF.Tet. 13.102.4 | (SEQ ID NO: 41) |
| 1 | P96.sTF.Van.9.103.4 | (SEQ ID NO: 42) |
| 1 | P96.sTF.Phl.12.99.6 | (SEQ ID NO: 43) |
| 1 | P96.sTF.Tet.1.106.4 | (SEQ ID NO: 44) |
| 1 | P96.sTF.Phl.7.11.7 | (SEQ ID NO: 45) |
| 1 | P96.sTF.Phl.5.107.4 | (SEQ ID NO: 46) |
| 2 | P96.sTF.Phl.5.40.8 | (SEQ ID NO: 47) |
| 2 | P96.sTF.Bm.9.118.8 | (SEQ ID NO: 48) |
| 2 | P96.sTF.Phl.12.25.7 | (SEQ ID NO: 49) |
| 2 | P96.sTF.Phl.5.109.8 | (SEQ ID NO: 50) |
| 2 | P96.sTF.Bm.13.100.7 | (SEQ ID NO: 51) |
| 2 | P96.sTF.Phl.12.17.9 | (SEQ ID NO: 52) |
| 2 | P96.sTF.Phl.9.107.7 | (SEQ ID NO: 53) |
| 3 | P96.sTF.Phl.5.41.10 | (SEQ ID NO: 54) |
| 3 | P96.sTF.Bm.5.23.11 | (SEQ ID NO: 55) |

TABLE 32
Name and DNA sequence (by SEQ ID NO) of the transcriptional terminator
used for the RFP-expressing transcriptional unit. Part types that are
considered optional for SES functionality are labeled ″Optional part type.″

| TT | |
|---|---|
| Optional part type | DNA sequence |
| T(AOX1) | (SEQ ID NO: 147) |

TABLE 33
DNA sequences (by SEQ ID NO) of the output promoter part types used in
SES variants identified by Assay 2 in Table 23, Table 24, and Table 25.

| Process in | | |
|---|---|---|
| which SES | | |
| variant was | SES variant identifier in | DNA sequence of |
| identified | Tables 23, 24, and 25 | output promoter |
| 1 | P96.sTF.Tet.13.102.4 | (SEQ ID NO: 56) |
| 1 | P96.sTF.Van.9.103.4 | (SEQ ID NO: 57) |
| 1 | P96.sTF.Phl.12.99.6 | (SEQ ID NO: 58) |
| 1 | P96.sTF.Tet.1.106.4 | (SEQ ID NO: 59) |
| 1 | P96.sTF.Phl.7.11.7 | (SEQ ID NO: 60) |
| 1 | P96.sTF.Phl.5.107.4 | (SEQ ID NO: 61) |
| 2 | P96.sTF.Phl.5.40.8 | (SEQ ID NO: 62) |
| 2 | P96.sTF.Bm.9.118.8 | (SEQ ID NO: 63) |
| 2 | P96.sTF.Phl.12.25.7 | (SEQ ID NO: 64) |
| 2 | P96.sTF.Phl.5.109.8 | (SEQ ID NO: 65) |
| 2 | P96.sTF.Bm.13.100.7 | (SEQ ID NO: 66) |
| 2 | P96.sTF.Phl.12.17.9 | (SEQ ID NO: 67) |
| 2 | P96.sTF.Phl.9.107.7 | (SEQ ID NO: 68) |
| 3 | P96.sTF.Phl.5.41.10 | (SEQ ID NO: 69) |
| 3 | P96.sTF.Bm.5.23.11 | (SEQ ID NO: 70) |

TABLE 34
DNA sequences (by SEQ ID NO) of the first transcriptional units of the SES
variants identified by Assay 2 in Table 23, Table 24, and Table 25. Each given first
transcriptional unit comprises (i) an input promoter variant, (ii) a coding sequence encoding
a synthetic transcription factor variant, and (iii) an invariant transcriptional terminator
T(RPS2).

| Process in which SES | SES variant identifier in | DNA sequence of the first |
|---|---|---|
| variant was identified | Tables 23, 24, and 25 | transcriptional unit |
| 1 | P96.sTF.Tet.13.102.4 | (SEQ ID NO: 71) |
| 1 | P96.sTF.Van.9.103.4 | (SEQ ID NO: 72) |
| 1 | P96.sTF.Phl.12.99.6 | (SEQ ID NO: 73) |
| 1 | P96.sTF.Tet.1.106.4 | (SEQ ID NO: 74) |
| 1 | P96.sTF.Phl.7.11.7 | (SEQ ID NO: 75) |
| 1 | P96.sTF.Phl.5.107.4 | (SEQ ID NO: 76) |
| 2 | P96.sTF.Phl.5.40.8 | (SEQ ID NO: 77) |
| 2 | P96.sTF.Bm.9.118.8 | (SEQ ID NO: 78) |
| 2 | P96.sTF.Phl.12.25.7 | (SEQ ID NO: 79) |
| 2 | P96.sTF.Phl.5.109.8 | (SEQ ID NO: 80) |
| 2 | P96.sTF.Bm.13.100.7 | (SEQ ID NO: 81) |
| 2 | P96.sTF.Phl.12.17.9 | (SEQ ID NO: 82) |
| 2 | P96.sTF.Phl.9.107.7 | (SEQ ID NO: 83) |
| 3 | P96.sTF.Phl.5.41.10 | (SEQ ID NO: 84) |
| 3 | P96.sTF.Bm.5.23.11 | (SEQ ID NO: 85) |

TABLE 35
DNA sequence (by SEQ ID NO) of the second transcriptional unit of SES variants
identified by Assay 2 in Table 23, Table 24, and Table 25. Each given second transcriptional
unit comprises (i) an output promoter variant, (ii) an invariant coding sequence encoding
RFP, and (iii) an invariant transcriptional terminator T(AOX1).

| Process in which SES | SES variant identifier in | DNA sequence of the |
|---|---|---|
| variant was identified | Tables 23, 24, and 25 | second transcriptional unit |
| 1 | P96.sTF.Tet.13.102.4 | (SEQ ID NO: 167) |
| 1 | P96.sTF.Van.9.103.4 | (SEQ ID NO: 168) |
| 1 | P96.sTF.Phl.12.99.6 | (SEQ ID NO: 169) |
| 1 | P96.sTF.Tet.1.106.4 | (SEQ ID NO: 170) |
| 1 | P96.sTF.Phl.7.11.7 | (SEQ ID NO: 171) |
| 1 | P96.sTF.Phl.5.107.4 | (SEQ ID NO: 172) |
| 2 | P96.sTF.Phl.5.40.8 | (SEQ ID NO: 173) |
| 2 | P96.sTF.Bm.9.118.8 | (SEQ ID NO: 174) |
| 2 | P96.sTF.Phl.12.25.7 | (SEQ ID NO: 175) |
| 2 | P96.sTF.Phl.5.109.8 | (SEQ ID NO: 176) |
| 2 | P96.sTF.Bm.13.100.7 | (SEQ ID NO: 177) |
| 2 | P96.sTF.Phl.12.17.9 | (SEQ ID NO: 178) |
| 2 | P96.sTF.Phl.9.107.7 | (SEQ ID NO: 179) |
| 3 | P96.sTF.Phl.5.41.10 | (SEQ ID NO: 180) |
| 3 | P96.sTF.Bm.5.23.11 | (SEQ ID NO: 181) |

### Example 4. Strain Library Construction with Two-Part SESs Expressing Payload Proteins

A subset of SESs tested in Example 3 were reconstructed as two parts, wherein Part I contained an independent transcription unit expressing the sTF under the transcriptional control of P(in) and Part II contained a transcription unit expressing a payload under the transcriptional control of P(out). Four variants of Part I (Table 36), each with a unique combination of P(in) and sTF, were assembled from parts synthesized for Example 3. Similarly, 72 variants of Part II were assembled, each with 1 of 8 P(out)s and 1 of 9 payloads. The 9 payloads were designed to be secreted, and were a combination of 3 unique secretion tags and 3 proteins of interest, each with a luminescent detection tag, designed to be associated with the protein after secretion (Table 37).

TABLE 36
Combinations of parts and test processes for Examples 3 and 4. Each combination
was tested in combination with nine payloads (Table 37).

| Process | P(in) | STF | P(out) |
|---|---|---|---|
| Process 1 | P(GQ6704499) | VanR_AM-SV40_NLS | 2xVanO- |
| | (SEQ ID NO: 20) | (SEQ ID NO: 182) | P(AOX1)_core_promoter |
| | | | (SEQ ID NO: 186) |
| Process 1 | P(GQ6704499) | VanR_AM-SV40_NLS | 4x VanO- |
| | (SEQ ID NO: 20) | (SEQ ID NO: 182) | P(AOX1)_core_promoter |
| | | | (SEQ ID NO: 187) |
| Process 2 | P(AOX2) | Bm3R1-SV40_NLS- | 1xBmO- |
| | (SEQ ID NO: 24) | Linker1_Long-VPH_TAD | P(AOX1)_core_promoter |
| | | (SEQ ID NO: 183) | (SEQ ID NO: 188) |
| Process 2 | P(AOX2) | Bm3R1-SV40_NLS- | 2xBmO- |
| | (SEQ ID NO: 24) | Linker1_Long-VPH_TAD | P(AOX1)_core_promoter |
| | | (SEQ ID NO: 183) | (SEQ ID NO: 189) |
| Process 2 | P(AOX2) | Bm3R1-SV40_NLS- | 4xBmO- |
| | (SEQ ID NO: 24) | Linker1_Long-VPH_TAD | P(AOX1)_core_promoter |
| | | (SEQ ID NO: 183) | (SEQ ID NO: 190) |
| Process 3 | P(THI13_short) | TetR-SV40_NLS- | 1xTetO- |
| | (SEQ ID NO: 16) | Linker1_Long-VPH_TAD | P(AOX1)_core_promoter |
| | | (SEQ ID NO: 184) | (SEQ ID NO: 191) |
| Process 3 | P(THI13_short) | TetR-SV40_NLS- | 4xTetO- |
| | (SEQ ID NO: 16) | Linker1_Long-VPH_TAD | P(AOX1)_core_promoter |
| | | (SEQ ID NO: 184) | (SEQ ID NO: 192) |
| Process 3 | P(THI13_short) | TetR-SV40_NLS- | 8xTetO- |
| | (SEQ ID NO: 16) | Linker1_Long-VPH_TAD | P(AOX1)_core_promoter |
| | | (SEQ ID NO: 184) | (SEQ ID NO: 193) |
| Process 3 | P(THI13_short) | VanR_AM-SV40_NLS- | 2x VanO- |
| | (SEQ ID NO: 16) | Linker1_Long-B112_TAD | P(AOX1)_core_promoter |
| | | (SEQ ID NO: 185) | (SEQ ID NO: 186) |
| Process 3 | P(THI13_short) | VanR_AM-SV40_NLS- | 4x VanO- |
| | (SEQ ID NO: 16) | Linker1_Long-B112_TAD | P(AOX1)_core_promoter |
| | | (SEQ ID NO: 185) | (SEQ ID NO: 187) |

TABLE 37
Combinations of secretion tag and protein comprising each payload.

| Secretion tag | Protein |
|---|---|
| α-amylase tag | α-amylase |
| α-amylase tag | β-lactoglobulin |
| α-amylase tag | ovalbumin |
| Sc Mf α1 tag | α-amylase |
| Sc Mf α1 tag | β-lactoglobulin |
| Sc Mf al tag | ovalbumin |
| Pre-Inulase tag | α-amylase |
| Pre-Inulase tag | β-lactoglobulin |
| Pre-Inulase tag | ovalbumin |

Each of the Part I integration constructs was used to transform P. pastoris host cells such that each transcription unit was integrated at a pre-defined locus in the genome, at single copy, resulting in four unique strains. The correct integration of Part I in each resulting strain was clonally verified by next generation sequencing (NGS) and strains were cryo-preserved in 25% glycerol. Each base strain was further transformed with a subset of the 72 variants of Part II, such that the P(out) in Part II was matched to its cognate sTF in Part I-containing base strains. The total possible combinations are summarized in Table 36. Part II was designed to integrate at a random genomic location. Not all transformations were successful, and 1 to 3 colonies were chosen from each successful transformation, for a total of 241 strains in the library. Final strains were cryo-preserved in 20% glycerol.

### Example 5. Assay 3: Expression in Deepwell Plates

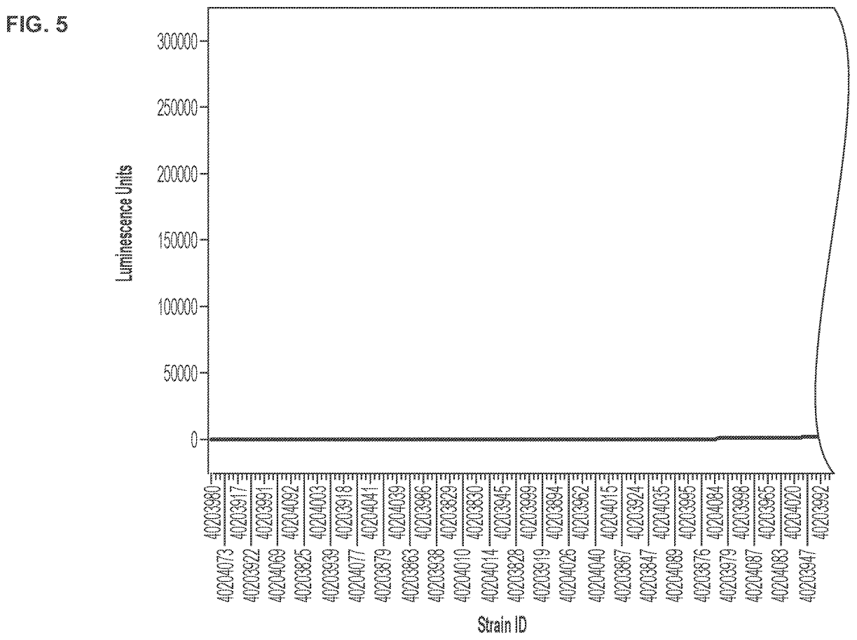

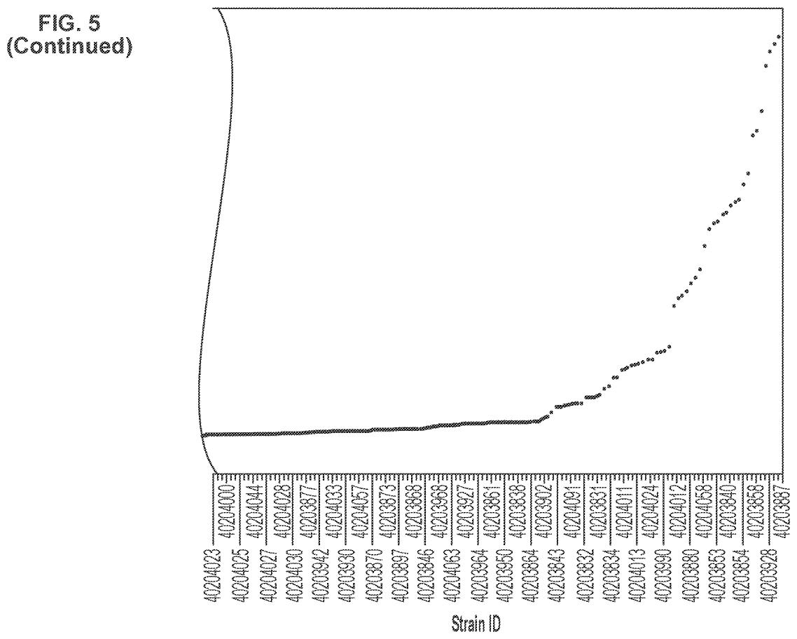

This Example demonstrates effective protein expression in a robustly diverse set of expression systems. The strain library generated in Example 4 was divided according to P(in) and assayed by a deepwell plate process corresponding to that P(in) (Table 36). A glycerol stock of each member of the library of transformed P. pastoris strains was spotted onto a process-specific spotting plate (described below) and allowed to grow at 30° C. for 24 hours. These spots were used to inoculate YEP with 2% glucose and 1% glycerol in a deepwell plate and allowed to grow at 30° C. for 14 hours. These cultures were then subcultured in 250 μl of a process-specific assay medium (described below) and grown at 30° C. for 72 hours. Cell density of the culture was measured in a plate reader using a small aliquot. Extracellular luminescence was measured from cell-free media using a plate reader, while intracellular luminescence was measured from cell lysates obtained by mechanical lysis in a similar manner. Remarkably, several diverse synthetic expression systems were shown to effectively drive bioproduct production (results are shown in FIG. 5).

Composition of deepwell plate culture media: YEP medium contains yeast extract, bacto peptone, and NaCl. Spotting plates for Process 1 contain yeast extract, peptone, yeast nitrogen base (without amino acids), potassium phosphate, biotin and agar with 2% glycerol. Spotting plates for Process 2 and 3 contain YEP with agar and 2% glucose. Assay medium contains Potassium phosphate monobasic, Ammonium sulfate, Calcium sulfate dihydrate, Potassium sulfate, Magnesium sulfate heptahydrate, Copper (II) Sulfate Pentahydrate, Sodium Iodide, Manganese (II) Sulfate Monohydrate, Sodium Molybdate Dihydrate, Boric Acid, Calcium Sulfate Dihydrate, Cobalt (II) Chloride Zinc Chloride, Iron (II) Sulfate Heptahydrate, Biotin and Sulfuric Acid with either 1% glycerol added for Process 1, 1% glucose added for Process 2 or 1% glucose with 100 nM Thiamine added for Process 3. YEP medium contains yeast extract, bacto peptone, and NaCl.

### Example 6. Assay 4: Expression in Lab-Scale Bioreactors

A subset of strains from Example 5 was subjected to a lab scale fermentation using one of the four processes described in Example 3. Samples were drawn 24 h after the start of fermentation and 12 hours thereafter, until 96 hours had elapsed, and were stored at 4° C. The extracellular and intracellular luminescence of each sample was measured as described above and are summarized in Table 38.

TABLE 38
Assay of expression in fermenters. Each column represents one unique strain
producing one of three proteins, in a specific fermentation process. Values represent the
maximum luminescence (Luminescence Units) observed during fermentation, in both the
secreted and intracellular fraction.

| POI | Ovalbumin | | | Amylase | | | beta-Lactoglobulin | | |
|---|---|---|---|---|---|---|---|---|---|
| Process No | 1 | 2 | 3 | 1 | 2 | 3 | 1 | 2 | 3 |
| Secreted | 7160 | 254980 | 220 | 6020 | 13360 | 26440 | 3015110 | 5444640 | 934010 |
| Luminescence | | | | | | | | | |
| Units | | | | | | | | | |
| Intracellular | 239 | 34346 | 29 | 23513 | 3660 | 2444 | 192909 | 256632 | 129865 |
| Luminescence | | | | | | | | | |
| Units | | | | | | | | | |

### Example 7. Assay of Expression of Myoglobin and Heme from Diploids, in a Deepwell Plate

Two DNA libraries were designed and synthesized, the first expressing one or more native heme biosynthetic enzymes (HEM1, HEM2, HEM3, HEM4, HEM12, HEM13, HEM14 and HEM15) under the transcriptional control of one or more SESs and a second containing one or more transcriptional units of myoglobin (MB) expressed under one or more P(out)s. The first DNA library was used to transform P. pastoris host cells such that each strain contained one or more transcription units integrated on the genome expressing eight heme biosynthetic genes and one or more additional P(in)-sTF-Terminator constructs resulting in 24 haploid strains. The second DNA library was used to transform separate P. pastoris host cells such that each strain contained one or more transcription units expressing myoglobin under different P(out)s, resulting in 15 haploid strains. Haploids with cognate parts within the two strain libraries were mated in an arrayed manner, resulting in 127 unique diploid strains.

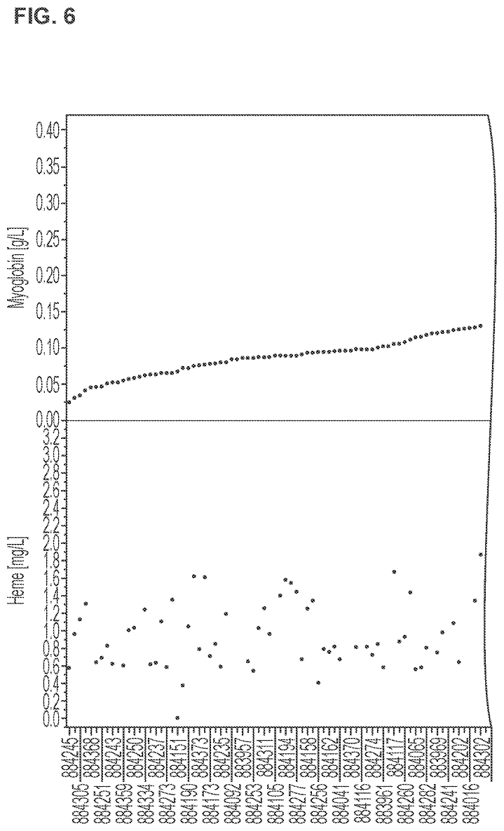

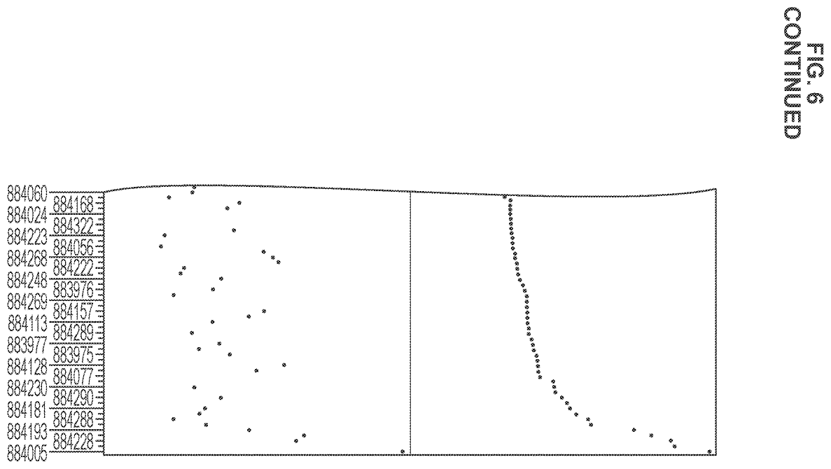

Each member of the library of diploid P. pastoris strains was spotted onto YEP with 2% dextrose and allowed to grow at 30° C. for 24 hours. These spots were used to inoculate YEP with 2% glucose in a deepwell plate and allowed to grow at 30° C. for 19 hours. These cultures were then subcultured in 240 μl of Assay medium supplemented with 2% glucose, 5 g/L monosodium glutamate and 100 nM Thiamine and grown at 30° C. for 24 hours. Cell lysates were obtained by mechanical lysis and concentrations of myoglobin and heme were obtained by size exclusion chromatography by comparing peak areas with known amounts of a commercial standard. Results are summarized in FIG. 6.

### Example 8. Assay of Transcriptional Expression of Myoglobin and Heme Biosynthesis Genes in Haploids, in Fermentation Conditions

One haploid strain expressing all eight heme biosynthetic genes under a particular SES and the myoglobin under a different SES was constructed and then subjected to a lab scale fermentation using Process 3 described in Example 3. Samples were drawn at various timepoints during fermentation and subjected to RNA sequencing. The transcripts were normalized to 1 million total transcripts and are summarized in Table 39 for a selected subset.

TABLE 39
Transcriptional level of nine genes expressed under a SES in one strain,
during two distinct phases of fermentation. Values represent number
of transcripts for each gene, per million total transcripts.

| Gene name/Process phase | Fed-batch phase | Production phase |
|---|---|---|
| HEM1 | 1311 | 3819 |
| HEM2 | 1891 | 5482 |
| HEM3 | 1996 | 6393 |
| HEM4 | 968 | 1909 |
| HEM12 | 2468 | 7514 |
| HEM13 | 2039 | 5659 |
| HEM14 | 1791 | 3934 |
| HEM15 | 2021 | 4918 |
| Myoglobin | 57628 | 187733 |

### EQUIVALENTS

Those skilled in the art will recognize, or be able to ascertain using no more than routine experimentation, many equivalents to the specific embodiments of the invention described in this application. Such equivalents are intended to be encompassed by the following claims. The definitions provided in any one section of this application are intended to apply to any other section, where applicable.

1. A methylotrophic host cell comprising a synthetic expression system that comprises:
(a) a first transcriptional unit comprising:
(i) an input promoter comprising an upstream activating sequence (UAS) and a core promoter element, and
(ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
wherein the input promoter drives expression of the at least one component of the synthetic transcription factor; and
(b) a second transcriptional unit comprising a synthetic output promoter operably linked to a gene of interest, wherein the synthetic transcription factor is an activator of the synthetic output promoter, and
wherein the gene of interest is expressed in the absence of exogenously provided methanol.

2. The methylotrophic host cell of claim 1, wherein the polynucleotide of the first transcriptional unit encodes all components of the synthetic transcription factor.

3. The methylotrophic host cell of claim 1, wherein the input promoter is synthetic.

4. The methylotrophic host cell of claim 3, wherein the input promoter has at least 90% sequence identity to a naturally occurring promoter.

5. The methylotrophic host cell of claim 1, wherein the input promoter is naturally occurring.

6. The methylotrophic host cell of claim 1, wherein the input promoter is native to the cell.

7. The methylotrophic host cell of claim 1, wherein the input promoter is a regulatable input promoter.

8. The methylotrophic host cell of claim 7, wherein the regulatable input promoter is inducible.

9. The methylotrophic host cell of claim 7, wherein the regulatable input promoter is repressible.

10. The methylotrophic host cell of claim 7, wherein the regulatable input promoter is responsive to nutrient addition, limitation, or depletion with respect to a cognate cultivation process.

11. The methylotrophic host cell of claim 10, wherein the regulatable input promoter is responsive to thiamine depletion, glycerol limitation, monosaccharide limitation, or to the limitation of a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate.

12. The methylotrophic host cell of claim 10, wherein the regulatable input promoter is responsive to the limitation or depletion of a combination of any two or more nutrients.

13. The methylotrophic host cell of claim 10, wherein activity of the regulatable input promoter is increased by the presence of exogenously provided formic acid.

14. The methylotrophic host cell of claim 7, wherein the regulatable input promoter is regulatable in the absence of exogenously provided methanol.

15. The methylotrophic host cell of claim 1, wherein the input promoter is not methanol inducible.

16. The methylotrophic host cell of claim 1, wherein the input promoter is a constitutive input promoter.

17. The methylotrophic host cell of claim 1, wherein the upstream activating sequence (UAS) and/or the core promoter element of the input promoter is not native to the methylotrophic host cell.

18. The methylotrophic host cell of claim 1, wherein the input promoter is P(JEN1), P(GQ6704499), P(GQ6700926), P(HGT1), P(FDH1), P(AOX2), P(RGI2), P(THI13)_short, P(THI13)_long, or P(THI4).

19. The methylotrophic host cell of claim 1, wherein the input promoter is polynucleotide having at least 90%, at least 95%, or at least 99% identity to a nucleic acid sequence of any one of SEQ ID NOs: 16-25.

20. The methylotrophic host cell of claim 1, wherein the input promoter is a polynucleotide having the nucleic acid sequence of any one of SEQ ID NOs: 16-25.

21. The methylotrophic host cell of claim 1, wherein the DNA binding domain (DBD) of the synthetic transcription factor is Bm3R1, TetR, PhlF_AM, or VanR_AM.

22. The methylotrophic host cell of claim 1, wherein the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD.

23. The methylotrophic host cell of claim 1, wherein the DNA-binding domain (DBD) of the synthetic transcription factor is Bm3R1, TetR, PhlF_AM, or VanR_AM, and the transcriptional activation domain (TAD) of the synthetic transcription factor is B112_TAD, B42_TAD, GAL4_TAD, miniVPR_TAD, Mxr1_TAD, PH_TAD, VP16_TAD, VP64_TAD, VP64v2_TAD, VPH_TAD, or VPR_TAD.

24. The methylotrophic host cell of claim 1, wherein the synthetic transcription factor is not an activator of the input promoter.

25. The methylotrophic host cell of claim 1, wherein the synthetic transcription factor is a one-component synthetic transcription factor.

26. The methylotrophic host cell of claim 1, wherein the synthetic transcription factor is a two-component or multi-component synthetic transcription factor.

27. The methylotrophic host cell of claim 26, wherein the two-component or multi-component synthetic transcription factor comprises at least two bioconjugate protein products.

28. The methylotrophic host cell of claim 27, wherein a first bioconjugate protein product (BPP1) is SpyTag002 and a second bioconjugate protein product (BPP2) is SpyCatcher002.

29. The methylotrophic host cell of claim 1, wherein the synthetic transcription factor comprises a nuclear localization signal (NLS).

30. The methylotrophic host cell of claim 29, wherein the nuclear localization signal is an SV40 nuclear localization signal.

31. The methylotrophic host cell of claim 1, wherein the synthetic transcription factor comprises a linker.

32. The methylotrophic host cell of claim 1, wherein the synthetic transcription factor comprises a self-cleaving polypeptide.

33. The methylotrophic host cell of claim 32, wherein the self-cleaving polypeptide is a 2A peptide.

34. The methylotrophic host cell of claim 32, wherein the self-cleaving polypeptide is ERBV_1_P2A.

35. The methylotrophic host cell of claim 1, wherein the synthetic transcription factor comprises an oligomerization domain.

36. The methylotrophic host cell of claim 35, wherein the oligomerization domain is Linker_only_for_oligomerization, Trimerization_domain, or Heptamerization_domain.

37. The methylotrophic host cell of claim 1, wherein the synthetic transcription factor comprises a polypeptide having the amino acid sequence of any one of SEQ ID NOs: 41-55.

38. The methylotrophic host cell of claim 1, wherein the first transcriptional unit comprises a polynucleotide having the nucleic acid sequence of any one of SEQ ID NOs: 26-40 or 182-185.

39. The methylotrophic host cell of claim 1, wherein the synthetic output promoter is not methanol inducible.

40. The methylotrophic host cell of claim 1, wherein the synthetic output promoter comprises an upstream activating sequence (UAS) and a core promoter element.

41. The methylotrophic host cell of claim 40, wherein the upstream activating sequence (UAS) of the synthetic output promoter is not native to the methylotrophic host cell.

42. The methylotrophic host cell of claim 40, wherein the core promoter element of the synthetic output promoter has a nucleic acid sequence that is no more than 300 base pairs in length.

43. The methylotrophic host cell of claim 40, wherein the core promoter element of the synthetic output promoter has a nucleic acid sequence that is from about 6 base pairs to about 300 base pairs, from about 25 base pairs to about 250 base pairs, from about 75 to about 225 base pairs, or from about 100 base pairs to about 175 base pairs in length.

44. The methylotrophic host cell of claim 40, wherein the distance between the 3′ end of the upstream activating sequence (UAS) and 5′ end of the core promoter element of the synthetic output promoter is from 0 to about 200 base pairs in length.

45. The methylotrophic host cell of claim 40, wherein the distance between the upstream activating sequence (UAS) and core promoter element of the synthetic output promoter is a nucleic acid sequence having from about 6 base pairs to about 200 base pairs, from about 6 base pairs to about 53 base pairs, from about 20 base pairs to about 150 base pairs, from about 50 base pairs to about 125 base pairs, or from about 50 base pairs to about 100 base pairs in length.

46. The methylotrophic host cell of claim 40, wherein the core promoter element of the synthetic output promoter comprises a core promoter sequence that is at least 90%, at least 95%, or 100% identical to a naturally occurring core promoter sequence.

47. The methylotrophic host cell of claim 40, wherein the core promoter element of the synthetic output promoter comprises a core promoter sequence that is at least 90%, at least 95%, or 100% identical to a core promoter sequence from P(AOX1), P(DAS2), P(HHF2), or P(PMP20).

48. The methylotrophic host cell of claim 40, wherein the upstream activating sequence (UAS) of the synthetic output promoter is bmO, tetO, phlO, or vanO.

49. The methylotrophic host cell of claim 40, wherein the synthetic output promoter further comprises one or more operators.

50. The methylotrophic host cell of claim 49, wherein the one or more operators of the synthetic output promoter are not native to the methylotrophic host cell.

51. The methylotrophic host cell of claim 40, wherein the synthetic transcription factor comprises the DNA-binding domain (DBD) Bm3R1, and the upstream activating sequence (UAS) of the synthetic output promoter comprises one or more copies of bmO.

52. The methylotrophic host cell of claim 40, wherein the synthetic transcription factor comprises the DNA-binding domain (DBD) PhlF_AM and the upstream activating sequence (UAS) of the synthetic output promoter comprises one or more copies of phlO.

53. The methylotrophic host cell of claim 40, wherein the synthetic transcription factor comprises the DNA-binding domain (DBD) TetR and the upstream activating sequence (UAS) of the synthetic output promoter comprises one or more copies of tetO.

54. The methylotrophic host cell of claim 40, wherein the synthetic transcription factor comprises the DNA-binding domain (DBD) VanR_AM and the upstream activating sequence (UAS) of the synthetic output promoter comprises one or more copies of vanO.

55. The methylotrophic host cell of claim 1, wherein the synthetic output promoter comprises a polynucleotide having the nucleic acid sequence of any one of SEQ ID NOs: 56-70 or 186-193.

56. The methylotrophic host cell of claim 1, wherein the gene of interest is expressed as an RNA.

57. The methylotrophic host cell of claim 1, wherein the gene of interest encodes a protein.

58. The methylotrophic host cell of claim 57, wherein the gene of interest encodes an enzyme, a structural protein, a signaling protein, a regulatory protein, a transport protein, a sensory protein, a motor protein, a defense protein, or a storage protein.

59. The methylotrophic host cell of claim 57, wherein the protein synthesizes, modifies, or converts a molecule.

60. The methylotrophic host cell of claim 59, wherein the molecule is heme or an intermediate in a heme biosynthesis pathway.

61. The methylotrophic host cell of claim 57, wherein the protein is a heme-binding protein.

62. The methylotrophic host cell of claim 61, wherein the heme-binding protein is hemoglobin, neuroglobin, cytoglobin, leghemoglobin, or myoglobin.

63. The methylotrophic host cell of claim 57, wherein the protein is vaccinia capping enzyme, T7 polymerase, or O-methyltransferase.

64. The methylotrophic host cell of claim 57, wherein the protein is an enzyme of a heme biosynthesis pathway.

65. The methylotrophic host cell of claim 64, wherein the enzyme of a heme biosynthesis pathway is cytochrome P450, 9-adenylate cyclase, soluble guanylate cyclase, peroxidase, catalase, and/or cytochrome oxidase.

66. The methylotrophic host cell of claim 1, further comprising in the second transcriptional unit a polynucleotide encoding a secretion tag.

67. The methylotrophic host cell of claim 66, wherein the secretion tag is an α-amylase secretion tag, an Sc Mf α1 secretion tag, or a pre-inulinase secretion tag.

68. The methylotrophic host cell of claim 66, wherein the gene of interest encodes a protein, and wherein the protein is secreted from the methylotrophic host cell.

69. The methylotrophic host cell of claim 68, wherein the secreted protein is an α-amylase, a β-lactoglobulin, or an ovalbumin.

70. The methylotrophic host cell of claim 1, wherein the first transcriptional unit and/or the second transcriptional unit further comprises a transcriptional terminator.

71. The methylotrophic host cell of claim 70, wherein the transcriptional terminator of the first and/or second transcriptional unit is naturally occurring.

72. The methylotrophic host cell of claim 70, wherein the transcriptional terminator of the first and/or second transcriptional unit is synthetic.

73. The methylotrophic host cell of claim 70, wherein the transcriptional terminator of the first and/or second transcriptional unit is from a gene encoding a ribosomal protein.

74. The methylotrophic host cell of claim 73, wherein the gene encodes the ribosomal protein S2 (RPS2).

75. The methylotrophic host cell of claim 73, wherein the transcriptional terminator comprises a polynucleotide having the nucleic acid sequence of either SEQ ID NO: 146 or SEQ ID NO: 147.

76. The methylotrophic host cell of claim 1, wherein the first transcriptional unit and the second transcriptional unit are separated by a spacer.

77. The methylotrophic host cell of claim 1, wherein the first and/or second transcriptional unit is present in multiple copies.

78. The methylotrophic host cell of claim 77, wherein the copy number ratio of the first transcriptional unit to the second transcriptional unit is 1:1.

79. The methylotrophic host cell of claim 77, wherein the copy number ratio of the first transcriptional unit to the second transcriptional unit is at least 2:1, at least 4:1, or at least 10:1.

80. The methylotrophic host cell of claim 77, wherein the copy number ratio of the second transcriptional unit to the first transcriptional unit is at least 2:1, at least 4:1, or at least 10:1.

81. The methylotrophic host cell of claim 77, wherein the first transcriptional unit is present in a single copy and the second transcriptional unit is present in multiple copies.

82. The methylotrophic host cell of claim 81, wherein at least two of the multiple second transcriptional units comprise different genes of interest.

83. The methylotrophic host cell of claim 81, wherein the synthetic transcription factor of the first transcriptional unit is an activator of each synthetic output promoter of the multiple second transcriptional units.

84. The methylotrophic host cell of claim 1, wherein the synthetic expression system comprises one or more sequences that are endogenous to the methylotrophic host cell.

85. The methylotrophic host cell of claim 1, wherein the first and second transcriptional units are located on a single plasmid.

86. The methylotrophic host cell of claim 1, wherein the first and second transcriptional units are located on different plasmids.

87. The methylotrophic host cell of claim 1, wherein the first and/or second transcriptional units are integrated into the genome of the methylotrophic host cell.

88. The methylotrophic host cell of claim 87, wherein the first and second transcriptional units are located on the same chromosome in the methylotrophic host cell genome.

89. The methylotrophic host cell of claim 1, wherein the first and second transcriptional units are oriented in the same direction.

90. The methylotrophic host cell of claim 1, wherein the first and second transcriptional units are oriented in different directions.

91. The methylotrophic host cell of claim 1, wherein the first and second transcriptional units are located on different chromosomes in the methylotrophic host cell genome.

92. The methylotrophic host cell of claim 1, wherein the methylotrophic host cell is a methylotrophic yeast cell.

93. The methylotrophic host cell of claim 1, wherein the methylotrophic host cell is from a genus selected from: Pichia, Komagataella, Hansenula, or Candida.

94. The methylotrophic host cell of claim 93, wherein the methylotrophic host cell is Pichia pastoris, Pichia pseudopastoris, Komagataella phaffii, Pichia stipitis, Pichia membranifaciens, Komagataella pseudopastoris, Komagataella pastoris, Komagataella kurtzmanii, Komagataella mondaviorum, Hansenula polymorpha, Candida boidinii, or Pichia methanolica.

95. The methylotrophic host cell of claim 93, wherein the methylotrophic host cell is Pichia pastoris.

96. The methylotrophic host cell of claim 1, wherein the synthetic expression system provides for production of a bioproduct encoded by the gene of interest at a level that is higher than the level of the bioproduct produced in a control host cell.

97. The methylotrophic host cell of claim 96, wherein the control host cell is of the same species as the methylotrophic host cell.

98. The methylotrophic host cell of claim 97, wherein the control host cell comprises a methanol-inducible promoter operably linked to a gene of interest.

99. The methylotrophic host cell of claim 98, wherein the gene of interest encoded by the control host cell is the same gene of interest encoded by the methylotrophic host cell.

100. The methylotrophic host cell of claim 98, wherein the methanol-inducible promoter is P(AOX1) of P. pastoris.

101. The methylotrophic host cell of claim 100, wherein the control cell is cultured in the presence of exogenously-added methanol.

102. The methylotrophic host cell of claim 1, wherein the methylotrophic host cell is cultured under conditions comprising a growth phase and a production phase.

103. The methylotrophic host cell of claim 102, wherein the quantity of transcripts of the gene of interest produced in the methylotrophic host cell in the production phase is at least 100% higher than the quantity of transcripts of the gene of interest produced in the methylotrophic host cell in the growth phase.

104. The methylotrophic host cell of claim 102, wherein the quantity of transcripts of the gene of interest produced in the methylotrophic host cell in the production phase is at least 200%, at least 300%, at least 400%, or at least 500% higher than the quantity of transcripts of the gene of interest produced in the methylotrophic host cell in the growth phase.

105. The methylotrophic host cell of claim 1, wherein the synthetic expression system provides for production of a bioproduct encoded by the gene of interest at a level that is at least 200% higher than the level of the bioproduct produced in a control host cell.

106. The methylotrophic host cell of claim 105, wherein the synthetic expression system provides for production of a bioproduct encoded by the gene of interest at a level that is at least 600%, at least 900%, at least 1200%, at least 1500%, at least 1800%, at least 2100%, at least 2400%, at least 2700%, or at least 3000% higher than the level of the bioproduct produced in a control host cell.

107. The methylotrophic host cell of claim 105, wherein the synthetic expression system provides for production of a bioproduct encoded by the gene of interest at a level that is from about 300% to about 600%, from about 500% to about 1000%, from about 800% to about 1500%, from about 1000% to about 2000%, from about 1200% to about 2000%, from about 1800% to about 2500%, from about 2000% to about 2500%, or from about 2200% to about 3000% higher than the level of the bioproduct produced in a control host cell.

108. A methylotrophic host cell comprising a synthetic expression system that comprises:
(a) a first transcriptional unit comprising:
(i) an input promoter comprising an upstream activating sequence (UAS) and a core promoter element, and
(ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter,
wherein the gene of interest is expressed in the absence of exogenously provided methanol,
wherein methylotrophic host cell is cultured under conditions comprising a growth phase and a production phase, and
wherein the quantity of transcripts of the gene of interest produced by methylotrophic host cell in the production phase is at least 100% higher than in the growth phase.

109. A methylotrophic host cell comprising a synthetic expression system that comprises:
(a) a first transcriptional unit comprising:
(i) an input promoter comprising an upstream activating sequence (UAS) and a core promoter element, and
(ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter, and a polynucleotide encoding a secretion tag;
wherein the gene of interest is expressed in the absence of exogenously provided methanol.

110. A methylotrophic host cell comprising a synthetic expression system that comprises:
(a) a first transcriptional unit comprising:
(i) an input promoter comprising an upstream activating sequence (UAS) and a core promoter element, and
(ii) a polynucleotide encoding at least one component of a synthetic transcription factor, wherein the synthetic transcription factor comprises a DNA binding domain (DBD) and a transcriptional activation domain (TAD), wherein the DBD and TAD are not native to the methylotrophic host cell,
wherein the input promoter drives expression of the at least one component of the synthetic transcription factor, and
(b) a second transcriptional unit comprising a synthetic output promoter operably linked to a gene of interest, wherein the synthetic transcription factor is an activator of the synthetic output promoter,
wherein the gene of interest is expressed in the absence of exogenously provided methanol, and
wherein the synthetic expression system provides for production of a bioproduct encoded by the gene of interest at a level that is at least 300% higher than the level of the bioproduct produced in a control host cell.

111. A method of expressing a gene of interest comprising culturing the methylotrophic host cell according to any one of claims 1-110.

112. The method of claim 111, wherein the gene of interest encodes a heme-binding protein or one or more enzymes of a heme biosynthesis pathway.

113. The method of claim 112, wherein the heme-binding protein is hemoglobin, myoglobin, neuroglobin, cytoglobin, or leghemoglobin.

114. The method of claim 112, wherein the one or more enzymes of a heme biosynthesis pathway is cytochrome P450, 9-adenylate cyclase, soluble guanylate cyclase, peroxidase, catalase, and/or cytochrome oxidase.

115. The method of claim 111, wherein the gene of interest encodes a vaccinia capping enzyme, T7 polymerase enzyme, or O-methyltransferase enzyme.

116. A method of manufacturing a molecule of interest comprising culturing the methylotrophic host cell according to any one of claims 1-110 and obtaining the molecule of interest from biomass or culture.

117. The method of claim 116, wherein the obtaining comprises extracting the molecule of interest from biomass.

118. The method of claim 116, wherein the obtaining comprises collecting the molecule from culture, culture medium, cell-free spent culture medium, and/or cell-containing culture medium.

119. A method of producing a molecule of interest comprising expressing a gene of interest according to any one of claims 111-115, wherein the gene of interest encodes an enzyme, the method comprising:
(a) purifying the enzyme encoded by the gene of interest; and
(b) using the purified enzyme for bioconversion of a substrate to the molecule of interest.

120. The method of any one of claims 116-119, wherein the molecule of interest is heme.

121. A method of expressing a gene of interest or producing a molecule of interest comprising steps of:
(a) culturing a methylotrophic host cell according to any one of claims 1-110 in a suitable medium for a period of time to allow cell growth, and
(b) changing one or more culture conditions to facilitate expression of the gene of interest or production of the molecule of interest.

122. The method of claim 121, wherein changing one or more culture conditions comprises changing the composition of the culture medium.

123. The method of claim 121 or claim 122, wherein step (b) comprises limiting, adding, and/or depleting a nutrient.

124. The method of any one of claims 121-123, wherein step (b) comprises thiamine depletion, glycerol limitation, monosaccharide limitation, or formic acid addition.

125. The method of any one of claims 121-124, wherein step (b) comprises limitation of any a carbon source, a sugar, a starch, galactose, maltose, glucose, sorbitol, inositol, glycerol, a vitamin, a steroid, a nitrogen source, nitrate, nitrite, ammonium, an amino acid, methionine, a heavy metal, copper, benzoic acid, hydrogen peroxide, a calcium-containing compound, and/or phosphate.

126. The method of any one of claims 121-125, wherein step (b) comprises the limitation of a combination of any two nutrients.

127. The method of claims 121-125, wherein step (b) comprises limitation of glucose and depletion of thiamine.

128. A synthetic expression system comprising a polynucleotide having at least 90%, at least 95%, or at least 99% identity to the nucleic acid sequence of any one of SEQ ID NOs: 1-15.

129. The synthetic expression of claim 128, wherein the synthetic expression system comprises an input promoter comprising a polynucleotide having at least 90%, at least 95%, or at least 99% identity to the nucleic acid sequence of any one of SEQ ID NOs: 16-25.

130. The synthetic expression of claim 128 or claim 129, wherein the synthetic expression system comprises a polynucleotide encoding at least one component of a synthetic transcription factor.

131. The synthetic expression system of claim 130, wherein the polynucleotide encoding at least one component of a synthetic transcription factor comprises a polynucleotide having at least 90%, at least 95%, or at least 99% identity to the nucleic acid sequence of any one of SEQ ID NOs: 26-40 or 182-185.

132. The synthetic expression system of claim 131, wherein the encoded synthetic transcription factor comprises a polypeptide having at least 90%, at least 95%, or at least 99% identity to the amino acid sequence of any one of SEQ ID NOs: 41-55.

133. The synthetic expression of any one of claims 128-132, wherein the synthetic expression system comprises a synthetic output promoter comprising a polynucleotide having at least 90%, at least 95%, or at least 99% identity to the nucleic acid sequence of any one of SEQ ID NOs: 56-70 or 186-193.

134. The methylotrophic host cell according to any one of claims 1-110 comprising a polynucleotide having at least 90%, at least 95%, or at least 99% identity to the nucleic acid sequence of any one of SEQ ID NOs: 16-25.

135. The methylotrophic host cell according to any one of claims 1-110 comprising a polynucleotide having at least 90%, at least 95%, or at least 99% identity to the nucleic acid sequence of any one of SEQ ID NOs: 56-70 or 186-193.

136. The methylotrophic host cell according to any one of claims 1-110, wherein the synthetic transcription factor is encoded by a polynucleotide having at least 90%, at least 95%, or at least 99% identity to the nucleic acid sequence of any one of SEQ ID NOs: 26-40 or 182-185.

137. The methylotrophic host cell according to any one of claims 1-110, wherein the synthetic transcription factor comprises a polypeptide having at least 90%, at least 95%, or at least 99% identity to the amino acid sequence of any one of SEQ ID NOs: 41-55.

138. A method of engineering a host cell for protein expression comprising:
transforming the host cell with the synthetic expression system according to any one of claims 128-133.
